SEQUENCE LISTING (2 sequences)

SEQ ID NO: 1 - DNA, 3634 bp, Homo sapiens
  source 1..3634: mol_type = unassigned DNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2024/0410016 A1
(43) Pub. Date: Dec. 12, 2024

(54) CD274 REARRANGEMENTS AS PREDICTORS OF RESPONSE TO IMMUNE CHECKPOINT INHIBITOR THERAPY

(71) Applicant: Foundation Medicine, Inc., Boston, MA (US)

(72) Inventors: Karthikeyan MURUGESAN, Boston, MA (US); Andrew David KELLY, Philadelphia, PA (US); Zheng KUANG, Boston, MA (US); Richard Sheng Poe HUANG, Cary, NC (US)

(73) Assignee: Foundation Medicine, Inc., Boston, MA (US)

(21) Appl. No.: 18/699,569

(22) PCT Filed: Oct. 11, 2022
(86) PCT No.: PCT/US2022/077927

§ 371 Date: Apr. 8, 2024

Related U.S. Application Data
(60) Provisional application No. 63/254,965, filed on Oct. 12, 2021.

(51) Int. Cl. C12Q 1/6886

(52) U.S. Cl. CPC C12Q 1/6886; C12Q 2600/106; C12Q 2600/156

(57) ABSTRACT

Provided herein are CD274 rearrangements. CD274 nucleic acid molecules, and PD-L1 polypeptides methods related to detecting CD274 rearrangements. CD274 nucleic acid molecules, and PD-L1 polypeptides in cancer, as well as methods of treatment and uses related thereto. Detection of CD274 rearrangements. CD274 nucleic acid molecules, and PD-L1 polypeptides of the disclosure can be used to identify individuals that may benefit from treatment with an anti-cancer therapy such as an immune checkpoint inhibitor.

### CROSS-REFERENCE TO RELATED APPLICATIONS

This application claims the benefit of U.S. Provisional Application No. 63/254,965, filed Oct. 12, 2021, which is hereby incorporated by reference in its entirety.

### REFERENCE TO AN ELECTRONIC SEQUENCE LISTING

The contents of the electronic sequence listing (197102007440seqlist.xml; Size: 6,706 bytes; and Date of Creation: Sep. 28, 2022) is herein incorporated by reference in its entirety.

### TECHNICAL FIELD

Provided herein are CD274 rearrangements, CD274 nucleic acid molecules, and PD-L1 polypeptides, methods related to detecting such CD274 rearrangements, CD274 nucleic acid molecules, and PD-L1 polypeptides, as well as methods of diagnosis/treatment and uses related thereto.

### BACKGROUND

Immune checkpoint inhibitor (ICI) therapy can benefit individuals having multiple different cancer types. PD-L1 (programmed cell death-ligand 1) is a transmembrane receptor that regulates adaptive immune responses, and overexpression of PD-L1 has been postulated as a mechanism of immune evasion by cancer cells. Detection of PD-L1 protein expression by immunohistochemistry (IHC) is commonly used as a companion diagnostic for ICI treatment in multiple tumor types (Topalian, S. L. et al. (2012) N. Engl. J.).

However, not all patients identified as PD-L1-positive by IHC testing respond to ICIs, while some patients who are PD-L1-negative respond to ICIs (Grossman, J. E., et al. (2021) Oncogene 40:1393-1395). For example, the PD-L1 pharmDx DAKO 22C3 assay is a U.S. Food and Drug Administration (FDA)-approved companion diagnostic for prescribing pembrolizumab for gastric cancer patients with a PD-L1 combined positive score (CPS) ≥1 (Fuchs, C. S. et al. (2018) JAMA Oncol. 4: e180013), but in a phase III trial, pembrolizumab failed to provide clinically meaningful value as a second-line therapy in advanced gastric cancer patients with a PD-L1 CPS score ≥1 (Shitara, K. et al. (2018) Lancet 392:123-133).

Attempts to standardize PD-L1 IHC testing have had little success due to variability of the different PD-L1 IHC assays and scoring methodologies (U.S. Food & Drug Administration. List of Cleared or Approved Companion Diagnostic Devices [In Vitro and Imaging Tools]. Accessible at www.fda.gov/medical-devices/vitro-diagnostics/list-cleared-or-approved-companion-diagnostic-devices-vitro-and-imaging-tools). Current PD-L1 IHC testing techniques also require a solid tumor specimen biopsy or a surgically-resected specimen, which can result in inefficient detection of PD-L1 expression due to tumor tissue inaccessibility, poor availability of archival tissue, low quality and quantity of the tumor sample, long sample processing times, and high cost. Other factors have also contributed to the imperfect prediction of response to ICIs using PD-L1 expression as a biomarker, including the spatial heterogeneity and temporal dynamics of PD-L1 protein expression, technical difficulties specific to certain tumor types (e.g., melanin pigmentation in melanoma samples), the use of continuous versus categorical scoring, the age of the archival biopsy used to evaluate PD-L1 status, the scoring concordance amongst pathologists, the complexity of tumor cells, the tumor microenvironment, and host characteristics. As such, PD-L1 IHC testing is complex and currently remains insufficient to consistently predict response to ICI (Remon, J., Besse, B. & Soria, J. C. (2017) BMC Medicine; Garon, E. B. (2017) N. Engl. J. Med.; Huang, R. S. P. et al. (2020) Mod. Pathol).

Accordingly, there is a need in the art for improved predictive biomarkers of response to ICI therapy in cancer to guide the treatment of cancer patients.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In one aspect, provided herein is a method of identifying an individual having a cancer who may benefit from a treatment comprising an immune checkpoint inhibitor, the method comprising detecting in a sample from the individual a cluster of differentiation 274 (CD274) nucleic acid molecule, or a programmed death-ligand 1 (PD-L1) polypeptide encoded by the CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; wherein detection of the CD274 nucleic acid molecule, or the PD-L1 polypeptide encoded by the CD274 nucleic acid molecule, in the sample identifies the individual as one who may benefit from a treatment comprising an immune checkpoint inhibitor.

In another aspect, provided herein is a method of selecting a treatment for an individual having a cancer, the method comprising detecting in a sample from the individual a CD274 nucleic acid molecule, or a PD-L1 polypeptide encoded by the CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; wherein detection of the CD274 nucleic acid molecule, or the PD-L1 polypeptide encoded by the CD274 nucleic acid molecule, in the sample identifies the individual as one who may benefit from a treatment comprising an immune checkpoint inhibitor.

In another aspect, provided herein is a method of identifying one or more treatment options for an individual having a cancer, the method comprising: detecting in a sample from the individual a CD274 nucleic acid molecule, or a PD-L1 polypeptide encoded by the CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and generating a report comprising one or more treatment options identified for the individual based at least in part on detection of the CD274 nucleic acid molecule, or the PD-L1 polypeptide encoded by the CD274 nucleic acid molecule, wherein the one or more treatment options comprise an immune checkpoint inhibitor.

In another aspect, provided herein is a method of identifying one or more treatment options for an individual having a cancer, the method comprising: acquiring knowledge of a CD274 nucleic acid molecule, or a PD-L1 polypeptide encoded by the CD274 nucleic acid molecule, in a sample from the individual, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and generating a report comprising one or more treatment options identified for the individual based at least in part on said knowledge, wherein the one or more treatment options comprise an immune checkpoint inhibitor.

In another aspect, provided herein is a method of selecting a treatment for an individual having cancer, comprising: acquiring knowledge of a CD274 nucleic acid molecule, or a PD-L1 polypeptide encoded by the CD274 nucleic acid molecule, in a sample from the individual, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; wherein responsive to the acquisition of said knowledge: (i) the individual is classified as a candidate to receive a treatment comprising an immune checkpoint inhibitor; and/or (ii) the individual is identified as likely to respond to a treatment that comprises an immune checkpoint inhibitor.

In another aspect, provided herein is a method of predicting survival of an individual having a cancer, comprising: acquiring knowledge of a CD274 nucleic acid molecule, or a PD-L1 polypeptide encoded by the CD274 nucleic acid molecule, in a sample from the individual, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; wherein responsive to the acquisition of said knowledge, the individual is predicted to have longer survival when treated with a treatment comprising an immune checkpoint inhibitor, as compared to survival of an individual whose cancer does not comprise the CD274 nucleic acid molecule, or the PD-L1 polypeptide encoded by the CD274 nucleic acid molecule.

In another aspect, provided herein is a method of predicting survival of an individual having a cancer treated with a treatment comprising an immune checkpoint inhibitor, the method comprising: acquiring knowledge of a CD274 nucleic acid molecule, or a PD-L1 polypeptide encoded by the CD274 nucleic acid molecule, in a sample from the individual, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; wherein responsive to the acquisition of said knowledge, the individual is predicted to have longer survival when treated with a treatment comprising an immune checkpoint inhibitor, as compared to an individual whose cancer does not exhibit the CD274 nucleic acid molecule, or the PD-L1 polypeptide encoded by the CD274 nucleic acid molecule.

In another aspect, provided herein is a method of treating or delaying progression of cancer, comprising: acquiring knowledge of a CD274 nucleic acid molecule, or a PD-L1 polypeptide encoded by the CD274 nucleic acid molecule, in a sample from an individual having a cancer, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and responsive to said knowledge, administering to the individual an effective amount of a treatment that comprises an immune checkpoint inhibitor.

In another aspect, provided herein is a method of treating or delaying progression of cancer, comprising administering to an individual having a cancer an effective amount of a treatment that comprises an immune checkpoint inhibitor, wherein the treatment is administered responsive to acquiring knowledge of a CD274 nucleic acid molecule, or a PD-L1 polypeptide encoded by the CD274 nucleic acid molecule, in a sample from the individual, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3.

In another aspect, provided herein is a method of monitoring, evaluating or screening an individual having a cancer, comprising: acquiring knowledge of a CD274 nucleic acid molecule, or a PD-L1 polypeptide encoded by the CD274 nucleic acid molecule, in a sample from the individual, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; wherein responsive to the acquisition of said knowledge, the individual is predicted to have an improved response to treatment with an immune checkpoint inhibitor as compared to an individual whose cancer does not comprise the CD274 nucleic acid molecule, or the PD-L1 polypeptide encoded by the CD274 nucleic acid molecule.

In another aspect, provided herein is a method of assessing a CD274 nucleic acid molecule or a PD-L1 polypeptide in a cancer in an individual, the method comprising: detecting in a sample from the individual a CD274 nucleic acid molecule, or a PD-L1 polypeptide encoded by the CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and providing an assessment of the CD274 nucleic acid molecule, or the PD-L1 polypeptide encoded by the CD274 nucleic acid molecule.

In another aspect, provided herein is a method of detecting a CD274 nucleic acid molecule or a PD-L1 polypeptide, the method comprising detecting in a sample from an individual having a cancer a CD274 nucleic acid molecule, or a PD-L1 polypeptide encoded by the CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3.

In another aspect, provided herein is a method of detecting the presence or absence of a cancer in an individual, the method comprising: detecting the presence or absence of a cancer in a sample from the individual; and detecting, in a sample from the individual, the presence or absence of a CD274 nucleic acid molecule, or a PD-L1 polypeptide encoded by the CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3. In some embodiments, the method comprises detecting the presence of the cancer in the sample. In some embodiments, the method comprises detecting the presence of the CD274 nucleic acid molecule, or the PD-L1 polypeptide encoded by the CD274 nucleic acid molecule, in the sample from the individual.

In another aspect, provided herein is a method for monitoring progression or recurrence of a cancer in an individual, the method comprising: detecting, in a first sample obtained from the individual at a first time point, the presence or absence of a CD274 nucleic acid molecule, or a PD-L1 polypeptide encoded by the CD274 nucleic acid molecule; detecting, in a second sample obtained from the individual at a second time point after the first time point, the presence or absence of a CD274 nucleic acid molecule, or a PD-L1 polypeptide encoded by the CD274 nucleic acid molecule; and providing an assessment of cancer progression or cancer recurrence in the individual based, at least in part, on the presence or absence of the CD274 nucleic acid molecule, or the PD-L1 polypeptide encoded by the CD274 nucleic acid molecule, in the first sample and/or in the second sample; wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3. In some embodiments, the presence of the CD274 nucleic acid molecule, or the PD-L1 polypeptide encoded by the CD274 nucleic acid molecule, in the first sample and/or in the second sample identifies the individual as having decreased risk of cancer progression or cancer recurrence when treated with a treatment comprising an immune checkpoint inhibitor. In some embodiments, the method further comprises selecting a treatment, administering a treatment, adjusting a treatment, adjusting a dose of a treatment, or applying a treatment to the individual based, at least in part, on detecting the presence of the CD274 nucleic acid molecule, or the PD-L1 polypeptide encoded by the CD274 nucleic acid molecule, in the first sample and/or in the second sample, wherein the treatment comprises an immune checkpoint inhibitor.

In another aspect, provided herein is a method of detecting a CD274 nucleic acid molecule, the method comprising: providing a plurality of nucleic acid molecules obtained from a sample from an individual having a cancer, wherein the plurality of nucleic acid molecules comprises nucleic acid molecules corresponding to a CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; optionally, ligating one or more adapters onto one or more nucleic acid molecules from the plurality of nucleic acid molecules; optionally, amplifying the one or more ligated nucleic acid molecules from the plurality of nucleic acid molecules; optionally, capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequence reads correspond to the CD274 nucleic acid molecule; analyzing the plurality of sequence reads; and based on the analysis, detecting the presence or absence of the CD274 nucleic acid molecule in the sample. In some embodiments, the method further comprises receiving, at one or more processors, sequence read data for the plurality of sequence reads. In some embodiments, analyzing the plurality of sequence reads comprises identifying, using the one or more processors, the presence or absence of sequence reads corresponding to the CD274 nucleic acid molecule. In some embodiments, the amplified nucleic acid molecules are captured by hybridization with one or more bait molecules.

In another aspect, provided herein is a method of detecting a CD274 nucleic acid molecule, the method comprising: providing a sample from an individual having a cancer, wherein the sample comprises a plurality of nucleic acid molecules; preparing a nucleic acid sequencing library from the plurality of nucleic acid molecules in the sample; amplifying said library; selectively enriching for one or more nucleic acid molecules in said library that comprise nucleotide sequences corresponding to a CD274 nucleic acid molecule to produce an enriched sample, wherein: (a) the CD274 nucleic acid molecule comprises or results from a rearrangement comprising a Breakpoint 1 and/or

Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; sequencing the enriched sample, thereby producing a plurality of sequence reads; analyzing the plurality of sequence reads for the presence of the CD274 nucleic acid molecule; detecting, based on the analyzing step, the presence or absence of the CD274 nucleic acid molecule in the sample from the individual.

In some embodiments, which may be combined with any of the preceding aspects or embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample. In some embodiments, the one or more adapters comprise amplification primers, flow cell adapter sequences, substrate adapter sequences, or sample index sequences. In some embodiments, the selectively enriching comprises: (a) combining one or more bait molecules with the library, thereby hybridizing the one or more bait molecules to one or more nucleic acid molecules comprising nucleotide sequences corresponding to the CD274 nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer. In some embodiments, the method further comprises generating a genomic profile for the individual, based, at least in part, on detecting the presence or absence of the CD274 nucleic acid molecule. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the genomic profile for the individual further comprises the level of tumor mutational burden (TMB), clonal TMB, indel TMB, or nonsense-mediated decay (NMD)-escape TMB; the presence or absence of, or the proportion of mutations fitting, a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof. In some embodiments, the method further comprises selecting a treatment, administering a treatment, or applying a treatment to the individual based on the generated genomic profile, wherein the treatment comprises an immune checkpoint inhibitor. In some embodiments, the method further comprises generating a report indicating the presence or absence of the CD274 nucleic acid molecule in the sample. In some embodiments, the method further comprises generating, by the one or more processors, a report indicating the presence or absence of the CD274 nucleic acid molecule in the sample. In some embodiments, the method further comprises transmitting the report to a healthcare provider. In some embodiments, the report is transmitted via a computer network or a peer-to-peer connection.

In another aspect, provided herein is a method of identifying a candidate treatment for a cancer in an individual in need thereof, comprising performing DNA sequencing on a sample obtained from the individual to determine a sequencing mutation profile on a CD274 gene, wherein the sequencing mutation profile identifies the presence or absence of a CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and wherein the candidate treatment comprises an immune checkpoint inhibitor. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the CD274 nucleic acid molecule comprising a breakpoint.

In another aspect, provided herein is a method of treating or delaying progression of cancer, comprising: detecting in a sample from an individual having a cancer a CD274 nucleic acid molecule, or a PD-L1 polypeptide encoded by the CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and administering to the individual an effective amount of a treatment that comprises an immune checkpoint inhibitor.

In some embodiments, which may be combined with any of the preceding aspects or embodiments, the CD274 nucleic acid molecule comprises or results from a rearrangement comprising a Breakpoint 1 and/or Breakpoint 2 within the chromosomal coordinates as listed in Table 1; or the CD274 fusion nucleic acid molecule comprises a fusion between a CD274 gene, or a portion thereof, and a fusion partner gene as listed in Table 2, or a portion thereof; and wherein the cancer is a carcinoma, a sarcoma, a lymphoma, a leukemia, a myeloma, a germ cell cancer, or a blastoma.

In some embodiments, which may be combined with any of the preceding aspects or embodiments, the CD274 nucleic acid molecule comprises or results from a rearrangement comprising a Breakpoint 1 and/or Breakpoint 2 within the chromosomal coordinates as listed in Table 1; or the CD274 fusion nucleic acid molecule comprises a fusion between a CD274 gene, or a portion thereof, and a fusion partner gene as listed in Table 2, or a portion thereof; and wherein the cancer is a solid tumor.

In some embodiments, which may be combined with any of the preceding aspects or embodiments, the CD274 nucleic acid molecule comprises or results from a rearrangement comprising a Breakpoint 1 and/or Breakpoint 2 within the chromosomal coordinates as listed in Table 1; or the CD274 fusion nucleic acid molecule comprises a fusion between a CD274 gene, or a portion thereof, and a fusion partner gene as listed in Table 2, or a portion thereof; and wherein the cancer is a hematologic malignancy.

In some embodiments, which may be combined with any of the preceding aspects or embodiments, the CD274 nucleic acid molecule comprises or results from a rearrangement comprising a Breakpoint 1 and/or Breakpoint 2 within the chromosomal coordinates as listed in Table 1; or the CD274 fusion nucleic acid molecule comprises a fusion between a CD274 gene, or a portion thereof, and a fusion partner gene as listed in Table 2, or a portion thereof; and wherein the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, which may be combined with any of the preceding aspects or embodiments, the CD274 nucleic acid molecule comprises or results from a rearrangement comprising a Breakpoint 1 and/or Breakpoint 2 within the chromosomal coordinates as listed in Table 1; or the CD274 fusion nucleic acid molecule comprises a fusion between a CD274 gene, or a portion thereof, and a fusion partner gene as listed in Table 2, or a portion thereof; and wherein the cancer is an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, unknown primary adenocarcinoma, gastric cancer or carcinoma, unknown primary carcinoma (CUP), unknown primary carcinoma (CUP) (NOS), unknown primary melanoma, stomach adenocarcinoma, or vagina squamous cell carcinoma (SCC).

In some embodiments, which may be combined with any of the preceding aspects or embodiments, the CD274 nucleic acid molecule comprises or results from a rearrangement comprising a Breakpoint 1 and/or Breakpoint 2 within the chromosomal coordinates as listed in Table 1, and wherein the cancer is the corresponding cancer as listed in Table 6; or the CD274 fusion nucleic acid molecule comprises a fusion between a CD274 gene, or a portion thereof, and a fusion partner gene as listed in Table 2, or a portion thereof, and wherein the cancer is the corresponding cancer as listed in Table 7.

In some embodiments, which may be combined with any of the preceding aspects or embodiments, the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4.

In some embodiments, which may be combined with any of the preceding aspects or embodiments, the CD274 fusion nucleic acid molecule comprises a fusion between a CD274 gene, or a portion thereof, and a fusion partner gene as listed in Table 2, or a portion thereof; wherein the CD274 fusion nucleic acid molecule comprises or results from a corresponding CD274 Breakpoint and/or Fusion Partner Gene Breakpoint within the chromosomal coordinates as listed in Table 8; and wherein the cancer is the corresponding cancer as listed in Table 8.

In some embodiments, which may be combined with any of the preceding aspects or embodiments, the CD274 fusion nucleic acid molecule comprises a fusion between a CD274 gene, or a portion thereof, and a fusion partner gene as listed in Table 3, or a portion thereof, and the cancer is the corresponding cancer as listed in Table 3; and wherein the CD274 fusion nucleic acid molecule comprises or results from a corresponding CD274 Breakpoint and/or Fusion Partner Gene Breakpoint within the chromosomal coordinates as listed in Table 5.

In some embodiments, which may be combined with any of the preceding aspects or embodiments, the cancer is metastatic.

In some embodiments, which may be combined with any of the preceding aspects or embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, or any combination thereof. In some embodiments, the immune checkpoint inhibitor is a PD-1-, or a PD-L1-targeted agent. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab. In some embodiments, the immune checkpoint inhibitor is a monotherapy. In some embodiments, the nucleic acid comprises a double-stranded RNA (dsRNA), a small interfering RNA (siRNA), or a small hairpin RNA (shRNA). In some embodiments, the treatment or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the treatment or the one or more treatment options comprise an immune checkpoint inhibitor in combination with one or more chemotherapeutic agents. In some embodiments, the one or more chemotherapeutic agents comprise a platinum-based chemotherapeutic agent. In some embodiments, the treatment or the one or more treatment options comprise atezolizumab and one or more chemotherapeutic agents. In some embodiments, the treatment or the one or more treatment options comprise atezolizumab, bevacizumab-Awwb or bevacizumab, carboplatin, and paclitaxel. In some embodiments, the treatment or the one or more treatment options comprise atezolizumab and paclitaxel or paclitaxel protein-bound. In some embodiments, the treatment or the one or more treatment options comprise nivolumab or pembrolizumab monotherapy. In some embodiments, the treatment or the one or more treatment options comprise pembrolizumab and one or more chemotherapeutic agents. In some embodiments, the treatment or the one or more treatment options comprise pembrolizumab, carboplatin and pemetrexed.

In some embodiments, which may be combined with any of the preceding aspects or embodiments, the encoded PD-L1 polypeptide is oncogenic. In some embodiments, the encoded PD-L1 polypeptide promotes cancer cell survival, angiogenesis, cancer cell proliferation, and any combination thereof.

In some embodiments, which may be combined with any of the preceding aspects or embodiments, the method further comprising acquiring knowledge of or detecting in a sample from the individual the presence or absence of a CD274 gene amplification. In some embodiments, which may be combined with any of the preceding aspects or embodiments, the cancer comprises a CD274 gene amplification. In some embodiments, which may be combined with any of the preceding aspects or embodiments, the cancer does not comprise a CD274 gene amplification.

In some embodiments, which may be combined with any of the preceding aspects or embodiments, the method further comprises acquiring knowledge of or detecting in a sample from the individual the presence or absence of a base substitution, a short insertion/deletion (indel), a copy number alteration, or a genomic rearrangement in one or more genes. In some embodiments, which may be combined with any of the preceding aspects or embodiments, the cancer comprises a base substitution, a small insertion/deletion (indel), a copy number alteration, or a genomic rearrangement in one or more genes. In some embodiments, the one or more genes comprise one or more of TP53, PIK3CA, CDKN2A, KRAS, CDKN2B, CD274, MYC, JAK2, RB1, PDCD1LG2, APC, ARID1A, PTEN, BRAF, CREBBP, PBRM1, KMT2D, CCND1, KDM6A, BCL2L1, ERBB2, FBXW7, NF1, BCORL1, BRCA2, FGF19, FGFR1, MAP2K1, PRKC1, ATM, CDK12, CTNNB1, DNMT3A, FGF3, FGF4, GNAS, LYN, MET, NOTCH1, RNF43, STK11, TET2, VHL, ZNF217, ASXL1, BRCA1, EGFR, KDM5C, KIT, NFE2L2, NOTCH2, NOTCH3, PIK3R1, SOX9, TERC, ZNF703, MTAP, BRIP1, CDC73, ACVR1B, ATRX, MLH1, BRD4, SMAD4, PALB2, RAD21, GATA6, CTCF, or any combination thereof.

In some embodiments, which may be combined with any of the preceding aspects or embodiments, the cancer comprises: (a) an S1601fs*4 frameshift mutation in an ATM gene, and/or a mutation in an ATM gene that results in a V2951F amino acid substitution in an encoded ATM polypeptide; (b) a PTEN gene mutation that results in a Y155C amino acid substitution in an encoded PTEN polypeptide, and/or a PTEN splice site mutation of 634+G>A; (c) a mutation in an RB1 gene that results in a K8* amino acid substitution in an encoded RB1 polypeptide, and/or a deletion of an RB1 gene, or of a portion thereof; (d) a mutation in a MAP2K1 gene that results in an F53C amino acid substitution in an encoded MAP2K1 polypeptide; (c) an amplification of an FGF19, FGF4, CCND1, FGF3, CDC274, or RAD21 gene, or any combination thereof; (f) a deletion of an MTAP gene, or of a portion thereof; (g) a K703fs*3 frameshift mutation in a BRIP1 gene; (h) an H77fs*53 frameshift mutation in a KMT2D gene, and/or a mutation in a KMT2D gene that results in a Q4284* and/or S2834* amino acid substitution in an encoded KMT2D polypeptide; (i) a mutation in an FBXW7 gene that results in a G437R and/or Q242* amino acid substitution in an encoded FBXW7 polypeptide; (j) a CDC73 rearrangement, and/or an M1fs*56 frameshift mutation in a CDC73 gene; (k) a K215fs*19 frameshift mutation in an ACVR1B gene; (l) a D1850fs*33 frameshift mutation in an ARID1A gene; (m) an R840fs*29 frameshift mutation in an ATRX gene; (n) a P798fs*97 frameshift mutation in a BRD4 gene; (o) a mutation in a SMAD4 gene resulting in an R515* amino acid substitution in an encoded SMAD4 polypeptide; (p) a K654fs*47 frameshift mutation in a BRCA1 gene; (q) a mutation in a CTNNB1 gene resulting in a G34R amino acid substitution in an encoded CTNNB1 polypeptide; (r) an M723fs*21 frameshift mutation in a PALB2 gene; (s) a T576fs*4 frameshift mutation in a PIK3R1 gene, and/or a deletion of a PIK3R1 gene, or a portion thereof; (t) a mutation in a GATA6 gene resulting in an E579K amino acid substitution in an encoded GATA6 polypeptide; (u) a mutation in a DNMT3A gene resulting in an R882H amino acid substitution in an encoded DNMT3A polypeptide; (v) an E363fs*5 frameshift mutation in a CTCF gene; or any combination of (a)-(v). In some embodiments, the CDC73 rearrangement results in a CDC73 gene fusion comprising exons 1-7 of CDC73 fused to exons 11-17 of CDC73; optionally wherein the CDC73 gene fusion comprises or results from a breakpoint in exon 7 of CDC73 and/or a breakpoint in intron 10 of CDC73. In some embodiments, the one or more genes comprise TP53, PIK3CA, CDKN2A, KRAS, CDKN2B, MYC, or any combination thereof.

In some embodiments, which may be combined with any of the preceding aspects or embodiments, the cancer comprises: (a) an R290fs*55 and/or H296fs*10 frameshift mutation in a TP53 gene; a mutation in a TP53 gene resulting in a G266V, E285K, C176Y, and/or P278S amino acid substitution in an encoded TP53 polypeptide; or a TP53 splice site mutation of 672+1G>T, or any combination thereof; (b) a CDKN2B deletion; (c) a MYC gene amplification; or any combination of (a)-(c).

In some embodiments, which may be combined with any of the preceding aspects or embodiments, the cancer comprises a deletion of a CDKN2A gene, or of a portion thereof.

In some embodiments, which may be combined with any of the preceding aspects or embodiments, the one or more genes comprise PIK3CA, JAK2, PDCD1LG2, CREBBP, PBRM1, or any combination thereof. In some embodiments, which may be combined with any of the preceding aspects or embodiments, the cancer comprises a mutation in a PIK3CA gene that results in an E545K and/or E542K amino acid substitution, and/or an E110del deletion, in an encoded PIK3CA polypeptide.

In some embodiments, which may be combined with any of the preceding aspects or embodiments, the cancer comprises: (a) an I279fs*4 frameshift mutation in a PBRM1 gene; (b) a PDCD1LG2 gene amplification; (c) a JAK2 gene amplification; or any combination of (a)-(c).

In some embodiments, which may be combined with any of the preceding aspects or embodiments, the one or more genes comprise a mismatch repair gene. In some embodiments, the mismatch repair gene is MLH1. In some embodiments, which may be combined with any of the preceding aspects or embodiments, the cancer comprises a mutation in an MLH1 gene resulting in a Y684* amino acid substitution in an encoded MLH1 polypeptide.

In some embodiments, which may be combined with any of the preceding aspects or embodiments, the method further comprises acquiring knowledge of or detecting in a sample from the individual the presence or absence of a genomic Epstein-Barr virus (EBV). In some embodiments, which may be combined with any of the preceding aspects or embodiments, the cancer or the individual comprises a genomic EBV. In some embodiments, which may be combined with any of the preceding aspects or embodiments, the cancer or the individual is positive for EBV. In some embodiments, the EBV is HHV-4. In some embodiments, the cancer is a gastric cancer. In some embodiments, the gastric cancer is an adenocarcinoma. In some embodiments, the gastric cancer is a stomach adenocarcinoma. In some embodiments, the gastric cancer is a Stage IV cancer.

In some embodiments, which may be combined with any of the preceding aspects or embodiments, the treatment or the one or more treatment options comprise pembrolizumab. In some embodiments, the pembrolizumab is pembrolizumab monotherapy. In some embodiments, which may be combined with any of the preceding aspects or embodiments, the treatment or the one or more treatment options are a second line treatment for cancer.

In some embodiments, which may be combined with any of the preceding aspects or embodiments, the method further comprises acquiring knowledge of or detecting a microsatellite instability status of the cancer in a sample from the individual. In some embodiments, which may be combined with any of the preceding aspects or embodiments, the cancer is microsatellite stable.

In some embodiments, which may be combined with any of the preceding aspects or embodiments, the method further comprises acquiring knowledge of or determining tumor mutational burden (TMB) in a sample from the individual. In some embodiments, which may be combined with any of the preceding aspects or embodiments, the cancer has a TMB of less than 6 mutations per megabase (mut/Mb), between 6 and 20 mut/Mb, or greater than 20 mut/Mb. In some embodiments, which may be combined with any of the preceding aspects or embodiments, the cancer has a high TMB. In some embodiments, which may be combined with any of the preceding aspects or embodiments, the cancer has a TMB of at least about 10 mut/Mb. In some embodiments, which may be combined with any of the preceding aspects or embodiments, the cancer has a TMB of about 7.0 mut/Mb. In some embodiments, TMB is assessed based on between about 0.83 Mb and about 1.14 Mb of sequenced DNA. In some embodiments, TMB is assessed based on about 0.79 Mb, about 0.80 Mb, or about 1.1 Mb of sequenced DNA. In some embodiments, TMB is assessed based on between about 0.8 Mb and about 1.1 Mb of sequenced DNA. In some embodiments, TMB is assessed based on up to about 1.1 Mb or up to about 1.24 Mb of sequenced DNA. In some embodiments, TMB is determined by sequencing, whole exome sequencing, whole genome sequencing, gene-targeted sequencing, or next-generation sequencing. In some embodiments, the cancer is a non-small cell lung carcinoma, a colorectal carcinoma, a carcinoma of unknown primary (CUP), or a gastric carcinoma.

In some embodiments, which may be combined with any of the preceding aspects or embodiments, the method further comprises acquiring knowledge of or determining the level of PD-L1 expression in a sample from the individual. In some embodiments, which may be combined with any of the preceding aspects or embodiments, the cancer is PD-L1 positive. In some embodiments, which may be combined with any of the preceding aspects or embodiments, the cancer is PD-L1-high positive. In some embodiments, which may be combined with any of the preceding aspects or embodiments, the cancer is PD-L1 negative. In some embodiments, PD-L1 expression is determined using an immunohistochemistry assay in a sample obtained from the individual.

In some embodiments, PD-L1 expression is assessed based on a tumor proportion score (TPS). In some embodiments, the cancer is PD-L1 positive. In some embodiments, the cancer has a TPS of at least about 1%. In some embodiments, the cancer has a TPS of between about 1% and about 49%. In some embodiments, the cancer is PD-L1 high positive. In some embodiments, the cancer has a TPS of at least about 50%. In some embodiments, the cancer has a TPS of at least about 1%, at least about 25%, at least about 50%, or at least about 75%. In some embodiments, the cancer has a TPS of at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or 100%. In some embodiments, the cancer is PD-L1 negative. In some embodiments, the cancer has a TPS of less than 1%. In some embodiments, the immunohistochemistry assay is a DAKO PD-L1 22C3 assay.

In some embodiments, PD-L1 expression is assessed based on a combined positive score (CPS). In some embodiments, the cancer has high PD-L1 expression. In some embodiments, the cancer has a CPS of at least about 1 or at least about 10. In some embodiments, the immunohistochemistry assay is a DAKO PD-L1 22C3 assay.

In some embodiments, PD-L1 expression is assessed based on the proportion of tumor area occupied by PD-L1 expressing tumor-infiltrating immune cells of any intensity (IC), or the percentage of PD-L1 expressing tumor cells of any intensity (TC). In some embodiments, the cancer has high PD-L1 expression. In some embodiments, the cancer has a TC of at least about 50%. In some embodiments, the cancer has an IC of at least about 10%. In some embodiments, the cancer has an IC of at least about 1% or at least about 5%. In some embodiments, the immunohistochemistry assay is a VENTANA SP 142 assay.

In some embodiments, which may be combined with any of the preceding aspects or embodiments, the method further comprises acquiring knowledge of or determining the clonality of the CD274 nucleic acid molecule in the cancer. In some embodiments, which may be combined with any of the preceding aspects or embodiments, the CD274 nucleic acid molecule results from a clonal or a sub-clonal rearrangement of a CD274 gene in the cancer. In some embodiments, the CD274 nucleic acid molecule results from a clonal rearrangement of a CD274 gene in the cancer. In some embodiments, which may be combined with any of the preceding aspects or embodiments, the CD274 nucleic acid molecule is clonal or sub-clonal in the cancer. In some embodiments, the CD274 nucleic acid molecule is clonal in the cancer. In some embodiments, acquiring knowledge of or determining that the CD274 nucleic acid molecule is clonal in the cancer or results from a clonal rearrangement of a CD274 gene in the cancer identifies the individual as likely to respond to a treatment comprising an immune checkpoint inhibitor. In some embodiments, responsive to acquiring knowledge of or determining that the CD274 nucleic acid molecule is clonal in the cancer or results from a clonal rearrangement of a CD274 gene in the cancer, the individual is predicted to have longer survival when treated with a treatment comprising an immune checkpoint inhibitor, as compared to survival of: (a) an individual whose cancer does not comprise a clonal CD274 nucleic acid molecule, or a CD274 nucleic acid molecule resulting from a clonal rearrangement of a CD274 gene; or (b) an individual whose cancer comprises a sub-clonal CD274 nucleic acid molecule, or a CD274 nucleic acid molecule resulting from a sub-clonal rearrangement of a CD274 gene. In some embodiments, responsive to acquiring knowledge of or determining that the CD274 nucleic acid molecule is clonal in the cancer or results from a clonal rearrangement of a CD274 gene in the cancer, the individual is predicted to have an improved response to treatment with an immune checkpoint inhibitor, as compared to: (a) an individual whose cancer does not comprise a clonal CD274 nucleic acid molecule, or a CD274 nucleic acid molecule resulting from a clonal rearrangement of a CD274 gene; or (b) an individual whose cancer comprises a sub-clonal CD274 nucleic acid molecule, or a CD274 nucleic acid molecule resulting from a sub-clonal rearrangement of a CD274 gene. In some embodiments, acquiring knowledge of or determining that the CD274 nucleic acid molecule is clonal in the cancer or results from a clonal rearrangement of a CD274 gene in the cancer identifies the cancer as likely to be PD-L1 positive or PD-L1 high positive. In some embodiments, acquiring knowledge of or determining that the CD274 nucleic acid molecule is clonal in the cancer or results from a clonal rearrangement of a CD274 gene in the cancer identifies the cancer as likely to have a TPS of at least about 50%, assessed based on an immunohistochemistry assay. In some embodiments, the immunohistochemistry assay is a DAKO PD-L1 22C3 assay. In some embodiments, clonality of the CD274 nucleic acid molecule is assessed by performing DNA sequencing on a sample obtained from the individual. In some embodiments, clonality of the CD274 nucleic acid molecule is assessed based on sequencing coverage of a CD274 gene and the number of sequence read pairs spanning a breakpoint of the CD274 nucleic acid molecule. In some embodiments, clonality of the CD274 nucleic acid molecule is assessed based on sequencing coverage of a CD274 gene and a threshold for the number of sequence read pairs spanning a breakpoint of the CD274 nucleic acid molecule, wherein the threshold is selected using a receiver operator characteristic (ROC) curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis. In some embodiments, clonality of the CD274 nucleic acid molecule is assessed based on the number of sequence read pairs spanning a breakpoint of the CD274 nucleic acid molecule. In some embodiments, clonality of the CD274 nucleic acid molecule is assessed based on a threshold for the number of sequence read pairs spanning a breakpoint of the CD274 nucleic acid molecule, wherein the threshold is selected using a receiver operator characteristic (ROC) curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis. In some embodiments, a sample from a tumor is PD-L1 high positive if it comprises a tumor proportion score (TPS) of at least about 50%. In some embodiments, the threshold for the number of sequence read pairs spanning a breakpoint of the CD274 nucleic acid molecule is at least about 20 read pairs, at least about 21 read pairs, at least about 22 read pairs, at least about 23 read pairs, at least about 24 read pairs, at least about 25 read pairs, at least about 26 read pairs, at least about 27 read pairs, at least about 28 read pairs, at least about 29 read pairs, at least about 30 read pairs, at least about 31 read pairs, at least about 32 read pairs, at least about 33 read pairs, at least about 34 read pairs, at least about 35 read pairs, at least about 36 read pairs, at least about 37 read pairs, at least about 38 read pairs, at least about 39 read pairs, at least about 40 read pairs, at least about 41 read pairs, at least about 42 read pairs, at least about 43 read pairs, at least about 44 read pairs, at least about 45 read pairs, at least about 46 read pairs, at least about 47 read pairs, at least about 48 read pairs, at least about 49 read pairs, or at least about 50 read pairs spanning a breakpoint of the CD274 nucleic acid molecule. In some embodiments, the threshold for the number of sequence read pairs spanning a breakpoint of the CD274 nucleic acid molecule is at least about 25 read pairs spanning a breakpoint of the CD274 nucleic acid molecule. In some embodiments, the sample is a bulk tumor sample derived from a single anatomic location.

In some embodiments, which may be combined with any of the preceding aspects or embodiments, the method further comprises obtaining the sample from the individual. In some embodiments, which may be combined with any of the preceding aspects or embodiments, the sample is obtained from the cancer. In some embodiments, which may be combined with any of the preceding aspects or embodiments, the sample comprises a tissue biopsy sample, a liquid biopsy sample, or a normal control. In some embodiments, which may be combined with any of the preceding aspects or embodiments, the sample is from a tumor biopsy, tumor specimen, or circulating tumor cell. In some embodiments, which may be combined with any of the preceding aspects or embodiments, the sample is a liquid biopsy sample and comprises blood, plasma, cerebrospinal fluid, sputum, stool, urine, or saliva. In some embodiments, which may be combined with any of the preceding aspects or embodiments, the sample comprises cells and/or nucleic acids from the cancer. In some embodiments, which may be combined with any of the preceding aspects or embodiments, the sample comprises mRNA, DNA, circulating tumor DNA (ctDNA), cell-free DNA, or cell-free RNA from the cancer. In some embodiments, which may be combined with any of the preceding aspects or embodiments, the sample is a liquid biopsy sample and comprises circulating tumor cells (CTCs). In some embodiments, which may be combined with any of the preceding aspects or embodiments, the sample is a liquid biopsy sample and comprises cell-free DNA (cfDNA), circulating tumor DNA (ctDNA), or any combination thereof. In some embodiments, which may be combined with any of the preceding aspects or embodiments, the method comprises acquiring knowledge of or detecting the CD274 nucleic acid molecule, or the PD-L1 polypeptide encoded by the CD274 nucleic acid molecule, in a tissue biopsy sample, in a liquid biopsy sample, or in both a tissue biopsy sample and a liquid biopsy sample, from the individual.

In some embodiments, which may be combined with any of the preceding aspects or embodiments, the acquiring knowledge comprises detecting the CD274 nucleic acid molecule, or the PD-L1 polypeptide encoded by the CD274 nucleic acid molecule, in the sample. In some embodiments, which may be combined with any of the preceding aspects or embodiments, the detecting comprises detecting a fragment of the CD274 nucleic acid molecule comprising a breakpoint. In some embodiments, which may be combined with any of the preceding aspects or embodiments, the CD274 nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS). In some embodiments, the sequencing comprises RNA-sequencing (RNA-seq). In some embodiments, the amplification-based assay comprises a polymerase chain reaction (PCR) amplification technique, a non-PCR amplification technique, or an isothermal amplification technique. In some embodiments, the amplification-based assay comprises a reverse transcription PCR (RT-PCR), a quantitative real-time PCR (qPCR), or a reverse transcription quantitative real-time PCR (RT-qPCR) assay. In some embodiments, the amplification-based assay comprises an RT-PCR assay.

In some embodiments, which may be combined with any of the preceding aspects or embodiments, detecting the PD-L1 polypeptide encoded by the CD274 nucleic acid molecule comprises detecting a portion of the polypeptide that is encoded by a fragment of the CD274 nucleic acid molecule that comprises a breakpoint. In some embodiments, which may be combined with any of the preceding aspects or embodiments, the PD-L1 polypeptide encoded by the CD274 nucleic acid molecule is detected in the sample by one or more of: immunoblotting, enzyme linked immunosorbent assay (ELISA), immunohistochemistry, or mass spectrometry.

In some embodiments, which may be combined with any of the preceding aspects or embodiments, the CD274 nucleic acid molecule, or the PD-L1 polypeptide encoded by the CD274 nucleic acid molecule, is detected using a digital pathology method.

In some embodiments, which may be combined with any of the preceding aspects or embodiments, the method further comprises selectively enriching for one or more nucleic acid molecules in the sample comprising nucleotide sequences corresponding to the CD274 nucleic acid molecule; wherein the selectively enriching produces an enriched sample. In some embodiments, the selectively enriching comprises: (a) combining one or more bait molecules with the sample, thereby hybridizing the one or more bait molecules to one or more nucleic acid molecules in the sample comprising nucleotide sequences corresponding to the CD274 nucleic acid molecule and producing nucleic acid hybrids; and (b) isolating the nucleic acid hybrids to produce the enriched sample.

In some embodiments, which may be combined with any of the preceding aspects or embodiments, the one or more bait molecules comprise a capture nucleic acid molecule configured to hybridize to a nucleotide sequence corresponding to the CD274 nucleic acid molecule. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, which may be combined with any of the preceding aspects or embodiments, the one or more bait molecules are conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule.

In some embodiments, which may be combined with any of the preceding aspects or embodiments, the selectively enriching comprises amplifying the one or more nucleic acid molecules comprising nucleotide sequences corresponding to the CD274 nucleic acid molecule using a polymerase chain reaction (PCR) to produce an enriched sample.

In some embodiments, which may be combined with any of the preceding aspects or embodiments, the method further comprises sequencing the enriched sample.

In some embodiments, which may be combined with any of the preceding aspects or embodiments, the individual is a human. In some embodiments, the human is a human patient, such as a human cancer patient, or a human patient suspected of having cancer, being tested for cancer, or being treated for cancer.

In another aspect, provided herein is a kit comprising a probe, a bait, or one or more oligonucleotides for detecting: (a) a CD274 nucleic acid molecule that comprises or results from a rearrangement comprising a Breakpoint 1 and/or Breakpoint 2 within the chromosomal coordinates as listed in Table 1; (b) a CD274 fusion nucleic acid molecule comprising a fusion between a CD274 gene, or a portion thereof, and a fusion partner gene as listed in Table 2, or a portion thereof; or (c) a CD274 fusion nucleic acid molecule comprising a fusion between a CD274 gene, or a portion thereof, and a fusion partner gene as listed in Table 3, or a portion thereof, in a sample from an individual having a corresponding cancer as listed in Table 3. In some embodiments, the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4; or the CD274 fusion nucleic acid molecule comprises a fusion between a CD274 gene, or a portion thereof, and a fusion partner gene as listed in Table 3, or a portion thereof, and wherein the CD274 fusion nucleic acid molecule comprises or results from a corresponding CD274 Breakpoint and/or Fusion Partner Gene Breakpoint within the chromosomal coordinates as listed in Table 5.

In another aspect, provided herein is a CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule comprising a fusion between a CD274 gene, or a portion thereof, and a fusion partner gene as listed in Table 2, or a portion thereof. In some embodiments, the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4.

In another aspect, provided herein is a vector comprising a CD274 nucleic acid molecule provided herein. In another aspect, provided herein is a host cell comprising a vector provided herein.

In another aspect, provided herein is an antibody or antibody fragment that specifically binds to a PD-L1 polypeptide, or to a portion thereof, encoded by: (a) a CD274 nucleic acid molecule that comprises or results from a rearrangement comprising a Breakpoint 1 and/or Breakpoint 2 within the chromosomal coordinates as listed in Table 1; or (b) a CD274 fusion nucleic acid molecule comprising a fusion between a CD274 gene, or a portion thereof, and a fusion partner gene as listed in Table 2, or a portion thereof. In some embodiments, the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4.

In another aspect, provided herein is a kit comprising an antibody or antibody fragment for detecting: (a) a PD-L1 polypeptide, or a portion thereof, encoded by a CD274 nucleic acid molecule that comprises or results from a rearrangement comprising a Breakpoint 1 and/or Breakpoint 2 within the chromosomal coordinates as listed in Table 1; (b) a PD-L1 polypeptide, or a portion thereof, encoded by a CD274 fusion nucleic acid molecule comprising a fusion between a CD274 gene, or a portion thereof, and a fusion partner gene as listed in Table 2, or a portion thereof; or (c) a PD-L1 polypeptide, or a portion thereof, encoded by a CD274 fusion nucleic acid molecule comprising a fusion between a CD274 gene, or a portion thereof, and a fusion partner gene as listed in Table 3, or a portion thereof, in a sample from an individual having a corresponding cancer as listed in Table 3. In some embodiments, the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4; or (b) the CD274 fusion nucleic acid molecule comprises a fusion between a CD274 gene, or a portion thereof, and a fusion partner gene as listed in Table 3, or a portion thereof, and wherein the CD274 fusion nucleic acid molecule comprises or results from a corresponding CD274 Breakpoint and/or Fusion Partner Gene Breakpoint within the chromosomal coordinates as listed in Table 5.

In another aspect, provided herein is an in vitro use of a probe, a bait, or one or more oligonucleotides for detecting: (a) a CD274 nucleic acid molecule that comprises or results from a rearrangement comprising a Breakpoint 1 and/or Breakpoint 2 within the chromosomal coordinates as listed in Table 1; (b) a CD274 fusion nucleic acid molecule comprising a fusion between a CD274 gene, or a portion thereof, and a fusion partner gene as listed in Table 2, or a portion thereof; or (c) a CD274 fusion nucleic acid molecule comprising a fusion between a CD274 gene, or a portion thereof, and a fusion partner gene as listed in Table 3, or a portion thereof, in a sample from an individual having a corresponding cancer as listed in Table 3.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of a CD274 nucleic acid molecule, wherein: (i) the CD274 nucleic acid molecule comprises or results from a rearrangement comprising a Breakpoint 1 and/or Breakpoint 2 within the chromosomal coordinates as listed in Table 1, or (ii) the CD274 nucleic acid molecule is a CD274 fusion nucleic acid molecule comprising a fusion between a CD274 gene, or a portion thereof, and a fusion partner gene as listed in Table 2, or a portion thereof; and (c) detect, based on the analyzing, the CD274 nucleic acid molecule in the sample.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acid molecules, wherein the one or more nucleic acid molecules are derived from a sample obtained from an individual; (b) analyzing, using the one or more processors, the plurality of sequence reads for the presence of a CD274 nucleic acid molecule, wherein: (i) the CD274 nucleic acid molecule comprises or results from a rearrangement comprising a Breakpoint 1 and/or

Breakpoint 2 within the chromosomal coordinates as listed in Table 1, or (ii) the CD274 nucleic acid molecule is a CD274 fusion nucleic acid molecule comprising a fusion between a CD274 gene, or a portion thereof, and a fusion partner gene as listed in Table 2, or a portion thereof; and (c) detecting, using the one or more processors and based on the analyzing, the CD274 nucleic acid molecule in the sample.

In some embodiments, which may be combined with any of the preceding aspects or embodiments, the sample is from an individual having a cancer. In some embodiments, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, lung non-small cell lung carcinoma (NSCLC), gastric cancer or carcinoma, head and neck cancer, small cell cancer, essential thrombocythemia, agnogenic myeloid metaplasia, hypereosinophilic syndrome, systemic mastocytosis, familiar hypercosinophilia, chronic eosinophilic leukemia, neuroendocrine cancers, or a carcinoid tumor. In some embodiments, the cancer is an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, gastric cancer or carcinoma, unknown primary adenocarcinoma, unknown primary carcinoma (CUP), unknown primary carcinoma (CUP) (NOS), unknown primary melanoma, stomach adenocarcinoma, or vagina squamous cell carcinoma (SCC). In some embodiments, which may be combined with any of the preceding aspects or embodiments, the CD274 nucleic acid molecule comprises or results from a rearrangement comprising a Breakpoint 1 and/or Breakpoint 2 within the chromosomal coordinates as listed in Table 1, and wherein the cancer is the corresponding cancer as listed in Table 6; or (b) the CD274 fusion nucleic acid molecule comprises a fusion between a CD274 gene, or a portion thereof, and a fusion partner gene as listed in Table 2, or a portion thereof, and wherein the cancer is the corresponding cancer as listed in Table 7. In some embodiments, which may be combined with any of the preceding aspects or embodiments, the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4. In some embodiments, which may be combined with any of the preceding aspects or embodiments, the CD274 fusion nucleic acid molecule comprises a fusion between a CD274 gene, or a portion thereof, and a fusion partner gene as listed in Table 2, or a portion thereof; wherein the CD274 fusion nucleic acid molecule comprises or results from a corresponding CD274 Breakpoint and/or Fusion Partner Gene Breakpoint within the chromosomal coordinates as listed in Table 8; and wherein the cancer is the corresponding cancer as listed in Table 8.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual having a cancer; (b) analyze the plurality of sequence reads for the presence of a CD274 nucleic acid molecule, wherein the CD274 nucleic acid molecule is a CD274 fusion nucleic acid molecule comprising a fusion between a CD274 gene, or a portion thereof, and a fusion partner gene as listed in Table 3, or a portion thereof, wherein the cancer is the corresponding cancer as listed in Table 3; and (c) detect, based on the analyzing, the CD274 nucleic acid molecule in the sample.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acid molecules, wherein the one or more nucleic acid molecules are derived from a sample obtained from an individual having a cancer; (b) analyzing, using the one or more processors, the plurality of sequence reads for the presence of a CD274 nucleic acid molecule, wherein the CD274 nucleic acid molecule is a CD274 fusion nucleic acid molecule comprising a fusion between a CD274 gene, or a portion thereof, and a fusion partner gene as listed in Table 3, or a portion thereof, wherein the cancer is the corresponding cancer as listed in Table 3; and (c) detecting, using the one or more processors and based on the analyzing, the CD274 nucleic acid molecule in the sample.

In some embodiments, which may be combined with any of the preceding aspects or embodiments, the CD274 fusion nucleic acid molecule comprises a fusion between a CD274 gene, or a portion thereof, and a fusion partner gene as listed in Table 3, or a portion thereof, and the cancer is the corresponding cancer as listed in Table 3; and wherein the CD274 fusion nucleic acid molecule comprises or results from a corresponding CD274 Breakpoint and/or Fusion Partner Gene Breakpoint within the chromosomal coordinates as listed in Table 5.

In some embodiments, which may be combined with any of the preceding aspects or embodiments, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments, which may be combined with any of the preceding aspects or embodiments, the one or more program instructions when executed by the one or more processors are further configured to generate, based at least in part on the detecting, a genomic profile for the sample. In some embodiments, which may be combined with any of the preceding aspects or embodiments, the method further comprises generating, based at least in part on the detecting, a genomic profile for the sample. In some embodiments, the individual is administered a treatment based at least in part on the genomic profile. In some embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test. In some embodiments, the genomic profile further comprises the level of tumor mutational burden (TMB), clonal TMB, indel TMB, or nonsense-mediated decay (NMD)-escape TMB; the presence or absence of, or the proportion of mutations fitting, a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof.

In some embodiments, which may be combined with any of the preceding aspects or embodiments, the one or more program instructions when executed by the one or more processors are further configured to generate a report indicating the presence or absence of the CD274 nucleic acid molecule in the sample. In some embodiments, which may be combined with any of the preceding aspects or embodiments, the method further comprises generating a report indicating the presence or absence of the CD274 nucleic acid molecule in the sample. In some embodiments, the report is generated by the one or more processors. In some embodiments, the method further comprises transmitting the report to a healthcare provider. In some embodiments, the report is transmitted via a computer network or a peer-to-peer connection.

In another aspect, provided herein is an immune checkpoint inhibitor for use in a method of treating or delaying progression of cancer, wherein the method comprises administering the immune checkpoint inhibitor to an individual, wherein: (a) a CD274 nucleic acid molecule that comprises or results from a rearrangement comprising a Breakpoint 1 and/or Breakpoint 2 within the chromosomal coordinates as listed in Table 1 is detected in a sample obtained from the individual; (b) a CD274 fusion nucleic acid molecule comprising a fusion between a CD274 gene, or a portion thereof, and a fusion partner gene as listed in Table 2, or a portion thereof, is detected in a sample obtained from the individual; or (c) a CD274 fusion nucleic acid molecule comprising a fusion between a CD274 gene, or a portion thereof, and a fusion partner gene as listed in Table 3, or a portion thereof, is detected in a sample from an individual having a corresponding cancer as listed in Table 3.

In another aspect, provided herein is an immune checkpoint inhibitor for use in the manufacture of a medicament for treating or delaying progression of cancer, wherein the medicament is to be administered to an individual, wherein: (a) a CD274 nucleic acid molecule that comprises or results from a rearrangement comprising a Breakpoint 1 and/or Breakpoint 2 within the chromosomal coordinates as listed in Table 1 is detected in a sample obtained from the individual; (b) a CD274 fusion nucleic acid molecule comprising a fusion between a CD274 gene, or a portion thereof, and a fusion partner gene as listed in Table 2, or a portion thereof, is detected in a sample obtained from the individual; or (c) a CD274 fusion nucleic acid molecule comprising a fusion between a CD274 gene, or a portion thereof, and a fusion partner gene as listed in Table 3, or a portion thereof, is detected in a sample from an individual having a corresponding cancer as listed in Table 3.

In another aspect, provided herein is a method of selecting a treatment for an individual having a cancer, the method comprising detecting or acquiring knowledge of a cluster of differentiation 274 (CD274) nucleic acid molecule, or a programmed death-ligand 1 (PD-L1) polypeptide encoded by the CD274 nucleic acid molecule, in a sample from the individual, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; wherein detecting or acquiring knowledge of the CD274 nucleic acid molecule, or the PD-L1 polypeptide encoded by the CD274 nucleic acid molecule, in the sample identifies the individual as one who may benefit from a treatment comprising an immune checkpoint inhibitor.

In another aspect, provided herein is a method of treating or delaying progression of cancer, comprising: detecting or acquiring knowledge of a CD274 nucleic acid molecule, or a PD-L1 polypeptide encoded by the CD274 nucleic acid molecule, in a sample from an individual having a cancer, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and administering to the individual an effective amount of a treatment that comprises an immune checkpoint inhibitor responsive to detecting or acquiring knowledge of the CD274 nucleic acid molecule, or the PD-L1 polypeptide encoded by the CD274 nucleic acid molecule, in the sample.

In another aspect, provided herein is a method of detecting a CD274 nucleic acid molecule, the method comprising: providing a plurality of nucleic acid molecules obtained from a sample from an individual having a cancer, wherein the plurality of nucleic acid molecules comprises nucleic acid molecules corresponding to a CD274 nucleic acid molecule, wherein: (a) the CD274 nucleic acid molecule comprises or results from a rearrangement comprising a Breakpoint 1 and/or Breakpoint 2 within the chromosomal coordinates as listed in Table 1; or (b) the CD274 nucleic acid molecule is a CD274 fusion nucleic acid molecule, wherein: (i) the CD274 fusion nucleic acid molecule comprises a fusion between a CD274 gene, or a portion thereof, and a fusion partner gene as listed in Table 2, or a portion thereof, or (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; ligating one or more adapters onto one or more nucleic acid molecules from the plurality of nucleic acid molecules; amplifying one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequence reads correspond to the CD274 nucleic acid molecule; analyzing the plurality of sequence reads; and based on the analysis, detecting the presence or absence of the CD274 nucleic acid molecule in the sample.

In some embodiments. (a) the method further comprises receiving, at one or more processors, sequence read data for the plurality of sequence reads; (b) the analyzing the plurality of sequence reads comprises identifying, using one or more processors, the presence or absence of sequence reads corresponding to the CD274 nucleic acid molecule; (c) the amplified nucleic acid molecules are captured by hybridization with one or more bait molecules; (d) the plurality of nucleic acid molecules comprises a mixture of cancer nucleic acid molecules and non-cancer nucleic acid molecules; (e) the one or more adapters comprise amplification primers, flow cell adapter sequences, substrate adapter sequences, or sample index sequences; (f) the amplifying comprises performing a polymerase chain reaction (PCR) amplification technique, a non-PCR amplification technique, or an isothermal amplification technique; (g) the sequencing comprises use of a massively parallel sequencing (MPS) technique, whole genome sequencing (WGS), whole exome sequencing, targeted sequencing, direct sequencing, a Sanger sequencing technique, or next generation sequencing (NGS); (h) the sequencer comprises a next generation sequencer; (i) the method further comprises generating a genomic profile for the individual, based, at least in part, on detecting the presence or absence of the CD274 nucleic acid molecule in the sample; and/or (j) the method further comprises generating a report indicating the presence or absence of the CD274 nucleic acid molecule in the sample.

In some embodiments. (a) the genomic profile for the individual further comprises: (i) results from a comprehensive genomic profiling (CGP) test, a gene expression profiling test, a cancer hotspot panel test, a DNA methylation test, a DNA fragmentation test, an RNA fragmentation test, a nucleic acid sequencing-based test, or any combination thereof; and/or (ii) the level of tumor mutational burden (TMB), clonal TMB, indel TMB, or nonsense-mediated decay (NMD)-escape TMB; the presence or absence of a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof; and/or (b) the method further comprises selecting a treatment, administering a treatment, or applying a treatment to the individual based on the generated genomic profile, wherein the treatment comprises an immune checkpoint inhibitor.

In some embodiments, the CD274 nucleic acid molecule comprises or results from a rearrangement comprising a Breakpoint 1 and/or Breakpoint 2 within the chromosomal coordinates as listed in Table 1, or the CD274 fusion nucleic acid molecule comprises a fusion between a CD274 gene, or a portion thereof, and a fusion partner gene as listed in Table 2, or a portion thereof, and wherein the cancer is: (a) a carcinoma, a sarcoma, a lymphoma, a leukemia, a myeloma, a germ cell cancer, or a blastoma; (b) a solid tumor or a hematologic malignancy; (c) a B cell cancer, melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypercosinophilia, chronic eosinophilic leukemia, neuroendocrine cancers, or a carcinoid tumor; or (d) an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, unknown primary adenocarcinoma, gastric cancer or carcinoma, unknown primary carcinoma (CUP), unknown primary carcinoma (CUP) (NOS), unknown primary melanoma, stomach adenocarcinoma, or vagina squamous cell carcinoma (SCC).

In some embodiments, (a) the CD274 nucleic acid molecule comprises or results from a rearrangement comprising a Breakpoint 1 and/or Breakpoint 2 within the chromosomal coordinates as listed in Table 1, and wherein the cancer is the corresponding cancer as listed in Table 6; or (b) the CD274 fusion nucleic acid molecule comprises a fusion between a CD274 gene, or a portion thereof, and a fusion partner gene as listed in Table 2, or a portion thereof, and wherein the cancer is the corresponding cancer as listed in Table 7.

In some embodiments, (a) the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4; (b) the CD274 fusion nucleic acid molecule comprises a fusion between a CD274 gene, or a portion thereof, and a fusion partner gene as listed in Table 2, or a portion thereof, wherein the CD274 fusion nucleic acid molecule comprises or results from a corresponding CD274 Breakpoint and/or Fusion Partner Gene Breakpoint within the chromosomal coordinates as listed in Table 8, and wherein the cancer is the corresponding cancer as listed in Table 8; or (c) the CD274 fusion nucleic acid molecule comprises a fusion between a CD274 gene, or a portion thereof, and a fusion partner gene as listed in Table 3, or a portion thereof, and the cancer is the corresponding cancer as listed in Table 3, and wherein the CD274 fusion nucleic acid molecule comprises or results from a corresponding CD274 Breakpoint and/or Fusion Partner Gene Breakpoint within the chromosomal coordinates as listed in Table 5.

In some embodiments, the cancer is metastatic.

In some embodiments, the immune checkpoint inhibitor: (a) comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, or any combination thereof; (b) is a PD-1-, or a PD-L1-targeted agent; (c) is a PD-1 inhibitor, a PD-L1 inhibitor, or a CTLA-4 inhibitor; and/or (d) is a monotherapy. In some embodiments, (a) the PD-1 inhibitor comprises one or more of nivolumab, pembrolizumab, cemiplimab, or dostarlimab; (b) the PD-L1-inhibitor comprises one or more of atezolizumab, avelumab, or durvalumab; (c) the CTLA-4 inhibitor comprises ipilimumab; or (d) the nucleic acid comprises a double-stranded RNA (dsRNA), a small interfering RNA (siRNA), or a small hairpin RNA (shRNA).

In some embodiments, the treatment comprises: (a) an additional anti-cancer therapy; and/or (b) an immune checkpoint inhibitor in combination with one or more chemotherapeutic agents. In some embodiments, (a) the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof; (b) the additional anti-cancer therapy comprises a cellular therapy, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy; (c) the additional anti-cancer therapy comprises a nucleic acid, wherein the nucleic acid comprises a double-stranded RNA (dsRNA), a small interfering RNA (siRNA), or a small hairpin RNA (shRNA); and/or (d) the one or more chemotherapeutic agents comprise a platinum-based chemotherapeutic agent, bevacizumab-Awwb, bevacizumab, carboplatin, paclitaxel, paclitaxel protein-bound, or pemetrexed.

In some embodiments, the encoded PD-L1 polypeptide is oncogenic, and/or promotes cancer cell survival, angiogenesis, cancer cell proliferation, and any combination thereof.

In some embodiments, the method further comprises: (a) acquiring knowledge of or detecting in a sample from the individual the presence or absence of a CD274 gene amplification; (b) acquiring knowledge of or detecting in a sample from the individual the presence or absence of a base substitution, a short insertion/deletion (indel), a copy number alteration, or a genomic rearrangement in one or more genes; (c) acquiring knowledge of or detecting in a sample from the individual the presence or absence of a genomic Epstein-Barr virus (EBV); (d) acquiring knowledge of or detecting a microsatellite instability status of the cancer in a sample from the individual; (c) acquiring knowledge of or determining tumor mutational burden (TMB) in a sample from the individual; (f) acquiring knowledge of or determining the level of PD-L1 expression in a sample from the individual; and/or (g) acquiring knowledge of or determining the clonality of the CD274 nucleic acid molecule in the cancer.

In some embodiments. (a) the one or more genes comprise one or more of: TP53, PIK3CA, CDKN2A, KRAS, CDKN2B, CD274, MYC, JAK2, RB1, PDCD1LG2, APC, ARID1A, PTEN, BRAF, CREBBP, PBRM1, KMT2D, CCND1, KDM6A, BCL2L1, ERBB2, FBXW7, NF1, BCORL1, BRCA2, FGF19, FGFR1, MAP2K1, PRKC1, ATM, CDK12, CTNNB1, DNMT3A, FGF3, FGF4, GNAS, LYN, MET, NOTCH1, RNF43, STK11, TET2, VHL, ZNF217, ASXL1, BRCA1, EGFR, KDM5C, KIT, NFE2L2, NOTCH2, NOTCH3, PIK3R1, SOX9, TERC, ZNF703, MTAP, BRIP1, CDC73, ACVR1B, ATRX, MLH1, BRD4, SMAD4, PALB2, RAD21, GATA6, CTCF, MLH1, a mismatch repair gene, or any combination thereof; (b) the cancer comprises a base substitution, a small insertion/deletion (indel), a copy number alteration, or a genomic rearrangement in the one or more genes; (c) the EBV is HHV-4; (d) the cancer or the individual comprises a genomic EBV or is positive for EBV; (e) the cancer is microsatellite stable; (f) the cancer has a TMB of less than 6 mutations per megabase (mut/Mb), between 6 and 20 mut/Mb, greater than 20 mut/Mb, a high TMB, a TMB of about 7.0 mut/Mb, or a TMB of at least about 10 mut/Mb; (g) the cancer is PD-L1 positive, PD-L1-high positive, or PD-L1 negative; (h) the cancer comprises a CD274 gene amplification, or the cancer does not comprise a CD274 gene amplification; and/or (i) the CD274 nucleic acid molecule results from a clonal or a sub-clonal rearrangement of a CD274 gene in the cancer, or the CD274 nucleic acid molecule is clonal or sub-clonal in the cancer.

In some embodiments, the cancer or the individual comprises a genomic EBV or is positive for EBV, and wherein: the cancer is a gastric cancer, a gastric adenocarcinoma, or a stomach adenocarcinoma; and/or the treatment comprises pembrolizumab.

In some embodiments, clonality of the CD274 nucleic acid molecule is assessed by performing DNA sequencing on a sample obtained from the individual. In some embodiments, (a) clonality of the CD274 nucleic acid molecule is assessed based on sequencing coverage of a CD274 gene and the number of sequence read pairs spanning a breakpoint of the CD274 nucleic acid molecule; or (b) clonality of the CD274 nucleic acid molecule is assessed based on the number of sequence read pairs spanning a breakpoint of the CD274 nucleic acid molecule. In some embodiments. (a) clonality of the CD274 nucleic acid molecule is assessed based on sequencing coverage of a CD274 gene and a threshold for the number of sequence read pairs spanning a breakpoint of the CD274 nucleic acid molecule, wherein the threshold is selected using a receiver operator characteristic (ROC) curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis; (b) clonality of the CD274 nucleic acid molecule is assessed based on the number of sequence read pairs spanning a breakpoint of the CD274 nucleic acid molecule, wherein the clonality is assessed based on a threshold for the number of sequence read pairs spanning a breakpoint of the CD274 nucleic acid molecule, wherein the threshold is selected using a ROC curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis; and/or c) the sample is a bulk tumor sample derived from a single anatomic location. In some embodiments. (a) a sample from a tumor is PD-L1 high positive if it comprises a tumor proportion score (TPS) of at least about 50%; and/or (b) the threshold for the number of sequence read pairs spanning a breakpoint of the CD274 nucleic acid molecule is at least about 20 read pairs, at least about 21 read pairs, at least about 22 read pairs, at least about 23 read pairs, at least about 24 read pairs, at least about 25 read pairs, at least about 26 read pairs, at least about 27 read pairs, at least about 28 read pairs, at least about 29 read pairs, at least about 30 read pairs, at least about 31 read pairs, at least about 32 read pairs, at least about 33 read pairs, at least about 34 read pairs, at least about 35 read pairs, at least about 36 read pairs, at least about 37 read pairs, at least about 38 read pairs, at least about 39 read pairs, at least about 40 read pairs, at least about 41 read pairs, at least about 42 read pairs, at least about 43 read pairs, at least about 44 read pairs, at least about 45 read pairs, at least about 46 read pairs, at least about 47 read pairs, at least about 48 read pairs, at least about 49 read pairs, or at least about 50 read pairs spanning a breakpoint of the CD274 nucleic acid molecule.

In some embodiments, (a) responsive to acquiring knowledge of or determining that the CD274 nucleic acid molecule is clonal in the cancer or results from a clonal rearrangement of a CD274 gene in the cancer: (i) the individual is identified as likely to respond to a treatment comprising an immune checkpoint inhibitor; (ii) the individual is predicted to have longer survival when treated with a treatment comprising an immune checkpoint inhibitor, as compared to survival of: an individual whose cancer does not comprise a clonal CD274 nucleic acid molecule, or a CD274 nucleic acid molecule resulting from a clonal rearrangement of a CD274 gene; or an individual whose cancer comprises a sub-clonal CD274 nucleic acid molecule, or a CD274 nucleic acid molecule resulting from a sub-clonal rearrangement of a CD274 gene; and/or (iii) the individual is predicted to have an improved response to treatment with an immune checkpoint inhibitor, as compared to: an individual whose cancer does not comprise a clonal CD274 nucleic acid molecule, or a CD274 nucleic acid molecule resulting from a clonal rearrangement of a CD274 gene; or an individual whose cancer comprises a sub-clonal CD274 nucleic acid molecule, or a CD274 nucleic acid molecule resulting from a sub-clonal rearrangement of a CD274 gene; and/or (b) acquiring knowledge of or determining that the CD274 nucleic acid molecule is clonal in the cancer or results from a clonal rearrangement of a CD274 gene in the cancer identifies the cancer as: (i) likely to be PD-L1 positive or PD-L1 high positive; and/or (ii) likely to have a TPS of at least about 50%, assessed based on an immunohistochemistry assay.

In some embodiments, (a) the method further comprises obtaining the sample from the individual, and/or the sample is obtained from the cancer; and/or (b) the sample: (i) comprises a tissue biopsy sample, a liquid biopsy sample, or a normal control, (ii) is from a tumor biopsy, tumor specimen, or circulating tumor cell, (iii) is a liquid biopsy sample and comprises blood, plasma, cerebrospinal fluid, sputum, stool, urine, or saliva, (iv) comprises cells and/or nucleic acids from the cancer, (v) comprises mRNA, DNA, circulating tumor DNA (ctDNA), cell-free DNA, or cell-free RNA from the cancer, (vi) is a liquid biopsy sample and comprises circulating tumor cells (CTCs), or (vii) is a liquid biopsy sample and comprises cell-free DNA (cfDNA), circulating tumor DNA (ctDNA), or any combination thereof.

In some embodiments. (a) the method comprises acquiring knowledge of or detecting the CD274 nucleic acid molecule, or the PD-L1 polypeptide encoded by the CD274 nucleic acid molecule, in a tissue biopsy sample, in a liquid biopsy sample, or in both a tissue biopsy sample and a liquid biopsy sample, from the individual; (b) acquiring knowledge of the CD274 nucleic acid molecule, or the PD-L1 polypeptide encoded by the CD274 nucleic acid molecule, comprises detecting the CD274 nucleic acid molecule, or the PD-L1 polypeptide encoded by the CD274 nucleic acid molecule, in the sample; (c) detecting the CD274 nucleic acid molecule comprises detecting a fragment of the CD274 nucleic acid molecule comprising a breakpoint; and/or (d) detecting the PD-L1 polypeptide encoded by the CD274 nucleic acid molecule comprises detecting a portion of the polypeptide that is encoded by a fragment of the CD274 nucleic acid molecule that comprises a breakpoint.

In some embodiments, (a) the CD274 nucleic acid molecule is detected in the sample by: (i) one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing; (ii) sequencing using a massively parallel sequencing (MPS) technique, whole genome sequencing (WGS), whole exome sequencing, targeted sequencing, direct sequencing, a Sanger sequencing technique, next-generation sequencing (NGS), or RNA-sequencing (RNA-seq); or (iii) a polymerase chain reaction (PCR) amplification technique, a non-PCR amplification technique, an isothermal amplification technique, a reverse transcription PCR (RT-PCR), a quantitative real-time PCR (qPCR), or a reverse transcription quantitative real-time PCR (RT-qPCR) assay; (b) the PD-L1 polypeptide encoded by the CD274 nucleic acid molecule is detected in the sample by one or more of: immunoblotting, enzyme linked immunosorbent assay (ELISA), immunohistochemistry, or mass spectrometry; and/or (c) the CD274 nucleic acid molecule, or the PD-L1 polypeptide encoded by the CD274 nucleic acid molecule, is detected using a digital pathology method.

In some embodiments, the method further comprises selectively enriching for one or more nucleic acid molecules in the sample comprising nucleotide sequences corresponding to the CD274 nucleic acid molecule, wherein the selectively enriching produces an enriched sample. In some embodiments. (a) the selectively enriching comprises: (i) combining one or more bait molecules with the sample, thereby hybridizing the one or more bait molecules to one or more nucleic acid molecules in the sample comprising nucleotide sequences corresponding to the CD274 nucleic acid molecule and producing nucleic acid hybrids, and isolating the nucleic acid hybrids to produce the enriched sample, or (ii) amplifying the one or more nucleic acid molecules comprising nucleotide sequences corresponding to the CD274 nucleic acid molecule using a polymerase chain reaction (PCR) to produce an enriched sample; and/or (b) the method further comprises sequencing the enriched sample. In some embodiments, the one or more bait molecules comprise a capture nucleic acid molecule configured to hybridize to a nucleotide sequence corresponding to the CD274 nucleic acid molecule; and/or the one or more bait molecules are conjugated to an affinity reagent or to a detection reagent. In some embodiments, (a) the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides; (b) the affinity reagent is an antibody, an antibody fragment, or biotin, or wherein the detection reagent is a fluorescent marker; and/or (c) the capture nucleic acid molecule comprises a DNA, RNA, or mixed DNA/RNA molecule.

In some embodiments, the individual is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 depicts a diagram of the CD274 gene on chromosome 9 (human genome assembly hg19). As indicated in the legend, introns are shown as thin lines and exons are shown as boxes. The 3′-UTR is indicated with a solid rectangle. The locations of CD274 gene rearrangement breakpoints identified in the studies described in Example 1 are shown as horizontal lines under the CD274 gene diagram.

FIG. 2 shows the frequency of alteration types (deletions, duplications, fusions, rearrangements, or truncations) in tumors harboring CD274 genomic alterations (GAs) identified in Example 1.

FIG. 3 shows the distribution of disease groups harboring CD274 rearrangements identified in the studies described in Example 1. The results shown are ranked by prevalence of CD274 rearrangements in the indicated cancer types.

FIG. 4 shows the prevalence (percentage) of co-occurring gene mutations in the indicated genes among cases with CD274 rearrangements.

FIG. 5 shows a volcano plot of co-occurring gene alterations in cases with CD274 rearrangements. Two-tailed Fisher's exact tests were used to estimate the p values and odds ratios (x-axis) of associations between the indicated gene alterations and CD274 rearrangements. The Benjamini-Hochberg procedure was used to estimate the adjusted p-values (y-axis). Only genes with a pan-cancer prevalence ≥0.5% and an adjusted p-value≤0.05 are labeled (PIK3CA, KDM6A, PBRM1, CDK12, BCL2L1, BCORL1, CREBBP, VHL, KDMSC, MAP2K1, JAK2, and PDCD1LG2).

FIG. 6 shows the distribution of tumor mutational burden (TMB) in the indicated tumor types in all cases of each tumor type, and in cases with CD274 rearrangements. Two-tailed Fisher's exact tests were used to estimate the p-values of the differences in the relative distributions of TMB status in the CD274-rearranged cohort as compared to molecularly unselected tumor type-matched cases. The p-values are listed on top of the bars corresponding to each cancer type. NSCLC, non-small cell lung cancer; CRC, colorectal cancer; CUP, carcinoma of unknown primary; Breast, breast cancer; Ovary, ovarian cancer; Eso, esophageal cancer; Kidney, renal cell cancer; Stom, stomach cancer; HN, head and neck cancer; TMB<6, TMB lower than 6 mutations per megabase (muts/Mb); TMB 6-20, TMB of 6-20 muts/Mb; and TMB>20, TMB higher than 20 muts/Mb.

FIG. 7 shows the distribution of PD-L1 staining by immunohistochemistry (IHC) of CD274-rearranged cases. PD-L1 IHC results were available for 43 out of the 145 cases in the CD274-rearranged cohort as described in Example 1. The percent PD-L1 staining by IHC is shown on the x-axis and the corresponding number of CD274-rearranged cases is shown in the y-axis.

FIG. 8 shows the time on immune checkpoint inhibitor (ICI) therapy in patients with CD274 rearrangements from a real world clinico-genomic database (CGDB). The depicted graph is a swimmer plot of time-on-treatment (x-axis, days) for eight CGDB patients with CD274 rearrangements treated with the treatments indicated in the legend on the right (+=atezolizumab and chemotherapy: *=nivolumab: ‡=pembrolizumab; and *=pembrolizumab and chemotherapy). The primary tumor type in each patient is indicated by the legend on the right (B=breast cancer; L=lung cancer; S=stomach cancer; C=colon cancer; O=ovarian cancer). The arrows indicate patients that continued on treatment as of the last available clinical follow-up.

FIGS. 9A-9C show the results from an analysis of the clonality of CD274 rearrangements. FIG. 9A shows a comparison of supporting sequencing read pairs between cases with PD-L1 tumor percentage score of <50% versus ≥50%. A t-test was performed to determine statistical significance (p-value=0.0086). FIG. 9B shows a comparison of sequencing coverage of CD274 between PD-L1 cases with tumor percentage score of <50% and ≥50%. A t-test was performed to determine statistical significance (p-value=0.64). FIG. 9C shows a receiver operating characteristic curve (ROC) analysis for prediction of tumor percentage score of ≥50% based on CD274 rearrangement read pairs. The ROC curve shows the number of read pairs as a predictor for TPS ≥50% in 43 CD274-rearranged samples with paired IHC and CGP data. As depicted in the figure: thres, threshold; sens, sensitivity; spec, specificity; and AUC, area under the curve.

FIG. 10 depicts an exemplary device, in accordance with some embodiments.

FIG. 11 depicts an exemplary system, in accordance with some embodiments.

FIG. 12 depicts a block diagram of an exemplary process for detecting a CD274 nucleic acid molecule of the disclosure in a sample, in accordance with some embodiments.

### DETAILED DESCRIPTION

The present disclosure relates generally to detecting CD274 rearrangements, CD274 nucleic acid molecules, and PD-L1 polypeptides in cancer, as well as methods of treatment, and uses related thereto.

The present disclosure describes a study of a real-world dataset comprising high-quality, validated hybrid capture-based next-generation sequencing (NGS) results that characterized rearrangements involving the CD274 gene in 283.050 tumor tissue samples corresponding to multiple cancer types. As described herein. Applicants discovered a multitude of CD274 rearrangements, spanning a diversity of cancer types and breakpoints. Applicants further discovered that the presence of CD274 rearrangements in cancer was associated with increased PD-L1 expression, higher tumor mutational burden, and clinical benefit of immune checkpoint inhibitor treatment in various cancer types. See, e.g., Example 1. Thus, without wishing to be bound by theory, it is thought that the presence of a CD274 nucleic acid molecule described herein, e.g., a CD274 nucleic acid molecule comprising or resulting from a CD274 rearrangement described herein, or a CD274 fusion nucleic acid molecule described herein, or of a PD-L1 polypeptide encoded by a CD274 nucleic acid of the disclosure, in sample from an individual having cancer, suspected of having cancer, being tested for cancer, or being treated for cancer may identify the individual as likely to exhibit clinical benefit to treatment comprising an immune checkpoint inhibitor.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising.” “consisting.” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer.” “cancerous,” and “tumor” are not mutually exclusive as referred to herein.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”). “(0)NR₂ (“amidate”), P(0)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“K”) and lambda (“2”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1. CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1 991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-1 13 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody.” “intact antibody.” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14(3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-31 0 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 1 1 9-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg et al., Intern. Rev. Immunol. 13:65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2, or Megalign (DNASTAR) software. Those skilled in the art can determine appropriate parameters for measuring alignment, including any algorithms needed to achieve maximal alignment over the full-length of the sequences being compared.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy, e.g., a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations, or other mutations or alterations), polypeptides, polypeptide and polynucleotide modifications (e.g., post-translational modifications), carbohydrates, and/or glycolipid-based molecular markers. In some embodiments, a biomarker is a CD274 nucleic acid molecule or a PD-L1 polypeptide described herein.

“Amplification.” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample.” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample.” “reference cell.” “reference tissue.” “control sample.” “control cell,” “control tissue,” “normal sample.” “normal cell,” or “normal tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to an embodiment of polypeptide analysis or protocol, one may use results of a polypeptide expression analysis or protocol to determine whether a specific therapeutic regimen should be performed. With respect to an embodiment of polynucleotide analysis or protocol, one may use results of a polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual.” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The terms “concurrently” or “in combination” are used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker (e.g., a CD274 nucleic acid molecule or PD-L1 polypeptide) described herein. In certain embodiments, the manufacture or kit is promoted, distributed, or sold as a unit for performing the methods described herein.

The phrase “based on” when used herein means that the information about one or more biomarkers (e.g., a CD274 nucleic acid molecule or PD-L1 polypeptide described herein) is used to inform a treatment decision, information provided on a package insert, or marketing/promotional guidance, etc.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

### III. Methods, Systems, and Devices

In certain aspects, provided herein are methods for selecting a treatment for an individual having a cancer; methods for identifying one or more treatment options for an individual having a cancer; methods for predicting survival of an individual having a cancer; methods for treating or delaying progression of cancer; methods for monitoring, evaluating or screening an individual having a cancer; methods for assessing a CD274 nucleic acid molecule or a PD-L1 polypeptide in a cancer in an individual; methods for detecting a CD274 nucleic acid molecule or a PD-L1 polypeptide; methods for detecting the presence or absence of a cancer in an individual; methods for monitoring progression or recurrence of a cancer in an individual; methods for identifying a candidate treatment for a cancer in an individual in need thereof; methods for identifying an individual having a cancer who may benefit from a treatment comprising an immune checkpoint inhibitor; and methods for predicting survival of an individual having a cancer treated with a treatment comprising an immune checkpoint inhibitor.

In some embodiments, the methods provided herein comprise detecting in a sample from an individual, e.g., an individual having cancer, suspected of having cancer, being treated for cancer, or being tested for cancer, a cluster of differentiation 274 (CD274) nucleic acid molecule, or a programmed death-ligand 1 (PD-L1) polypeptide encoded by the CD274 nucleic acid molecule. In some embodiments, detection of the CD274 nucleic acid molecule, or the PD-L1 polypeptide encoded by the CD274 nucleic acid molecule, in the sample identifies the individual as one who may benefit from a treatment comprising an immune checkpoint inhibitor. In some embodiments, the methods comprise selecting an immune checkpoint inhibitor as a treatment for an individual having cancer. e.g., responsive to detection of a CD274 nucleic acid molecule, or a PD-L1 polypeptide encoded by the CD274 nucleic acid molecule, in the sample. In some embodiments, the methods comprise generating a report comprising one or more treatment options identified for an individual based at least in part on detection of a CD274 nucleic acid molecule, or a PD-L1 polypeptide encoded by the CD274 nucleic acid molecule, in a sample from the individual. In some embodiments, the one or more treatment options comprise an immune checkpoint inhibitor. In some embodiments, the methods comprise administering to an individual an effective amount of a treatment that comprises an immune checkpoint inhibitor responsive to detecting a CD274 nucleic acid molecule, or a PD-L1 polypeptide encoded by the CD274 nucleic acid molecule, in a sample from the individual. In some embodiments, responsive to detection of a CD274 nucleic acid molecule, or a PD-L1 polypeptide encoded by the CD274 nucleic acid molecule, in a sample from an individual, the individual is predicted to have an improved response to treatment with an immune checkpoint inhibitor as compared to an individual whose cancer does not comprise a CD274 nucleic acid molecule, or a PD-L1 polypeptide encoded by the CD274 nucleic acid molecule. In some embodiments, the methods comprise providing an assessment of a CD274 nucleic acid molecule, or a PD-L1 polypeptide encoded by the CD274 nucleic acid molecule, e.g., responsive to detecting the presence or absence of the CD274 nucleic acid molecule, or the PD-L1 polypeptide encoded by the CD274 nucleic acid molecule, in the sample. In some embodiments, the methods comprise detecting or acquiring knowledge of the presence or absence of a cancer in a sample from the individual. In some embodiments, the methods comprise detecting, in a first sample obtained from an individual at a first time point, the presence or absence of a CD274 nucleic acid molecule, or a PD-L1 polypeptide encoded by the CD274 nucleic acid molecule; detecting, in a second sample obtained from the individual at a second time point after the first time point, the presence or absence of a CD274 nucleic acid molecule, or a PD-L1 polypeptide encoded by the CD274 nucleic acid molecule; and providing an assessment of cancer progression or cancer recurrence in the individual based, at least in part, on the presence or absence of the CD274 nucleic acid molecule, or the PD-L1 polypeptide encoded by the CD274 nucleic acid molecule, in the first sample and/or in the second sample. In some embodiments, the methods comprise performing DNA sequencing on a sample obtained from an individual to determine a sequencing mutation profile on a CD274 gene, wherein the sequencing mutation profile identifies the presence or absence of a CD274 nucleic acid molecule. In some embodiments, the methods comprise identifying a candidate treatment based, at least in part, on a sequencing mutation profile on a CD274 gene. In some embodiments, the candidate treatment comprises an immune checkpoint inhibitor. In some embodiments, the candidate treatment comprises an immune checkpoint inhibitor and is identified based, at least in part, on the presence of a CD274 nucleic acid molecule as identified in the sequencing mutation profile.

In some embodiments, the methods provided herein comprise acquiring knowledge of a CD274 nucleic acid molecule, or a PD-L1 polypeptide encoded by the CD274 nucleic acid molecule, in a sample from an individual, e.g., an individual having cancer, suspected of having cancer, being treated for cancer, or being tested for cancer. In some embodiments, knowledge of the presence of a CD274 nucleic acid molecule, or a PD-L1 polypeptide encoded by the CD274 nucleic acid molecule, in a sample from an individual identifies the individual as one who may benefit from a treatment comprising an immune checkpoint inhibitor. In some embodiments, the methods comprise selecting an immune checkpoint inhibitor as a treatment for an individual having cancer, e.g., responsive to knowledge of the presence of a CD274 nucleic acid molecule, or a PD-L1 polypeptide encoded by the CD274 nucleic acid molecule, in a sample from the individual. In some embodiments, the methods comprise generating a report comprising one or more treatment options identified for an individual based at least in part on knowledge of the presence of a CD274 nucleic acid molecule, or a PD-L1 polypeptide encoded by the CD274 nucleic acid molecule in sample from the individual. In some embodiments, the one or more treatment options comprise an immune checkpoint inhibitor. In some embodiments, responsive to acquisition of knowledge of the presence of a CD274 nucleic acid molecule, or a PD-L1 polypeptide encoded by the CD274 nucleic acid molecule, in a sample from an individual, the individual is classified as a candidate to receive a treatment comprising an immune checkpoint inhibitor. In some embodiments, responsive to acquisition of knowledge of the presence of a CD274 nucleic acid molecule, or a PD-L1 polypeptide encoded by the CD274 nucleic acid molecule, in a sample from an individual, the individual is identified as likely to respond to a treatment that comprises an immune checkpoint inhibitor. In some embodiments, responsive to acquisition of knowledge of the presence of a CD274 nucleic acid molecule, or a PD-L1 polypeptide encoded by the CD274 nucleic acid molecule, in a sample from an individual, the individual is predicted to have longer survival when treated with a treatment comprising an immune checkpoint inhibitor, e.g., as compared to survival of an individual whose cancer does not comprise the CD274 nucleic acid molecule, or the PD-L1 polypeptide encoded by the CD274 nucleic acid molecule. In some embodiments, responsive to acquisition of knowledge of the presence of a CD274 nucleic acid molecule, or a PD-L1 polypeptide encoded by the CD274 nucleic acid molecule, in a sample from an individual, the individual is predicted to have longer survival when treated with a treatment comprising an immune checkpoint inhibitor, as compared to an individual whose cancer does not exhibit the CD274 nucleic acid molecule, or the PD-L1 polypeptide encoded by the CD274 nucleic acid molecule. In some embodiments, the methods comprise administering to an individual an effective amount of a treatment that comprises an immune checkpoint inhibitor responsive to acquiring knowledge of a CD274 nucleic acid molecule, or a PD-L1 polypeptide encoded by the CD274 nucleic acid molecule, in a sample from the individual. In some embodiments, responsive to acquiring knowledge of a CD274 nucleic acid molecule, or a PD-L1 polypeptide encoded by the CD274 nucleic acid molecule, in a sample from an individual, the individual is predicted to have an improved response to treatment with an immune checkpoint inhibitor as compared to an individual whose cancer does not comprise a CD274 nucleic acid molecule, or a PD-L1 polypeptide encoded by the CD274 nucleic acid molecule. In some embodiments, the methods comprise providing an assessment of a CD274 nucleic acid molecule, or a PD-L1 polypeptide encoded by the CD274 nucleic acid molecule, e.g., responsive to acquiring knowledge of the presence or absence of the CD274 nucleic acid molecule, or the PD-L1 polypeptide encoded by the CD274 nucleic acid molecule, in a sample from an individual. In some embodiments, the methods comprise detecting or acquiring knowledge of the presence or absence of a cancer in a sample from an individual.

In other aspects, provided herein are systems. In some embodiments, a system of the disclosure comprises a memory configured to store one or more program instructions; and one or more processors configured to execute the one or more program instructions. In some embodiments, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of a CD274 nucleic acid molecule; and (c) detect, based on the analyzing, the CD274 nucleic acid molecule in the sample. In some embodiments, the sample is from an individual having a cancer, suspected of having cancer, being treated for cancer, or being tested for cancer.

In other aspects, provided herein are non-transitory computer readable storage media. In some embodiments, a non-transitory computer readable storage medium of the disclosure comprises one or more programs executable by one or more computer processors for performing a method. In some embodiments, the method comprises (a) obtaining, using the one or more processors, a plurality of sequence reads of one or more nucleic acid molecules, wherein the one or more nucleic acid molecules are derived from a sample obtained from an individual; (b) analyzing, using the one or more processors, the plurality of sequence reads for the presence of a CD274 nucleic acid molecule; and (c) detecting, using the one or more processors and based on the analyzing, the CD274 nucleic acid molecule in the sample. In some embodiments, the sample is from an individual having a cancer, suspected of having cancer, being treated for cancer, or being tested for cancer.

In some embodiments of any of the methods, systems, or non-transitory computer readable storage media provided herein, the CD274 nucleic acid molecule comprises or results from a rearrangement comprising a Breakpoint 1 and/or Breakpoint 2 within the chromosomal coordinates as listed in Table 1. In some embodiments of any of the methods, systems, or non-transitory computer readable storage media provided herein, the CD274 nucleic acid molecule is a CD274 fusion nucleic acid molecule comprising a fusion between a CD274 gene, or a portion thereof, and a fusion partner gene as listed in Table 2, or a portion thereof. In some embodiments of any of the methods, systems, or non-transitory computer readable storage media provided herein, the CD274 fusion nucleic acid molecule comprises a fusion between a CD274 gene, or a portion thereof, and a fusion partner gene as listed in Table 2, or a portion thereof, and the CD274 fusion nucleic acid molecule comprises or results from a corresponding CD274 Breakpoint and/or Fusion Partner Gene Breakpoint within the chromosomal coordinates as listed in Table 4. In some embodiments of any of the methods, systems, or non-transitory computer readable storage media provided herein, the cancer is a carcinoma, a sarcoma, a lymphoma, a leukemia, a myeloma, a germ cell cancer, or a blastoma. In some embodiments of any of the methods, systems, or non-transitory computer readable storage media provided herein, the cancer is a solid tumor or a hematologic malignancy. In some embodiments of any of the methods, systems, or non-transitory computer readable storage media provided herein, the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments of any of the methods, systems, or non-transitory computer readable storage media provided herein, the cancer is an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, unknown primary adenocarcinoma, gastric cancer or carcinoma, unknown primary carcinoma (CUP), unknown primary carcinoma (CUP) (NOS), unknown primary melanoma, stomach adenocarcinoma, or vagina squamous cell carcinoma (SCC). In some embodiments of any of the methods, systems, or non-transitory computer readable storage media provided herein, the CD274 nucleic acid molecule comprises or results from a rearrangement comprising a Breakpoint 1 and/or Breakpoint 2 within the chromosomal coordinates as listed in Table 1, and the cancer is the corresponding cancer as listed in Table 6. In some embodiments of any of the methods, systems, or non-transitory computer readable storage media provided herein, the CD274 fusion nucleic acid molecule comprises a fusion between a CD274 gene, or a portion thereof, and a fusion partner gene as listed in Table 2, or a portion thereof, and the cancer is the corresponding cancer as listed in Table 7.

In some embodiments of any of the methods, systems, or non-transitory computer readable storage media provided herein, the CD274 fusion nucleic acid molecule comprises a fusion between a CD274 gene, or a portion thereof, and a fusion partner gene as listed in Table 2, or a portion thereof; the CD274 fusion nucleic acid molecule comprises or results from a corresponding CD274 Breakpoint and/or Fusion Partner Gene Breakpoint within the chromosomal coordinates as listed in Table 8; and the cancer is the corresponding cancer as listed in Table 8.

In some embodiments of any of the methods, systems, or non-transitory computer readable storage media provided herein, the CD274 nucleic acid molecule is a CD274 fusion nucleic acid molecule comprising a fusion between a CD274 gene, or a portion thereof, and a fusion partner gene as listed in Table 3, or a portion thereof, wherein the cancer is the corresponding cancer as listed in Table 3. In some embodiments of any of the methods, systems, or non-transitory computer readable storage media provided herein, the CD274 fusion nucleic acid molecule comprises a fusion between a CD274 gene, or a portion thereof, and a fusion partner gene as listed in Table 3, or a portion thereof, the cancer is the corresponding cancer as listed in Table 3; and the CD274 fusion nucleic acid molecule comprises or results from a corresponding CD274 Breakpoint and/or Fusion Partner Gene Breakpoint within the chromosomal coordinates as listed in Table 5.

### A. CD274 Rearrangements and Nucleic Acid Molecules

Certain aspects of the present disclosure relate to genomic rearrangements involving a CD274 gene. A CD274 rearrangement of the present disclosure may relate to any chromosomal translocation, fusion, or rearrangement involving the locus of a CD274 gene. In some aspects, provided herein are CD274 nucleic acid molecules that result from or comprise any CD274 rearrangement described herein, as well as PD-L1 polypeptides encoded by such CD274 nucleic acid molecules. In some embodiments, the CD274 rearrangements of the disclosure result in a CD274 fusion nucleic acid molecule that comprises at least a portion of a CD274 gene fused to at least a portion of another gene. Accordingly, certain aspects of the present disclosure relate to CD274 fusion nucleic acid molecules comprising at least a portion of a CD274 gene fused to at least a portion of another gene, as well as to PD-L1 polypeptides encoded by such CD274 fusion nucleic acid molecules.

As used herein “cluster of differentiation 274” or “CD274” refer to a gene encoding a CD274 mRNA, or a Programmed death-ligand 1 (PD-L1) polypeptide. CD274 is also known as PD-L1. PDL1, B7-H1, B7-H, CD274 molecule, PDCD1L1, PDCD1LG1, cluster of differentiation 274, programmed death-ligand 1, and B7 homolog 1. In some embodiments, a CD274 gene is a human CD274 gene. An exemplary CD274 gene is represented by NCBI Gene ID No. 29126. An exemplary CD274 mRNA sequence is represented by NCBI Ref. Seq. NM_014143, provided below as SEQ ID NO: 1.

(SEQ ID NO: 1)
AGTTCTGCGCAGCTTCCCGAGGCTCCGCACCAGCCGCGCTTCTGTCCGCCTGCAGGGCATTCCAGAAAGA
TGAGGATATTTGCTGTCTTTATATTCATGACCTACTGGCATTTGCTGAACGCATTTACTGTCACGGTTCC
CAAGGACCTATATGTGGTAGAGTATGGTAGCAATATGACAATTGAATGCAAATTCCCAGTAGAAAAACAA
TTAGACCTGGCTGCACTAATTGTCTATTGGGAAATGGAGGATAAGAACATTATTCAATTTGTGCATGGAG
AGGAAGACCTGAAGGTTCAGCATAGTAGCTACAGACAGAGGGCCCGGCTGTTGAAGGACCAGCTCTCCCT
GGGAAATGCTGCACTTCAGATCACAGATGTGAAATTGCAGGATGCAGGGGTGTACCGCTGCATGATCAGC
TATGGTGGTGCCGACTACAAGCGAATTACTGTGAAAGTCAATGCCCCATACAACAAAATCAACCAAAGAA
TTTTGGTTGTGGATCCAGTCACCTCTGAACATGAACTGACATGTCAGGCTGAGGGCTACCCCAAGGCCGA
AGTCATCTGGACAAGCAGTGACCATCAAGTCCTGAGTGGTAAGACCACCACCACCAATTCCAAGAGAGAG
GAGAAGCTTTTCAATGTGACCAGCACACTGAGAATCAACACAACAACTAATGAGATTTTCTACTGCACTT
TTAGGAGATTAGATCCTGAGGAAAACCATACAGCTGAATTGGTCATCCCAGAACTACCTCTGGCACATCC
TCCAAATGAAAGGACTCACTTGGTAATTCTGGGAGCCATCTTATTATGCCTTGGTGTAGCACTGACATTC
ATCTTCCGTTTAAGAAAAGGGAGAATGATGGATGTGAAAAAATGTGGCATCCAAGATACAAACTCAAAGA
AGCAAAGTGATACACATTTGGAGGAGACGTAATCCAGCATTGGAACTTCTGATCTTCAAGCAGGGATTCT
CAACCTGTGGTTTAGGGGTTCATCGGGGCTGAGCGTGACAAGAGGAAGGAATGGGCCCGTGGGATGCAGG
CAATGTGGGACTTAAAAGGCCCAAGCACTGAAAATGGAACCTGGCGAAAGCAGAGGAGGAGAATGAAGAA
AGATGGAGTCAAACAGGGAGCCTGGAGGGAGACCTTGATACTTTCAAATGCCTGAGGGGCTCATCGACGC
CTGTGACAGGGAGAAAGGATACTTCTGAACAAGGAGCCTCCAAGCAAATCATCCATTGCTCATCCTAGGA
AGACGGGTTGAGAATCCCTAATTTGAGGGTCAGTTCCTGCAGAAGTGCCCTTTGCCTCCACTCAATGCCT
CAATTTGTTTTCTGCATGACTGAGAGTCTCAGTGTTGGAACGGGACAGTATTTATGTATGAGTTTTTCCT
ATTTATTTTGAGTCTGTGAGGTCTTCTTGTCATGTGAGTGTGGTTGTGAATGATTTCTTTTGAAGATATA
TTGTAGTAGATGTTACAATTTTGTCGCCAAACTAAACTTGCTGCTTAATGATTTGCTCACATCTAGTAAA
ACATGGAGTATTTGTAAGGTGCTTGGTCTCCTCTATAACTACAAGTATACATTGGAAGCATAAAGATCAA
ACCGTTGGTTGCATAGGATGTCACCTTTATTTAACCCATTAATACTCTGGTTGACCTAATCTTATTCTCA
GACCTCAAGTGTCTGTGCAGTATCTGTTCCATTTAAATATCAGCTTTACAATTATGTGGTAGCCTACACA
CATAATCTCATTTCATCGCTGTAACCACCCTGTTGTGATAACCACTATTATTTTACCCATCGTACAGCTG
AGGAAGCAAACAGATTAAGTAACTTGCCCAAACCAGTAAATAGCAGACCTCAGACTGCCACCCACTGTCC
TTTTATAATACAATTTACAGCTATATTTTACTTTAAGCAATTCTTTTATTCAAAAACCATTTATTAAGTG
CCCTTGCAATATCAATCGCTGTGCCAGGCATTGAATCTACAGATGTGAGCAAGACAAAGTACCTGTCCTC
AAGGAGCTCATAGTATAATGAGGAGATTAACAAGAAAATGTATTATTACAATTTAGTCCAGTGTCATAGC
ATAAGGATGATGCGAGGGGAAAACCCGAGCAGTGTTGCCAAGAGGAGGAAATAGGCCAATGTGGTCTGGG
ACGGTTGGATATACTTAAACATCTTAATAATCAGAGTAATTTTCATTTACAAAGAGAGGTCGGTACTTAA
AATAACCCTGAAAAATAACACTGGAATTCCTTTTCTAGCATTATATTTATTCCTGATTTGCCTTTGCCAT
ATAATCTAATGCTTGTTTATATAGTGTCTGGTATTGTTTAACAGTTCTGTCTTTTCTATTTAAATGCCAC
TAAATTTTAAATTCATACCTTTCCATGATTCAAAATTCAAAAGATCCCATGGGAGATGGTTGGAAAATCT
CCACTTCATCCTCCAAGCCATTCAAGTTTCCTTTCCAGAAGCAACTGCTACTGCCTTTCATTCATATGTT
CTTCTAAAGATAGTCTACATTTGGAAATGTATGTTAAAAGCACGTATTTTTAAAATTTTTTTCCTAAATA
GTAACACATTGTATGTCTGCTGTGTACTTTGCTATTTTTATTTATTTTAGTGTTTCTTATATAGCAGATG
GAATGAATTTGAAGTTCCCAGGGCTGAGGATCCATGCCTTCTTTGTTTCTAAGTTATCTTTCCCATAGCT
TTTCATTATCTTTCATATGATCCAGTATATGTTAAATATGTCCTACATATACATTTAGACAACCACCATT
TGTTAAGTATTTGCTCTAGGACAGAGTTTGGATTTGTTTATGTTTGCTCAAAAGGAGACCCATGGGCTCT
CCAGGGTGCACTGAGTCAATCTAGTCCTAAAAAGCAATCTTATTATTAACTCTGTATGACAGAATCATGT
CTGGAACTTTTGTTTTCTGCTTTCTGTCAAGTATAAACTTCACTTTGATGCTGTACTTGCAAAATCACAT
TTTCTTTCTGGAAATTCCGGCAGTGTACCTTGACTGCTAGCTACCCTGTGCCAGAAAAGCCTCATTCGTT
GTGCTTGAACCCTTGAATGCCACCAGCTGTCATCACTACACAGCCCTCCTAAGAGGCTTCCTGGAGGTTT
CGAGATTCAGATGCCCTGGGAGATCCCAGAGTTTCCTTTCCCTCTTGGCCATATTCTGGTGTCAATGACA
AGGAGTACCTTGGCTTTGCCACATGTCAAGGCTGAAGAAACAGTGTCTCCAACAGAGCTCCTTGTGTTAT
CTGTTTGTACATGTGCATTTGTACAGTAATTGGTGTGACAGTGTTCTTTGTGTGAATTACAGGCAAGAAT
TGTGGCTGAGCAAGGCACATAGTCTACTCAGTCTATTCCTAAGTCCTAACTCCTCCTTGTGGTGTTGGAT
TTGTAAGGCACTTTATCCCTTTTGTCTCATGTTTCATCGTAAATGGCATAGGCAGAGATGATACCTAATT
CTGCATTTGATTGTCACTTTTTGTACCTGCATTAATTTAATAAAATATTCTTATTTATTTTGTTACTTGG
TACACCAGCATGTCCATTTTCTTGTTTATTTTGTGTTTAATAAAATGTTCAGTTTAACATCCCA

An exemplary amino acid sequence of a PD-L1 polypeptide is represented by NCBI Ref. Seq. NP_054862, provided below as SEQ ID NO: 2.

(SEQ ID NO: 2)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

### (i) CD274 Rearrangements and Nucleic Acid Molecules

In some aspects, provided herein are CD274 rearrangements involving a CD274 locus. In some embodiments, such rearrangements comprise a Breakpoint 1 and/or Breakpoint 2 within the chromosomal coordinates as listed in Table 1 and/or described in the Examples herein. In certain other aspects, also provided herein are CD274 nucleic acid molecules comprising or resulting from a rearrangement involving a CD274 locus, such as a CD274 rearrangement described herein. In some embodiments, such CD274 nucleic acid molecules result from or comprise a rearrangement comprising a Breakpoint 1 and/or Breakpoint 2 within the chromosomal coordinates as listed in Table 1 and/or described in the Examples herein.

TABLE 1
Exemplary CD274 rearrangements.

| Gene | Breakpoint 1 | Breakpoint 2 |
|---|---|---|
| CD274 | chr9: 5466758-5466912 | chr9: 7819480-7819610 |
| CD274 | chr9: 5467780-5467988 | chr9: 6511502-6511679 |
| CD274 | chr9: 5467646-5467904 | chr9: 5447459-5447604 |
| CD274 | chr9: 5467655-5467913 | chr9: 5489388-5489533 |
| CD274 | chr9: 5466698-5466964 | chr9: 5582855-5583098 |
| CD274 | chr9: 5467714-5467907 | chr18: 28214456-28214689 |
| CD274 | chr9: 5466689-5466956 | chr9: 6676528-6676754 |
| CD274 | chr9: 5467714-5467957 | chr9: 4316216-4316474 |
| CD274 | chr9: 5467678-5467931 | chr9: 3590897-3591102 |
| CD274 | chr9: 5465440-5465671 | chr9: 5479019-5479384 |
| CD274 | chr9: 5465342-5465658 | chr9: 5270435-5270947 |
| CD274 | chr9: 5467863-5468050 | chr9: 5500043-5500268 |
| CD274 | chr9: 5467659-5467922 | chr2: 157519407-157519682 |
| CD274 | chr9: 5467787-5468003 | chr9: 5471671-5471863 |
| CD274 | chr9: 5467642-5467900 | chr6: 94722507-94722813 |
| CD274 | chr9: 5467648-5467991 | chr18: 27844666-27844942 |
| CD274 | chr9: 5466585-5466876 | chr9: 5201780-5202054 |
| CD274 | chr9: 5467638-5467928 | chr9: 5441513-5441883 |
| CD274 | chr9: 5467681-5467968 | chr9: 5198653-5198919 |
| CD274 | chr9: 5466590-5466996 | chr9: 5149992-5150287 |
| CD274 | chr9: 5467660-5467942 | chr9: 5479655-5479937 |
| CD274 | chr9: 5465343-5465699 | chr9: 8143594-8144044 |
| CD274 | chr9: 5467637-5467886 | chr9: 5484662-5485082 |
| CD274 | chr9: 5466636-5466908 | chr9: 5848084-5848343 |
| CD274 | chr9: 5467939-NA | chr9: 5486358-NA |
| CD274 | chr9: 5467786-5467955 | chr9: 5442048-5442165 |
| CD274 | chr9: 5467697-5467980 | chr4: 131103841-131104134 |
| CD274 | chr9: 5467704-5467951 | chr9: 5470872-5471004 |

In some embodiments, a CD274 rearrangement provided herein comprises a breakpoint within chromosomal coordinates chr9: 5466758-5466912 and/or chr9: 7819480-7819610. In some embodiments, a CD274 rearrangement provided herein comprises a breakpoint within chromosomal coordinates chr9: 5467780-5467988 and/or chr9: 6511502-6511679. In some embodiments, a CD274 rearrangement provided herein comprises a breakpoint within chromosomal coordinates chr9: 5467646-5467904 and/or chr9: 5447459-5447604. In some embodiments, a CD274 rearrangement provided herein comprises a breakpoint within chromosomal coordinates chr9: 5467655-5467913 and/or chr9: 5489388-5489533. In some embodiments, a CD274 rearrangement provided herein comprises a breakpoint within chromosomal coordinates chr9: 5466698-5466964 and/or chr9: 5582855-5583098. In some embodiments, a CD274 rearrangement provided herein comprises a breakpoint within chromosomal coordinates chr9: 5467714-5467907 and/or chr18: 28214456-28214689. In some embodiments, a CD274 rearrangement provided herein comprises a breakpoint within chromosomal coordinates chr9: 5466689-5466956 and/or chr9: 6676528-6676754. In some embodiments, a CD274 rearrangement provided herein comprises a breakpoint within chromosomal coordinates chr9: 5467714-5467957 and/or chr9: 4316216-4316474. In some embodiments, a CD274 rearrangement provided herein comprises a breakpoint within chromosomal coordinates chr9: 5467678-5467931 and/or chr9: 3590897-3591102. In some embodiments, a CD274 rearrangement provided herein comprises a breakpoint within chromosomal coordinates chr9: 5465440-5465671 and/or chr9: 5479019-5479384. In some embodiments, a CD274 rearrangement provided herein comprises a breakpoint within chromosomal coordinates chr9: 5465342-5465658 and/or chr9: 5270435-5270947. In some embodiments, a CD274 rearrangement provided herein comprises a breakpoint within chromosomal coordinates chr9: 5467863-5468050 and/or chr9: 5500043-5500268. In some embodiments, a CD274 rearrangement provided herein comprises a breakpoint within chromosomal coordinates chr9: 5467659-5467922 and/or chr2: 157519407-157519682. In some embodiments, a CD274 rearrangement provided herein comprises a breakpoint within chromosomal coordinates chr9: 5467787-5468003 and/or chr9: 5471671-5471863. In some embodiments, a CD274 rearrangement provided herein comprises a breakpoint within chromosomal coordinates chr9: 5467642-5467900 and/or chr6: 94722507-94722813. In some embodiments, a CD274 rearrangement provided herein comprises a breakpoint within chromosomal coordinates chr9: 5467648-5467991 and/or chr18: 27844666-27844942. In some embodiments, a CD274 rearrangement provided herein comprises a breakpoint within chromosomal coordinates chr9: 5466585-5466876 and/or chr9: 5201780-5202054. In some embodiments, a CD274 rearrangement provided herein comprises a breakpoint within chromosomal coordinates chr9: 5467638-5467928 and/or chr9: 5441513-5441883. In some embodiments, a CD274 rearrangement provided herein comprises a breakpoint within chromosomal coordinates chr9: 5467681-5467968 and/or chr9: 5198653-5198919. In some embodiments, a CD274 rearrangement provided herein comprises a breakpoint within chromosomal coordinates chr9: 5466590-5466996 and/or chr9: 5149992-5150287. In some embodiments, a CD274 rearrangement provided herein comprises a breakpoint within chromosomal coordinates chr9: 5467660-5467942 and/or chr9: 5479655-5479937. In some embodiments, a CD274 rearrangement provided herein comprises a breakpoint within chromosomal coordinates chr9: 5465343-5465699 and/or chr9: 8143594-8144044. In some embodiments, a CD274 rearrangement provided herein comprises a breakpoint within chromosomal coordinates chr9: 5467637-5467886 and/or chr9: 5484662-5485082. In some embodiments, a CD274 rearrangement provided herein comprises a breakpoint within chromosomal coordinates chr9: 5466636-5466908 and/or chr9: 5848084-5848343. In some embodiments, a CD274 rearrangement provided herein comprises a breakpoint within chromosomal coordinates chr9:5467939-NA and/or chr9:5486358-NA. In some embodiments, a CD274 rearrangement provided herein comprises a breakpoint within chromosomal coordinates chr9:5467939 and/or chr9:5486358. In some embodiments, a CD274 rearrangement provided herein comprises a breakpoint within chromosomal coordinates chr9: 5467786-5467955 and/or chr9: 5442048-5442165. In some embodiments, a CD274 rearrangement provided herein comprises a breakpoint within chromosomal coordinates chr9: 5467697-5467980 and/or chr4: 131103841-131104134. In some embodiments, a CD274 rearrangement provided herein comprises a breakpoint within chromosomal coordinates chr9: 5467704-5467951 and/or chr9: 5470872-5471004. In some embodiments, a CD274 rearrangement provided herein results from a truncation rearrangement, optionally wherein the CD274 rearrangement comprises a chromosome 9 inversion fragment involving the CD274 locus with a 3′ rearrangement breakpoint in intron 6 of CD274; and optionally, wherein the rearrangement comprises a breakpoint within chromosomal coordinates chr9: 5466636-5466908 and/or chr9: 5848084-5848343. In some embodiments, a CD274 rearrangement provided herein results from a rearrangement comprising a chromosome 9 deletion fragment involving the CD274 locus with a 3′ rearrangement breakpoint in exon 7 of CD274; and optionally, wherein the rearrangement comprises a breakpoint within chromosomal coordinates chr9:5467939-NA (or chr9:5467939) and/or chr9:5486358-NA (or chr9:5486358). In some embodiments, a CD274 rearrangement provided herein results from a rearrangement comprising a chromosome 9 duplication fragment involving the CD274 locus with a 5′ rearrangement breakpoint in exon 7 of CD274; and optionally, wherein the rearrangement comprises a breakpoint within chromosomal coordinates chr9: 5467786-5467955 and/or chr9: 5442048-5442165. In some embodiments, a CD274 rearrangement provided herein results from a rearrangement comprising a chromosome 9 translocation involving the CD274 locus with a 5′ rearrangement breakpoint in exon 7 of CD274; and optionally, wherein the rearrangement comprises a breakpoint within chromosomal coordinates chr9: 5467697-5467980 and/or chr4: 131103841-131104134. In some embodiments, a CD274 rearrangement provided herein results from a rearrangement comprising a chromosome 9 deletion fragment involving the CD274 locus with a 3′ deletion and a breakpoint in exon 7 and/or the untranslated region (UTR) of CD274; and optionally, wherein the rearrangement comprises a breakpoint within chromosomal coordinates chr9: 5467704-5467951 and/or chr9: 5470872-5471004. In some embodiments, a CD274 rearrangement provided herein results from a rearrangement comprising translocation with a 5′ breakpoint in exon 7 of CD274.

In some embodiments, a CD274 nucleic acid molecule provided herein comprises or results from a breakpoint within chromosomal coordinates chr9: 5466758-5466912 and/or chr9: 7819480-7819610. In some embodiments, a CD274 nucleic acid molecule provided herein comprises or results from a breakpoint within chromosomal coordinates chr9: 5467780-5467988 and/or chr9: 6511502-6511679. In some embodiments, a CD274 nucleic acid molecule provided herein comprises or results from a breakpoint within chromosomal coordinates chr9: 5467646-5467904 and/or chr9: 5447459-5447604. In some embodiments, a CD274 nucleic acid molecule provided herein comprises or results from a breakpoint within chromosomal coordinates chr9: 5467655-5467913 and/or chr9: 5489388-5489533. In some embodiments, a CD274 nucleic acid molecule provided herein comprises or results from a breakpoint within chromosomal coordinates chr9: 5466698-5466964 and/or chr9: 5582855-5583098. In some embodiments, a CD274 nucleic acid molecule provided herein comprises or results from a breakpoint within chromosomal coordinates chr9: 5467714-5467907 and/or chr18: 28214456-28214689. In some embodiments, a CD274 nucleic acid molecule provided herein comprises or results from a breakpoint within chromosomal coordinates chr9: 5466689-5466956 and/or chr9: 6676528-6676754. In some embodiments, a CD274 nucleic acid molecule provided herein comprises or results from a breakpoint within chromosomal coordinates chr9: 5467714-5467957 and/or chr9: 4316216-4316474. In some embodiments, a CD274 nucleic acid molecule provided herein comprises or results from a breakpoint within chromosomal coordinates chr9: 5467678-5467931 and/or chr9: 3590897-3591102. In some embodiments, a CD274 nucleic acid molecule provided herein comprises or results from a breakpoint within chromosomal coordinates chr9: 5465440-5465671 and/or chr9: 5479019-5479384. In some embodiments, a CD274 nucleic acid molecule provided herein comprises or results from a breakpoint within chromosomal coordinates chr9: 5465342-5465658 and/or chr9: 5270435-5270947. In some embodiments, a CD274 nucleic acid molecule provided herein comprises or results from a breakpoint within chromosomal coordinates chr9: 5467863-5468050 and/or chr9: 5500043-5500268. In some embodiments, a CD274 nucleic acid molecule provided herein comprises or results from a breakpoint within chromosomal coordinates chr9: 5467659-5467922 and/or chr2: 157519407-157519682. In some embodiments, a CD274 nucleic acid molecule provided herein comprises or results from a breakpoint within chromosomal coordinates chr9: 5467787-5468003 and/or chr9: 5471671-5471863. In some embodiments, a CD274 nucleic acid molecule provided herein comprises or results from a breakpoint within chromosomal coordinates chr9: 5467642-5467900 and/or chr6: 94722507-94722813. In some embodiments, a CD274 nucleic acid molecule provided herein comprises or results from a breakpoint within chromosomal coordinates chr9: 5467648-5467991 and/or chr18: 27844666-27844942. In some embodiments, a CD274 nucleic acid molecule provided herein comprises or results from a breakpoint within chromosomal coordinates chr9: 5466585-5466876 and/or chr9: 5201780-5202054. In some embodiments, a CD274 nucleic acid molecule provided herein comprises or results from a breakpoint within chromosomal coordinates chr9: 5467638-5467928 and/or chr9: 5441513-5441883. In some embodiments, a CD274 nucleic acid molecule provided herein comprises or results from a breakpoint within chromosomal coordinates chr9: 5467681-5467968 and/or chr9: 5198653-5198919. In some embodiments, a CD274 nucleic acid molecule provided herein comprises or results from a breakpoint within chromosomal coordinates chr9: 5466590-5466996 and/or chr9: 5149992-5150287. In some embodiments, a CD274 nucleic acid molecule provided herein comprises or results from a breakpoint within chromosomal coordinates chr9: 5467660-5467942 and/or chr9: 5479655-5479937. In some embodiments, a CD274 nucleic acid molecule provided herein comprises or results from a breakpoint within chromosomal coordinates chr9: 5465343-5465699 and/or chr9: 8143594-8144044. In some embodiments, a CD274 nucleic acid molecule provided herein comprises or results from a breakpoint within chromosomal coordinates chr9: 5467637-5467886 and/or chr9: 5484662-5485082. In some embodiments, a CD274 nucleic acid molecule provided herein comprises or results from a breakpoint within chromosomal coordinates chr9: 5466636-5466908 and/or chr9: 5848084-5848343. In some embodiments, a CD274 nucleic acid molecule provided herein comprises or results from a breakpoint within chromosomal coordinates chr9:5467939-NA and/or chr9:5486358-NA. In some embodiments, a CD274 nucleic acid molecule provided herein comprises or results from a breakpoint within chromosomal coordinates chr9:5467939 and/or chr9:5486358. In some embodiments, a CD274 nucleic acid molecule provided herein comprises or results from a breakpoint within chromosomal coordinates chr9: 5467786-5467955 and/or chr9: 5442048-5442165. In some embodiments, a CD274 nucleic acid molecule provided herein comprises or results from a breakpoint within chromosomal coordinates chr9: 5467697-5467980 and/or chr4: 131103841-131104134. In some embodiments, a CD274 nucleic acid molecule provided herein comprises or results from a breakpoint within chromosomal coordinates chr9: 5467704-5467951 and/or chr9: 5470872-5471004. In some embodiments, a CD274 nucleic acid molecule provided herein comprises or results from a truncation rearrangement, optionally wherein the rearrangement comprises a chromosome 9 inversion fragment involving the CD274 locus with a 3′ rearrangement breakpoint in intron 6 of CD274; and optionally, wherein the rearrangement comprises a breakpoint within chromosomal coordinates chr9: 5466636-5466908 and/or chr9: 5848084-5848343. In some embodiments, a CD274 nucleic acid molecule provided herein comprises or results from a rearrangement comprising a chromosome 9 deletion fragment involving the CD274 locus with a 3′ rearrangement breakpoint in exon 7 of CD274; and optionally, wherein the rearrangement comprises a breakpoint within chromosomal coordinates chr9:5467939-NA (or chr9:5467939) and/or chr9:5486358-NA (or chr9:5486358). In some embodiments, a CD274 nucleic acid molecule provided herein comprises or results from a rearrangement comprising a chromosome 9 duplication fragment involving the CD274 locus with a 5′ rearrangement breakpoint in exon 7 of CD274; and optionally, wherein the rearrangement comprises a breakpoint within chromosomal coordinates chr9: 5467786-5467955 and/or chr9: 5442048-5442165. In some embodiments, a CD274 nucleic acid molecule provided herein comprises or results from a rearrangement comprising a chromosome 9 translocation involving the CD274 locus with a 5′ rearrangement breakpoint in exon 7 of CD274; and optionally, wherein the rearrangement comprises a breakpoint within chromosomal coordinates chr9: 5467697-5467980 and/or chr4: 131103841-131104134. In some embodiments, a CD274 nucleic acid molecule provided herein comprises or results from a rearrangement comprising a chromosome 9 deletion fragment involving the CD274 locus with a 3′ deletion and a breakpoint in exon 7 and/or the untranslated region (UTR) of CD274; and optionally, wherein the rearrangement comprises a breakpoint within chromosomal coordinates chr9: 5467704-5467951 and/or chr9: 5470872-5471004. In some embodiments, a CD274 nucleic acid molecule provided herein comprises or results from a rearrangement comprising translocation with a 5′ breakpoint in exon 7 of CD274.

### (ii) CD274 Fusion Nucleic Acid Molecules

In some aspects, provided herein are CD274 fusion nucleic acid molecules. In some embodiments, a CD274 fusion nucleic acid molecule comprises at least a portion of a CD274 gene, and at least a portion of another gene.

In some embodiments, a CD274 fusion nucleic acid molecule of the disclosure comprises at least a portion of a CD274 gene and at least a portion of a CD274, CFAP52, MYLK, PTPRD, RIC1, HIPK2, PDCD1LG2, ERMP1, CHMP5, PTPRB, PLGRKT, or JAK2 gene. For example, in some embodiments, the CD274 fusion nucleic acid molecule is a CD274-CD274, CD274-CFAP52, CD274-MYLK, CD274-PTPRD, CD274-RIC1, CD274-HIPK2, CD274-PDCD1LG2, CD274-ERMP1, CD274-CHMP5, CD274-PTPRB, CD274-PLGRKT, or CD274-JAK2 fusion nucleic acid molecule.

Certain exemplary CD274 fusion nucleic acid molecules are provided in Tables 2-3, below.

TABLE 2
Exemplary CD274 fusion nucleic acid molecules.

| Gene | Fusion Partner Gene |
|---|---|
| CD274 | CFAP52 |
| CD274 | MYLK |
| CD274 | PTPRD |
| CD274 | RIC1 |
| CD274 | HIPK2 |
| CD274 | PDCD1LG2 |
| CD274 | ERMP1 |
| CD274 | CHMP5 |
| CD274 | PTPRB |

TABLE 3
Exemplary CD274 fusion nucleic acid molecules,
identified in the indicated cancer types.

| Cancer Type | Gene | Fusion Partner Gene |
|---|---|---|
| Unknown primary | CD274 | PLGRKT |
| adenocarcinoma | | |
| Bladder carcinoma (NOS) | CD274 | CD274 |
| Skin melanoma | CD274 | PLGRKT |
| Cervix adenocarcinoma | CD274 | PLGRKT |
| Lung adenocarcinoma | CD274 | PLGRKT |
| Vagina squamous cell | CD274 | JAK2 |
| carcinoma (SCC) | | |
| Unknown primary carcinoma | CD274 | PLGRKT |
| (CUP) (NOS) | | |
| Unknown primary | CD274 | PLGRKT |
| adenocarcinoma | | |
| Kidney renal cell carcinoma | CD274 | PLGRKT |
| Lung non-squamous cell lung | CD274 | PLGRKT |
| adenocarcinoma | | |
| Lung squamous cell | CD274 | CD274 |
| carcinoma | | |

SCC: squamous cell carcinoma;
CUP: carcinoma of unknown primary;
NOS: not otherwise specified.

As used herein “CFAP52” refers to a gene encoding a CFAP52 mRNA or polypeptide. The CFAP52 gene encodes the cilia and flagella associated protein 52 protein. CFAP52 is also known as WDR16 and WDRPUH. In some embodiments, a CFAP52 gene is a human CFAP52 gene. An exemplary CFAP52 gene is represented by NCBI Gene ID No. 146845. An exemplary CFAP52 mRNA sequence is represented by NCBI Ref. Seq. NM_001080556. An exemplary amino acid sequence of a CFAP52 polypeptide is represented by NCBI Ref. Seq. NP_001074025.

As used herein “MYLK” refers to a gene encoding a MYLK mRNA or polypeptide. The MYLK gene encodes the myosin light chain kinase. MYLK is also known as KRP, AAT7, MLCK, MLCK1, MMIHS, MYLK1, MMIHS1, smMLCK, MLCK108, MLCK210, and MSTP083. In some embodiments, a MYLK gene is a human MYLK gene. An exemplary MYLK gene is represented by NCBI Gene ID No. 4638. An exemplary MYLK mRNA sequence is represented by NCBI Ref. Seq. NM_001321309. An exemplary amino acid sequence of a MYLK polypeptide is represented by NCBI Ref. Seq. NP_001308238.

As used herein “PTPRD” refers to a gene encoding a PTPRD mRNA or polypeptide. The PTPRD gene encodes the protein tyrosine phosphatase receptor type D protein. PTPRD is also known as HPTP, PTPD, HPTPD, HPTPDELTA, RPTPDELTA, and R-PTP-delta. In some embodiments, a PTPRD gene is a human PTPRD gene. An exemplary PTPRD gene is represented by NCBI Gene ID No. 5789. An exemplary PTPRD mRNA sequence is represented by NCBI Ref. Seq. NM_001040712. An exemplary amino acid sequence of a PTPRD polypeptide is represented by NCBI Ref. Seq. NP_001035802.

As used herein “RIC1” refers to a gene encoding a RIC1 mRNA or polypeptide. The RIC1 gene encodes the RIC1 homolog, RAB6A GEF complex partner 1 protein. RIC1 is also known as CATIFA, CIP150, KIAA1432, and bA207C16.1. In some embodiments, a RIC1 gene is a human RIC1 gene. An exemplary RIC1 gene is represented by NCBI Gene ID No. 57589. An exemplary RIC1 mRNA sequence is represented by NCBI Ref. Seq. NM_001135920. An exemplary amino acid sequence of a RIC1 polypeptide is represented by NCBI Ref. Seq. NP_001129392.

As used herein “HIPK2” refers to a gene encoding a HIPK2 mRNA or polypeptide. The HIPK2 gene encodes the homeodomain interacting protein kinase 2 protein. HIPK2 is also known as PRO0593. In some embodiments, a HIPK2 gene is a human HIPK2 gene. An exemplary HIPK2 gene is represented by NCBI Gene ID No. 28996. An exemplary HIPK2 mRNA sequence is represented by NCBI Ref. Seq. NM_001113239. An exemplary amino acid sequence of a HIPK2 polypeptide is represented by NCBI Ref. Seq. NP_001106710.

As used herein “PDCD1LG2” refers to a gene encoding a PDCD1LG2 mRNA or polypeptide. The PDCD1LG2 gene encodes the programed cell death 1 ligand 2 protein. PDCD1LG2 is also known as B7DC, Btdc, PDL2, CD273, PD-L2, PDCD1L2, and bA574F11.2. In some embodiments, a PDCD1LG2 gene is a human PDCD1LG2 gene. An exemplary PDCD1LG2 gene is represented by NCBI Gene ID No. 80380. An exemplary PDCD1LG2 mRNA sequence is represented by NCBI Ref. Seq. NM_025239. An exemplary amino acid sequence of a PDCD1LG2 polypeptide is represented by NCBI Ref. Seq. NP_079515.

As used herein “ERMP1” refers to a gene encoding an ERMP1 mRNA or polypeptide. The ERMP1 gene encodes the endoplasmic reticulum metallopeptidase 1 protein. ERMP1 is also known as FXNA, KIAA1815, and bA207C16.3. In some embodiments, an ERMP1 gene is a human ERMP1 gene. An exemplary ERMP1 gene is represented by NCBI Gene ID No. 79956. An exemplary ERMP1 mRNA sequence is represented by NCBI Ref. Seq. NM_024896. An exemplary amino acid sequence of an ERMP1 polypeptide is represented by NCBI Ref. Seq. NP_079172.

As used herein “CHMP5” refers to a gene encoding a CHMP5 mRNA or polypeptide. The CHMP5 gene encodes the charged multivesicular body protein 5. CHMP5 is also known as Vps60, CGI-34, PNAS-2, C9orf83, HSPC177, and SNF7DC2. In some embodiments, a CHMP5 gene is a human CHMP5 gene. An exemplary CHMP5 gene is represented by NCBI Gene ID No. 51510. An exemplary CHMP5 mRNA sequence is represented by NCBI Ref. Seq. NM_001195536. An exemplary amino acid sequence of a CHMP5 polypeptide is represented by NCBI Ref. Seq. NP_001182465.

As used herein “PTPRB” refers to a gene encoding a PTPRB mRNA or polypeptide. The PTPRB gene encodes the protein tyrosine phosphatase receptor type B protein. PTPRB is also known as PTPB, HPTPB, VEPTP, HPTP-BETA, and R-PTP-BETA. In some embodiments, a PTPRB gene is a human PTPRB gene. An exemplary PTPRB gene is represented by NCBI Gene ID No. 5787. An exemplary PTPRB mRNA sequence is represented by NCBI Ref. Seq. NM_001109754. An exemplary amino acid sequence of a PTPRB polypeptide is represented by NCBI Ref. Seq. NP_001103224.

As used herein “PLGRKT” refers to a gene encoding a PLGRKT mRNA or polypeptide. The PLGRKT gene encodes the plasminogen receptor with a C-terminal lysine protein. PLGRKT is also known as AD025, MDS030, C9orf46, PLG-RKT, and Plg-R (KT). In some embodiments, a PLGRKT gene is a human PLGRKT gene. An exemplary PLGRKT gene is represented by NCBI Gene ID No. 55848. An exemplary PLGRKT mRNA sequence is represented by NCBI Ref. Seq. NM_018465. An exemplary amino acid sequence of a PLGRKT polypeptide is represented by NCBI Ref. Seq. NP_060935.

As used herein “JAK2” refers to a gene encoding a JAK2 mRNA or polypeptide. The JAK2 gene encodes the Janus kinase 2 protein. JAK2 is also known as JKT10. In some embodiments, a JAK2 gene is a human JAK2 gene. An exemplary JAK2 gene is represented by NCBI Gene ID No. 3717. An exemplary JAK2 mRNA sequence is represented by NCBI Ref. Seq. NM_004972. An exemplary amino acid sequence of a JAK2 polypeptide is represented by NCBI Ref. Seq. NP_004963.

In some embodiments, a CD274 fusion nucleic acid molecule provided herein is a CD274-CFAP52 fusion nucleic acid molecule comprising or resulting from a breakpoint within chromosomal coordinates chr9: 5466613-5466970 and/or chr17:9496835-9497088. In some embodiments, a CD274 fusion nucleic acid molecule provided herein is a CD274-MYLK fusion nucleic acid molecule comprising or resulting from a breakpoint within chromosomal coordinates chr9: 5465373-5465731 and/or chr3: 123592212-123592310. In some embodiments, a CD274 fusion nucleic acid molecule provided herein is a CD274-PTPRD fusion nucleic acid molecule comprising or resulting from a breakpoint within chromosomal coordinates chr9: 5467743-5467994 and/or chr9: 9745527-9745899. In some embodiments, a CD274 fusion nucleic acid molecule provided herein is a CD274-RIC1 fusion nucleic acid molecule comprising or resulting from a breakpoint within chromosomal coordinates chr9: 5466596-5466853 and/or chr9: 5731619-5731896. In some embodiments, a CD274 fusion nucleic acid molecule provided herein is a CD274-HIPK2 fusion nucleic acid molecule comprising or resulting from a breakpoint within chromosomal coordinates chr9: 5466598-5467002 and/or chr7: 139280432-139281022. In some embodiments, a CD274 fusion nucleic acid molecule provided herein is a CD274-PTPRD fusion nucleic acid molecule comprising or resulting from a breakpoint within chromosomal coordinates chr9: 5467715-5467879 and/or chr9: 8809122-8809281. In some embodiments, a CD274 fusion nucleic acid molecule provided herein is a CD274-PDCD1LG2 fusion nucleic acid molecule comprising or resulting from a breakpoint within chromosomal coordinates chr9: 5466586-5466770 and/or chr9: 5543484-5543698. In some embodiments, a CD274 fusion nucleic acid molecule provided herein is a CD274-ERMP1 fusion nucleic acid molecule comprising or resulting from a breakpoint within chromosomal coordinates chr9: 5467653-5467891 and/or chr9: 5789739-5790086. In some embodiments, a CD274 fusion nucleic acid molecule provided herein is a CD274-ERMP1 fusion nucleic acid molecule comprising or resulting from a rearrangement comprising a chromosome 9 deletion fragment involving the CD274 locus with a 3′ breakpoint in exon 7 of CD274; optionally wherein the CD274-ERMP1 fusion nucleic acid molecule comprises or results from a breakpoint within chromosomal coordinates chr9: 5467653-5467891 and/or chr9: 5789739-5790086. In some embodiments, a CD274 fusion nucleic acid molecule provided herein is a CD274-CHMP5 fusion nucleic acid molecule comprising or resulting from a breakpoint within chromosomal coordinates chr9: 5465378-5465756 and/or chr9: 33281589-33281786. In some embodiments, a CD274 fusion nucleic acid molecule provided herein is a CD274-PTPRB fusion nucleic acid molecule comprising or resulting from a breakpoint within chromosomal coordinates chr9: 5467734-5467932 and/or chr12: 70937973-70938263. In some embodiments, a CD274 fusion nucleic acid molecule provided herein is a CD274 fusion nucleic acid molecule listed in any of Tables 2, 4, 7 or 8, comprising or resulting from a breakpoint (e.g., a CD274 Breakpoint and/or Fusion Partner Gene Breakpoint) within the corresponding chromosomal coordinates as listed in any of Tables 4 or 8.

TABLE 4
Exemplary CD274 fusion nucleic acid molecules and corresponding breakpoints.

| Gene | Fusion Partner Gene | CD274 Breakpoint | Fusion Partner Gene Breakpoint |
|---|---|---|---|
| CD274 | CFAP52 | chr9: 5466613-5466970 | chr17: 9496835-9497088 |
| CD274 | MYLK | chr9: 5465373-5465731 | chr3: 123592212-123592310 |
| CD274 | PTPRD | chr9: 5467743-5467994 | chr9: 9745527-9745899 |
| CD274 | RIC1 | chr9: 5466596-5466853 | chr9: 5731619-5731896 |
| CD274 | HIPK2 | chr9: 5466598-5467002 | chr7: 139280432-139281022 |
| CD274 | PTPRD | chr9: 5467715-5467879 | chr9: 8809122-8809281 |
| CD274 | PDCD1LG2 | chr9: 5466586-5466770 | chr9: 5543484-5543698 |
| CD274 | ERMP1 | chr9: 5467653-5467891 | chr9: 5789739-5790086 |
| CD274 | CHMP5 | chr9: 5465378-5465756 | chr9: 33281589-33281786 |
| CD274 | PTPRB | chr9: 5467734-5467932 | chr12: 70937973-70938263 |

In some embodiments, a CD274 fusion nucleic acid molecule provided herein is a CD274-PLGRKT fusion nucleic acid molecule comprising or resulting from a breakpoint within chromosomal coordinates chr9: 5465378-5465661 and/or chr9: 5428506-5428752. In some embodiments, a CD274 fusion nucleic acid molecule provided herein is a CD274-CD274 fusion nucleic acid molecule comprising or resulting from a breakpoint within chromosomal coordinates chr9: 5467755-5467993 and/or chr9: 5470492-5470836. In some embodiments, a CD274 fusion nucleic acid molecule provided herein is a CD274-PLGRKT fusion nucleic acid molecule comprising or resulting from a breakpoint within chromosomal coordinates chr9: 5466638-5466892 and/or chr9: 5399714-5400057. In some embodiments, a CD274 fusion nucleic acid molecule provided herein is a CD274-PLGRKT fusion nucleic acid molecule comprising or resulting from a breakpoint within chromosomal coordinates chr9: 5465459-5465671 and/or chr9: 5421268-5421464. In some embodiments, a CD274 fusion nucleic acid molecule provided herein is a CD274-PLGRKT fusion nucleic acid molecule comprising or resulting from a breakpoint within chromosomal coordinates chr9: 5467639-5467873 and/or chr9: 5420662-5421059. In some embodiments, a CD274 fusion nucleic acid molecule provided herein is a CD274-JAK2 fusion nucleic acid molecule comprising or resulting from a breakpoint within chromosomal coordinates chr9: 5467855-5467998 and/or chr9: 5005621-5005795. In some embodiments, a CD274 fusion nucleic acid molecule provided herein is a CD274-JAK2 fusion nucleic acid molecule comprising a fusion of exon 4, or a portion thereof, of JAK2 fused to exon 7, or a portion thereof, of CD274; optionally wherein the CD274-JAK2 fusion nucleic acid molecule comprises, in the 5′ to 3′ direction, exons 1-3, and exon 4 or a portion thereof, of JAK2 and exon 7 or a portion thereof of CD274. In some embodiments, a CD274 fusion nucleic acid molecule provided herein is a CD274-PLGRKT fusion nucleic acid molecule comprising or resulting from a breakpoint within chromosomal coordinates chr9: 5467709-5467965 and/or chr9: 5429881-5430057. In some embodiments, a CD274 fusion nucleic acid molecule provided herein is a CD274-PLGRKT fusion nucleic acid molecule comprising or resulting from a breakpoint within chromosomal coordinates chr9: 5467661-5467911 and/or chr9: 5424930-5425336. In some embodiments, a CD274 fusion nucleic acid molecule provided herein is a CD274-PLGRKT fusion nucleic acid molecule comprising or resulting from a breakpoint within chromosomal coordinates chr9: 5467674-5467950 and/or chr9: 5425490-5425913. In some embodiments, a CD274 fusion nucleic acid molecule provided herein is a CD274-PLGRKT fusion nucleic acid molecule comprising or resulting from a breakpoint within chromosomal coordinates chr9:5467965-NA and/or chr9:5407366-NA. In some embodiments, a CD274 fusion nucleic acid molecule provided herein is a CD274-PLGRKT fusion nucleic acid molecule comprising or resulting from a breakpoint within chromosomal coordinates chr9:5467965 and/or chr9:5407366. In some embodiments, a CD274 fusion nucleic acid molecule provided herein is a CD274-PLGRKT fusion nucleic acid molecule comprising or resulting from a rearrangement comprising a chromosome 9 duplication fragment involving the CD274 locus with a 5′ breakpoint in exon 7 of CD274; optionally wherein the CD274-PLGRKT fusion nucleic acid molecule comprises or results from a breakpoint within chromosomal coordinates chr9:5467965-NA (or chr9:5467965) and/or chr9:5407366-NA (or chr9:5407366). In some embodiments, a CD274 fusion nucleic acid molecule provided herein is a CD274-CD274 fusion nucleic acid molecule comprising or resulting from a breakpoint within chromosomal coordinates chr9: 5465342-5465658 and/or chr9: 5270435-5270947. In some embodiments, a CD274 fusion nucleic acid molecule provided herein is a CD274-CD274 fusion nucleic acid molecule comprising or resulting from a rearrangement comprising a chromosome 9 duplication fragment involving the CD274 locus with a 5′ breakpoint in intron 5 of CD274; optionally wherein the CD274-CD274 fusion nucleic acid molecule comprises or results from a breakpoint within chromosomal coordinates chr9: 5465342-5465658 and/or chr9: 5270435-5270947. In some embodiments, a CD274 fusion nucleic acid molecule provided herein is a CD274 fusion nucleic acid molecule listed in any of Tables 3 or 5, comprising or resulting from a breakpoint (e.g., a CD274 Breakpoint and/or Fusion Partner Gene Breakpoint) within the corresponding chromosomal coordinates as listed in Table 5.

TABLE 5
Exemplary CD274 fusion nucleic acid molecules and corresponding breakpoints,
identified in the indicated cancer types.

| | | Fusion | | Fusion Partner |
|---|---|---|---|---|
| Cancer Type | Gene | Partner Gene | CD274 Breakpoint | Gene Breakpoint |
| Unknown | CD274 | PLGRKT | chr9: 5465378-5465661 | chr9: 5428506-5428752 |
| primary | | | | |
| adenocarcinoma | | | | |
| Bladder | CD274 | CD274 | chr9: 5467755-5467993 | chr9: 5470492-5470836 |
| carcinoma | | | | |
| (NOS) | | | | |
| Skin melanoma | CD274 | PLGRKT | chr9: 5466638-5466892 | chr9: 5399714-5400057 |
| Cervix | CD274 | PLGRKT | chr9: 5465459-5465671 | chr9: 5421268-5421464 |
| adenocarcinoma | | | | |
| Lung | CD274 | PLGRKT | chr9: 5467639-5467873 | chr9: 5420662-5421059 |
| adenocarcinoma | | | | |
| Vagina | CD274 | JAK2 | chr9: 5467855-5467998 | chr9: 5005621-5005795 |
| squamous cell | | | | |
| carcinoma | | | | |
| (SCC) | | | | |
| Unknown | CD274 | PLGRKT | chr9: 5467709-5467965 | chr9: 5429881-5430057 |
| primary | | | | |
| carcinoma | | | | |
| (CUP) (NOS) | | | | |
| Unknown | CD274 | PLGRKT | chr9: 5467661-5467911 | chr9: 5424930-5425336 |
| primary | | | | |
| adenocarcinoma | | | | |
| Kidney renal | CD274 | PLGRKT | chr9: 5467674-5467950 | chr9: 5425490-5425913 |
| cell carcinoma | | | | |
| Lung non- | CD274 | PLGRKT | chr9: 5467965-NA | chr9: 5407366-NA |
| squamous cell | | | | |
| lung | | | | |
| adenocarcinoma | | | | |
| Lung | CD274 | CD274 | chr9: 5465342-5465658 | chr9: 5270435-5270947 |
| squamous cell | | | | |
| carcinoma | | | | |

SCC: squamous cell carcinoma;
CUP: carcinoma of unknown primary;
NOS: not otherwise specified.

(iii) PD-L1 Polypeptides

In certain aspects, provided herein are PD-L1 polypeptides which comprise at least a portion of a PD-L1 polypeptide encoded by a CD274 gene. In some embodiments, a PD-L1 polypeptide of the disclosure is a PD-L1 polypeptide encoded by any of the CD274 nucleic acid molecules provided herein, or by a nucleic acid molecule comprising or resulting from any of the CD274 rearrangements described herein.

In some embodiments, a PD-L1 polypeptide of the disclosure is encoded by a CD274 nucleic acid molecule resulting from or comprising a rearrangement comprising a Breakpoint 1 and/or Breakpoint 2 within the chromosomal coordinates as listed in Tables 1 or 6.

In some embodiments, a PD-L1 polypeptide provided herein is encoded by a CD274 fusion nucleic acid molecule described herein. In some embodiments, such a PD-L1 polypeptide comprises at least a portion of a PD-L1 polypeptide fused to at least a portion of a polypeptide encoded by another gene, e.g., by a CD274, CFAP52, MYLK, PTPRD, RIC1, HIPK2, PDCD1LG2, ERMP1, CHMP5, PTPRB, PLGRKT, or JAK2 gene. For example, in some embodiments, provided herein are PD-L1 fusion polypeptides encoded by a CD274-CD274, CD274-CFAP52, CD274-MYLK, CD274-PTPRD, CD274-RIC1, CD274-HIPK2, CD274-PDCD1LG2, CD274-ERMP1, CD274-CHMP5, CD274-PTPRB, CD274-PLGRKT, or CD274-JAK2 fusion nucleic acid molecule of the disclosure.

In some embodiments, a PD-L1 polypeptide of the disclosure is encoded by any of the CD274 fusion nucleic acid molecules described herein, and/or in any of Tables 2-5 and 7-8, and/or in the Examples herein. In some embodiments, a PD-L1 polypeptide of the disclosure is encoded by any of the CD274 fusion nucleic acid molecules described herein, comprising or resulting from a CD274 Breakpoint and/or Fusion Partner Gene Breakpoint described herein, and/or in any of Tables 4, 5 or 8, and/or in the Examples herein.

Also provided herein are fragments of any of the PD-L1 polypeptides of the disclosure.

In some embodiments, a PD-L1 polypeptide provided herein, or a fragment thereof, is oncogenic. In some embodiments, a PD-L1 polypeptide provided herein, or a fragment thereof, promotes cancer cell survival, angiogenesis, cancer cell proliferation, and any combination thereof.

### (iv) Cancers and Methods Related Thereto

Certain aspects of the present disclosure relate to methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; assessing a CD274 nucleic acid molecule or a PD-L1 polypeptide in a cancer in an individual; detecting the presence or absence of a cancer in an individual; monitoring progression or recurrence of a cancer in an individual; or identifying a candidate treatment for a cancer in an individual in need thereof.

In some embodiments of any of the methods provided herein, the methods comprise acquiring knowledge of or detecting in a sample from an individual (e.g., an individual having cancer, suspected of having cancer, being tested for cancer, or being treated for cancer) a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein). In other embodiments, the methods comprise acquiring knowledge of or detecting in a sample from an individual (e.g., an individual having cancer, suspected of having cancer, being tested for cancer, or being treated for cancer) a PD-L1 polypeptide of the disclosure, e.g., a PD-L1 polypeptide encoded by a CD274 nucleic acid molecule comprising or resulting from a rearrangement described herein, or a CD274 fusion nucleic acid molecule described herein (e.g., in Tables 1-8, and/or in the Examples herein). In some embodiments of any of the methods provided herein, detection of a CD274 nucleic acid molecule or PD-L1 polypeptide of the disclosure in the sample identifies the individual as one who may benefit from a treatment comprising an anti-cancer therapy, such as an anti-cancer therapy provided herein, e.g., an immune checkpoint inhibitor.

In some embodiments, the methods comprise detecting, in a first sample obtained from the individual at a first time point, the presence or absence of a CD274 nucleic acid molecule of the disclosure (e.g., a CD274 nucleic acid molecule comprising or resulting from a rearrangement described herein, or a CD274 fusion nucleic acid molecule described herein) or a PD-L1 polypeptide of the disclosure (e.g., a PD-L1 polypeptide encoded by a CD274 nucleic acid molecule comprising or resulting from a rearrangement described herein, or a CD274 fusion nucleic acid molecule described herein). In some embodiments, the methods further comprise detecting, in a second sample obtained from the individual at a second time point after the first time point, the presence or absence of a CD274 nucleic acid molecule or PD-L1 polypeptide of the disclosure. In some embodiments, the methods further comprise providing an assessment of cancer progression or cancer recurrence in the individual based, at least in part, on the presence or absence of the CD274 nucleic acid molecule or PD-L1 polypeptide in the first sample and/or in the second sample. In some embodiments, the presence of the CD274 nucleic acid molecule or PD-L1 polypeptide in the first sample and/or in the second sample identifies the individual as having increased risk of cancer progression or cancer recurrence. In some embodiments, the presence of the CD274 nucleic acid molecule or the PD-L1 polypeptide of the disclosure in the first sample and/or in the second sample identifies the individual as having decreased risk of cancer progression or cancer recurrence when treated with a treatment comprising an immune checkpoint inhibitor. In some embodiments, the methods further comprise selecting a treatment, administering a treatment, adjusting a treatment, adjusting the dose of a treatment, or applying a treatment to the individual based, at least in part, on detecting the presence of the CD274 nucleic acid molecule or PD-L1 polypeptide in the first sample and/or in the second sample, wherein the treatment comprises an anti-cancer therapy, such as an anti-cancer therapy provided herein, e.g., an immune checkpoint inhibitor.

In some embodiments, the methods comprise performing DNA sequencing on a sample obtained from the individual to determine a sequencing mutation profile of the CD274 gene. In some embodiments, the sequencing mutation profile identifies the presence or absence of a CD274 nucleic acid molecule of the disclosure, e.g., a CD274 nucleic acid molecule comprising or resulting from a rearrangement described herein, or a CD274 fusion nucleic acid molecule described herein. In some embodiments, the methods further comprise identifying a candidate treatment for a cancer in an individual, based at least in part on the sequencing mutation profile. In some embodiments, the candidate treatment comprises an anti-cancer therapy, such as an anti-cancer therapy provided herein, e.g., an immune checkpoint inhibitor. In some embodiments, the sequencing mutation profile identifies the presence or absence of a fragment of the CD274 nucleic acid molecule comprising a breakpoint or fusion junction, e.g., one or more of the corresponding breakpoints described herein. In some embodiments,

In some embodiments of any of the methods provided herein, the methods further comprise generating a report comprising one or more treatment options identified for the individual based at least in part on detection of the CD274 nucleic acid molecule of the disclosure (e.g., a CD274 nucleic acid molecule comprising or resulting from a rearrangement described herein, or a CD274 fusion nucleic acid molecule described herein) or the PD-L1 polypeptide of the disclosure (e.g., a PD-L1 polypeptide encoded by a CD274 nucleic acid molecule comprising or resulting from a rearrangement described herein, or a CD274 fusion nucleic acid molecule described herein) in the sample, wherein the one or more treatment options comprise an anti-cancer therapy, such as an anti-cancer therapy provided herein, e.g., an immune checkpoint inhibitor.

In some embodiments of any of the methods provided herein, responsive to acquisition of knowledge of a CD274 nucleic acid molecule of the disclosure (e.g., a CD274 nucleic acid molecule comprising or resulting from a rearrangement described herein, or a CD274 fusion nucleic acid molecule described herein) or a PD-L1 polypeptide of the disclosure (e.g., a PD-L1 polypeptide encoded by a CD274 nucleic acid molecule comprising or resulting from a rearrangement described herein, or a CD274 fusion nucleic acid molecule described herein) in a sample from the individual: (i) the individual is classified as a candidate to receive a treatment comprising an anti-cancer therapy, such as an anti-cancer therapy provided herein, e.g., an immune checkpoint inhibitor; and/or (ii) the individual is identified as likely to respond to a treatment that comprises an anti-cancer therapy, such as an anti-cancer therapy provided herein, e.g., an immune checkpoint inhibitor. In some embodiments, responsive to acquisition of knowledge of the CD274 nucleic acid molecule or PD-L1 polypeptide in a sample from the individual, the individual is predicted to have longer survival when treated with a treatment comprising an anti-cancer therapy, such as an anti-cancer therapy provided herein, e.g., an immune checkpoint inhibitor, as compared to survival of an individual whose cancer does not comprise or exhibit the CD274 nucleic acid molecule or PD-L1 polypeptide. In some embodiments, responsive to acquisition of knowledge of the CD274 nucleic acid molecule or PD-L1 polypeptide in a sample from the individual, the individual is predicted to have increased risk of cancer recurrence, aggressive cancer, resistance to an anti-cancer treatment, or poor prognosis, as compared to an individual whose cancer does not comprise the CD274 nucleic acid molecule or PD-L1 polypeptide. In some embodiments, responsive to acquisition of knowledge of the CD274 nucleic acid molecule or PD-L1 polypeptide in a sample from the individual, the individual is predicted to have an improved response to treatment with an immune checkpoint inhibitor as compared to an individual whose cancer does not comprise a CD274 nucleic acid molecule or a PD-L1 polypeptide of the disclosure.

In some embodiments, responsive to acquisition of knowledge of a CD274 nucleic acid molecule of the disclosure (e.g., a CD274 nucleic acid molecule comprising or resulting from a rearrangement described herein, or a CD274 fusion nucleic acid molecule described herein) or a PD-L1 polypeptide of the disclosure (e.g., a PD-L1 polypeptide encoded by a CD274 nucleic acid molecule comprising or resulting from a rearrangement described herein, or a CD274 fusion nucleic acid molecule described herein) in a sample from the individual, the methods comprise administering to the individual an effective amount of a treatment that comprises an anti-cancer therapy, such as an anti-cancer therapy provided herein, e.g., an immune checkpoint inhibitor.

In some embodiments of any of the methods provided herein, the methods further comprise generating a report comprising one or more treatment options identified for the individual based at least in part on knowledge of a CD274 nucleic acid molecule of the disclosure (e.g., a CD274 nucleic acid molecule comprising or resulting from a rearrangement described herein, or a CD274 fusion nucleic acid molecule described herein) or a PD-L1 polypeptide of the disclosure (e.g., a PD-L1 polypeptide encoded by a CD274 nucleic acid molecule comprising or resulting from a rearrangement described herein, or a CD274 fusion nucleic acid molecule described herein) in a sample from the individual, wherein the one or more treatment options comprise an anti-cancer therapy, such as an anti-cancer therapy provided herein, e.g., an immune checkpoint inhibitor.

In some embodiments, acquiring knowledge of a CD274 nucleic acid molecule of the disclosure (e.g., a CD274 nucleic acid molecule comprising or resulting from a rearrangement described herein, or a CD274 fusion nucleic acid molecule described herein) or a PD-L1 polypeptide of the disclosure (e.g., a PD-L1 polypeptide encoded by a CD274 nucleic acid molecule comprising or resulting from a rearrangement described herein, or a CD274 fusion nucleic acid molecule described herein) in a sample comprises detecting the CD274 nucleic acid molecule or PD-L1 polypeptide in the sample. In some embodiments of any of the methods provided herein, detecting a CD274 nucleic acid molecule of the disclosure comprises detecting a fragment of the CD274 nucleic acid molecule comprising a breakpoint or fusion junction, e.g., one or more of the corresponding breakpoints described herein. In some embodiments of any of the methods provided herein, detecting a PD-L1 polypeptide of the disclosure comprises detecting a portion of the PD-L1 polypeptide that is encoded by a fragment of a CD274 nucleic acid molecule that comprises a breakpoint or a fusion junction, e.g., one or more of the corresponding breakpoints described herein.

In some embodiments, the methods further comprise providing an assessment of a CD274 nucleic acid molecule of the disclosure (e.g., a CD274 nucleic acid molecule comprising or resulting from a rearrangement described herein, or a CD274 fusion nucleic acid molecule described herein) or a PD-L1 polypeptide of the disclosure (e.g., a PD-L1 polypeptide encoded by a CD274 nucleic acid molecule comprising or resulting from a rearrangement described herein, or a CD274 fusion nucleic acid molecule described herein), e.g., responsive to detecting or acquiring knowledge of the CD274 nucleic acid molecule of PD-L1 polypeptide in a sample from an individual.

In some embodiments of any of the methods provided herein, the anti-cancer therapy comprises an immune checkpoint inhibitor. In some embodiments, the immune checkpoint inhibitor comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising a CD274 nucleic acid molecule or PD-L1 polypeptide of the disclosure, a treatment for CD274- or PD-L1-rearranged cancer, a treatment for cancer being tested in a clinical trial, a targeted therapy, a treatment being tested in a clinical trial for cancer comprising a CD274 nucleic acid molecule or PD-L1 polypeptide of the disclosure, a treatment being tested in a clinical trial for CD274- or PD-L1-rearranged cancer, or any combination thereof, e.g., a described in further detail below. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of a CD274 nucleic acid molecule or PD-L1 polypeptide of the disclosure. In some embodiments, the nucleic acid comprises a double-stranded RNA (dsRNA), a small interfering RNA (siRNA), or a small hairpin RNA (shRNA), e.g., as described herein. In some embodiments, the treatment or the one or more treatment options comprise nivolumab or pembrolizumab monotherapy.

In some embodiments of any of the methods provided herein, the treatment or the one or more treatment options further comprise an additional anti-cancer therapy. In some embodiments of any of the methods provided herein, the methods comprise administering an additional anti-cancer therapy to the individual (e.g., in addition to, in combination with, or concurrently with an immune checkpoint inhibitor). In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the treatment or the one or more treatment options comprise an immune checkpoint inhibitor in combination with one or more chemotherapeutic agents, e.g., any chemotherapeutic agent known in the art or described herein. In some embodiments, the one or more chemotherapeutic agents comprise a platinum-based chemotherapeutic agent, such as any platinum-based chemotherapeutic agent known in the art or described herein. n some embodiments, the treatment or the one or more treatment options comprise atezolizumab and one or more chemotherapeutic agents, e.g., any chemotherapeutic agent known in the art or described herein. In some embodiments, the treatment or the one or more treatment options comprise atezolizumab, bevacizumab-Awwb or bevacizumab, carboplatin, and paclitaxel. In some embodiments, the atezolizumab, bevacizumab-Awwb or bevacizumab, carboplatin, and paclitaxel are a first line treatment for cancer. In some embodiments, the treatment or the one or more treatment options comprise atezolizumab and paclitaxel or paclitaxel protein-bound. In some embodiments, the atezolizumab and paclitaxel or paclitaxel protein-bound are a first line or a subsequent line of treatment for cancer. In some embodiments, the treatment or the one or more treatment options comprise pembrolizumab and one or more chemotherapeutic agents, e.g., any chemotherapeutic agent known in the art or described herein. In some embodiments, the treatment or the one or more treatment options comprise pembrolizumab, carboplatin and pemetrexed. In some embodiments the pembrolizumab, carboplatin and pemetrexed are first line treatment for cancer. In some embodiments, the treatment or the one or more treatment options comprise pembrolizumab monotherapy. In some embodiments, the pembrolizumab monotherapy is a first line or a second line treatment for cancer. In some embodiments, the pembrolizumab monotherapy is a second line treatment for cancer. In some embodiments, the treatment or the one or more treatment options comprise nivolumab monotherapy. In some embodiments, the nivolumab monotherapy is a first line or a subsequent line of treatment for cancer.

In some embodiments of any of the methods provided herein, therapeutic or clinical responses to treatment (e.g., a treatment provided herein) are assessed using any suitable method known in the art. For example, therapeutic or clinical responses may be assessed according to the Response Evaluation Criteria in Solid Tumors (RECIST), see, e.g., the website recist[dot]eortc[dot]org for additional information. In some embodiments of any of the methods provided herein, treatment of an individual having cancer according to any of the methods provided herein results in at least stable disease, at least a partial response, or a complete response. In some embodiments of any of the methods provided herein, treatment of an individual having cancer according to any of the methods provided herein results in remission such as complete remission. In some embodiments of any of the methods provided herein, treatment of an individual having cancer according to any of the methods provided herein results in a duration of response or a duration of treatment of at least about 10 days, at least about 20 days, at least about 30 days, at least about 40 days, at least about 50 days, at least about 60 days, at least about 70 days, at least about 80 days, at least about 90 days, at least about 100 days, at least about 110 days, at least about 120 days, at least about 130 days, at least about 140 days, at least about 150 days, at least about 160 days, at least about 170 days, at least about 180 days, at least about 190 days, at least about 200 days, at least about 210 days, at least about 220 days, at least about 230 days, at least about 240 days, at least about 250 days, at least about 260 days, at least about 270 days, at least about 280 days, at least about 290 days, at least about 300 days, at least about 310 days, at least about 320 days, at least about 330 days, at least about 340 days, at least about 350 days, at least about 360 days, at least about 370 days, at least about 380 days, at least about 390 days, at least about 400 days, at least about 410 days, at least about 420 days, at least about 430 days, at least about 440 days, at least about 450 days, at least about 460 days, at least about 470 days, at least about 480 days, at least about 490 days, at least about 500 days, at least about 510 days, at least about 520 days, at least about 530 days, at least about 540 days, at least about 550 days, at least about 560 days, at least about 570 days, at least about 580 days, at least about 590 days, at least about 600 days, at least about 610 days, at least about 620 days, at least about 630 days, at least about 640 days, at least about 650 days, at least about 660 days, at least about 670 days, at least about 680 days, at least about 690 days, at least about 700 days, at least about 710 days, at least about 720 days, at least about 730 days, at least about 740 days, at least about 750 days, at least about 760 days, at least about 770 days, at least about 780 days, at least about 790 days, at least about 800 days, or more.

In some embodiments, the individual has been previously treated, or is being treated, for cancer with a treatment for cancer, e.g., an anti-cancer therapy described herein or any other anti-cancer therapy or treatment known in the art. In some embodiments, a CD274 nucleic acid molecule of the disclosure, and/or a PD-L1 polypeptide of the disclosure, confers resistance of the cancer to the treatment for cancer.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the cancer is an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, unknown primary adenocarcinoma, gastric cancer or carcinoma, unknown primary carcinoma (CUP), unknown primary carcinoma (CUP) (NOS), unknown primary melanoma, stomach adenocarcinoma, or vagina squamous cell carcinoma (SCC).

In some embodiments, the cancer is a stage IV B lung non-squamous cell lung adenocarcinoma. In some embodiments, the cancer is a stage II B lung squamous cell carcinoma. In some embodiments, the cancer is a stage III C breast carcinoma (NOS). In some embodiments, the cancer is a stage III C breast invasive ductal carcinoma (IDC). In some embodiments, the cancer is a stage IV or stage III C stomach adenocarcinoma. In some embodiments, the cancer is a stage IV A ovary serous carcinoma.

In some embodiments, the cancer is metastatic.

In some embodiments, the methods further comprise detecting the presence or absence of a cancer in a sample from the individual. In some embodiments, the methods further comprise administering an effective amount of anti-cancer therapy to the individual, e.g., an anti-cancer therapy described herein, e.g., an immune checkpoint inhibitor.

In some embodiments, any of the cancers described herein may comprise any of the CD274 nucleic acid molecules of the disclosure (e.g., a CD274 nucleic acid molecule comprising or resulting from a rearrangement described herein, or a CD274 fusion nucleic acid molecule described herein), or PD-L1 polypeptides of the disclosure (e.g., a PD-L1 polypeptide encoded by a CD274 nucleic acid molecule comprising or resulting from a rearrangement described herein, or a CD274 fusion nucleic acid molecule described herein). Also included in this disclosure is any cancer known in the art that may comprise any of the CD274 nucleic acid molecules of the disclosure or PD-L1 polypeptides of the disclosure.

In some embodiments, the methods provided herein comprise acquiring knowledge of or detecting any of the CD274 nucleic acid molecules of the disclosure (e.g., a CD274 nucleic acid molecule comprising or resulting from a rearrangement described herein, or a CD274 fusion nucleic acid molecule described herein), or PD-L1 polypeptides of the disclosure (e.g., a PD-L1 polypeptide encoded by a CD274 nucleic acid molecule comprising or resulting from a rearrangement described herein, or a CD274 fusion nucleic acid molecule described herein) in a sample from an individual having any cancer known in the art, or any of the cancers described herein.

In some embodiments, a cancer provided in Table 6 comprises a CD274 nucleic acid molecule comprising or resulting from a rearrangement comprising a Breakpoint 1 and/or Breakpoint 2 within the chromosomal coordinates as listed in Table 1 or Table 6, or a PD-L1 polypeptide encoded by the CD274 nucleic acid molecule.

TABLE 6
Exemplary CD274 rearrangements and corresponding breakpoints, identified in the
indicated cancer types.

| Cancer Type | Gene | Breakpoint 1 | Breakpoint 2 |
|---|---|---|---|
| Adrenal gland | CD274 | chr9: 5466758-5466912 | chr9: 7819480-7819610 |
| cortical carcinoma | | | |
| Breast invasive | CD274 | chr9: 5467780-5467988 | chr9: 6511502-6511679 |
| ductal carcinoma | | | |
| (IDC) | | | |
| Lung | CD274 | chr9: 5467646-5467904 | chr9: 5447459-5447604 |
| adenocarcinoma | | | |
| Colon | CD274 | chr9: 5467655-5467913 | chr9: 5489388-5489533 |
| adenocarcinoma | | | |
| (CRC) | | | |
| Colon | CD274 | chr9: 5466698-5466964 | chr9: 5582855-5583098 |
| adenocarcinoma | | | |
| (CRC) | | | |
| Lung non-small cell | CD274 | chr9: 5467714-5467907 | chr18: 28214456-28214689 |
| lung carcinoma | | | |
| (NSCLC) (NOS) | | | |
| Lung | CD274 | chr9: 5466689-5466956 | chr9: 6676528-6676754 |
| adenocarcinoma | | | |
| Prostate acinar | CD274 | chr9: 5467714-5467957 | chr9: 4316216-4316474 |
| adenocarcinoma | | | |
| Lung | CD274 | chr9: 5467678-5467931 | chr9: 3590897-3591102 |
| adenocarcinoma | | | |
| Lung non-small cell | CD274 | chr9: 5465440-5465671 | chr9: 5479019-5479384 |
| lung carcinoma | | | |
| (NSCLC) (NOS) | | | |
| Lung squamous cell | CD274 | chr9: 5465342-5465658 | chr9: 5270435-5270947 |
| carcinoma (SCC) | | | |
| Head and neck | CD274 | chr9: 5467863-5468050 | chr9: 5500043-5500268 |
| squamous cell | | | |
| carcinoma (HNSCC) | | | |
| Ovary epithelial | CD274 | chr9: 5467659-5467922 | chr2: 157519407-157519682 |
| carcinoma | | | |
| Lung non-small cell | CD274 | chr9: 5467787-5468003 | chr9: 5471671-5471863 |
| lung carcinoma | | | |
| (NSCLC) (NOS) | | | |
| Ovary serous | CD274 | chr9: 5467642-5467900 | chr6: 94722507-94722813 |
| carcinoma | | | |
| Lung | CD274 | chr9: 5467648-5467991 | chr18: 27844666-27844942 |
| adenocarcinoma | | | |
| Liver hepatocellular | CD274 | chr9: 5466585-5466876 | chr9: 5201780-5202054 |
| carcinoma (HCC) | | | |
| Lung non-small cell | CD274 | chr9: 5467638-5467928 | chr9: 5441513-5441883 |
| lung carcinoma | | | |
| (NSCLC) (NOS) | | | |
| Ovary serous | CD274 | chr9: 5467681-5467968 | chr9: 5198653-5198919 |
| carcinoma | | | |
| Esophagus | CD274 | chr9: 5466590-5466996 | chr9: 5149992-5150287 |
| adenocarcinoma | | | |
| Esophagus | CD274 | chr9: 5467660-5467942 | chr9: 5479655-5479937 |
| squamous cell | | | |
| carcinoma (SCC) | | | |
| Bladder urothelial | CD274 | chr9: 5465343-5465699 | chr9: 8143594-8144044 |
| (transitional cell) | | | |
| carcinoma | | | |
| Cervix squamous | CD274 | chr9: 5467637-5467886 | chr9: 5484662-5485082 |
| cell carcinoma | | | |
| (SCC) | | | |
| Breast carcinoma | CD274 | chr9: 5466636-5466908 | chr9: 5848084-5848343 |
| (NOS) | | | |
| Breast invasive | CD274 | chr9: 5467939-NA | chr9: 5486358-NA |
| ductal carcinoma | | | |
| (IDC) | | | |
| Stomach | CD274 | chr9: 5467786-5467955 | chr9: 5442048-5442165 |
| adenocarcinoma | | | |
| Stomach | CD274 | chr9: 5467697-5467980 | chr4: 131103841-131104134 |
| adenocarcinoma | | | |
| Ovary serous | CD274 | chr9: 5467704-5467951 | chr9: 5470872-5471004 |
| carcinoma | | | |

SCC: squamous cell carcinoma;
NOS: not otherwise specified;
HCC: hepatocellular carcinoma;
HNSCC: head and neck squamous cell carcinoma;
NSCLC: non-small cell lung cancer;
CRC: colorectal cancer;
IDC: invasive ductal carcinoma

In some embodiments, a cancer provided in Table 3 comprises a CD274 fusion nucleic acid molecule comprising a fusion between a CD274 gene, or a portion thereof, and a fusion partner gene as listed in Table 3, or a portion thereof; or a PD-L1 polypeptide encoded by the CD274 fusion nucleic acid molecule. In some embodiments, a cancer provided in Table 3 comprises a CD274 fusion nucleic acid molecule comprising a fusion between a CD274 gene, or a portion thereof, and a fusion partner gene as listed in Table 3, or a portion thereof, and the CD274 fusion nucleic acid molecule comprises or results from a corresponding CD274 Breakpoint and/or Fusion Partner Gene Breakpoint within the chromosomal coordinates as listed in Table 5; or a PD-L1 polypeptide encoded by the CD274 nucleic acid molecule. In some embodiments, a cancer provided in Table 5 comprises a CD274 fusion nucleic acid molecule comprising a fusion between a CD274 gene, or a portion thereof, and a fusion partner gene as listed in Table 5, or a portion thereof, and the CD274 fusion nucleic acid molecule comprises or results from a corresponding CD274 Breakpoint and/or Fusion Partner Gene Breakpoint within the chromosomal coordinates as listed in Table 5; or a PD-L1 polypeptide encoded by the CD274 nucleic acid molecule.

In some embodiments, a cancer provided in Table 7 comprises a CD274 fusion nucleic acid molecule comprising a fusion between a CD274 gene, or a portion thereof, and a fusion partner gene as listed in Table 7, or a portion thereof; or a PD-L1 polypeptide encoded by the CD274 fusion nucleic acid molecule.

TABLE 7
Exemplary CD274 fusion nucleic acid molecules,
identified in the indicated cancer types.

| | | Fusion |
|---|---|---|
| Cancer Type | Gene | Partner Gene |
| Lung small cell undifferentiated carcinoma | CD274 | CFAP52 |
| Colon adenocarcinoma (CRC) | CD274 | MYLK |
| Unknown primary melanoma | CD274 | PTPRD |
| Lung squamous cell carcinoma (SCC) | CD274 | RIC1 |
| Eye lacrimal duct carcinoma | CD274 | HIPK2 |
| Lung non-small cell lung carcinoma (NSCLC) | CD274 | PTPRD |
| (NOS) | | |
| Cervix squamous cell carcinoma (SCC) | CD274 | PDCD1LG2 |
| Colon adenocarcinoma (CRC) | CD274 | ERMP1 |
| Lung adenocarcinoma | CD274 | CHMP5 |
| Lung adenocarcinoma | CD274 | PTPRB |

SCC: squamous cell carcinoma;
NOS: not otherwise specified;
NSCLC: non-small cell lung cancer;
CRC: colorectal cancer

In some embodiments, a cancer provided in Table 7 comprises a CD274 fusion nucleic acid molecule comprising a fusion between a CD274 gene, or a portion thereof, and a fusion partner gene as listed in Table 7, or a portion thereof, and the CD274 fusion nucleic acid molecule comprises or results from a corresponding CD274 Breakpoint and/or Fusion Partner Gene Breakpoint within the chromosomal coordinates as listed in Tables 4 or 8; or a PD-L1 polypeptide encoded by the CD274 nucleic acid molecule. In some embodiments, a cancer provided in Table 8 comprises a CD274 fusion nucleic acid molecule comprising a fusion between a CD274 gene, or a portion thereof, and a fusion partner gene as listed in Table 8, or a portion thereof, and the CD274 fusion nucleic acid molecule comprises or results from a corresponding CD274 Breakpoint and/or Fusion Partner Gene Breakpoint within the chromosomal coordinates as listed in Table 8; or a PD-L1 polypeptide encoded by the CD274 nucleic acid molecule.

TABLE 8
Exemplary CD274 fusion nucleic acid molecules and corresponding breakpoints,
identified in the indicated cancer types.

| | | Fusion | CD274 | Fusion Partner |
|---|---|---|---|---|
| Cancer Type | Gene | Partner Gene | Breakpoint | Gene Breakpoint |
| Lung small cell | CD274 | CFAP52 | chr9: 5466613- | chr17: 9496835-9497088 |
| undifferentiated | | | 5466970 | |
| carcinoma | | | | |
| Colon | CD274 | MYLK | chr9: 5465373- | chr3: 123592212- |
| adenocarcinoma | | | 5465731 | 123592310 |
| (CRC) | | | | |
| Unknown primary | CD274 | PTPRD | chr9: 5467743- | chr9: 9745527-9745899 |
| melanoma | | | 5467994 | |
| Lung squamous | CD274 | RIC1 | chr9: 5466596- | chr9: 5731619-5731896 |
| cell carcinoma | | | 5466853 | |
| (SCC) | | | | |
| Eye lacrimal duct | CD274 | HIPK2 | chr9: 5466598- | chr7: 139280432- |
| carcinoma | | | 5467002 | 139281022 |
| Lung non-small | CD274 | PTPRD | chr9: 5467715- | chr9: 8809122-8809281 |
| cell lung carcinoma | | | 5467879 | |
| (NSCLC) (NOS) | | | | |
| Cervix squamous | CD274 | PDCD1LG2 | chr9: 5466586- | chr9: 5543484-5543698 |
| cell carcinoma | | | 5466770 | |
| (SCC) | | | | |
| Colon | CD274 | ERMP1 | chr9: 5467653- | chr9: 5789739-5790086 |
| adenocarcinoma | | | 5467891 | |
| (CRC) | | | | |
| Lung | CD274 | CHMP5 | chr9: 5465378- | chr9: 33281589- |
| adenocarcinoma | | | 5465756 | 33281786 |
| Lung | CD274 | PTPRB | chr9: 5467734- | chr12: 70937973- |
| adenocarcinoma | | | 5467932 | 70938263 |

SCC: squamous cell carcinoma;
NOS: not otherwise specified;
NSCLC: non-small cell lung cancer;
CRC: colorectal cancer

In some embodiments, the methods further comprise acquiring knowledge of or detecting in a sample from the individual the presence or absence of a CD274 gene amplification. In some embodiments, a cancer of the disclosure comprises a CD274 gene amplification. In some embodiments, a cancer of the disclosure does not comprise a CD274 gene amplification.

In some embodiments of any of the methods provided herein, the methods further comprise acquiring knowledge of or detecting in a sample from the individual a base substitution, a short insertion/deletion (indel), a copy number alteration, or a genomic fusion/rearrangement in one or more genes. In some embodiments, the one or more genes comprise TP53, PIK3CA, CDKN2A. KRAS, CDKN2B, CD274, MYC, JAK2, RB1, PDCD1LG2, APC, ARID1A, PTEN, BRAF, CREBBP, PBRM1, KMT2D, CCND1, KDM6A, BCL2L1, ERBB2, FBXW7, NF1, BCORL1, BRCA2, FGF19, FGFR1, MAP2K1, PRKC1, ATM, CDK12, CTNNB1, DNMT3A, FGF3, FGF4, GNAS, LYN, MET, NOTCH1, RNF43, STK11, TET2, VHL, ZNF217, ASXL1, BRCA1, EGFR, KDM5C, KIT, NFE2L2, NOTCH2, NOTCH3, PIK3R1, SOX9, TERC, ZNF703, MTAP, BRIP1, CDC73, ACVR1B, ATRX, MLH1, BRD4, SMAD4, PALB2, RAD21, GATA6, CTCF, or any combination thereof. In some embodiments, the one or more genes comprise one or more of TP53, PIK3CA, CDKN2A, KRAS, CDKN2B, and MYC, or any combination thereof. In some embodiments, the one or more genes comprise one or more of PIK3CA, JAK2, PDCD1LG2, CREBBP, PBRM1, or any combination thereof. In some embodiments, the one or more genes comprise one or more mismatch repair genes. In some embodiments, the mismatch repair gene is an MLH1 gene.

In some embodiments, the methods comprise acquiring knowledge of or detecting, in a sample from the individual, the presence of: (a) an S1601fs*4 frameshift mutation in an ATM gene, and/or a mutation in an ATM gene that results in a V2951F amino acid substitution in an encoded ATM polypeptide; (b) a PTEN gene mutation that results in a Y155C amino acid substitution in an encoded PTEN polypeptide, and/or a PTEN splice site mutation of 634+G>A; (c) a mutation in an RB1 gene that results in a K8* amino acid substitution in an encoded RB1 polypeptide, and/or a deletion of an RB1 gene, or of a portion thereof; (d) a mutation in a MAP2K1 gene that results in an F53C amino acid substitution in an encoded MAP2K1 polypeptide; (c) an amplification of an FGF19, FGF4, CCND1, FGF3, CDC274, or RAD21 gene, or any combination thereof; (f) a deletion of an MTAP gene, or of a portion thereof; (g) a K703fs*3 frameshift mutation in a BRIP1 gene; (h) an H77fs*53 frameshift mutation in a KMT2D gene, and/or a mutation in a KMT2D gene that results in a Q4284* and/or S2834* amino acid substitution in an encoded KMT2D polypeptide; (i) a mutation in an FBXW7 gene that results in a G437R and/or Q242* amino acid substitution in an encoded FBXW7 polypeptide; (j) a CDC73 rearrangement, and/or an M1fs*56 frameshift mutation in a CDC73 gene; (k) a K215fs*19 frameshift mutation in an ACVR1B gene; (l) a D1850fs*33 frameshift mutation in an ARID1A gene; (m) an R840fs*29 frameshift mutation in an ATRX gene; (n) a P798fs*97 frameshift mutation in a BRD4 gene; (o) a mutation in a SMAD4 gene resulting in an R515* amino acid substitution in an encoded SMAD4 polypeptide; (p) a K654fs*47 frameshift mutation in a BRCA1 gene; (q) a mutation in a CTNNB1 gene resulting in a G34R amino acid substitution in an encoded CTNNB1 polypeptide; (r) an M723fs*21 frameshift mutation in a PALB2 gene; (s) a T576fs*4 frameshift mutation in a PIK3R1 gene, and/or a deletion of a PIK3R1 gene, or a portion thereof; (t) a mutation in a GATA6 gene resulting in an E579K amino acid substitution in an encoded GATA6 polypeptide; (u) a mutation in a DNMT3A gene resulting in an R882H amino acid substitution in an encoded DNMT3A polypeptide; (v) an E363fs*5 frameshift mutation in a CTCF gene; or any combination of (a)-(v). In some embodiments, the CDC73 rearrangement results in a CDC73 gene fusion comprising exons 1-7 of CDC73 fused to exons 11-17 of CDC73. In some embodiments, the CDC73 gene fusion comprises or results from a breakpoint in exon 7 of CDC73 and/or a breakpoint in intron 10 of CDC73.

In some embodiments, the methods comprise acquiring knowledge of or detecting, in a sample from the individual, the presence of: (a) an R290fs*55 and/or H296fs*10 frameshift mutation in a TP53 gene; a mutation in a TP53 gene resulting in a G266V, E285K, C176Y, and/or P278S amino acid substitution in an encoded TP53 polypeptide; or a TP53 splice site mutation of 672+1G>T, or any combination thereof; (b) a CDKN2B deletion; (c) a MYC gene amplification; or any combination of (a)-(c). In some embodiments, the methods comprise acquiring knowledge of or detecting, in a sample from the individual, the presence of a deletion of the CDKN2A gene, or a portion thereof.

In some embodiments, the methods comprise acquiring knowledge of or detecting, in a sample from the individual, the presence of a PIK3CA mutation resulting in an E545K and/or E542K amino acid substitution, and/or an E110del deletion, in an encoded PIK3CA polypeptide. In some embodiments, the methods comprise acquiring knowledge of or detecting, in a sample from the individual, the presence of: (a) an I279fs*4 frameshift mutation in a PBRM1 gene; (b) a PDCD1LG2 gene amplification; (c) a JAK2 gene amplification; or any combination of (a)-(c).

In some embodiments, the methods comprise acquiring knowledge of or detecting, in a sample from the individual, the presence of an MLH1 mutation resulting an Y684* amino acid substitution in an encoded MLH1 polypeptide.

In some embodiments of any of the methods provided herein, the sample is a sample described below. In some embodiments, the sample is obtained from the individual or from the cancer. In some embodiments, the methods further comprise obtaining the sample, e.g., from the individual or from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the CD274 nucleic acid molecule or PD-L1 polypeptide of the disclosure is detected in a tissue biopsy sample, in a liquid biopsy sample, or in both a tissue biopsy sample and a liquid biopsy sample, from the individual.

In some embodiments of any of the CD274 nucleic acid molecules provided herein, the CD274 nucleic acid molecule is a genomic nucleic acid molecule (i.e., genomic DNA or fragments thereof), or a transcribed nucleic acid molecule, e.g., an RNA such as mRNA, or a cDNA, or fragments thereof.

In some embodiments of any of the CD274 nucleic acid molecules provided herein, the chromosomal coordinates corresponding to any of the breakpoints described herein correspond to Homo sapiens (human) genome assembly GRCh37 (hg19).

### Epstein-Barr Virus

Epstein-Barr virus (EBV) infection is a risk factor for certain cancers such as gastric cancer. For example, around 10% of gastric cancers are EBV positive (Sasaki, S. et al. (2019) Gastric Cancer). Upregulation of PD-L1 expression in EBV-positive gastric cancer has been described as a potential immune evasion mechanism (Nakano, H. et al. (2021) Sci. Rep. 11:1982). Specifically, T-cell evasion in EBV-positive gastric cancer may be driven by modulation of PD-L1 expression, controlled by the interplay between a miRNA and the 3′ UTR of the CD274 gene (Sasaki, S. et al. (2019) Gastric Cancer). Accordingly, without wishing to be bound by theory, it is believed that CD274 rearrangements of the disclosure may be used as biomarkers predictive of response to immune checkpoint inhibitor treatment in EBV-positive cancers such as gastric cancers.

In some embodiments of any of the methods provided herein, the methods comprise acquiring knowledge of or detecting, in a sample from an individual, the presence or absence of a genomic Epstein-Barr virus (EBV). In some embodiments of any of the methods provided herein, the cancer or individual comprises a genomic EBV. In some embodiments of any of the methods provided herein, the cancer or individual is positive for genomic EBV. In some embodiments, the EBV is HHV-4. In some embodiments, the cancer is a gastric cancer. In some embodiments, the gastric cancer is an adenocarcinoma. In some embodiments, the gastric cancer is a stomach adenocarcinoma. In some embodiments, the treatment or the one or more treatment options comprise pembrolizumab. In some embodiments, the pembrolizumab is pembrolizumab monotherapy. In some embodiments, the treatment or the one or more treatment options are a first line treatment for cancer. In some embodiments, the treatment or the one or more treatment options are a second line treatment for cancer. In some embodiments, the gastric cancer is a Stage IV cancer. See, e.g., www[dot]cancer[dot]gov/types/stomach/hp/stomach-treatment-pdq#_17 for information about gastric cancer and staging.

### B. Detection of CD274 Fusion Nucleic Acid Molecules and Polypeptides

Certain aspects of the present disclosure relate to detection of a CD274 rearrangement described herein, and/or a CD274 nucleic acid molecule of the disclosure, e.g., a CD274 nucleic acid molecule comprising or resulting from a rearrangement described herein, or a CD274 fusion nucleic acid molecule described herein, e.g., in a sample such as a patient sample. In some embodiments, the rearrangement or nucleic acid molecule is detected in vitro.

Other aspects of the present disclosure relate to detection of a PD-L1 polypeptide encoded by a CD274 nucleic acid molecule of the disclosure, e.g., a CD274 nucleic acid molecule comprising or resulting from a rearrangement described herein, or a CD274 fusion nucleic acid molecule described herein, e.g., in a sample such as a patient sample. In some embodiments, the PD-L1 polypeptide is detected in vitro.

### (i) Detection of CD274 Nucleic Acid Molecules

Methods for detecting a CD274 nucleic acid molecule of the disclosure, e.g., a CD274 nucleic acid molecule comprising or resulting from a rearrangement described herein, or a CD274 fusion nucleic acid molecule described herein, are known in the art. For example, in some embodiments, a CD274 nucleic acid molecule is detected by sequencing part or all of a gene (e.g., a gene involved in the rearrangement or fusion resulting in the CD274 nucleic acid molecule, e.g., a CD274 gene, and/or a corresponding fusion partner gene described herein, e.g., as described in any of Tables 1-8, and/or in the Examples herein), by next-generation or other sequencing of DNA, RNA, or cDNA. In some embodiments, a CD274 nucleic acid molecule of the disclosure is detected by PCR amplification of DNA, RNA, or cDNA. In some embodiments, a CD274 nucleic acid molecule of the disclosure is detected by in situ hybridization using one or more polynucleotides that hybridize to a locus involved in the rearrangement or fusion, e.g., a CD274 locus, and/or a corresponding fusion partner gene locus described herein (e.g., as described in Tables 1-8, and/or in the Examples herein), e.g., using fluorescence in situ hybridization (FISH). In some embodiments, a CD274 nucleic acid molecule of the disclosure is detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting a CD274 nucleic acid molecule of the disclosure, e.g., a CD274 nucleic acid molecule comprising or resulting from a rearrangement described herein, or a CD274 fusion nucleic acid molecule described herein, are provided below.

In some embodiments, a CD274 nucleic acid molecule of the disclosure, e.g., a CD274 nucleic acid molecule comprising or resulting from a rearrangement described herein, or a CD274 fusion nucleic acid molecule described herein, is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, a CD274 nucleic acid molecule of the disclosure is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS). In some embodiments, the sequencing comprises RNA-sequencing (RNA-seq). In some embodiments, the amplification-based assay comprises a polymerase chain reaction (PCR) amplification technique, a non-PCR amplification technique, or an isothermal amplification technique. In some embodiments, the amplification-based assay comprises a reverse transcription PCR (RT-PCR), a quantitative real-time PCR (qPCR), or a reverse transcription quantitative real-time PCR (RT-qPCR) assay. In some embodiments, the amplification-based assay comprises an RT-PCR assay.

In some embodiments, a CD274 nucleic acid molecule of the disclosure, e.g., a CD274 nucleic acid molecule comprising or resulting from a rearrangement described herein, or a CD274 fusion nucleic acid molecule described herein, is detected using an in situ hybridization method, such as a fluorescence in situ hybridization (FISH) method.

In some embodiments, FISH analysis is used to identify the chromosomal rearrangement or fusion resulting in a CD274 nucleic acid molecule as described herein, e.g., a CD274 nucleic acid molecule comprising or resulting from a rearrangement described herein, or a CD274 fusion nucleic acid molecule described herein. In some embodiments, FISH analysis is used to identify an RNA molecule comprising or encoding a CD274 nucleic acid molecule of the disclosure. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion or rearrangement, e.g., at one or more exons and or introns of the gene, are utilized. In normal cells (i.e., cells not having a CD274 nucleic acid molecule described herein), both probes are observed (or a secondary color is observed due to the close proximity of the two genes of the fusion or rearrangement); and in cells having a CD274 nucleic acid molecule described herein, only a single gene probe is observed due to the presence of a fusion or rearrangement resulting in the CD274 nucleic acid molecule.

In some embodiments, a CD274 nucleic acid molecule of the disclosure, e.g., a CD274 nucleic acid molecule comprising or resulting from a rearrangement described herein, or a CD274 fusion nucleic acid molecule described herein, is detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, a CD274 nucleic acid molecule of the disclosure, e.g., a CD274 nucleic acid molecule comprising or resulting from a rearrangement described herein, or a CD274 fusion nucleic acid molecule described herein, is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of a CD274 nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, a CD274 nucleic acid molecule of the disclosure, e.g., a CD274 nucleic acid molecule comprising or resulting from a rearrangement described herein, or a CD274 fusion nucleic acid molecule described herein, is detected using a sequencing method. Any method of sequencing known in the art can be used to detect a CD274 nucleic acid molecule provided herein. Exemplary sequencing methods that may be used to detect a CD274 nucleic acid molecule provided herein include those based on techniques developed by Maxam and Gilbert or Sanger. Automated sequencing procedures may also be used, e.g., including sequencing by mass spectrometry.

In some embodiments, a CD274 nucleic acid molecule of the disclosure, e.g., a CD274 nucleic acid molecule comprising or resulting from a rearrangement described herein, or a CD274 fusion nucleic acid molecule described herein, is detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, a CD274 nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 105 molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a CD274 nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLID sequencer from Applied Biosystems (Foster City, CA, USA). Additional exemplary methods and platforms that may be used to detect a CD274 nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments, a CD274 nucleic acid molecule of the disclosure, e.g., a CD274 nucleic acid molecule comprising or resulting from a rearrangement described herein, or a CD274 fusion nucleic acid molecule described herein, is detected using a digital pathology method, such as any digital pathology method known in the art and/or described or provided in Cucoranu et al., J Pathol Inform. 2014; 5(1): 16; Jahn et al., J Clin Med. 2020; Coudray et al., Nat Med 24, 1559-1567 (2018); and/or Echle et al., Br J Cancer 124, 686-696 (2021).

In some embodiments of any of the methods provided herein, the methods may comprise one or more of the steps of: (i) obtaining a sample from an individual (e.g., an individual suspected of having or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual suspected of having or determined to have cancer), wherein the plurality of nucleic acid molecules comprises nucleic acid molecules corresponding to a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein); (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the CD274 nucleic acid molecule; (f) analyzing the plurality of sequence reads; and (g) based on the analysis, detecting the presence or absence of the CD274 nucleic acid molecule in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the CD274 nucleic acid molecule. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein) in said library to produce an enriched sample; (e) sequencing the enriched sample, thereby producing a plurality of sequence reads; (f) analyzing the plurality of sequence reads for the presence of the CD274 nucleic acid molecule; (g) detecting, based on the analyzing step, the presence or absence of the CD274 nucleic acid molecule in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample. In some embodiments, the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample. In some embodiments, the sample comprises a liquid biopsy sample, and the non-cancer nucleic acid molecules are derived from a non-tumor, cell-free DNA (cfDNA) fraction of the liquid biopsy sample.

In some embodiments of any of the methods, the one or more adapters comprise amplification primers, flow cell adaptor sequences, substrate adapter sequences, or sample index sequences.

In some embodiments of any of the methods, the selectively enriching comprises: (a) combining one or more bait molecules with the library, thereby hybridizing the one or more bait molecules to one or more nucleic acid molecules comprising nucleotide sequences corresponding to the CD274 nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer.

In some embodiments of any of the methods provided herein, the methods further comprise selectively enriching for one or more nucleic acids in the sample comprising nucleotide sequences corresponding to the CD274 nucleic acid molecule of the disclosure (e.g., a CD274 nucleic acid molecule comprising or resulting from a rearrangement described herein, or a CD274 fusion nucleic acid molecule described herein, e.g., in Tables 1-8, and/or in the Examples herein). In some embodiments, the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the CD274 nucleic acid molecule and producing nucleic acid hybrids; and (b) isolating the nucleic acid hybrids to produce the enriched sample. In some embodiments, the selectively enriching comprises amplifying the one or more nucleic acids comprising nucleotide sequences corresponding to the CD274 nucleic acid molecule using a polymerase chain reaction (PCR) to produce an enriched sample.

In some embodiments of any of the methods provided herein, the methods further comprise sequencing the enriched sample.

In some embodiments of any of the methods provided herein, the methods further comprise generating a genomic profile for the individual or the sample, based, at least in part, on detecting the presence or absence of the CD274 nucleic acid molecule. In some embodiments, the genomic profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test.

In some embodiments, the genomic profile for the individual further comprises/indicates/comprises information on the level of tumor mutational burden (TMB), clonal TMB, indel TMB, or nonsense-mediated decay (NMD)-escape TMB; the presence or absence of, or the proportion of mutations fitting, a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof. See, e.g., Litchfield et al., Cell. 2021 Feb. 4; 184(3): 596-614.e14 for additional information.

In some embodiments of any of the methods provided herein, the methods further comprise selecting a treatment, administering a treatment, or applying a treatment to the individual based on the generated genomic profile, wherein the treatment comprises an anti-cancer therapy, e.g., as described herein, e.g., an immune checkpoint inhibitor.

In some embodiments of any of the methods provided herein, the methods further comprise generating a report indicating the presence or absence of the CD274 nucleic acid molecule in the sample. In some embodiments, the methods further comprise generating, by the one or more processors, a report indicating the presence or absence of the CD274 nucleic acid molecule in the sample. In some embodiments, the methods further comprise transmitting the report to a healthcare provider. In some embodiments, the report is transmitted via a computer network or a peer-to-peer connection.

In some embodiments of any of the methods provided herein, the methods further comprise acquiring knowledge of or detecting in a sample from the individual a base substitution, a short insertion/deletion (indel), a copy number alteration, or a genomic fusion in one or more genes.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample.

In some embodiments of any of the methods provided herein, the method further comprises determining the circulating tumor DNA (ctDNA) fraction of a liquid biopsy sample.

In some embodiments of any of the methods provided herein, detecting a CD274 nucleic acid molecule of the disclosure comprises detecting a fragment of the CD274 nucleic acid molecule. In some embodiments, the fragment comprises a breakpoint or fusion junction, e.g., one or more of the corresponding breakpoints described herein.

### (ii) Detection of PD-L1 Polypeptides

Also provided herein are methods of detecting a PD-L1 polypeptide of the disclosure, or a fragment or portion thereof, e.g., a PD-L1 polypeptide encoded by a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein), or a fragment thereof.

A PD-L1 polypeptide provided herein, or a fragment or portion thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a PD-L1 polypeptide provided herein, or a fragment or portion thereof, can be distinguished from a reference polypeptide, e.g., a non-mutant or wild type protein or polypeptide, with an antibody or antibody fragment that reacts differentially with a mutant protein or polypeptide (e.g., a PD-L1 polypeptide provided herein or a fragment or portion thereof) as compared to a reference protein or polypeptide. In some embodiments, a PD-L1 polypeptide of the disclosure, or a fragment or portion thereof, can be distinguished from a reference polypeptide, e.g., a non-mutant or wild type protein or polypeptide, by reaction with a detection reagent, e.g., a substrate, e.g., a substrate for catalytic activity.

In some embodiments, detecting a PD-L1 polypeptide of the disclosure, or a fragment or portion thereof, comprises detecting a fragment or portion of the polypeptide that is encoded by a fragment of a CD274 nucleic acid molecule of the disclosure that comprises a fusion junction or breakpoint, e.g., one or more of the corresponding breakpoints described herein.

In some embodiments, a PD-L1 polypeptide of the disclosure, or a fragment or portion thereof, is detected using a digital pathology method, such as any digital pathology method known in the art and/or described or provided in Cucoranu et al., J Pathol Inform. 2014; 5(1): 16; or Jahn et al., J Clin Med. 2020.

In some aspects, methods of detection of a PD-L1 polypeptide of the disclosure (e.g., a PD-L1 polypeptide encoded by a CD274 nucleic acid molecule of the disclosure, e.g., a CD274 nucleic acid molecule comprising or resulting from a rearrangement described herein, or a CD274 fusion nucleic acid molecule described herein, e.g., as described in any of Tables 1-8, and/or in the Examples herein), or a fragment or portion thereof, are provided, comprising contacting a sample, e.g., a sample described herein, comprising a PD-L1 polypeptide described herein, with a detection reagent provided herein (e.g., an antibody of the disclosure), and determining if the PD-L1 polypeptide is present in the sample.

In some embodiments of any of the methods provided herein, the methods further comprise generating a genomic profile for the individual or the sample, based, at least in part, on detecting the presence or absence of a PD-L1 polypeptide of the disclosure in the sample. In some embodiments, the genomic profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test. In some embodiments, the genomic profile for the individual further comprises/indicates/comprises information on the level of tumor mutational burden (TMB), clonal TMB, indel TMB, or nonsense-mediated decay (NMD)-escape TMB; the presence or absence of, or the proportion of mutations fitting, a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof. See, e.g., Litchfield et al., Cell. 2021 Feb. 4; 184(3): 596-614.e14 for additional information. In some embodiments of any of the methods provided herein, the methods further comprise selecting a treatment, administering a treatment, or applying a treatment to the individual based on the generated genomic profile, wherein the treatment comprises an anti-cancer therapy, e.g., as described herein, e.g., an immune checkpoint inhibitor. In some embodiments of any of the methods provided herein, the methods further comprise generating a report indicating the presence or absence of a PD-L1 polypeptide of the disclosure in the sample.

(iii) Detection Reagents

In some aspects, provided herein are reagents for detecting a CD274 nucleic acid molecule of the disclosure, e.g., a CD274 nucleic acid molecule comprising or resulting from a rearrangement described herein, or a CD274 fusion nucleic acid molecule described herein, or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid molecule, e.g., a nucleic acid molecule that is or comprises a CD274 nucleic acid molecule described herein or a fragment or portion thereof.

In other aspects, provided herein are reagents for detecting a PD-L1 polypeptide of the disclosure, or a fragment thereof, e.g., a PD-L1 polypeptide encoded by a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein), or a fragment thereof, e.g., according to the methods of detection provided herein. In some embodiments, a detection reagent provided herein comprises an antibody or antibody fragment that specifically binds to a PD-L1 polypeptide of the disclosure, or to a fragment thereof.

### Baits

In some embodiments, nucleic acids corresponding to a gene involved in a CD274 nucleic acid molecule described herein, e.g., a CD274 nucleic acid molecule comprising or resulting from a rearrangement described herein, or a CD274 fusion nucleic acid molecule described herein (e.g., in Tables 1-8, and/or in the Examples herein), are captured (e.g., from amplified nucleic acids) by hybridization with a bait molecule. Provided herein are bait molecules suitable for the detection of a CD274 nucleic acid molecule of the disclosure.

In some embodiments, a bait molecule comprises a capture nucleic acid molecule configured to hybridize to a target nucleic acid molecule comprising a CD274 nucleic acid molecule of the disclosure, e.g., a CD274 nucleic acid molecule comprising or resulting from a rearrangement described herein, or a CD274 fusion nucleic acid molecule described herein, or a fragment or portion thereof.

In some embodiments, the capture nucleic acid molecule is configured to hybridize to a fragment of a CD274 nucleic acid molecule of the disclosure, e.g., a CD274 nucleic acid molecule comprising or resulting from a rearrangement described herein, or a CD274 fusion nucleic acid molecule described herein. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises a breakpoint or fusion junction of a CD274 nucleic acid molecule of the disclosure.

In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a breakpoint of a CD274 nucleic acid molecule of the disclosure, e.g., a CD274 nucleic acid molecule comprising or resulting from a rearrangement described herein, or a CD274 fusion nucleic acid molecule described herein, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is configured to hybridize to a nucleotide sequence in an intron or an exon of a CD274gene, or in a breakpoint joining the introns or exons of a CD274 gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene (e.g., a corresponding gene fusion partner as described herein).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, an affinity reagent, a tag or detection reagent. In some embodiments, the label, tag, affinity reagent, or detection reagent is a radiolabel, an antibody or fragment thereof, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art. e.g., as described in WO2012092426A1 and/or or in Frampton et al (2013) Nat Biotechnol. 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, a bait provided herein hybridizes to a nucleotide sequence corresponding to an intron or an exon of one gene of a CD274 molecule described herein (e.g., a CD274 gene), in an intron or an exon of the other gene of a CD274 molecule described herein (e.g., a corresponding gene fusion partner as described herein, and/or a breakpoint joining the introns and/or exons.

The baits described herein can be used for selection of exons and short target sequences.

In some embodiments, a bait of the disclosure distinguishes a nucleic acid molecule, e.g., a genomic or transcribed nucleic acid molecule, e.g., a cDNA or RNA, having a breakpoint of a CD274 nucleic acid molecule described herein, e.g., a CD274 nucleic acid molecule comprising or resulting from a rearrangement described herein, or a CD274 fusion nucleic acid molecule described herein, from a reference nucleotide sequence, e.g., a nucleotide sequence not having the breakpoint.

In some embodiments, the bait hybridizes to a breakpoint of a CD274 nucleic acid molecule described herein, e.g., a CD274 nucleic acid molecule comprising or resulting from a rearrangement described herein, or a CD274 fusion nucleic acid molecule described herein, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Probes

Also provided herein are probes, e.g., nucleic acid molecules, suitable for the detection of a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein). In some embodiments, a probe provided herein comprises a nucleic acid sequence configured to hybridize to a target nucleic acid molecule that is or comprises a CD274 nucleic acid molecule of the disclosure, or a fragment or portion thereof. In some embodiments, the probe comprises a nucleic acid sequence configured to hybridize to the CD274 nucleic acid molecule of the disclosure, or the fragment or portion thereof, of the target nucleic acid molecule. In some embodiments, the probe comprises a nucleic acid sequence configured to hybridize to a fragment or portion of the CD274 nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a breakpoint of a CD274 nucleic acid molecule of the disclosure, e.g., a CD274 nucleic acid molecule comprising or resulting from a rearrangement described herein, or a CD274 fusion nucleic acid molecule described herein, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the probe comprises a nucleotide sequence configured to hybridize to a nucleotide sequence in an intron or an exon of a gene involved in a CD274 nucleic acid molecule described herein, e.g., a CD274 gene, or in a breakpoint joining the introns or exons of the gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene (e.g., a corresponding gene fusion partner as described herein).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of CD274 nucleic acid molecules provided herein. For example, a probe provided herein may be used for detecting a CD274 nucleic acid molecule of the disclosure, e.g., a CD274 nucleic acid molecule comprising or resulting from a rearrangement described herein, or a CD274 fusion nucleic acid molecule described herein, in a sample, e.g., a sample obtained from an individual. In some embodiments, the probe may be used for identifying cells or tissues that express a CD274 nucleic acid molecule of the disclosure, e.g., by measuring levels of the CD274 nucleic acid molecule. In some embodiments, the probe may be used for detecting levels of a CD274 nucleic acid molecule of the disclosure, e.g., mRNA levels, in a sample of cells from an individual.

In some embodiments, a probe provided herein specifically hybridizes to a nucleic acid molecule comprising a rearrangement (e.g., a deletion, inversion, insertion, duplication, translocation, or other fusion) resulting in a CD274 nucleic acid molecule of the disclosure, e.g., a CD274 nucleic acid molecule comprising or resulting from a rearrangement described herein, or a CD274 fusion nucleic acid molecule described herein.

In some embodiments, a probe of the disclosure distinguishes a nucleic acid, e.g., a genomic or transcribed nucleic acid, e.g., a cDNA or RNA, having a breakpoint of a CD274 nucleic acid molecule of the disclosure, e.g., a CD274 nucleic acid molecule comprising or resulting from a rearrangement described herein, or a CD274 fusion nucleic acid molecule described herein, from a reference nucleotide sequence, e.g., a nucleotide sequence not having the breakpoint.

Also provided herein are isolated pairs of allele-specific probes, wherein, for example, the first probe of the pair specifically hybridizes to a CD274 nucleic acid molecule of the disclosure, e.g., a CD274 nucleic acid molecule comprising or resulting from a rearrangement described herein, or a CD274 fusion nucleic acid molecule described herein, and the second probe of the pair specifically hybridizes to a corresponding wild type sequence (e.g., a wild type CD274 nucleic acid molecule; and/or a wild type nucleic acid molecule corresponding to a gene fusion partner as described herein). Probe pairs can be designed and produced for any of the CD274 nucleic acid molecules described herein and are useful in detecting a somatic mutation in a sample. In some embodiments, a first probe of a pair specifically hybridizes to a mutation (e.g., the breakpoint of an alteration, fusion, inversion, duplication, deletion, insertion or translocation resulting in a CD274 nucleic acid molecule described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 105 nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA that is or comprises a fusion nucleic acid molecule of the disclosure, e.g., a CD274 fusion nucleic acid molecule described herein.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a CD274 nucleic acid molecule of the disclosure, e.g., a CD274 nucleic acid molecule comprising or resulting from a rearrangement described herein, or a CD274 fusion nucleic acid molecule described herein. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP. TexasRed-6-dUTP, Cy3-6-dUTP, Cy5-dUTP, Biotin (BIO)-11-dUTP. Digoxygenin (DIG)-11-dUTP and Dinitrophenyl (DNP)-11-dUTP. Probes can also be indirectly labeled with biotin or digoxygenin, or labeled with radioactive isotopes such as 32P and 3H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a breakpoint of a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8 and/or in the Examples herein), and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that is or comprises a CD274 nucleic acid molecule of the disclosure, e.g., a CD274 nucleic acid molecule comprising or resulting from a rearrangement described herein, or a CD274 fusion nucleic acid molecule described herein (e.g., in any of Tables 1-8, and/or in the Examples herein), or a fragment or portion thereof. In some embodiments, the oligonucleotide comprises a nucleotide sequence configured to hybridize to the CD274 nucleic acid molecule of the target nucleic acid molecule. In some embodiments, the oligonucleotide comprises a nucleotide sequence configured to hybridize to a fragment or portion of the CD274 nucleic acid molecule of the target nucleic acid molecule.

In some embodiments, the oligonucleotide, e.g., the primer, comprises a nucleotide sequence configured to hybridize to a breakpoint of a CD274 nucleic acid molecule of the disclosure, e.g., a CD274 nucleic acid molecule comprising or resulting from a rearrangement described herein, or a CD274 fusion nucleic acid molecule described herein, and may be further configured to hybridize to between about 10 and about 12, about 12 and about 15, about 15 and about 17, about 17 and about 20, about 20 and about 25, or about 25 and about 30, or more nucleotides flanking either side of the breakpoint.

In some embodiments, the oligonucleotide, e.g., the primer, comprises a nucleotide sequence configured to hybridize to a nucleotide sequence in an intron or an exon of a gene involved in a CD274 nucleic acid mole of the disclosure (e.g., a CD274 gene), to a breakpoint of a CD274 nucleic acid molecule described herein (e.g., a CD274 nucleic acid molecule comprising or resulting from a rearrangement described herein, or a CD274 fusion nucleic acid molecule described herein), and/or to an intron or exon of another gene (e.g., a corresponding gene fusion partner as described herein).

In some embodiments, the oligonucleotide comprises a nucleotide sequence corresponding to a CD274 nucleic acid molecule of the disclosure, e.g., a CD274 nucleic acid molecule comprising or resulting from a rearrangement described herein, or a CD274 fusion nucleic acid molecule described herein (e.g., in any of Tables 1-8, and/or in the Examples herein). In some embodiments, the oligonucleotide comprises a nucleotide sequence corresponding to a fragment or a portion of the CD274 nucleic acid molecule. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a CD274 nucleic acid molecule provided herein. In some embodiments, the oligonucleotide comprises a nucleotide sequence complementary to a fragment or a portion of the CD274 nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that is or comprises a CD274 nucleic acid molecule of the disclosure, e.g., a CD274 nucleic acid molecule comprising or resulting from a rearrangement described herein, or a CD274 fusion nucleic acid molecule described herein (e.g., in any of Tables 1-8, and/or in the Examples herein), or a fragment thereof, e.g., using PCR. In some embodiments, the oligonucleotide may be used to sequence a nucleic acid molecule that is or comprises a CD274 nucleic acid molecule provided herein, or a fragment thereof. In some embodiments, the oligonucleotide may be used to amplify a nucleic acid molecule comprising a breakpoint of a CD274 nucleic acid molecule described herein, e.g., using PCR. In some embodiments, the oligonucleotide may be used to sequence a nucleic acid molecule comprising a breakpoint of a CD274 nucleic acid molecule described herein.

In some embodiments, pairs of oligonucleotides, e.g., pairs of primers, are provided herein, which are configured to hybridize to a nucleic acid molecule that is or comprises a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein), or a fragment thereof. In some embodiments, a pair of oligonucleotides of the disclosure may be used for directing amplification of the CD274 nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a breakpoint of a CD274 nucleic acid molecule described herein, e.g., for use in directing amplification of the corresponding CD274 nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein), or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a breakpoint of a CD274 nucleic acid molecule described herein, e.g., a CD274 nucleic acid molecule comprising or resulting from a rearrangement described herein, or a CD274 fusion nucleic acid molecule described herein, from a reference nucleotide sequence, e.g., a nucleotide sequence not having the breakpoint.

In one aspect, provided herein is a primer or primer set for amplifying a nucleic acid molecule comprising a cytogenetic abnormality such as an alteration, fusion, chromosomal inversion, deletion, translocation, duplication, or other fusion resulting in a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein). In another aspect, provided herein is a primer or primer set for amplifying a nucleic acid molecule comprising an alteration, fusion, chromosomal inversion, insertion, deletion, translocation, duplication or other fusion resulting in a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein). In certain aspects, provided herein are allele-specific oligonucleotides, e.g., primers, wherein a first oligonucleotide of a pair specifically hybridizes to a mutation (e.g., a breakpoint of a CD274 nucleic acid molecule described herein, e.g., a CD274 nucleic acid molecule comprising or resulting from a rearrangement described herein, or a CD274 fusion nucleic acid molecule described herein), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., a breakpoint of a CD274 nucleic acid molecule described herein, e.g., a CD274 nucleic acid molecule comprising or resulting from a rearrangement described herein, or a CD274 fusion nucleic acid molecule described herein), and a second oligonucleotide of the pair specifically hybridizes to a sequence downstream of the mutation.

In some embodiments, the oligonucleotide, e.g., the primer, hybridizes to a breakpoint of a CD274 nucleic acid molecule described herein, e.g., a CD274 nucleic acid molecule comprising or resulting from a rearrangement described herein, or a CD274 fusion nucleic acid molecule described herein, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Antibodies

Provided herein are antibodies or antibody fragments that specifically bind to a PD-L1 polypeptide of the disclosure, or a fragment thereof, e.g., a PD-L1 polypeptide encoded by a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein), or a fragment thereof.

The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity, e.g., binding to a PD-L1 polypeptide of the disclosure, or a fragment thereof, e.g., a PD-L1 polypeptide encoded by a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein), or a fragment thereof.

In some embodiments, a PD-L1 polypeptide of the disclosure, or a fragment thereof, e.g., a PD-L1 polypeptide encoded by a CD274 nucleic acid molecule of the disclosure, e.g., a CD274 nucleic acid molecule comprising or resulting from a rearrangement described herein, or a CD274 fusion nucleic acid molecule described herein (e.g., as described in any of Tables 1-8, and/or in the Examples herein), or a fragment thereof, is used as an immunogen to generate one or more antibodies of the disclosure, e.g., using standard techniques for polyclonal and monoclonal antibody preparation. In some embodiments, a PD-L1 polypeptide provided herein, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., a PD-L1 polypeptide of the disclosure, or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope. e.g., an epitope on a PD-L1 polypeptide provided herein. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., a PD-L1 polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184.187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate a PD-L1 polypeptide provided herein, or a fragment thereof, by standard techniques, such as affinity chromatography or immunoprecipitation. In some embodiments, an antibody of the disclosure can be used to detect a PD-L1 polypeptide provided herein, or a fragment thereof, e.g., in a tissue sample, cellular lysate, or cell supernatant, in order to evaluate the level and/or pattern of expression of the PD-L1 polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of a PD-L1 polypeptide provided herein) in tissues or body fluids (e.g., in a tumor cell-containing tissue or body fluid), e.g., according to the methods provided herein.

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M) for its antigen (e.g., a PD-L1 polypeptide provided herein). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., a PD-L1 polypeptide provided herein). In some embodiments, a RIA is performed with the Fab version of an antibody of the disclosure and its antigen (e.g., a PD-L1 polypeptide provided herein).

In certain embodiments, an antibody provided herein is an antibody fragment. Antibody fragments include, but are not limited to, Fab, Fab′, Fab′-SH, F(ab′)₂, Fv, and single-chain antibody molecule (e.g., scFv) fragments, and other fragments described herein or known in the art.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are known in the art and described herein.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self-antigens without any immunization. Naive libraries can also be made synthetically by cloning un-rearranged V-gene segments from stem cells, and using PCR primers containing random sequences to amplify the highly variable CDR3 regions and to accomplish fusion in vitro. Antibodies or antibody fragments isolated from human antibody libraries are considered human antibodies or human antibody fragments herein. In certain embodiments, an antibody provided herein is a multispecific antibody, e.g., a bispecific antibody. Multispecific antibodies are monoclonal antibodies that have binding specificities for at least two different sites or at least two different antigens. For example, one of the binding specificities can be to a PD-L1 polypeptide of the disclosure, and the other can be to any other antigen. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fes; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF.” e.g., comprising an antigen binding site that binds to a PD-L1 polypeptide of the disclosure as well as another, different antigen.

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) C1q binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag. Thioredoxin-tag, Glu-tag, Nus-tag, V5-tag, calmodulin-binding protein (CBP)-tag. Maltose binding protein (MBP)-tag. Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag. DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### (iv) Samples

A variety of materials can be the source of, or serve as, samples for use in any of the methods of the disclosure, such as the methods for detection of a CD274 nucleic acid molecule or a PD-L1 polypeptide of the disclosure, or fragments thereof.

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics or the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid.

sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, nucleic acids (e.g., for use in any of the methods for detection of CD274 nucleic acid molecules provided herein) or proteins (e.g., for use in any of the methods for detection of PDL1 polypeptides provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the sample comprises a CD274 nucleic acid molecule or a PD-L1 polypeptide of the disclosure.

In some embodiments, the sample is a control sample or a reference sample, e.g., not containing a CD274 nucleic acid molecule or a PD-L1 polypeptide described herein. In certain embodiments, the reference sample is purified or isolated (e.g., it is removed from its natural state). In certain embodiments, the reference or control sample comprises a wild type or a non-mutated nucleic acid molecule or polypeptide counterpart to any of the CD274 nucleic acid molecules or PD-L1 polypeptides described herein. In other embodiments, the reference sample is from a non-tumor or cancer sample, e.g., a blood control, a normal adjacent tumor (NAT), or any other non-cancerous sample from the same or a different individual.

In some embodiments, a CD274 nucleic acid molecule of the disclosure is detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual. In some embodiments, the sample comprises cDNA derived from an mRNA sample or from a sample comprising mRNA. In some embodiments, a CD274 nucleic acid molecule of the disclosure is detected in a sample comprising cell-free DNA (cfDNA), cell-free RNA, and/or circulating tumor DNA (ctDNA). In some embodiments, a CD274 nucleic acid molecule of the disclosure is detected in a sample comprising cell-free DNA (cfDNA) and/or circulating tumor DNA (ctDNA). In some embodiments, a CD274 nucleic acid molecule of the disclosure is detected in a sample comprising circulating tumor DNA (ctDNA).

### C. Anti-Cancer Therapies

Certain aspects of the present disclosure relate to anti-cancer therapies, as well as methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; detecting the presence or absence of a cancer in an individual; monitoring progression or recurrence of a cancer in an individual; or identifying a candidate treatment for a cancer in an individual in need thereof. The present disclosure also provides uses for anti-cancer therapies (e.g., in methods of treating or delaying progression of cancer in an individual, or in methods for manufacturing a medicament for treating or delaying progression of cancer). In some instances, the methods of the disclosure can include administering an anti-cancer therapy or applying an anti-cancer therapy to an individual based on a generated genomic and/or sequencing mutation profile. An anti-cancer therapy can refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

Certain methods and uses of the disclosure, e.g., methods of treating or delaying progression of cancer in an individual, or methods for manufacturing a medicament for treating or delaying progression of cancer, are based, at least in part, on the observations demonstrated herein that certain genomic rearrangements involving a CD274 gene were found in various cancers. Without wishing to be bound to theory, it is thought that these genomic alterations can identify cancer patients that are likely to benefit from treatment with immune checkpoint inhibitors, e.g., as described herein. Accordingly, in some embodiments, an anti-cancer therapy or a treatment of the disclosure comprises an immune checkpoint inhibitor, such as an immune checkpoint inhibitor known in the art and/or described herein.

In some embodiments, an immune checkpoint inhibitor of the disclosure comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising a CD274 rearrangement or fusion nucleic acid molecule or a PD-L1 polypeptide encoded by such a CD274 rearrangement or fusion nucleic acid molecule (e.g., a CD274 nucleic acid molecule comprising or resulting from a rearrangement described herein, or a CD274 fusion nucleic acid molecule described herein), a treatment for cancer being tested in a clinical trial, a targeted immunotherapy, a treatment being tested in a clinical trial for cancer comprising a CD274 rearrangement or fusion nucleic acid molecule or a PD-L1 polypeptide encoded by such a CD274 rearrangement or fusion nucleic acid molecule (e.g., a CD274 nucleic acid molecule comprising or resulting from a rearrangement described herein, or a CD274 fusion nucleic acid molecule described herein), or any combination thereof, e.g., a described in further detail below.

As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1.” “CD274,” “B7-H.” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC.” “Btdc.” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis). REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences). ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108. STI-A1110. AM0001. BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70. MPDL3280A (atezolizumab), MDX-1105, MEDI4736 (durvalumab), MSB0010718C (avelumab), LY3300054, STI-A1014, KN035, FAZ053, or CX-072.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv. or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70. MPDL3280A (atezolizumab), MDX-1 105. MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the immune checkpoint inhibitor comprises a LAG-3 inhibitor (e.g., an antibody, an antibody conjugate, or an antigen-binding fragment thereof) . In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017. AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is a PD-1-, or a PD-L1-targeted agent, such as a PD-1-, or PD-L1-targeted agent known in the art or described herein. In some embodiments, the PD-1-targeted agent is a PD-1 inhibitor. In some embodiments, the PD-1 inhibitor comprises one or more of nivolumab, pembrolizumab, cemiplimab, or dostarlimab. In some embodiments, the PD-L1-targeted agent is a PD-L1 inhibitor. In some embodiments, the PD-L1 inhibitor comprises one or more of atezolizumab, avelumab, or durvalumab. In some embodiments, the immune checkpoint inhibitor is a CTLA-4 inhibitor, such as a CTLA-4 inhibitor known in the art or described herein. In some embodiments, the CTLA-4 inhibitor comprises ipilimimab.

In some embodiments, the immune checkpoint inhibitor is monovalent and/or monospecific. In some embodiments, the immune checkpoint inhibitor is multivalent and/or multispecific.

In some embodiments, the methods provided herein comprise administering to an individual an effective amount of an immune checkpoint inhibitor.

In some embodiments, an anti-cancer therapy, e.g., an immune checkpoint inhibitor, of the disclosure is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, the cancer immunotherapy is a checkpoint inhibitor, cancer vaccine, cell-based therapy, T cell receptor (TCR)-based therapy, adjuvant immunotherapy, cytokine immunotherapy, or oncolytic virus therapy, e.g., alone or in combination with an immune checkpoint inhibitor. In some embodiments, the cancer immunotherapy comprises a small molecule, nucleic acid, polypeptide, carbohydrate, toxin, cell-based agent, or cell-binding agent, e.g., alone or in combination with an immune checkpoint inhibitor. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen corresponding to a CD274 nucleic acid molecule or PD-L1 polypeptide of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine, e.g., alone or in combination with an immune checkpoint inhibitor. A range of cancer vaccines have been tested that employ different approaches to promoting an immune response against a cancer (see, e.g., Emens LA, Expert Opin Emerg Drugs 13(2): 295-308 (2008) and US20190367613). Approaches have been designed to enhance the response of B cells. T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly (DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196:17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104:14-21, 2013). In some embodiments, such cancer vaccines augment the anti-cancer response.

In some embodiments, the cancer vaccine comprises a polynucleotide that encodes a neoantigen, e.g., a neoantigen corresponding to a CD274 nucleic acid molecule or PD-L1polypeptide of the disclosure. In some embodiments, the cancer vaccine comprises DNA that encodes a neoantigen, e.g., a neoantigen corresponding to a CD274 nucleic acid molecule or PD-L1polypeptide of the disclosure. In some embodiments, the cancer vaccine comprises RNA that encodes a neoantigen, e.g., a neoantigen corresponding to a CD274 nucleic acid molecule or PD-L1 polypeptide of the disclosure. In some embodiments, the cancer vaccine comprises a polynucleotide that encodes a neoantigen, e.g., a neoantigen corresponding to a CD274 nucleic acid molecule or PD-L1 polypeptide of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelarcorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine, e.g., alone or in combination with an immune checkpoint inhibitor. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12:771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine, e.g., alone or in combination with an immune checkpoint inhibitor. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy, e.g., alone or in combination with an immune checkpoint inhibitor. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy, e.g., alone or in combination with an immune checkpoint inhibitor. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to a CD274 nucleic acid molecule or PD-L1 polypeptide of the disclosure

In some embodiments, the T cell-based therapy comprises T cells expressing a recombinant T cell receptor (TCR), e.g., alone or in combination with an immune checkpoint inhibitor. This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to a CD274 nucleic acid molecule or PD-L1 polypeptide of the disclosure.

In some embodiments, the T cell-based therapy comprises tumor-infiltrating lymphocytes (TILs), e.g., alone or in combination with an immune checkpoint inhibitor. For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to a CD274 nucleic acid molecule or PD-L1 polypeptide of the disclosure, in vitro after isolation. TILs are then administered to the patient (optionally in combination with one or more cytokines or other immune-stimulating substances).

In some embodiments, the cell-based therapy comprises a natural killer (NK) cell-based therapy, e.g., alone or in combination with an immune checkpoint inhibitor. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments. NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine, e.g., alone or in combination with an immune checkpoint inhibitor. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy, e.g., alone or in combination with an immune checkpoint inhibitor. In some embodiments, the cancer immunotherapy comprises administration of one or more TCRs or TCR-based therapeutics that specifically bind an antigen expressed by a cancer of the present disclosure, e.g., a neoantigen corresponding to a CD274 nucleic acid molecule or PD-L1 polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, an anti-cancer therapy of the disclosure comprises adjuvant immunotherapy. Adjuvant immunotherapy comprises the use of one or more agents that activate components of the innate immune system, e.g., HILTONOL® (imiquimod), which targets the TLR7 pathway.

In some embodiments, an anti-cancer therapy of the disclosure comprises cytokine immunotherapy, e.g., alone or in combination with an immune checkpoint inhibitor. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, an anti-cancer therapy of the disclosure comprises oncolytic virus therapy, e.g., alone or in combination with an immune checkpoint inhibitor. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, an anti-cancer therapy of the disclosure comprises an immunoregulatory molecule or a cytokine, e.g., alone or in combination with an immune checkpoint inhibitor. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, glp10, TCA-3, MCP-1, MIF, MIP-1a, MIP-1B, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, an anti-cancer therapy of the disclosure comprises an agent that inhibits expression of a nucleic acid that comprises or encodes a CD274 nucleic acid molecule of the disclosure or a portion thereof, or a PD-L1 polypeptide of the disclosure, or a portion thereof. In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA), dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes a CD274 nucleic acid molecule of the disclosure or a portion thereof comprising a breakpoint. Methods for designing, optimizing, producing, and using dsRNAs, siRNAs, or shRNAs, are known in the art.

In some embodiments, an anti-cancer therapy of the disclosure comprises a chemotherapy, e.g., alone or in combination with an immune checkpoint inhibitor. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, tricthiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, an anti-cancer therapy of the disclosure comprises a kinase inhibitor, e.g., alone or in combination with an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSF1R, c-Met, ROS1, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, an anti-cancer therapy of the disclosure comprises an anti-angiogenic agent, e.g., alone or in combination with an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin. VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin. VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v B3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF. VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, an anti-cancer therapy of the disclosure comprises an anti-DNA repair therapy, e.g., alone or in combination with an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, an anti-cancer therapy of the disclosure comprises a radiosensitizer, e.g., alone or in combination with an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, an anti-cancer therapy of the disclosure comprises an anti-inflammatory agent, e.g., alone or in combination with an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, an anti-cancer therapy of the disclosure comprises an anti-hormonal agent, e.g., alone or in combination with an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, an anti-cancer therapy of the disclosure comprises an antimetabolite chemotherapeutic agent, e.g., alone or in combination with an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCL is sold by Eli Lilly under the trademark GEMZAR®.

In some embodiments, an anti-cancer therapy of the disclosure comprises a platinum-based chemotherapeutic agent, e.g., alone or in combination with an immune checkpoint inhibitor. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein (e.g., an immune checkpoint inhibitor known in the art or described herein, and/or any other anti-cancer therapy known in the art or described herein), and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly (2-hydroxyethyl-methacrylate), or poly (vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an anti-cancer therapy of the disclosure (e.g., an immune checkpoint inhibitor) is administered as a monotherapy. In some embodiments, the anti-cancer therapy (e.g., an immune checkpoint inhibitor) is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy (e.g., an immune checkpoint inhibitor) may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy (e.g., an immune checkpoint inhibitor) may be administered in conjunction with a radiation therapy.

### D. Tumor Mutational Burden

In some embodiments, the methods provided herein comprise acquiring knowledge of or determining tumor mutational burden in a cancer of the disclosure, e.g., a cancer comprising a CD274 nucleic acid molecule of the disclosure, or a PD-L1 polypeptide of the disclosure.

Tumor mutational burden may be assessed using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6): 661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183. In some embodiments, tumor mutational burden is measured according to the methods provided in WO2017151524A1, which is hereby incorporated by reference in its entirety.

In some embodiments, tumor mutational burden is assessed based on the number of non-driver somatic coding mutations/megabase (mut/Mb) of genome sequenced. See, e.g., Chalmers et al., Genome Med 9, 34 (2017).

In some embodiments, tumor mutational burden is measured on between about 0.8 Mb and about 1.3 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, about 1.1 Mb, about 1.2 Mb, or about 1.3 Mb of sequenced DNA. In some embodiments, tumor mutational burden is measured on about 0.8 Mb of sequenced DNA. In some embodiments, tumor mutational burden is measured on between about 0.83 Mb and about 1.14 Mb of sequenced DNA. In some embodiments, tumor mutational burden is measured on up to about 1.24 Mb of sequenced DNA. In some embodiments, tumor mutational burden is measured on up to about 1.1 Mb of sequenced DNA. In some embodiments, tumor mutational burden is measured on about 0.79 Mb of sequenced DNA.

In some embodiments, a cancer of the disclosure, e.g., a cancer comprising a CD274 nucleic acid molecule or a PD-L1 polypeptide of the disclosure, has a tumor mutational burden of about 20 mut/Mb or less, e.g., any of about 20 mut/Mb, about 19 mut/Mb, about 18 mut/Mb, about 17 mut/Mb, about 16 mut/Mb, about 15 mut/Mb, about 14 mut/Mb, about 13 mut/Mb, about 12 mut/Mb, about 11 mut/Mb, about 10 mut/Mb, about 9.9 mut/Mb, about 9.8 mut/Mb, about 9.6 mut/Mb, about 9.4 mut/Mb, about 9.2 mut/Mb, about 9 mut/Mb, about 8.8 mut/Mb, about 8.6 mut/Mb, about 8.4 mut/Mb, about 8.2 mut/Mb, about 8 mut/Mb, about 7.8 mut/Mb, about 7.6 mut/Mb, about 7.4 mut/Mb, about 7.2 mut/Mb, about 7 mut/Mb, about 6.8 mut/Mb, about 6.6 mut/Mb, about 6.4 mut/Mb, about 6.2 mut/Mb, about 6 mut/Mb, about 5.8 mut/Mb, about 5.6 mut/Mb, about 5.4 mut/Mb, about 5.2 mut/Mb, about 5 mut/Mb, about 4.8 mut/Mb, about 4.6 mut/Mb, about 4.4 mut/Mb, about 4.2 mut/Mb, about 4 mut/Mb, about 3.8 mut/Mb, about 3.6 mut/Mb, about 3.4 mut/Mb, about 3.2 mut/Mb, about 3 mut/Mb, about 2.8 mut/Mb, about 2.6 mut/Mb, about 2.4 mut/Mb, about 2.2 mut/Mb, about 2 mut/Mb, about 1.8 mut/Mb, about 1.6 mut/Mb, about 1.4 mut/Mb, about 1.2 mut/Mb, about 1 mut/Mb, about 0.8 mut/Mb, about 0.6 mut/Mb, about 0.4 mut/Mb, about 0.2 mut/Mb, or less. In some embodiments, a cancer of the disclosure, e.g., a cancer comprising a CD274 nucleic acid molecule or a PD-L1 polypeptide of the disclosure, has a tumor mutational burden of less than about 10 mut/Mb. In some embodiments, a cancer of the disclosure. e.g., a cancer comprising a CD274 nucleic acid molecule or a PD-L1 polypeptide of the disclosure. has a tumor mutational burden of less than about 6 mut/Mb. In some embodiments, the cancer has a tumor mutational burden of between 6 and 20 mut/Mb,

In some embodiments, a cancer of the disclosure, e.g., a cancer comprising a CD274 nucleic acid molecule or a PD-L1 polypeptide of the disclosure, has a high tumor mutational burden. e.g., of at least about 10 mut/Mb. In some embodiments, the cancer has a tumor mutational burden of at least about 20 mut/Mb. In some embodiments, the cancer has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the cancer has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, the cancer has a TMB of at least about 100 mut/Mb, at least about 110 mut/Mb, at least about 120 mut/Mb, at least about 130 mut/Mb, at least about 140 mut/Mb, at least about 150 mut/Mb, or more.

In some embodiments, a cancer of the disclosure, e.g., a cancer comprising a CD274 nucleic acid molecule or a PD-L1 polypeptide of the disclosure, has a tumor mutational burden of approximately 7 mut/Mb,

In some embodiments, a cancer of the disclosure is a non-small cell lung carcinoma, a colorectal carcinoma, a carcinoma of unknown primary (CUP), a breast cancer, an esophageal cancer, a renal cell cancer or kidney cancer, a stomach or gastric carcinoma, or a head and neck cancer. In some embodiments, the cancer comprises a high tumor mutational burden, assessed using any suitable method known in the art or described herein. In some embodiments, the cancer comprises a tumor mutational burden of less than 6 mut/Mb, assessed using any suitable method known in the art or described herein. In some embodiments, the cancer comprises a tumor mutational burden of between 6 mut/Mb and 20 mut/Mb, assessed using any suitable method known in the art or described herein. In some embodiments, the cancer comprises a tumor mutational burden of greater than 20 mut/Mb, assessed using any suitable method known in the art or described herein. In some embodiments, the cancer comprises a tumor mutational burden of about 7 mut/Mb, assessed using any suitable method known in the art or described herein. In some embodiments, the cancer comprises a tumor mutational burden of at least 10 mut/Mb, assessed using any suitable method known in the art or described herein. In some embodiments, the cancer is a lung squamous cell carcinoma and comprises a tumor mutational burden of about 17.5 mut/Mb. In some embodiments, the cancer is a breast carcinoma (NOS) and comprises a tumor mutational burden of about 0 mut/Mb. In some embodiments, the cancer is a stomach adenocarcinoma and comprises a tumor mutational burden of between about 1.25 mut/Mb and about 13.75 mut/Mb. In some embodiments, the cancer is a colon adenocarcinoma and comprises a tumor mutational burden of about 7.5 mut/Mb,

In some embodiments, the methods of the disclosure comprise acquiring knowledge of or detecting a CD274 nucleic acid molecule or PD-L1 polypeptide of the disclosure, and acquiring knowledge of or determining tumor mutational burden, in a sample from an individual having cancer. In some embodiments, a cancer of the disclosure comprises a CD274 nucleic acid molecule or PD-L1 polypeptide of the disclosure, and a high tumor mutational burden.

In some embodiments, high tumor mutational burden refers to a tumor mutational burden of greater than or equal to 10 mutations/Mb. In some embodiments, high tumor mutational burden refers to a tumor mutational burden of greater than or equal to the median tumor mutational burden of the corresponding cancer type, e.g., as known in the art or determined according to any of the methods for assessing tumor mutational burden described herein.

In some embodiments, acquiring knowledge of or determining tumor mutational burden in a cancer of the disclosure comprises measuring the level of tumor mutational burden in a sample, e.g., in a sample from a cancer or a tumor, obtained from an individual. In some embodiments, the sample from the individual comprises fluid, cells, or tissue, e.g., from a liquid biopsy or a tissue biopsy such as a tumor biopsy, e.g., as described in greater detail herein. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA.

In some embodiments, tumor mutational burden is assessed in a sample derived from a cancer in an individual. In some embodiments, tumor mutational burden is assessed in a sample derived from a cancer in an individual and in a matched normal sample, e.g., a sample from the individual derived from a tissue or other source that is free of the cancer.

In some embodiments, tumor mutational burden is assessed based on a plurality of sequence reads, e.g., a plurality of sequence reads obtained by sequencing nucleic acids corresponding to at least a portion of a genome (such as from an enriched or unenriched sample). In some embodiments, tumor mutational burden is determined based on the number of non-driver somatic coding mutations per megabase of genome sequenced.

In some embodiments of any of the methods of the disclosure, the samples used to detect a CD274 nucleic acid molecule or PD-L1 polypeptide of the disclosure, and tumor mutational burden are the same sample. In some embodiments of any of the methods of the disclosure, the samples used to detect a CD274 nucleic acid molecule or PD-L1 polypeptide of the disclosure, and tumor mutational burden are different samples.

### E. PD-L1 Expression

In some embodiments, the methods provided herein comprise acquiring knowledge of or determining the level of PD-L1 expression in a cancer, e.g., a cancer described herein, e.g., comprising a CD274 nucleic acid molecule of the disclosure (e.g., a CD274 nucleic acid molecule comprising or resulting from a rearrangement described herein, or a CD274 fusion nucleic acid molecule described herein) or a PD-L1 polypeptide of the disclosure (e.g., a PD-L1 polypeptide encoded by a CD274 nucleic acid molecule of the disclosure). In some embodiments, the level of PD-L1 expression is assessed or determined in a sample, e.g., in a sample from an individual having a cancer, e.g., a cancer described herein, e.g., comprising a CD274 nucleic acid molecule of the disclosure or a PD-L1 polypeptide of the disclosure. In some embodiments, the same is obtained or derived from the cancer.

Any suitable method for measuring PD-L1 expression in a sample from an individual may be used. For example, the level of PD-L1 expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used.

In some embodiments, PD-L1 expression in a sample from an individual is measured based on the level of mRNA encoding PD-L1 in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, PD-L1 expression in a sample from an individual is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”).

In some embodiments, PD-L1 expression is measured using an immunohistochemistry assay in a sample obtained from the individual. In some embodiments, PD-L1 expression is measured by immunohistochemistry using an anti-PD-L1 antibody, such as a commercially available antibody, e.g., antibody clones 22C3 (Dako/Agilent) or SP142 (Ventana), e.g., according to methods known in the art and/or described herein.

In some embodiments, PD-L1 expression is assessed based on PD-L1 expression in tumor-infiltrating immune cells (IC) and/or tumor cells (TC). In some embodiments, a cancer provided herein, e.g., comprising a CD274 nucleic acid molecule or PD-L1 polypeptide of the disclosure, is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs), e.g., in a sample from an individual, express PD-L1 protein and/or PD-L1 mRNA (e.g., are positive for PD-L1 protein and/or PD-L1 mRNA). In some embodiments, a cancer provided herein, e.g., comprising a CD274 nucleic acid molecule or PD-L1 polypeptide of the disclosure, is determined to be positive for PD-L1 if the proportion of tumor area occupied by PD-L1 expressing tumor-infiltrating immune cells of any intensity (IC), or the percentage of PD-L1 expressing tumor cells of any intensity (TC), is at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%). In some embodiments, the cancer has a high PD-L1 expression. In some embodiments, high PD-L1 expression comprises a percentage of PD-L1 expressing tumor cells of any intensity of at least about 50%. In some embodiments, high PD-L1 expression comprises a proportion of tumor area occupied by PD-L1 expressing tumor-infiltrating immune cells of any intensity of at least about 10%. In some embodiments, the cancer is PD-L1 positive. In some embodiments, a PD-L1 positive cancer has a proportion of tumor area occupied by PD-L1 expressing tumor-infiltrating immune cells of any intensity of at least about 1% or at least about 5%. In some embodiments, PD-L1 expression, e.g., based on PD-L1 expression in tumor-infiltrating immune cells (IC) and/or tumor cells (TC) is assessed using a VENTANA SP 142 assay. Additional information about the VENTANA SP 142 assay and PD-L1 expression level scoring may be found in the website: www[dot]accessdata[dot]fda[dot]gov/cdrh_docs/pdf16/P160002c.pdf.

In some embodiments, PD-L1 expression is assessed based on a tumor proportion score (TPS). TPS refers to the percentage of viable tumor cells showing partial or complete PD-L1 membrane staining at any intensity (e.g., at a >1+ intensity on a 0, 1+, 2+, and 3 scale) relative to all viable tumor cells present in the sample. See, e.g., www[dot]agilent[dot]com/en/product/pharmdx/pd-11-ihc-22c3-pharmdx-overview #pink1. In some embodiments, the TPS is calculated as: the number of PD-L1-positive tumor cells/Total number of PD-L1-positive tumor cells+Total number of PD-L1-negative tumor cells. A PD-L1 low positive status refers to a TPS of between 1% and 49%, a PD-L1 high positive status refers to a TPS of 50% or greater, and a PD-L1 negative status refers to a TPS of less than 1%. In some embodiments, a cancer of the disclosure is PD-L1 positive if it has a PD-L1 low positive status or a PD-L1 high positive status. In some embodiments, a cancer of the disclosure is PD-L1 positive (e.g., the cancer has TPS of any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%). In some embodiments, a cancer of the disclosure is PD-L1 low positive (e.g., the cancer has a TPS of any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49%). In some embodiments, a cancer of the disclosure is PD-L1 high positive (e.g., the cancer has a TPS of any of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%). In some embodiments, a cancer of the disclosure is PD-L1 negative (e.g., the cancer has a TPS of less than 1%). In some embodiments, PD-L1 expression based on TPS is assessed using a DAKO 22C3 assay. Additional information about the DAKO 22C3 assay and the TPS score may be found, e.g., in the websites: www[dot]agilent[dot]com/en/product/pharmdx/pd-11-ihc-22c3-pharmdx-overview #pink3; www[dot]agilent[dot]com/cs/library/usermanuals/public/29171_22C3-ihc-pharmdx-interpretation-manual-eu.pdf; www[dot]agilent[dot]com/cs/library/usermanuals/public/13350a_eu_urothelial_carcinoma_interpretation_manual_r3v9_fin_150_single.pdf.pdf; and www[dot]agilent[dot]com/cs/library/usermanuals/public/29324_pd_11_ihc_22c3_hnscc_interpretation manual_kn048.pdf.

In some embodiments, PD-L1 expression is assessed based on a combined positive score (CPS). The CPS refers to the number of PD-L1 staining cells (e.g., tumor cells, lymphocytes, or macrophages) divided by the total number of viable tumor cells, and multiplied by 100. See, e.g., www[dot]agilent[dot]com/en/product/pharmdx/pd-11-ihc-22c3-pharmdx-overview #pink3. In some embodiments, a cancer of the disclosure has a CPS of at least about 1, at least about 2, at least about 3, at least about 4, at least about 5, at least about 6, at least about 7, at least about 8, at least about 9, or at least about 10. In some embodiments, a cancer of the disclosure has high PD-L1 expression, e.g., with a CPS of at least about 1, such as between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, or about 100. In some embodiments, PD-L1 expression based on CPS is assessed using a DAKO 22C3 assay. Additional information about the DAKO 22C3 assay and the CPS may be found, e.g., in the websites: www[dot]agilent[dot]com/en/product/pharmdx/pd-11-ihc-22c3-pharmdx-overview #pink3; www[dot]agilent[dot]com/cs/library/usermanuals/public/29171_22C3-ihc-pharmdx-interpretation-manual-eu.pdf; www[dot]agilent[dot]com/cs/library/usermanuals/public/13350a_eu_urothelial_carcinoma_interpretation_manual_r3v9_fin_150_single.pdf.pdf; and www[dot]agilent[dot]com/cs/library/usermanuals/public/29324_pd_11_ihc_22c3_hnscc_interpretation manual_kn048.pdf.

In some embodiments of any of the methods provided herein, PD-L1 expression is assessed using a companion diagnostic device, e.g., as provided in www[dot]fda[dot]gov/medical-devices/in-vitro-diagnostics/list-cleared-or-approved-companion-diagnostic-devices-in-vitro-and-imaging-tools. In some embodiments of any of the methods provided herein, PD-L1 expression is assessed as described in Huang et al., Mod Pathol 34, 252-263 (2021).

In some embodiments, a cancer of the disclosure (e.g., comprising a CD274 nucleic acid molecule or PD-L1 polypeptide of the disclosure) has a TPS of at least about 1% and the treatment or the one or more treatment options comprise pembrolizumab. In some embodiments, a cancer of the disclosure (e.g., comprising a CD274 nucleic acid molecule or PD-L1 polypeptide of the disclosure) has a TPS of at least about 50% and the treatment or the one or more treatment options comprise pembrolizumab. In some embodiments, the cancer is non-small cell lung cancer (NSCLC).

In some embodiments, a cancer of the disclosure (e.g., comprising a CD274 nucleic acid molecule or PD-L1 polypeptide of the disclosure) has a CPS of at least about 1 and the treatment or the one or more treatment options comprise pembrolizumab. In some embodiments, a cancer of the disclosure (e.g., comprising a CD274 nucleic acid molecule or PD-L1 polypeptide of the disclosure) has a TPS of at least about 50% and the treatment or the one or more treatment options comprise pembrolizumab. In some embodiments, the cancer is a gastric or gastroesophageal junction adenocarcinoma. In some embodiments, the cancer is a cervical cancer. In some embodiments, the cancer is a head and neck squamous cell carcinoma (HNSCC).

In some embodiments, a cancer of the disclosure (e.g., comprising a CD274 nucleic acid molecule or PD-L1 polypeptide of the disclosure) has a CPS of at least about 10 and the treatment or the one or more treatment options comprise pembrolizumab. In some embodiments, the cancer is a urothelial carcinoma. In some embodiments, the cancer is an esophageal squamous cell carcinoma.

In some embodiments, a cancer of the disclosure (e.g., comprising a CD274 nucleic acid molecule or PD-L1 polypeptide of the disclosure) comprises a proportion of tumor area occupied by PD-L1 expressing tumor-infiltrating immune cells of any intensity (IC) of at least about 1%, and the treatment or the one or more treatment options comprise atezolizumab. In some embodiments, the cancer is a triple negative breast cancer.

In some embodiments, a cancer of the disclosure (e.g., comprising a CD274 nucleic acid molecule or PD-L1 polypeptide of the disclosure) comprises a proportion of tumor area occupied by PD-L1 expressing tumor-infiltrating immune cells of any intensity (IC) of at least about 5%, and the treatment or the one or more treatment options comprise atezolizumab. In some embodiments, the cancer is a urothelial carcinoma.

In some embodiments, a cancer of the disclosure (e.g., comprising a CD274 nucleic acid molecule or PD-L1 polypeptide of the disclosure) comprises a proportion of tumor area occupied by PD-L1 expressing tumor-infiltrating immune cells of any intensity (IC) of at least about 10%, and the treatment or the one or more treatment options comprise atezolizumab. In some embodiments, a cancer of the disclosure (e.g., comprising a CD274 nucleic acid molecule or PD-L1 polypeptide of the disclosure) comprises a percentage of PD-L1 expressing tumor cells of any intensity (TC) of at least about 50%, and the treatment or the one or more treatment options comprise atezolizumab. In some embodiments, the cancer is a non-small cell lung cancer (NSCLC).

In some embodiments, the cancer is a lung non-squamous cell lung adenocarcinoma and is high positive for PD-L1 expression, e.g., with a TPS of between about 50% and about 59%, e.g., assessed using a DAKO 22C3 assay. In some embodiments, the cancer is a lung squamous cell carcinoma and is high positive for PD-L1 expression, e.g., with a TPS of about 70%, e.g., assessed using a DAKO 22C3 assay. In some embodiments, the cancer is a breast carcinoma (NOS) and is positive for PD-L1 expression, e.g., with an IC of about 1%, e.g., assessed using a VENTANA SP 142 assay. In some embodiments, the cancer is a colon adenocarcinoma and is high positive for PD-L1 expression, e.g., with a TPS of about 90%, e.g., assessed using a DAKO 22C3 assay.

In some embodiments, the level of PD-L1 protein and/or PD-L1 mRNA is assessed in a sample from an individual, such as a sample described herein. In some embodiments, the sample from the individual comprises fluid, cells, or tissue, e.g., from a liquid biopsy or a tissue biopsy such as a tumor biopsy as described in further detail herein. In some embodiments, the sample from the individual comprises a tumor biopsy or a circulating tumor cell, e.g., as described in further detail herein. In some embodiments, the sample is obtained or derived from a cancer of the disclosure, e.g., comprising a CD274 nucleic acid molecule or PD-L1 polypeptide of the disclosure.

### F. Microsatellite Instability Status

In some embodiments, the methods provided herein comprise acquiring knowledge of or determining a microsatellite instability status in a cancer of the disclosure, e.g., a cancer comprising a CD274 nucleic acid molecule or a PD-L1 polypeptide described herein.

Microsatellite instability may be assessed using any suitable method known in the art. For example, microsatellite instability may be measured using next generation sequencing (see, e.g., Hempelmann et al., J Immunother Cancer (2018) 6(1): 29), Fluorescent multiplex PCR and capillary electrophoresis (see, e.g., Arulananda et al., J Thorac Oncol (2018) 13(10): 1588-94), immunohistochemistry (see, e.g., Cheah et al., Malays J Pathol (2019) 41(2): 91-100), or single-molecule molecular inversion probes (smMIPs, see, e.g., Waalkes et al., Clin Chem (2018) 64(6): 950-8). In some embodiments, microsatellite instability is assessed based on DNA sequencing (e.g., next generation sequencing) of up to about 114 loci. In some embodiments, microsatellite instability is assessed based on DNA sequencing (e.g., next generation sequencing) of intronic homopolymer repeat loci for length variability. In some embodiments, microsatellite instability is assessed based on DNA sequencing (e.g., next generation sequencing) about 114 intronic homopolymer repeat loci for length variability. In some embodiments, microsatellite instability status (e.g., microsatellite instability high) is defined as described in Trabucco et al., J Mol Diagn. 2019 November; 21(6): 1053-1066.

In some embodiments, the methods of the disclosure comprise acquiring knowledge of or detecting a CD274 nucleic acid molecule or PD-L1 polypeptide of the disclosure, and acquiring knowledge of or determining microsatellite instability status, e.g., in a sample from an individual having cancer. In some embodiments, a cancer of the disclosure comprises a CD274 nucleic acid molecule or PD-L1 polypeptide described herein, and a high microsatellite instability status. In some embodiments, a cancer of the disclosure comprises a CD274 nucleic acid molecule or PD-L1 polypeptide described herein, and is microsatellite stable. In some embodiments, a cancer of the disclosure comprises a CD274 nucleic acid molecule or PD-L1 polypeptide described herein, and a low microsatellite instability status.

In some embodiments, acquiring knowledge of or determining a microsatellite instability status in a cancer of the disclosure comprises determining the microsatellite instability status in a sample, e.g., in a sample from a cancer or a tumor, obtained from an individual.

In some embodiments, microsatellite instability is assessed in sample from an individual, such as a sample described herein. In some embodiments, the sample from the individual comprises fluid, cells, or tissue, e.g., from a liquid biopsy or a tissue biopsy such as a tumor biopsy, e.g., a described in greater detail herein. In some embodiments, the sample from the individual comprises a tumor biopsy or a circulating tumor cell. In some embodiments, the sample from the individual comprises nucleic acids. In some embodiments, the sample from the individual comprises mRNA. DNA, circulating tumor DNA, cell-free DNA, or cell-free RNA. In some embodiments of any of the methods of the disclosure, the samples used to detect a CD274 nucleic acid molecule or PD-L1 polypeptide described herein, and a microsatellite instability status are the same sample, or are different samples.

In some embodiments, the cancer is a lung squamous cell carcinoma and is microsatellite stable. In some embodiments, the cancer is a breast carcinoma (NOS) and is microsatellite stable. In some embodiments, the cancer is a stomach adenocarcinoma and has high microsatellite instability (MSI-H). In some embodiments, the cancer is a stomach adenocarcinoma and is microsatellite stable. In some embodiments, the cancer is a colon adenocarcinoma and is microsatellite stable.

### G. Clonality

In some embodiments, the methods provided herein comprise acquiring knowledge of or determining the clonality of a CD274 nucleic acid molecule of the disclosure (e.g., a CD274 nucleic acid molecule comprising or resulting from a rearrangement described herein, or a CD274 fusion nucleic acid molecule described herein. e.g., in Tables 1-8, and/or in the Examples herein) in a cancer of the disclosure.

Clonality of a CD274 nucleic acid molecule of the disclosure may be assessed using any suitable method known in the art. In some embodiments, clonality of a CD274 nucleic acid molecule of the disclosure is assessed by performing DNA sequencing (e.g., according to any DNA sequencing methods known in the art or described herein) on a sample obtained from an individual.

In some embodiments, clonality of a CD274 nucleic acid molecule of the disclosure is assessed based on the number of sequence read pairs spanning a breakpoint of the CD274 nucleic acid molecule, e.g., a breakpoint as described herein. For example, in some embodiments, clonality of a CD274 nucleic acid molecule of the disclosure may be assessed based on a suitable threshold for the number of sequence read pairs spanning a breakpoint of the CD274 nucleic acid molecule, e.g., a threshold that is predictive of a sample from a tumor or cancer being PD-L1 high positive (e.g., having a TPS of at least about 50%). In some embodiments, determination of the clonality of a CD274 nucleic acid molecule of the disclosure is also based on the sequencing coverage of CD274. Thus, in some embodiments, clonality of a CD274 nucleic acid molecule of the disclosure may be assessed based on the sequencing coverage of CD274 gene and a suitable threshold for the number of sequence read pairs spanning a breakpoint of the CD274 nucleic acid molecule, e.g., a threshold that is predictive of a sample from a tumor or cancer being PD-L1 high positive (e.g., having a TPS of at least about 50%).

In some embodiments, the threshold for the number of sequence read pairs spanning a breakpoint of the CD274 nucleic acid molecule is selected using any suitable method known in the art. In some embodiments, the threshold is selected using a receiver operator characteristic (ROC) curve analysis. As is known in the art, ROC curve analysis is a method for determining how a threshold affects the sensitivity and specificity of a test. For example, in a test that predicts whether a sample from a tumor or cancer is PD-L1 high positive based on the number of sequence read pairs spanning a breakpoint of a CD274 nucleic acid molecule present in the sample, ROC curve analysis may be used for testing multiple cutoffs for the number of sequence read pairs spanning a breakpoint of the CD274 nucleic acid molecule to achieve a desired specificity and sensitivity for the test (i.e., specificity and sensitivity for predicting a sample from a tumor or cancer being PD-L1 high positive). See, e.g., Xia et al., Metabolomics. 2013 9(2): 280-299 and Habibzadeh et al., Biochem Med (Zagreb). 2016 26(3): 297-307, which are incorporated herein by reference, for discussion of ROC curve analysis and selection of suitable thresholds using ROC curve analysis. Accordingly, ROC curve analysis may be used in the methods of the disclosure for testing a multitude of thresholds for the number of sequence read pairs spanning a breakpoint of a CD274 nucleic acid molecule for their effect on specificity and sensitivity for predicting that a sample from a tumor or cancer is PD-L1 high positive. In some embodiments, the threshold is selected using the ROC curve analysis such that it maximizes the sum of sensitivity and specificity in the ROC curve analysis for predicting that a sample from a tumor or cancer is PD-L1 high positive.

In some embodiments, the threshold for the number of sequence read pairs spanning a breakpoint of a CD274 nucleic acid molecule is at least about 20 read pairs, at least about 21 read pairs, at least about 22 read pairs, at least about 23 read pairs, at least about 24 read pairs, at least about 25 read pairs, at least about 26 read pairs, at least about 27 read pairs, at least about 28 read pairs, at least about 29 read pairs, at least about 30 read pairs, at least about 31 read pairs, at least about 32 read pairs, at least about 33 read pairs, at least about 34 read pairs, at least about 35 read pairs, at least about 36 read pairs, at least about 37 read pairs, at least about 38 read pairs, at least about 39 read pairs, at least about 40 read pairs, at least about 41 read pairs, at least about 42 read pairs, at least about 43 read pairs, at least about 44 read pairs, at least about 45 read pairs, at least about 46 read pairs, at least about 47 read pairs, at least about 48 read pairs, at least about 49 read pairs, or at least about 50 read pairs spanning a breakpoint of the CD274 nucleic acid molecule. In some embodiments, the threshold for the number of sequence read pairs spanning a breakpoint of a CD274 nucleic acid molecule is at least about 25 sequence read pairs.

In some embodiments, a CD274 nucleic acid molecule of the disclosure is determined to be clonal in a cancer, or to result from a clonal rearrangement of a CD274 gene in a cancer, if DNA sequencing of a sample from the cancer results in a number of sequence read pairs spanning a breakpoint of the CD274 nucleic acid molecule that is equal to or greater than a threshold, e.g., a threshold for the number of sequence read pairs spanning a breakpoint of the CD274 nucleic acid molecule provided above, or selected as described above. In some embodiments, a CD274 nucleic acid molecule of the disclosure is determined to be sub-clonal in a cancer, or to result from a sub-clonal rearrangement of a CD274 gene in a cancer, if DNA sequencing of a sample from the cancer results in a number of sequence read pairs spanning a breakpoint of the CD274 nucleic acid molecule that is less than a threshold, e.g., a threshold for the number of sequence read pairs spanning a breakpoint of the CD274 nucleic acid molecule provided above, or selected as described above.

In some embodiments of any of the methods provided herein, clonality of a CD274 nucleic acid molecule of the disclosure is assessed according to the methods as described in Cmero et al., Nat Commun 11, 730 (2020), which is incorporated herein by reference.

In some embodiments, any of the CD274 nucleic acid molecules of the disclosure may result from a clonal or a sub-clonal rearrangement of a CD274 gene in a cancer, e.g., a rearrangement described herein. In some embodiments, any of the CD274 nucleic acid molecules of the disclosure result from a clonal rearrangement of a CD274 gene in a cancer, e.g., a rearrangement described herein. In some embodiments, any of the CD274 nucleic acid molecules of the disclosure result from a sub-clonal rearrangement of a CD274 gene in a cancer, e.g., a rearrangement described herein.

In some embodiments, any of the CD274 nucleic acid molecules of the disclosure may be clonal or sub-clonal in a cancer. In some embodiments, any of the CD274 nucleic acid molecules of the disclosure are clonal in a cancer. In some embodiments, any of the CD274 nucleic acid molecules of the disclosure are sub-clonal in a cancer.

In some embodiments, the presence of a clonal CD274 nucleic acid molecule of the disclosure in a cancer, or of a CD274 nucleic acid molecule that results from a clonal rearrangement of a CD274 gene in the cancer, identifies the individual as: (a) likely to respond to a treatment comprising an immune checkpoint inhibitor; (b) likely to have longer survival when treated with a treatment comprising an immune checkpoint inhibitor, as compared to: (i) survival of an individual whose cancer does not comprise a clonal CD274 nucleic acid molecule, or a CD274 nucleic acid molecule resulting from a clonal rearrangement of a CD274 gene, or (ii) survival of an individual whose cancer comprises a sub-clonal CD274 nucleic acid molecule, or a CD274 nucleic acid molecule resulting from a sub-clonal rearrangement of a CD274 gene; (c) likely to have an improved response to treatment with an immune checkpoint inhibitor, as compared to: (i) an individual whose cancer does not comprise a clonal CD274 nucleic acid molecule, or a CD274 nucleic acid molecule resulting from a clonal rearrangement of a CD274 gene, or (ii) an individual whose cancer comprises a sub-clonal CD274 nucleic acid molecule, or a CD274 nucleic acid molecule resulting from a sub-clonal rearrangement of a CD274 gene.

In some embodiments, the presence of a clonal CD274 nucleic acid molecule of the disclosure in a cancer, or of a CD274 nucleic acid molecule that results from a clonal rearrangement of a CD274 gene in the cancer, identifies the cancer as likely to be PD-L1 positive or PD-L1 high positive, or to have a tumor proportion score (TPS) of at least about 50%, e.g., as described herein, e.g., in the Examples and/or in the PD-L1 Expression section herein.

In some embodiments, clonality of a CD274 nucleic acid molecule is assessed in sample from an individual, such as sample described herein. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA. In some embodiments, the sample is a bulk tumor sample derived from a single anatomic location.

### H. Reporting

In some embodiments, the methods provided herein comprise generating a report, and/or providing a report to party.

In some embodiments, a report according to the present disclosure comprises information about one or more of: a CD274 nucleic acid molecule of the disclosure (e.g., a CD274 nucleic acid molecule comprising or resulting from a rearrangement described herein, or a CD274 fusion nucleic acid molecule described herein, e.g., in Tables 1-8, and/or in the Examples herein), or a PD-L1 polypeptide encoded by such a CD274 nucleic acid molecule; a cancer of the disclosure, e.g., comprising a CD274 nucleic acid molecule or PD-L1 polypeptide of the disclosure; or a treatment, a therapy, or one or more treatment options for an individual having a cancer, such as a cancer of the disclosure (e.g., comprising a CD274 nucleic acid molecule or a PD-L1 polypeptide described herein).

In some embodiments, a report according to the present disclosure comprises information about the presence or absence of a CD274 nucleic acid molecule of the disclosure (e.g., a CD274 nucleic acid molecule comprising or resulting from a rearrangement described herein, or a CD274 fusion nucleic acid molecule described herein, e.g., in Tables 1-8, and/or in the Examples herein), or a PD-L1 polypeptide encoded by such a CD274 nucleic acid molecule, in a sample obtained from an individual, such as an individual having a cancer, e.g., a cancer provided herein. In one embodiment, a report according to the present disclosure indicates that a CD274 nucleic acid molecule or a PD-L1 polypeptide of the disclosure is present in a sample obtained from the individual. In one embodiment, a report according to the present disclosure indicates that a CD274 nucleic acid molecule or a PD-L1 polypeptide of the disclosure is not present in a sample obtained from the individual. In one embodiment, a report according to the present disclosure indicates that a CD274 nucleic acid molecule or a PD-L1 polypeptide of the disclosure has been detected in a sample obtained from the individual. In one embodiment, a report according to the present disclosure indicates that a CD274 nucleic acid molecule or a PD-L1 polypeptide of the disclosure has not been detected in a sample obtained from the individual. In some embodiments, the report comprises an identifier for the individual from which the sample was obtained.

In some embodiments, the report includes information on the role of a CD274 nucleic acid molecule of the disclosure (e.g., a CD274 nucleic acid molecule comprising or resulting from a rearrangement described herein, or a CD274 fusion nucleic acid molecule described herein, e.g., in Tables 1-8, and/or in the Examples herein), or a PD-L1 polypeptide encoded by such a CD274 nucleic acid molecule, or its wild type counterparts, in disease, such as in cancer. Such information can include one or more of: information on prognosis of a cancer, such as a cancer provided herein. e.g., comprising a CD274 nucleic acid molecule or a PD-L1 polypeptide described herein; information on resistance of a cancer, such as a cancer provided herein, e.g., comprising a CD274 nucleic acid molecule or a PD-L1 polypeptide described herein, to one or more treatments; information on potential or suggested therapeutic options (e.g., such as an anti-cancer therapy provided herein, or a treatment selected or identified according to the methods provided herein, e.g., an immune checkpoint inhibitor); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein, e.g., an immune checkpoint inhibitor) to an individual having a cancer, such as a cancer provided herein, e.g., comprising a CD274 nucleic acid molecule or a PD-L1 polypeptide described herein and identified in the report. In some embodiments, the report includes information or a recommendation on the administration of a treatment (e.g., an anti-cancer therapy provided herein, or a treatment selected or identified according to the methods provided herein, e.g., an immune checkpoint inhibitor). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting a CD274 nucleic acid molecule of the disclosure (e.g., a CD274 nucleic acid molecule comprising or resulting from a rearrangement described herein, or a CD274 fusion nucleic acid molecule described herein, e.g., in Tables 1-8, and/or in the Examples herein), or a PD-L1 polypeptide encoded by such a CD274 nucleic acid molecule, in the sample, or acquiring knowledge of the presence of the CD274 nucleic acid molecule or PD-L1 polypeptide of the disclosure in the sample; and generating a report. In some embodiments, a report generated according to the methods provided herein comprises one or more of: information about the presence or absence of a CD274 nucleic acid molecule of the disclosure (e.g., a CD274 nucleic acid molecule comprising or resulting from a rearrangement described herein, or a CD274 fusion nucleic acid molecule described herein, e.g., in Tables 1-8, and/or in the Examples herein), or a PD-L1 polypeptide encoded by such a CD274 nucleic acid molecule, in the sample; an identifier for the individual from which the sample was obtained; information on the role of the CD274 nucleic acid molecule or PD-L1 polypeptide of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein, e.g., an immune checkpoint inhibitor); information on the likely effectiveness, acceptability, or the advisability of applying a therapeutic option (such as an anti-cancer therapy provided herein, or a treatment selected or identified according to the methods provided herein, e.g., an immune checkpoint inhibitor) to the individual; a recommendation or information on the administration of a treatment (such as an anti-cancer therapy provided herein, or a treatment selected or identified according to the methods provided herein, e.g., an immune checkpoint inhibitor); or a recommendation or information on the dosage or treatment regimen of a treatment (such as an anti-cancer therapy provided herein, or a treatment selected or identified according to the methods provided herein, e.g., an immune checkpoint inhibitor), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., comprising a CD274 nucleic acid molecule or a PD-L1 polypeptide of the disclosure), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a CD274 nucleic acid molecule or a PD-L1 polypeptide of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a CD274 nucleic acid molecule or PD-L1 polypeptide of the disclosure in a sample obtained from an individual (e.g., an individual having a cancer).

### I. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform a method according to any of the embodiments described herein.

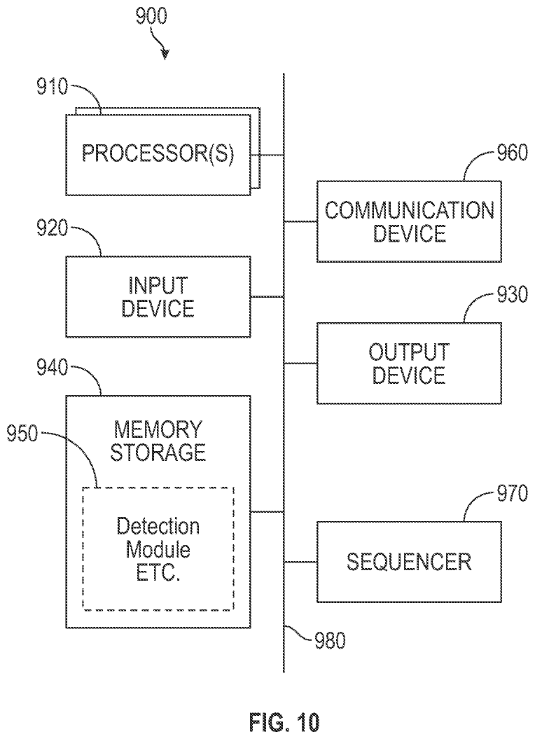

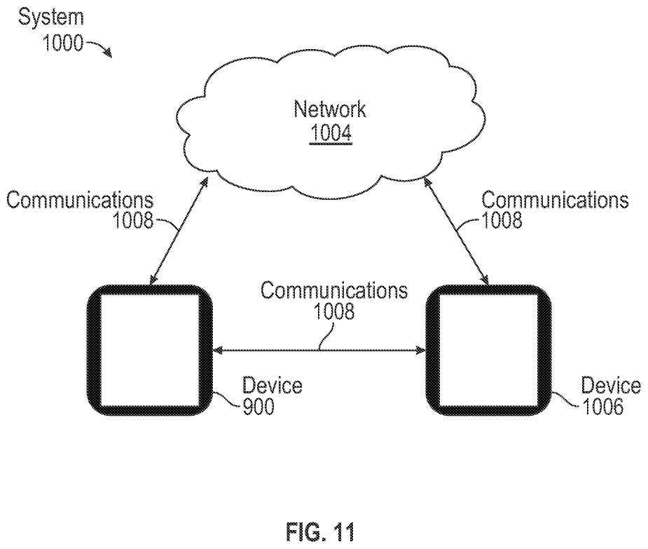

FIG. 10 illustrates an example of a computing device or system in accordance with one embodiment. Device 900 can be a host computer connected to a network. Device 900 can be a client computer or a server. As shown in FIG. 10, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting a CD274 nucleic acid molecule of the disclosure. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting a CD274 nucleic acid molecule of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 900 may be connected to a network (e.g., network 1004, as shown in FIG. 11 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C. C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

FIG. 11 illustrates an example of a computing system in accordance with one embodiment. In computing system 1000, device 900 (e.g., as described above and illustrated in FIG. 10) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

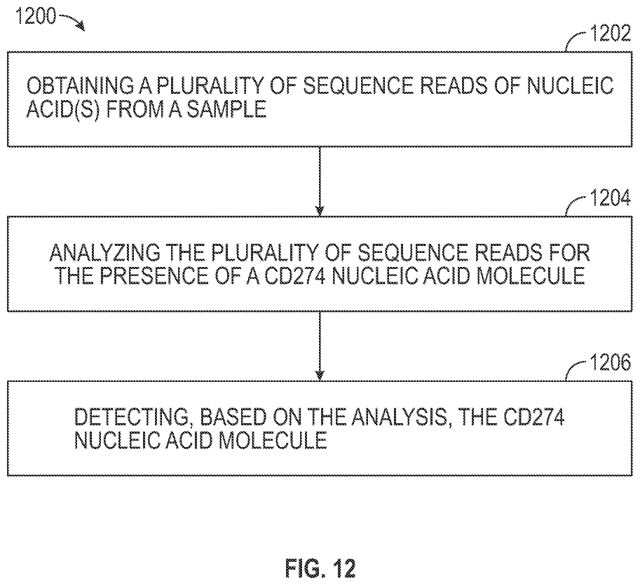

FIG. 12 illustrates an exemplary process 1200 for detecting a CD274 nucleic acid molecule of the disclosure in a sample, in accordance with some embodiments of the present disclosure. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In some embodiments, the executed steps can be executed across many systems, e.g., in a cloud environment. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of sequence reads of one or more nucleic acid molecules is obtained, wherein the one or more nucleic acid molecules are derived from a sample obtained from an individual, e.g., as described herein. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to: a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein); or a gene involved in a CD274 nucleic acid molecule of the disclosure; or fragments thereof. Optionally, prior to obtaining the sequence reads, the sample is purified, enriched (e.g., for nucleic acid(s) corresponding to: a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein); or a gene involved in a CD274 nucleic acid molecule of the disclosure; or fragments thereof), and/or subjected to PCR amplification. At block 1204, an exemplary system (e.g., one or more electronic devices) analyzes the plurality of sequence reads for the presence of a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein), or a fragment thereof. At block 1206, the system detects (e.g., based on the analysis) a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein), or a fragment thereof, in the sample.

In some embodiments of any of the methods, systems, devices, non-transitory computer readable storage media, or processes of the disclosure, the CD274 nucleic acid molecule comprises or results from a rearrangement comprising a Breakpoint 1 and/or Breakpoint 2 within the chromosomal coordinates as listed in Table 1. In some embodiments of any of the methods, systems, devices, non-transitory computer readable storage media, or processes of the disclosure, the CD274 nucleic acid molecule is a CD274 fusion nucleic acid molecule comprising a fusion between a CD274 gene, or a portion thereof, and a fusion partner gene as listed in Table 2, or a portion thereof. In some embodiments of any of the methods, systems, devices, non-transitory computer readable storage media, or processes of the disclosure, the cancer is a carcinoma, a sarcoma, a lymphoma, a leukemia, a myeloma, a germ cell cancer, or a blastoma. In some embodiments of any of the methods, systems, devices, non-transitory computer readable storage media, or processes of the disclosure, the cancer is a solid tumor or a hematologic malignancy. In some embodiments of any of the methods, systems, devices, non-transitory computer readable storage media, or processes of the disclosure, the cancer is any cancer described herein or known in the art. In some embodiments of any of the methods, systems, devices, non-transitory computer readable storage media, or processes of the disclosure, the cancer is a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, lung non-small cell lung carcinoma (NSCLC), gastric cancer or carcinoma, head and neck cancer, small cell cancer, essential thrombocythemia, agnogenic myeloid metaplasia, hypereosinophilic syndrome, systemic mastocytosis, familiar hypercosinophilia, chronic eosinophilic leukemia, neuroendocrine cancers, or a carcinoid tumor. In some embodiments of any of the methods, systems, devices, non-transitory computer readable storage media, or processes of the disclosure, the cancer is an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, gastric cancer or carcinoma, unknown primary adenocarcinoma, unknown primary carcinoma (CUP), unknown primary carcinoma (CUP) (NOS), unknown primary melanoma, stomach adenocarcinoma, or vagina squamous cell carcinoma (SCC). In some embodiments of any of the methods, systems, devices, non-transitory computer readable storage media, or processes of the disclosure, the CD274 nucleic acid molecule comprises or results from a rearrangement comprising a Breakpoint 1 and/or Breakpoint 2 within the chromosomal coordinates as listed in Table 1, and the cancer is the corresponding cancer as listed in Table 6. In some embodiments of any of the methods, systems, devices, non-transitory computer readable storage media, or processes of the disclosure, the CD274 fusion nucleic acid molecule comprises a fusion between a CD274 gene, or a portion thereof, and a fusion partner gene as listed in Table 2, or a portion thereof, and the cancer is the corresponding cancer as listed in Table 7. In some embodiments of any of the methods, systems, devices, non-transitory computer readable storage media, or processes of the disclosure, the CD274 fusion nucleic acid molecule comprises a fusion between a CD274 gene, or a portion thereof, and a fusion partner gene as listed in Table 2, or a portion thereof, and the CD274 fusion nucleic acid molecule comprises or results from a corresponding CD274 Breakpoint and/or Fusion Partner Gene Breakpoint within the chromosomal coordinates as listed in Table 4. In some embodiments of any of the methods, systems, devices, non-transitory computer readable storage media, or processes of the disclosure, the CD274 fusion nucleic acid molecule comprises a fusion between a CD274 gene, or a portion thereof, and a fusion partner gene as listed in Table 2, or a portion thereof; the CD274 fusion nucleic acid molecule comprises or results from a corresponding CD274 Breakpoint and/or Fusion Partner Gene Breakpoint within the chromosomal coordinates as listed in Table 8; and the cancer is the corresponding cancer as listed in Table 8.

In some embodiments of any of the methods, systems, devices, non-transitory computer readable storage media, or processes of the disclosure, the CD274 nucleic acid molecule is a CD274 fusion nucleic acid molecule comprising a fusion between a CD274 gene, or a portion thereof, and a fusion partner gene as listed in Table 3, or a portion thereof, and the cancer is the corresponding cancer as listed in Table 3. In some embodiments of any of the methods, systems, devices, non-transitory computer readable storage media, or processes of the disclosure, the CD274 fusion nucleic acid molecule comprises a fusion between a CD274 gene, or a portion thereof, and a fusion partner gene as listed in Table 3, or a portion thereof, the cancer is the corresponding cancer as listed in Table 3; and the CD274 fusion nucleic acid molecule comprises or results from a corresponding CD274 Breakpoint and/or Fusion Partner Gene Breakpoint within the chromosomal coordinates as listed in Table 5.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA. DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the disclosed methods for determining the presence or absence of a CD274 nucleic acid molecule of the disclosure may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of a CD274 nucleic acid molecule of the disclosure as part of a genomic profiling process can improve the validity of, e.g., disease detection calls, made on the basis of the genomic profile by, for example, independently confirming the presence of the CD274 nucleic acid molecule of the disclosure in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the individual may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

Accordingly, in some embodiments of any of the methods, systems, devices, non-transitory computer readable storage media, or processes of the disclosure, a genomic profile for the sample or for the individual is generated based at least in part on detecting a CD274 nucleic acid molecule of the disclosure, or a fragment thereof, in the sample. In some embodiments, the individual is administered a treatment based at least in part on the genomic profile, e.g., a treatment described herein. In some embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile comprises results from a nucleic acid sequencing-based test. In some embodiments, the genomic profile comprises/indicates/comprises information on presence or absence of mutations in one or more additional genes, e.g., a panel of known oncogenes and/or tumor suppressors. In some embodiments, the genomic profile is obtained from a genomic profiling assay (such as a cancer- or tumor-related genomic profiling assay), e.g., as obtained using any of the sequencing methodologies described herein. In some embodiments, the genomic profile includes information from whole-genome or whole-exome sequencing. In some embodiments, the genomic profile includes information from targeted sequencing. In some embodiments, the genomic profile includes information from NGS. In some embodiments, the genomic profile comprises/indicates/comprises information on presence or absence of mutations such as short variant alterations (e.g., a base substitution, insertion, or deletion), copy-number alterations (e.g., an amplification or a homozygous deletion), and/or fusions (e.g., a gene fusion or other genomic or chromosomal fusion) of one or more genes, e.g., a panel of known oncogenes and/or tumor suppressors. In some embodiments, the genomic profile comprises/indicates/comprises information on the level of tumor mutational burden (TMB), clonal TMB, indel TMB, or nonsense-mediated decay (NMD)-escape TMB; the presence or absence of, or the proportion of mutations fitting, a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1. CD8A, and/or CXCL9; or any combination thereof, e.g., as known in the art or described herein. See, e.g., Litchfield et al., Cell. 2021 Feb. 4; 184(3): 596-614.e14 for additional information.

In some embodiments of any of the methods, systems, devices, non-transitory computer readable storage media, or processes, a report is generated indicating the presence or absence of the CD274 nucleic acid molecule in the sample. In some embodiments, the report is generated by the one or more processors of any of the systems, devices, or non-transitory computer readable storage media of the disclosure. In some embodiments, the report is transmitted to a healthcare provider or another entity, e.g., as described herein. In some embodiments, the report is transmitted via a computer network or a peer-to-peer connection.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. Articles of Manufacture or Kits

Provided herein are kits or articles of manufacture comprising one or more reagents for detecting a CD274 nucleic acid molecule of the disclosure in a sample, e.g., a CD274 nucleic acid molecule comprising or resulting from a rearrangement described herein, or a CD274 fusion nucleic acid molecule described herein (e.g., in Tables 1-8, and/or in the Examples herein); or a PD-L1 polypeptide of the disclosure, e.g., a PD-L1 polypeptide encoded by a CD274 nucleic acid molecule of the disclosure.

In some embodiments, the kits or articles of manufacture comprise one or more probes of the disclosure for detecting a CD274 nucleic acid molecule of the disclosure in a sample, e.g., according to any detection method known in the art or described herein. In some embodiments, the kits or articles of manufacture comprise one or more baits (e.g., one or more bait molecules) of the disclosure for detecting a CD274 nucleic acid molecule of the disclosure in a sample, e.g., according to any detection method known in the art or described herein. In some embodiments, the kits or articles of manufacture comprise one or more oligonucleotides (e.g., one or more primers) of the disclosure for detecting a CD274 nucleic acid molecule of the disclosure in a sample, e.g., according to any detection method known in the art or described herein. In some embodiments of any of the kits or articles of manufacture provided herein, the kit or article of manufacture comprises a reagent (e.g., one or more oligonucleotides, primers, probes or baits of the present disclosure) for detecting a wild-type counterpart of a CD274 nucleic acid molecule of the disclosure (e.g., a wild type CD274 gene, and/or a wild type fusion partner gene described herein). In some embodiments, one or more oligonucleotides, primers, probes or baits are capable of hybridizing to a CD274 nucleic acid molecule of the disclosure, or to a wild-type counterpart of the CD274 nucleic acid molecule (e.g., a wild type CD274 gene, and/or a wild type fusion partner gene described herein). In some embodiments, the one or more oligonucleotides, primers, probes or baits of the present disclosure are capable of distinguishing a CD274 nucleic acid molecule of the disclosure from a wild-type counterpart of the CD274 nucleic acid molecule (e.g., a wild type CD274 gene, and/or a wild type fusion partner gene described herein). In some embodiments, the kit is for use according to any method of detecting nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a CD274 nucleic acid molecule of the disclosure, e.g., using one or more oligonucleotides, primers, probes or baits of the present disclosure.

In some embodiments, the kits or articles of manufacture comprise one or more antibodies or antibody fragments of the disclosure for detecting a PD-L1 polypeptide of the disclosure, e.g., a PD-L1 polypeptide encoded by a CD274 nucleic acid molecule of the disclosure, in a sample, e.g., according to any detection method known in the art or described herein. In some embodiments, the kit or article of manufacture comprises a reagent (e.g., one or more antibodies of the present disclosure) for detecting the wild-type counterparts of a PD-L1 polypeptide provided herein (e.g., a wild type PD-L1 polypeptide, and/or a wild type polypeptide encoded by a fusion partner gene described herein). In some embodiments, the kits or articles of manufacture comprise one or more antibodies of the present disclosure capable of binding to a PD-L1 polypeptide provided herein, or to wild-type counterparts of the PD-L1 polypeptide provided herein (e.g., a wild type PD-L1 polypeptide, and/or a wild type polypeptide encoded by a fusion partner gene described herein). In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a PD-L1 polypeptide of the disclosure, e.g., using one or more antibodies of the present disclosure.

Further provided herein are kits or articles of manufacture comprising an anti-cancer therapy, such as an anti-cancer therapy described herein (e.g., an immune checkpoint inhibitor), and a package insert comprising instructions for using the anti-cancer therapy in a method of treating or delaying progression of cancer, e.g., by administration to an individual from whom a sample comprising a CD274 nucleic acid molecule or a PD-L1 polypeptide of the disclosure has been obtained. In some embodiments, the anti-cancer therapy is any of the anti-cancer therapies described herein for use in any of the methods for treating or delaying progression of cancer of the disclosure.

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting a CD274 nucleic acid molecule or a PD-L1 polypeptide of the disclosure (e.g., one or more oligonucleotides, primers, probes, baits, antibodies or antibody fragments of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents are used for detecting a CD274 nucleic acid molecule or a PD-L1 polypeptide of the disclosure, or indicating that the one or more anti-cancer therapies are used for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. Expression Vectors, Host Cells and Recombinant Cells

Provided herein are vectors comprising or encoding a CD274 nucleic acid molecule of the disclosure, e.g., a CD274 nucleic acid molecule comprising or resulting from a rearrangement described herein, or a CD274 fusion nucleic acid molecule described herein (e.g., in Tables 1-8, and/or in the Examples herein), or a bait, a probe, or an oligonucleotide described herein, or fragments thereof.

In some embodiments, a vector provided herein comprises or encodes a CD274 nucleic acid molecule of the disclosure, e.g., a CD274 nucleic acid molecule comprising or resulting from a rearrangement described herein, or a CD274 fusion nucleic acid molecule described herein, (e.g., in Tables 1-8, and/or in the Examples herein), or a nucleic acid molecule encoding a PD-L1 polypeptide described herein (e.g., encoded by a CD274 nucleic acid molecule of the disclosure).

In some embodiments, a vector provided herein is a nucleic acid molecule capable of transporting another nucleic acid to which it has been linked (e.g., CD274 nucleic acid molecules, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising CD274 nucleic acid molecules, baits, probes, or oligonucleotides described herein, or fragments thereof) are also contemplated herein, including, e.g., replication defective retroviruses, adenoviruses and adeno-associated viruses.

In some embodiments, a vector provided herein comprises a CD274 nucleic acid molecule, a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., CD274 nucleic acid molecules, baits, probes, or oligonucleotides described herein, or fragments thereof). In some embodiments, a regulatory sequence directs tissue-specific expression of a nucleotide sequence (e.g., CD274 nucleic acid molecules, baits, probes, or oligonucleotides described herein, or fragments thereof). In some embodiments, a regulatory sequence directs inducible expression of a nucleotide sequence (e.g., CD274 nucleic acid molecules, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises or encodes a CD274 nucleic acid molecule, a bait, a probe, or an oligonucleotide of the disclosure in the sense or the anti-sense orientation. In some embodiments, a vector (e.g., an expression vector) provided herein is introduced into host cells to thereby produce a polypeptide, e.g., a PD-L1 polypeptide described herein, or a fragment or mutant form thereof.

In some embodiments, the design of a vector provided herein depends on such factors as the choice of the host cell to be transformed, the level of expression desired, and the like. In some embodiments, expression vectors are designed for the expression of the CD274 nucleic acid molecules, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises or encodes a CD274 nucleic acid molecule described herein, wherein the nucleotide sequence of the CD274 nucleic acid molecule described herein has been altered (e.g., codon optimized) so that the individual codons for each encoded amino acid are those preferentially utilized in the host cell.

Also provided herein are host cells, e.g., comprising CD274 nucleic acid molecules, PD-L1 polypeptides, baits, probes, vectors, or oligonucleotides of the disclosure. In some embodiments, a host cell (e.g., a recombinant host cell or recombinant cell) comprises a vector described herein (e.g., an expression vector described herein). In some embodiments, a CD274 nucleic acid molecule, bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

CD274 nucleic acid molecules, baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing a PD-L1 polypeptide of the disclosure, e.g., by culturing a host cell described herein (e.g., into which a recombinant expression vector encoding a polypeptide has been introduced) in a suitable medium such that the PD-L1 polypeptide is produced. In another embodiment, the method further includes isolating a PD-L1 polypeptide from the medium or the host cell.

### VI. Exemplary Clauses

The following exemplary clauses are representative of some aspects of the invention:

Exemplary Clause 1: A method of identifying an individual having a cancer who may benefit from a treatment comprising an immune checkpoint inhibitor, the method comprising detecting in a sample from the individual a cluster of differentiation 274 (CD274) nucleic acid molecule, or a programmed death-ligand 1 (PD-L1) polypeptide encoded by the CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
      wherein detection of the CD274 nucleic acid molecule, or the PD-L1 polypeptide encoded by the CD274 nucleic acid molecule, in the sample identifies the individual as one who may benefit from a treatment comprising an immune checkpoint inhibitor.

Exemplary Clause 2: A method of selecting a treatment for an individual having a cancer, the method comprising detecting in a sample from the individual a CD274 nucleic acid molecule, or a PD-L1 polypeptide encoded by the CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
      wherein detection of the CD274 nucleic acid molecule, or the PD-L1 polypeptide encoded by the CD274 nucleic acid molecule, in the sample identifies the individual as one who may benefit from a treatment comprising an immune checkpoint inhibitor.

Exemplary Clause 3: A method of identifying one or more treatment options for an individual having a cancer, the method comprising:

detecting in a sample from the individual a CD274 nucleic acid molecule, or a PD-L1 polypeptide encoded by the CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and
      generating a report comprising one or more treatment options identified for the individual based at least in part on detection of the CD274 nucleic acid molecule, or the PD-L1 polypeptide encoded by the CD274 nucleic acid molecule, wherein the one or more treatment options comprise an immune checkpoint inhibitor.

Exemplary Clause 4: A method of identifying one or more treatment options for an individual having a cancer, the method comprising:

acquiring knowledge of a CD274 nucleic acid molecule, or a PD-L1 polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and
      generating a report comprising one or more treatment options identified for the individual based at least in part on said knowledge, wherein the one or more treatment options comprise an immune checkpoint inhibitor.

Exemplary Clause 5: A method of selecting a treatment for an individual having cancer, comprising:

acquiring knowledge of a CD274 nucleic acid molecule, or a PD-L1 polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
      wherein responsive to the acquisition of said knowledge: (i) the individual is classified as a candidate to receive a treatment comprising an immune checkpoint inhibitor; and/or (ii) the individual is identified as likely to respond to a treatment that comprises an immune checkpoint inhibitor.

Exemplary Clause 6: A method of predicting survival of an individual having a cancer, comprising:

acquiring knowledge of a CD274 nucleic acid molecule, or a PD-L1 polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
      wherein responsive to the acquisition of said knowledge, the individual is predicted to have longer survival when treated with a treatment comprising an immune checkpoint inhibitor, as compared to survival of an individual whose cancer does not comprise the CD274 nucleic acid molecule, or the PD-L1 polypeptide encoded by the CD274 nucleic acid molecule.

Exemplary Clause 7: A method of predicting survival of an individual having a cancer treated with a treatment comprising an immune checkpoint inhibitor, the method comprising:

acquiring knowledge of a CD274 nucleic acid molecule, or a PD-L1 polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
      wherein responsive to the acquisition of said knowledge, the individual is predicted to have longer survival when treated with a treatment comprising an immune checkpoint inhibitor, as compared to an individual whose cancer does not exhibit the CD274 nucleic acid molecule, or the PD-L1 polypeptide encoded by the CD274 nucleic acid molecule.

Exemplary Clause 8: A method of treating or delaying progression of cancer, comprising:

acquiring knowledge of a CD274 nucleic acid molecule, or a PD-L1 polypeptide encoded by the CD274 nucleic acid molecule, in a sample from an individual having a cancer, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and
      responsive to said knowledge, administering to the individual an effective amount of a treatment that comprises an immune checkpoint inhibitor.

Exemplary Clause 9: A method of treating or delaying progression of cancer, comprising administering to an individual having a cancer an effective amount of a treatment that comprises an immune checkpoint inhibitor, wherein the treatment is administered responsive to acquiring knowledge of a CD274 nucleic acid molecule, or a PD-L1 polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3.

Exemplary Clause 10: A method of monitoring, evaluating or screening an individual having a cancer, comprising:

acquiring knowledge of a CD274 nucleic acid molecule, or a PD-L1 polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
      wherein responsive to the acquisition of said knowledge, the individual is predicted to have an improved response to treatment with an immune checkpoint inhibitor as compared to an individual whose cancer does not comprise the CD274 nucleic acid molecule, or the PD-L1 polypeptide encoded by the CD274 nucleic acid molecule.

Exemplary Clause 11: A method of assessing a CD274 nucleic acid molecule or a PD-L1 polypeptide in a cancer in an individual, the method comprising:

detecting in a sample from the individual a CD274 nucleic acid molecule, or a PD-L1 polypeptide encoded by the CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and
      providing an assessment of the CD274 nucleic acid molecule, or the PD-L1 polypeptide encoded by the CD274 nucleic acid molecule.

Exemplary Clause 12: A method of detecting a CD274 nucleic acid molecule or a PD-L1 polypeptide, the method comprising detecting in a sample from an individual having a cancer a CD274 nucleic acid molecule, or a PD-L1 polypeptide encoded by the CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3.

Exemplary Clause 13: A method of detecting the presence or absence of a cancer in an individual, the method comprising:

detecting the presence or absence of a cancer in a sample from the individual; and
detecting, in a sample from the individual, the presence or absence of a CD274 nucleic acid molecule, or a PD-L1 polypeptide encoded by the CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3.

Exemplary Clause 14: The method of clause 13, comprising detecting the presence of the cancer in the sample.

Exemplary Clause 15: The method of clause 13 or clause 14, comprising detecting the presence of the CD274 nucleic acid molecule, or the PD-L1 polypeptide encoded by the CD274 nucleic acid molecule, in the sample from the individual.

Exemplary Clause 16: A method for monitoring progression or recurrence of a cancer in an individual, the method comprising:
detecting, in a first sample obtained from the individual at a first time point, the presence or absence of a CD274 nucleic acid molecule, or a PD-L1 polypeptide encoded by the CD274 nucleic acid molecule;
detecting, in a second sample obtained from the individual at a second time point after the first time point, the presence or absence of a CD274 nucleic acid molecule, or a PD-L1 polypeptide encoded by the CD274 nucleic acid molecule; and
providing an assessment of cancer progression or cancer recurrence in the individual based, at least in part, on the presence or absence of the CD274 nucleic acid molecule, or the PD-L1 polypeptide encoded by the CD274 nucleic acid molecule, in the first sample and/or in the second sample;
   wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3.

Exemplary Clause 17: The method of clause 16, wherein the presence of the CD274 nucleic acid molecule, or the PD-L1 polypeptide encoded by the CD274 nucleic acid molecule, in the first sample and/or in the second sample identifies the individual as having decreased risk of cancer progression or cancer recurrence when treated with a treatment comprising an immune checkpoint inhibitor.

Exemplary Clause 18: The method of clause 16 or clause 17, further comprising selecting a treatment, administering a treatment, adjusting a treatment, adjusting a dose of a treatment, or applying a treatment to the individual based, at least in part, on detecting the presence of the CD274 nucleic acid molecule, or the PD-L1 polypeptide encoded by the CD274 nucleic acid molecule, in the first sample and/or in the second sample, wherein the treatment comprises an immune checkpoint inhibitor.

Exemplary Clause 19: A method of detecting a CD274 nucleic acid molecule, the method comprising:

providing a plurality of nucleic acid molecules obtained from a sample from an individual having a cancer, wherein the plurality of nucleic acid molecules comprises nucleic acid molecules corresponding to a CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
optionally, ligating one or more adapters onto one or more nucleic acid molecules from the plurality of nucleic acid molecules;
optionally, amplifying the one or more ligated nucleic acid molecules from the plurality of nucleic acid molecules;
optionally, capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequence reads correspond to the CD274 nucleic acid molecule;
analyzing the plurality of sequence reads; and
based on the analysis, detecting the presence or absence of the CD274 nucleic acid molecule in the sample.

Exemplary Clause 20: The method of clause 19, further comprising receiving, at one or more processors, sequence read data for the plurality of sequence reads.

Exemplary Clause 21: The method of clause 20, wherein the analyzing the plurality of sequence reads comprises identifying, using the one or more processors, the presence or absence of sequence reads corresponding to the CD274 nucleic acid molecule.

Exemplary Clause 22: The method of any one of clauses 19-21, wherein the amplified nucleic acid molecules are captured by hybridization with one or more bait molecules.

Exemplary Clause 23: A method of detecting a CD274 nucleic acid molecule, the method comprising:
providing a sample from an individual having a cancer, wherein the sample comprises a plurality of nucleic acid molecules;
preparing a nucleic acid sequencing library from the plurality of nucleic acid molecules in the sample;
amplifying said library;
selectively enriching for one or more nucleic acid molecules in said library that comprise nucleotide sequences corresponding to a CD274 nucleic acid molecule to produce an enriched sample, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; sequencing the enriched sample, thereby producing a plurality of sequence reads;
analyzing the plurality of sequence reads for the presence of the CD274 nucleic acid molecule;
detecting, based on the analyzing step, the presence or absence of the CD274 nucleic acid molecule in the sample from the individual.

Exemplary Clause 24: The method of any one of clauses 19-23, wherein the plurality of nucleic acid molecules comprises a mixture of cancer nucleic acid molecules and non-cancer nucleic acid molecules.

Exemplary Clause 25: The method of clause 24, wherein the cancer nucleic acid molecules are derived from a tumor portion of a heterogeneous tissue biopsy sample.

Exemplary Clause 26: The method of clause 24, wherein the sample comprises a liquid biopsy sample, and wherein the cancer nucleic acid molecules are derived from a circulating tumor DNA (ctDNA) fraction of the liquid biopsy sample.

Exemplary Clause 27: The method of any one of clauses 19-22 and 24-26, wherein the one or more adapters comprise amplification primers, flow cell adapter sequences, substrate adapter sequences, or sample index sequences.

Exemplary Clause 28: The method of any one of clauses 23-27, wherein the selectively enriching comprises: (a) combining one or more bait molecules with the library, thereby hybridizing the one or more bait molecules to one or more nucleic acid molecules comprising nucleotide sequences corresponding to the CD274 nucleic acid molecule and producing nucleic acid hybrids; and (b) isolating the nucleic acid hybrids to produce the enriched sample.

Exemplary Clause 29: The method of any one of clauses 19-22 and 24-27, wherein the captured nucleic acid molecules are captured from the amplified nucleic acid molecules by hybridization to one or more bait molecules.

Exemplary Clause 30: The method of any one of clauses 19-29, wherein the amplifying comprises performing a polymerase chain reaction (PCR) amplification technique, a non-PCR amplification technique, or an isothermal amplification technique.

Exemplary Clause 31: The method of any one of clauses 19-30, wherein the sequencing comprises use of a massively parallel sequencing (MPS) technique, whole genome sequencing (WGS), whole exome sequencing, targeted sequencing, direct sequencing, or a Sanger sequencing technique.

Exemplary Clause 32: The method of clause 31, wherein the sequencing comprises a massively parallel sequencing technique, and the massively parallel sequencing technique comprises next generation sequencing (NGS).

Exemplary Clause 33: The method of any one of clauses 19-22, 24-27 and 29-32, wherein the sequencer comprises a next generation sequencer.

Exemplary Clause 34: The method of any one of clauses 19-33, further comprising generating a genomic profile for the individual, based, at least in part, on detecting the presence or absence of the CD274 nucleic acid molecule.

Exemplary Clause 35: The method of clause 34, wherein the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Clause 36: The method of clause 34 or clause 35, wherein the genomic profile for the individual further comprises results from a nucleic acid sequencing-based test.

Exemplary Clause 37: The method of any one of clauses 34-36, wherein the genomic profile for the individual further comprises the level of tumor mutational burden (TMB), clonal TMB, indel TMB, or nonsense-mediated decay (NMD)-escape TMB; the presence or absence of a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof.

Exemplary Clause 38: The method of any one of clauses 34-37, further comprising selecting a treatment, administering a treatment, or applying a treatment to the individual based on the generated genomic profile, wherein the treatment comprises an immune checkpoint inhibitor.

Exemplary Clause 39: The method of any one of clauses 19-38, further comprising generating a report indicating the presence or absence of the CD274 nucleic acid molecule in the sample.

Exemplary Clause 40: The method of clause 20 or clause 21, further comprising generating, by the one or more processors, a report indicating the presence or absence of the CD274 nucleic acid molecule in the sample.

Exemplary Clause 41: The method of clause 39 or clause 40, further comprising transmitting the report to a healthcare provider.

Exemplary Clause 42: The method of clause 41, wherein the report is transmitted via a computer network or a peer-to-peer connection.

Exemplary Clause 43: A method of identifying a candidate treatment for a cancer in an individual in need thereof, comprising performing DNA sequencing on a sample obtained from the individual to determine a sequencing mutation profile on a CD274 gene, wherein the sequencing mutation profile identifies the presence or absence of a CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and
      wherein the candidate treatment comprises an immune checkpoint inhibitor.

Exemplary Clause 44: The method of clause 43, wherein the sequencing comprises use of a massively parallel sequencing (MPS) technique, whole genome sequencing (WGS), whole exome sequencing, targeted sequencing, direct sequencing, or a Sanger sequencing technique.

Exemplary Clause 45: The method of clause 44, wherein the sequencing comprises a massively parallel sequencing technique, and the massively parallel sequencing technique comprises next generation sequencing (NGS).

Exemplary Clause 46: The method of any one of clauses 43-45, wherein the sequencing mutation profile identifies the presence or absence of a fragment of the CD274 nucleic acid molecule comprising a breakpoint.

Exemplary Clause 47: A method of treating or delaying progression of cancer, comprising: detecting in a sample from an individual having a cancer a CD274 nucleic acid molecule, or a PD-L1 polypeptide encoded by the CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof,
wherein the cancer is the corresponding cancer as listed in Table 3; and administering to the individual an effective amount of a treatment that comprises an immune checkpoint inhibitor.

Exemplary Clause 48: The method of any one of clauses 1-47, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 fusion nucleic acid molecule comprises a fusion between a CD274 gene, or a portion thereof, and a fusion partner gene as listed in Table 2, or a portion thereof; and
   wherein the cancer is a carcinoma, a sarcoma, a lymphoma, a leukemia, a myeloma, a germ cell cancer, or a blastoma.

Exemplary Clause 49: The method of any one of clauses 1-48, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 fusion nucleic acid molecule comprises a fusion between a CD274 gene, or a portion thereof, and a fusion partner gene as listed in Table 2, or a portion thereof; and
   wherein the cancer is a solid tumor.

Exemplary Clause 50: The method of any one of clauses 1-48, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 fusion nucleic acid molecule comprises a fusion between a CD274 gene, or a portion thereof, and a fusion partner gene as listed in Table 2, or a portion thereof; and
wherein the cancer is a hematologic malignancy.

Exemplary Clause 51: The method of any one of clauses 1-48, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 fusion nucleic acid molecule comprises a fusion between a CD274 gene, or a portion thereof, and a fusion partner gene as listed in Table 2, or a portion thereof; and
wherein the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Clause 52: The method of any one of clauses 1-48, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 fusion nucleic acid molecule comprises a fusion between a CD274 gene, or a portion thereof, and a fusion partner gene as listed in Table 2, or a portion thereof; and
wherein the cancer is an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, unknown primary adenocarcinoma, gastric cancer or carcinoma, unknown primary carcinoma (CUP), unknown primary carcinoma (CUP) (NOS), unknown primary melanoma, stomach adenocarcinoma, or vagina squamous cell carcinoma (SCC).

Exemplary Clause 53: The method of any one of clauses 1-48, wherein:
(a) the CD274 nucleic acid molecule comprises or results from a rearrangement comprising a Breakpoint 1 and/or Breakpoint 2 within the chromosomal coordinates as listed in Table 1, and wherein the cancer is the corresponding cancer as listed in Table 6; or
(b) the CD274 fusion nucleic acid molecule comprises a fusion between a CD274 gene, or a portion thereof, and a fusion partner gene as listed in Table 2, or a portion thereof, and wherein the cancer is the corresponding cancer as listed in Table 7.

Exemplary Clause 54: The method of any one of clauses 1-53, wherein the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4.

Exemplary Clause 55: The method of any one of clauses 1-47, wherein the CD274 fusion nucleic acid molecule comprises a fusion between a CD274 gene, or a portion thereof, and a fusion partner gene as listed in Table 2, or a portion thereof; wherein the CD274 fusion nucleic acid molecule comprises or results from a corresponding CD274 Breakpoint and/or Fusion Partner Gene Breakpoint within the chromosomal coordinates as listed in Table 8; and wherein the cancer is the corresponding cancer as listed in Table 8.

Exemplary Clause 56: The method of any one of clauses 1-47, wherein the CD274 fusion nucleic acid molecule comprises a fusion between a CD274 gene, or a portion thereof, and a fusion partner gene as listed in Table 3, or a portion thereof, and the cancer is the corresponding cancer as listed in Table 3; and wherein the CD274 fusion nucleic acid molecule comprises or results from a corresponding CD274 Breakpoint and/or Fusion Partner Gene Breakpoint within the chromosomal coordinates as listed in Table 5.

Exemplary Clause 57: The method of any one of clauses 1-56, wherein the cancer is metastatic.

Exemplary Clause 58: The method of any one of clauses 1-10, 17-18, 38-39, and 41-57, wherein the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, or any combination thereof.

Exemplary Clause 59: The method of any one of clauses 1-10, 17-18, 38-39, and 41-58, wherein the immune checkpoint inhibitor is a PD-1-, or a PD-L1-targeted agent.

Exemplary Clause 60: The method of clause 59, wherein the immune checkpoint inhibitor is a PD-1 inhibitor.

Exemplary Clause 61: The method of clause 60, wherein the immune checkpoint inhibitor comprises one or more of nivolumab, pembrolizumab, cemiplimab, or dostarlimab.

Exemplary Clause 62: The method of clause 59, wherein the immune checkpoint inhibitor is a PD-L1-inhibitor.

Exemplary Clause 63: The method of clause 62, wherein the immune checkpoint inhibitor comprises one or more of atezolizumab, avelumab, or durvalumab.

Exemplary Clause 64: The method of any one of clauses 1-10, 17-18, 38-39, and 41-58, wherein the immune checkpoint inhibitor is a CTLA-4 inhibitor.

Exemplary Clause 65: The method of clause 64, wherein the CTLA-4 inhibitor comprises ipilimumab.

Exemplary Clause 66: The method of any one of clauses 58-65, wherein the immune checkpoint inhibitor is a monotherapy.

Exemplary Clause 67: The method of clause 58, wherein the nucleic acid comprises a double-stranded RNA (dsRNA), a small interfering RNA (siRNA), or a small hairpin RNA (shRNA).

Exemplary Clause 68: The method of any one of clauses 1-10, 17-18, 38-39, and 41-67, wherein the treatment or the one or more treatment options further comprise an additional anti-cancer therapy.

Exemplary Clause 69: The method of clause 68, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Exemplary Clause 70: The method of clause 69, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 71: The method of clause 69, wherein the nucleic acid comprises a double-stranded RNA (dsRNA), a small interfering RNA (siRNA), or a small hairpin RNA (shRNA).

Exemplary Clause 72: The method of any one of clauses 1-10, 17-18, 38-39, and 41-67, wherein the treatment or the one or more treatment options comprise an immune checkpoint inhibitor in combination with one or more chemotherapeutic agents.

Exemplary Clause 73: The method of clause 72, wherein the one or more chemotherapeutic agents comprise a platinum-based chemotherapeutic agent.

Exemplary Clause 74: The method of any one of clauses 1-10, 17-18, 38-39, and 41-57, wherein the treatment or the one or more treatment options comprise atezolizumab and one or more chemotherapeutic agents.

Exemplary Clause 75: The method of clause 74, wherein the treatment or the one or more treatment options comprise atezolizumab, bevacizumab-Awwb or bevacizumab, carboplatin, and paclitaxel.

Exemplary Clause 76: The method of clause 74, wherein the treatment or the one or more treatment options comprise atezolizumab and paclitaxel or paclitaxel protein-bound.

Exemplary Clause 77: The method of any one of clauses 1-10, 17-18, 38-39, and 41-57, wherein the treatment or the one or more treatment options comprise nivolumab or pembrolizumab monotherapy.

Exemplary Clause 78: The method of any one of clauses 1-10, 17-18, 38-39, and 41-57, wherein the treatment or the one or more treatment options comprise pembrolizumab and one or more chemotherapeutic agents.

Exemplary Clause 79: The method of clause 78, wherein the treatment or the one or more treatment options comprise pembrolizumab, carboplatin and pemetrexed.

Exemplary Clause 80: The method of any one of clauses 1-18 and 47-79, wherein the encoded PD-L1 polypeptide is oncogenic.

Exemplary Clause 81: The method of any one of clauses 1-18 and 47-80, wherein the encoded PD-L1 polypeptide promotes cancer cell survival, angiogenesis, cancer cell proliferation, and any combination thereof.

Exemplary Clause 82: The method of any one of clauses 1-81, further comprising acquiring knowledge of or detecting in a sample from the individual the presence or absence of a CD274 gene amplification.

Exemplary Clause 83: The method of any one of clauses 1-82, wherein the cancer comprises a CD274 gene amplification.

Exemplary Clause 84: The method of any one of clauses 1-82, wherein the cancer does not comprise a CD274 gene amplification.

Exemplary Clause 85: The method of any one of clauses 1-84, further comprising acquiring knowledge of or detecting in a sample from the individual the presence or absence of a base substitution, a short insertion/deletion (indel), a copy number alteration, or a genomic rearrangement in one or more genes.

Exemplary Clause 86: The method of any one of clauses 1-85, wherein the cancer comprises a base substitution, a small insertion/deletion (indel), a copy number alteration, or a genomic rearrangement in one or more genes.

Exemplary Clause 87: The method of clause 85 or clause 86, wherein the one or more genes comprise one or more of TP53, PIK3CA, CDKN2A, KRAS, CDKN2B, CD274, MYC, JAK2, RB1, PDCD1LG2, APC, ARID1A, PTEN, BRAF, CREBBP, PBRM1, KMT2D, CCND1, KDM6A, BCL2L1, ERBB2, FBXW7, NF1, BCORL1, BRCA2, FGF19, FGFR1, MAP2K1, PRKC1, ATM, CDK12, CTNNB1, DNMT3A, FGF3, FGF4, GNAS, LYN, MET, NOTCH1, RNF43, STK11, TET2, VHL, ZNF217, ASXL1, BRCA1, EGFR, KDMSC, KIT, NFE2L2, NOTCH2, NOTCH3, PIK3R1, SOX9, TERC, ZNF703, MTAP, BRIP1, CDC73, ACVR1B, ATRX, MLH1, BRD4, SMAD4, PALB2, RAD21, GATA6, CTCF, or any combination thereof.

Exemplary Clause 88: The method of clause 87, wherein the cancer comprises:
(a) an S1601fs*4 frameshift mutation in an ATM gene, and/or a mutation in an ATM gene that results in a V2951F amino acid substitution in an encoded ATM polypeptide;
(b) a PTEN gene mutation that results in a Y155C amino acid substitution in an encoded PTEN polypeptide, and/or a PTEN splice site mutation of 634+G>A;
(c) a mutation in an RB1 gene that results in a K8* amino acid substitution in an encoded RB1 polypeptide, and/or a deletion of an RB1 gene, or of a portion thereof;
(d) a mutation in a MAP2K1 gene that results in an F53C amino acid substitution in an encoded MAP2K1 polypeptide;
(e) an amplification of an FGF19, FGF4, CCND1, FGF3, CDC274, or RAD21 gene, or any combination thereof;
(f) a deletion of an MTAP gene, or of a portion thereof;
(g) a K703fs*3 frameshift mutation in a BRIP1 gene;
(h) an H77fs*53 frameshift mutation in a KMT2D gene, and/or a mutation in a KMT2D gene that results in a Q4284* and/or S2834* amino acid substitution in an encoded KMT2D polypeptide;
(i) a mutation in an FBXW7 gene that results in a G437R and/or Q242* amino acid substitution in an encoded FBXW7 polypeptide;
(j) a CDC73 rearrangement, and/or an M1fs*56 frameshift mutation in a CDC73 gene;
(k) a K215fs*19 frameshift mutation in an ACVR1B gene;
(l) a D1850fs*33 frameshift mutation in an ARID1A gene;
(m) an R840fs*29 frameshift mutation in an ATRX gene;
(n) a P798fs*97 frameshift mutation in a BRD4 gene;
(o) a mutation in a SMAD4 gene resulting in an R515* amino acid substitution in an encoded SMAD4 polypeptide;
(p) a K654fs*47 frameshift mutation in a BRCA1 gene;
(q) a mutation in a CTNNB1 gene resulting in a G34R amino acid substitution in an encoded CTNNB1 polypeptide;
(r) an M723fs*21 frameshift mutation in a PALB2 gene;
(s) a T576fs*4 frameshift mutation in a PIK3R1 gene, and/or a deletion of a PIK3R1 gene, or a portion thereof;
(t) a mutation in a GATA6 gene resulting in an E579K amino acid substitution in an encoded GATA6 polypeptide;
(u) a mutation in a DNMT3A gene resulting in an R882H amino acid substitution in an encoded DNMT3A polypeptide;
(v) an E363fs*5 frameshift mutation in a CTCF gene;
   or any combination of (a)-(v).

Exemplary Clause 89: The method of clause 88, wherein the CDC73 rearrangement results in a CDC73 gene fusion comprising exons 1-7 of CDC73 fused to exons 11-17 of CDC73; optionally wherein the CDC73 gene fusion comprises or results from a breakpoint in exon 7 of CDC73 and/or a breakpoint in intron 10 of CDC73.

Exemplary Clause 90: The method of clause 85 or clause 86, wherein the one or more genes comprise TP53, PIK3CA, CDKN2A, KRAS, CDKN2B, MYC, or any combination thereof.

Exemplary Clause 91: The method of any one of clauses 87-90, wherein the cancer comprises:
(a) an R290fs*55 and/or H296fs*10 frameshift mutation in a TP53 gene; a mutation in a TP53 gene resulting in a G266V, E285K, C176Y, and/or P278S amino acid substitution in an encoded TP53 polypeptide; or a TP53 splice site mutation of 672+1G>T, or any combination thereof;
(b) a CDKN2B deletion;
(c) a MYC gene amplification;
   or any combination of (a)-(c).

Exemplary Clause 92: The method of any one of clauses 87-91, wherein the cancer comprises a deletion of a CDKN2A gene, or of a portion thereof.

Exemplary Clause 93: The method of clause 85 or clause 86, wherein the one or more genes comprise PIK3CA, JAK2, PDCD1LG2, CREBBP, PBRM1, or any combination thereof.

Exemplary Clause 94: The method of any one of clauses 87-93, wherein the cancer comprises a mutation in a PIK3CA gene that results in an E545K and/or E542K amino acid substitution, and/or an E110del deletion, in an encoded PIK3CA polypeptide.

Exemplary Clause 95: The method of any one of clauses 87-89 and 91-94, wherein the cancer comprises:
(a) an I279fs*4 frameshift mutation in a PBRM1 gene;
(b) a PDCD1LG2 gene amplification;
(c) a JAK2 gene amplification;
   or any combination of (a)-(c).

Exemplary Clause 96: The method of clause 85 or clause 86, wherein the one or more genes comprise a mismatch repair gene.

Exemplary Clause 97: The method of clause 96, wherein the mismatch repair gene is MLH1.

Exemplary Clause 98: The method of any one of clauses 87-89, 91-92, 94-95, and 97, wherein the cancer comprises a mutation in an MLH1 gene resulting in a Y684* amino acid substitution in an encoded MLH1 polypeptide.

Exemplary Clause 99: The method of any one of clauses 1-98, further comprising acquiring knowledge of or detecting in a sample from the individual the presence or absence of a genomic Epstein-Barr virus (EBV).

Exemplary Clause 100: The method of any one of clauses 1-99, wherein the cancer or the individual comprises a genomic EBV.

Exemplary Clause 101: The method of any one of clauses 1-100, wherein the cancer or the individual is positive for EBV.

Exemplary Clause 102: The method of any one of clauses 99-101, wherein the EBV is HHV-4.

Exemplary Clause 103: The method of clause 100 or clause 101, wherein the cancer is a gastric cancer.

Exemplary Clause 104: The method of clause 103, wherein the gastric cancer is an adenocarcinoma.

Exemplary Clause 105: The method of clause 103 or clause 104, wherein the gastric cancer is a stomach adenocarcinoma.

Exemplary Clause 106: The method of any one of clauses 103-105, wherein the treatment or the one or more treatment options comprise pembrolizumab.

Exemplary Clause 107: The method of clause 106, wherein the pembrolizumab is pembrolizumab monotherapy.

Exemplary Clause 108: The method of clause 106 or clause 107, wherein the treatment or the one or more treatment options are a second line treatment for cancer.

Exemplary Clause 109: The method of any one of clauses 103-108, wherein the gastric cancer is a Stage IV cancer.

Exemplary Clause 110: The method of any one of clauses 1-109, further comprising acquiring knowledge of or detecting a microsatellite instability status of the cancer in a sample from the individual.

Exemplary Clause 111: The method of any one of clauses 1-110, wherein the cancer is microsatellite stable.

Exemplary Clause 112: The method of any one of clauses 1-111, further comprising acquiring knowledge of or determining tumor mutational burden (TMB) in a sample from the individual.

Exemplary Clause 113: The method of any one of clauses 1-112, wherein the cancer has a TMB of less than 6 mutations per megabase (mut/Mb), between 6 and 20 mut/Mb, or greater than 20 mut/Mb,

Exemplary Clause 114: The method of any one of clauses 1-112, wherein the cancer has a high TMB.

Exemplary Clause 115: The method of clause 114, wherein the cancer has a TMB of at least about 10 mut/Mb,

Exemplary Clause 116: The method of any one of clauses 1-112, wherein the cancer has a TMB of about 7.0 mut/Mb,

Exemplary Clause 117: The method of any one of clauses 112-116, wherein TMB is assessed based on between about 0.83 Mb and about 1.14 Mb of sequenced DNA.

Exemplary Clause 118: The method of any one of clauses 112-116, wherein TMB is assessed based on about 0.79 Mb, about 0.80 Mb, or about 1.1 Mb of sequenced DNA.

Exemplary Clause 119: The method of any one of clauses 112-116, wherein TMB is assessed based on between about 0.8 Mb and about 1.1 Mb of sequenced DNA.

Exemplary Clause 120: The method of any one of clauses 112-116, wherein TMB is assessed based on up to about 1.1 Mb or up to about 1.24 Mb of sequenced DNA.

Exemplary Clause 121: The method of any one of clauses 112-120, wherein TMB is determined by sequencing, whole exome sequencing, whole genome sequencing, gene-targeted sequencing, or next-generation sequencing.

Exemplary Clause 122: The method of any one of clauses 112-121, wherein the cancer is a non-small cell lung carcinoma, a colorectal carcinoma, a carcinoma of unknown primary (CUP), or a gastric carcinoma.

Exemplary Clause 123: The method of any one of clauses 1-122, further comprising acquiring knowledge of or determining the level of PD-L1 expression in a sample from the individual.

Exemplary Clause 124: The method of any one of clauses 1-123, wherein the cancer is PD-L1 positive.

Exemplary Clause 125: The method of clause 124, wherein the cancer is PD-L1-high positive.

Exemplary Clause 126: The method of any one of clauses 1-123, wherein the cancer is PD-L1 negative.

Exemplary Clause 127: The method of any one of clauses 123-126, wherein PD-L1 expression is determined using an immunohistochemistry assay in a sample obtained from the individual.

Exemplary Clause 128: The method of clause 127, wherein the immunohistochemistry assay is a DAKO PD-L1 22C3 assay.

Exemplary Clause 129: The method of clause 128, wherein PD-L1 expression is assessed based on a tumor proportion score (TPS).

Exemplary Clause 130: The method of clause 129, wherein the cancer is PD-L1 positive.

Exemplary Clause 131: The method of clause 130, wherein the cancer has a TPS of at least about 1%.

Exemplary Clause 132: The method of clause 130, wherein the cancer has a TPS of between about 1% and about 49%.

Exemplary Clause 133: The method of clause 129, wherein the cancer is PD-L1 high positive.

Exemplary Clause 134: The method of clause 133, wherein the cancer has a TPS of at least about 50%.

Exemplary Clause 135: The method of clause 129, wherein the cancer has a TPS of at least about 1%, at least about 25%, at least about 50%, or at least about 75%.

Exemplary Clause 136: The method of clause 129, wherein the cancer has a TPS of at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or 100%.

Exemplary Clause 137: The method of clause 129, wherein the cancer is PD-L1 negative.

Exemplary Clause 138: The method of clause 137, wherein the cancer has a TPS of less than 1%.

Exemplary Clause 139: The method of clause 128, wherein PD-L1 expression is assessed based on a combined positive score (CPS).

Exemplary Clause 140: The method of clause 139, wherein the cancer has high PD-L1 expression.

Exemplary Clause 141: The method of clause 139 or clause 140, wherein the cancer has a CPS of at least about 1 or at least about 10.

Exemplary Clause 142: The method of clause 127, wherein the immunohistochemistry assay is a VENTANA SP 142 assay.

Exemplary Clause 143: The method of clause 142, wherein PD-L1 expression is assessed based on the proportion of tumor area occupied by PD-L1 expressing tumor-infiltrating immune cells of any intensity (IC), or the percentage of PD-L1 expressing tumor cells of any intensity (TC).

Exemplary Clause 144: The method of clause 143, wherein the cancer has high PD-L1 expression.

Exemplary Clause 145: The method of clause 143 or clause 144, wherein the cancer has a TC of at least about 50%.

Exemplary Clause 146: The method of clause 143 or clause 144, wherein the cancer has an IC of at least about 10%.

Exemplary Clause 147: The method of clause 143, wherein the cancer has an IC of at least about 1% or at least about 5%.

Exemplary Clause 148: The method of any one of clauses 1-147, further comprising acquiring knowledge of or determining the clonality of the CD274 nucleic acid molecule in the cancer.

Exemplary Clause 149: The method of any one of clauses 1-148, wherein the CD274 nucleic acid molecule results from a clonal or a sub-clonal rearrangement of a CD274 gene in the cancer.

Exemplary Clause 150: The method of any one of clauses 1-149, wherein the CD274 nucleic acid molecule results from a clonal rearrangement of a CD274 gene in the cancer,

Exemplary Clause 151: The method of any one of clauses 1-149, wherein the CD274 nucleic acid molecule is clonal or sub-clonal in the cancer.

Exemplary Clause 152: The method of clause 151, wherein the CD274 nucleic acid molecule is clonal in the cancer.

Exemplary Clause 153: The method of clause 150 or clause 152, wherein acquiring knowledge of or determining that the CD274 nucleic acid molecule is clonal in the cancer or results from a clonal rearrangement of a CD274 gene in the cancer identifies the individual as likely to respond to a treatment comprising an immune checkpoint inhibitor.

Exemplary Clause 154: The method of clause 150 or clause 152, wherein responsive to acquiring knowledge of or determining that the CD274 nucleic acid molecule is clonal in the cancer or results from a clonal rearrangement of a CD274 gene in the cancer, the individual is predicted to have longer survival when treated with a treatment comprising an immune checkpoint inhibitor, as compared to survival of: (a) an individual whose cancer does not comprise a clonal CD274 nucleic acid molecule, or a CD274 nucleic acid molecule resulting from a clonal rearrangement of a CD274 gene; or (b) an individual whose cancer comprises a sub-clonal CD274 nucleic acid molecule, or a CD274 nucleic acid molecule resulting from a sub-clonal rearrangement of a CD274 gene.

Exemplary Clause 155: The method of clause 150 or clause 152, wherein responsive to acquiring knowledge of or determining that the CD274 nucleic acid molecule is clonal in the cancer or results from a clonal rearrangement of a CD274 gene in the cancer, the individual is predicted to have an improved response to treatment with an immune checkpoint inhibitor, as compared to: (a) an individual whose cancer does not comprise a clonal CD274 nucleic acid molecule, or a CD274 nucleic acid molecule resulting from a clonal rearrangement of a CD274 gene; or (b) an individual whose cancer comprises a sub-clonal CD274 nucleic acid molecule, or a CD274 nucleic acid molecule resulting from a sub-clonal rearrangement of a CD274 gene.

Exemplary Clause 156: The method of any one of clauses 150 and 152-155, wherein acquiring knowledge of or determining that the CD274 nucleic acid molecule is clonal in the cancer or results from a clonal rearrangement of a CD274 gene in the cancer identifies the cancer as likely to be PD-L1 positive or PD-L1 high positive.

Exemplary Clause 157: The method of clause 156, wherein acquiring knowledge of or determining that the CD274 nucleic acid molecule is clonal in the cancer or results from a clonal rearrangement of a CD274 gene in the cancer identifies the cancer as likely to have a TPS of at least about 50%, assessed based on an immunohistochemistry assay.

Exemplary Clause 158: The method of clause 157, wherein the immunohistochemistry assay is a DAKO PD-L1 22C3 assay.

Exemplary Clause 159: The method of any one of clauses 148-158, wherein clonality of the CD274 nucleic acid molecule is assessed by performing DNA sequencing on a sample obtained from the individual.

Exemplary Clause 160: The method of clause 159, wherein clonality of the CD274 nucleic acid molecule is assessed based on sequencing coverage of a CD274 gene and the number of sequence read pairs spanning a breakpoint of the CD274 nucleic acid molecule.

Exemplary Clause 161: The method of clause 160, wherein clonality of the CD274 nucleic acid molecule is assessed based on sequencing coverage of a CD274 gene and a threshold for the number of sequence read pairs spanning a breakpoint of the CD274 nucleic acid molecule, wherein the threshold is selected using a receiver operator characteristic (ROC) curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis.

Exemplary Clause 162: The method of clause 159, wherein clonality of the CD274 nucleic acid molecule is assessed based on the number of sequence read pairs spanning a breakpoint of the CD274 nucleic acid molecule.

Exemplary Clause 163: The method of clause 162, wherein clonality of the CD274 nucleic acid molecule is assessed based on a threshold for the number of sequence read pairs spanning a breakpoint of the CD274 nucleic acid molecule, wherein the threshold is selected using a receiver operator characteristic (ROC) curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis.

Exemplary Clause 164: The method of clause 161 or clause 163, wherein a sample from a tumor is PD-L1 high positive if it comprises a tumor proportion score (TPS) of at least about 50%.

Exemplary Clause 165: The method of any one of clauses 161 and 163-164, wherein the threshold for the number of sequence read pairs spanning a breakpoint of the CD274 nucleic acid molecule is at least about 20 read pairs, at least about 21 read pairs, at least about 22 read pairs, at least about 23 read pairs, at least about 24 read pairs, at least about 25 read pairs, at least about 26 read pairs, at least about 27 read pairs, at least about 28 read pairs, at least about 29 read pairs, at least about 30 read pairs, at least about 31 read pairs, at least about 32 read pairs, at least about 33 read pairs, at least about 34 read pairs, at least about 35 read pairs, at least about 36 read pairs, at least about 37 read pairs, at least about 38 read pairs, at least about 39 read pairs, at least about 40 read pairs, at least about 41 read pairs, at least about 42 read pairs, at least about 43 read pairs, at least about 44 read pairs, at least about 45 read pairs, at least about 46 read pairs, at least about 47 read pairs, at least about 48 read pairs, at least about 49 read pairs, or at least about 50 read pairs spanning a breakpoint of the CD274 nucleic acid molecule.

Exemplary Clause 166: The method of any one of clauses 161 and 163-165, wherein the threshold for the number of sequence read pairs spanning a breakpoint of the CD274 nucleic acid molecule is at least about 25 sequence read pairs spanning a breakpoint of the CD274 nucleic acid molecule.

Exemplary Clause 167: The method of any one of clauses 159-166, wherein the sample is a bulk tumor sample derived from a single anatomic location.

Exemplary Clause 168: The method of any one of clauses 1-167, further comprising obtaining the sample from the individual.

Exemplary Clause 169: The method of any one of clauses 1-168, wherein the sample is obtained from the cancer.

Exemplary Clause 170: The method of any one of clauses 1-169, wherein the sample comprises a tissue biopsy sample, a liquid biopsy sample, or a normal control.

Exemplary Clause 171: The method of clause 170, wherein the sample is from a tumor biopsy, tumor specimen, or circulating tumor cell.

Exemplary Clause 172: The method of any one of clauses 1-170, wherein the sample is a liquid biopsy sample and comprises blood, plasma, cerebrospinal fluid, sputum, stool, urine, or saliva.

Exemplary Clause 173: The method of any one of clauses 1-172, wherein the sample comprises cells and/or nucleic acids from the cancer.

Exemplary Clause 174: The method of clause 173, wherein the sample comprises mRNA, DNA, circulating tumor DNA (ctDNA), cell-free DNA, or cell-free RNA from the cancer.

Exemplary Clause 175: The method of clause 172, wherein the sample is a liquid biopsy sample and comprises circulating tumor cells (CTCs).

Exemplary Clause 176: The method of clause 172, wherein the sample is a liquid biopsy sample and comprises cell-free DNA (cfDNA), circulating tumor DNA (ctDNA), or any combination thereof.

Exemplary Clause 177: The method of any one of clauses 1-176, comprising acquiring knowledge of or detecting the CD274 nucleic acid molecule, or the PD-L1 polypeptide encoded by the CD274 nucleic acid molecule, in a tissue biopsy sample, in a liquid biopsy sample, or in both a tissue biopsy sample and a liquid biopsy sample, from the individual.

Exemplary Clause 178: The method of any one of clauses 4-10 and 48-177, wherein the acquiring knowledge comprises detecting the CD274 nucleic acid molecule, or the PD-L1 polypeptide encoded by the CD274 nucleic acid molecule, in the sample.

Exemplary Clause 179: The method of any one of clauses 1-3, 11-42, and 47-178, wherein the detecting comprises detecting a fragment of the CD274 nucleic acid molecule comprising a breakpoint.

Exemplary Clause 180: The method of any one of clauses 1-3, 11-42, and 47-179, wherein the CD274 nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Exemplary Clause 181: The method of clause 180, wherein the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

Exemplary Clause 182: The method of clause 180, wherein the sequencing comprises RNA-sequencing (RNA-seq).

Exemplary Clause 183: The method of clause 180, wherein the amplification-based assay comprises a polymerase chain reaction (PCR) amplification technique, a non-PCR amplification technique, or an isothermal amplification technique.

Exemplary Clause 184: The method of clause 180 or clause 183, wherein the amplification-based assay comprises a reverse transcription PCR (RT-PCR), a quantitative real-time PCR (qPCR), or a reverse transcription quantitative real-time PCR (RT-qPCR) assay.

Exemplary Clause 185: The method of clause 184, wherein the amplification-based assay comprises an RT-PCR assay.

Exemplary Clause 186: The method of any one of clauses 1-3, 11-42, and 47-178, wherein detecting the PD-L1 polypeptide encoded by the CD274 nucleic acid molecule comprises detecting a portion of the polypeptide that is encoded by a fragment of the CD274 nucleic acid molecule that comprises a breakpoint.

Exemplary Clause 187: The method of any one of clauses 1-3, 11-42, 47-178, and 186, wherein the PD-L1 polypeptide encoded by the CD274 nucleic acid molecule is detected in the sample by one or more of: immunoblotting, enzyme linked immunosorbent assay (ELISA), immunohistochemistry, or mass spectrometry.

Exemplary Clause 188: The method of any one of clauses 1-3, 11-42, and 47-187, wherein the CD274 nucleic acid molecule, or the PD-L1 polypeptide encoded by the CD274 nucleic acid molecule, is detected using a digital pathology method.

Exemplary Clause 189: The method of any one of clauses 1-3, 11-18, 47-185 and 188, further comprising selectively enriching for one or more nucleic acid molecules in the sample comprising nucleotide sequences corresponding to the CD274 nucleic acid molecule; wherein the selectively enriching produces an enriched sample.

Exemplary Clause 190: The method of clause 189, wherein the selectively enriching comprises: (a) combining one or more bait molecules with the sample, thereby hybridizing the one or more bait molecules to one or more nucleic acid molecules in the sample comprising nucleotide sequences corresponding to the CD274 nucleic acid molecule and producing nucleic acid hybrids; and (b) isolating the nucleic acid hybrids to produce the enriched sample.

Exemplary Clause 191: The method of any one of clauses 22, 28-29, and 190, wherein the one or more bait molecules comprise a capture nucleic acid molecule configured to hybridize to a nucleotide sequence corresponding to the CD274 nucleic acid molecule.

Exemplary Clause 192: The method of clause 191, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Exemplary Clause 193: The method of any one of clauses 22, 28-29, and 190-192, wherein the one or more bait molecules are conjugated to an affinity reagent or to a detection reagent.

Exemplary Clause 194: The method of clause 193, wherein the affinity reagent is an antibody, an antibody fragment, or biotin, or wherein the detection reagent is a fluorescent marker.

Exemplary Clause 195: The method of any one of clauses 191-194, wherein the capture nucleic acid molecule comprises a DNA, RNA, or mixed DNA/RNA molecule.

Exemplary Clause 196: The method of clause 23 or clause 189, wherein the selectively enriching comprises amplifying the one or more nucleic acid molecules comprising nucleotide sequences corresponding to the CD274 nucleic acid molecule using a polymerase chain reaction (PCR) to produce an enriched sample.

Exemplary Clause 197: The method of any one of clauses 189-196, further comprising sequencing the enriched sample.

Exemplary Clause 198: The method of any one of clauses 1-197, wherein the individual is a human.

Exemplary Clause 199: A kit comprising a probe, a bait, or one or more oligonucleotides for detecting:
(a) a CD274 nucleic acid molecule that comprises or results from a rearrangement comprising a Breakpoint 1 and/or Breakpoint 2 within the chromosomal coordinates as listed in Table 1;
(b) a CD274 fusion nucleic acid molecule comprising a fusion between a CD274 gene, or a portion thereof, and a fusion partner gene as listed in Table 2, or a portion thereof; or
(c) a CD274 fusion nucleic acid molecule comprising a fusion between a CD274 gene, or a portion thereof, and a fusion partner gene as listed in Table 3, or a portion thereof, in a sample from an individual having a corresponding cancer as listed in Table 3.

Exemplary Clause 200: The kit of clause 199, wherein:
(a) the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4; or
(b) the CD274 fusion nucleic acid molecule comprises a fusion between a CD274 gene, or a portion thereof, and a fusion partner gene as listed in Table 3, or a portion thereof, and wherein the CD274 fusion nucleic acid molecule comprises or results from a corresponding CD274 Breakpoint and/or Fusion Partner Gene Breakpoint within the chromosomal coordinates as listed in Table 5.

Exemplary Clause 201: A CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule comprising a fusion between a CD274 gene, or a portion thereof, and a fusion partner gene as listed in Table 2, or a portion thereof.

Exemplary Clause 202: The CD274 nucleic acid molecule of clause 201, wherein the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4.

Exemplary Clause 203: A vector comprising the nucleic acid molecule of clause 201 or clause 202.

Exemplary Clause 204: A host cell comprising the vector of clause 203.

Exemplary Clause 205: An antibody or antibody fragment that specifically binds to a PD-L1 polypeptide, or to a portion thereof, encoded by:
(a) a CD274 nucleic acid molecule that comprises or results from a rearrangement comprising a Breakpoint 1 and/or Breakpoint 2 within the chromosomal coordinates as listed in Table 1; or
(b) a CD274 fusion nucleic acid molecule comprising a fusion between a CD274 gene, or a portion thereof, and a fusion partner gene as listed in Table 2, or a portion thereof.

Exemplary Clause 206: The antibody or antibody fragment of clause 205, wherein the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4.

Exemplary Clause 207: A kit comprising an antibody or antibody fragment for detecting:
(a) a PD-L1 polypeptide, or a portion thereof, encoded by a CD274 nucleic acid molecule that comprises or results from a rearrangement comprising a Breakpoint 1 and/or Breakpoint 2 within the chromosomal coordinates as listed in Table 1;
(b) a PD-L1 polypeptide, or a portion thereof, encoded by a CD274 fusion nucleic acid molecule comprising a fusion between a CD274 gene, or a portion thereof, and a fusion partner gene as listed in Table 2, or a portion thereof; or
(c) a PD-L1 polypeptide, or a portion thereof, encoded by a CD274 fusion nucleic acid molecule comprising a fusion between a CD274 gene, or a portion thereof, and a fusion partner gene as listed in Table 3, or a portion thereof, in a sample from an individual having a corresponding cancer as listed in Table 3.

Exemplary Clause 208: The kit of clause 207, wherein:
(a) the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4; or
(b) the CD274 fusion nucleic acid molecule comprises a fusion between a CD274 gene, or a portion thereof, and a fusion partner gene as listed in Table 3, or a portion thereof, and wherein the CD274 fusion nucleic acid molecule comprises or results from a corresponding CD274 Breakpoint and/or Fusion Partner Gene Breakpoint within the chromosomal coordinates as listed in Table 5.

Exemplary Clause 209: In vitro use of a probe, a bait, or one or more oligonucleotides for detecting:
(a) a CD274 nucleic acid molecule that comprises or results from a rearrangement comprising a Breakpoint 1 and/or Breakpoint 2 within the chromosomal coordinates as listed in Table 1;
(b) a CD274 fusion nucleic acid molecule comprising a fusion between a CD274 gene, or a portion thereof, and a fusion partner gene as listed in Table 2, or a portion thereof; or
(c) a CD274 fusion nucleic acid molecule comprising a fusion between a CD274 gene, or a portion thereof, and a fusion partner gene as listed in Table 3, or a portion thereof, in a sample from an individual having a corresponding cancer as listed in Table 3.

Exemplary Clause 210: A system, comprising:

a memory configured to store one or more program instructions; and
one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from a sample obtained from an individual;
(b) analyze the plurality of sequence reads for the presence of a CD274 nucleic acid molecule, wherein:
   (i) the CD274 nucleic acid molecule comprises or results from a rearrangement comprising a Breakpoint 1 and/or Breakpoint 2 within the chromosomal coordinates as listed in Table 1, or
   (ii) the CD274 nucleic acid molecule is a CD274 fusion nucleic acid molecule comprising a fusion between a CD274 gene, or a portion thereof, and a fusion partner gene as listed in Table 2, or a portion thereof; and
(c) detect, based on the analyzing, the CD274 nucleic acid molecule in the sample.

Exemplary Clause 211: A non-transitory computer readable storage medium comprising one or more programs executable by one or more computer processors for performing a method, comprising:
(a) obtaining, using the one or more processors, a plurality of sequence reads of one or more nucleic acid molecules, wherein the one or more nucleic acid molecules are derived from a sample obtained from an individual;
(b) analyzing, using the one or more processors, the plurality of sequence reads for the presence of a CD274 nucleic acid molecule, wherein:
   (i) the CD274 nucleic acid molecule comprises or results from a rearrangement comprising a Breakpoint 1 and/or Breakpoint 2 within the chromosomal coordinates as listed in Table 1, or
   (ii) the CD274 nucleic acid molecule is a CD274 fusion nucleic acid molecule comprising a fusion between a CD274 gene, or a portion thereof, and a fusion partner gene as listed in Table 2, or a portion thereof; and
(c) detecting, using the one or more processors and based on the analyzing, the CD274 nucleic acid molecule in the sample.

Exemplary Clause 212: The system of clause 210, or the non-transitory computer readable storage medium of clause 211, wherein the sample is from an individual having a cancer.

Exemplary Clause 213: The system or the non-transitory computer readable storage medium of clause 212, wherein the cancer is a carcinoma, a sarcoma, a lymphoma, a leukemia, a myeloma, a germ cell cancer, or a blastoma.

Exemplary Clause 214: The system or the non-transitory computer readable storage medium of clause 212, wherein the cancer is a solid tumor.

Exemplary Clause 215: The system or the non-transitory computer readable storage medium of clause 212, wherein the cancer is a hematologic malignancy.

Exemplary Clause 216: The system or the non-transitory computer readable storage medium of clause 212, wherein the cancer is a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, lung non-small cell lung carcinoma (NSCLC), gastric cancer or carcinoma, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Clause 217: The system or the non-transitory computer readable storage medium of clause 212, wherein the cancer is an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, gastric cancer or carcinoma, unknown primary adenocarcinoma, unknown primary carcinoma (CUP), unknown primary carcinoma (CUP) (NOS), unknown primary melanoma, stomach adenocarcinoma, or vagina squamous cell carcinoma (SCC).

Exemplary Clause 218: The system or the non-transitory computer readable storage medium of clause 212, wherein:
(a) the CD274 nucleic acid molecule comprises or results from a rearrangement comprising a Breakpoint 1 and/or Breakpoint 2 within the chromosomal coordinates as listed in Table 1, and wherein the cancer is the corresponding cancer as listed in Table 6; or
(b) the CD274 fusion nucleic acid molecule comprises a fusion between a CD274 gene, or a portion thereof, and a fusion partner gene as listed in Table 2, or a portion thereof, and wherein the cancer is the corresponding cancer as listed in Table 7.

Exemplary Clause 219: The system of any one of clauses 210 and 212-217, or the non-transitory computer readable storage medium of any one of clauses 211-217, wherein the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4.

Exemplary Clause 220: The system or the non-transitory computer readable storage medium of clause 212, wherein the CD274 fusion nucleic acid molecule comprises a fusion between a CD274 gene, or a portion thereof, and a fusion partner gene as listed in Table 2, or a portion thereof; wherein the CD274 fusion nucleic acid molecule comprises or results from a corresponding CD274 Breakpoint and/or Fusion Partner Gene Breakpoint within the chromosomal coordinates as listed in Table 8; and wherein the cancer is the corresponding cancer as listed in Table 8.

Exemplary Clause 221: A system, comprising:

a memory configured to store one or more program instructions; and
one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from a sample obtained from an individual having a cancer;
(b) analyze the plurality of sequence reads for the presence of a CD274 nucleic acid molecule, wherein the CD274 nucleic acid molecule is a CD274 fusion nucleic acid molecule comprising a fusion between a CD274 gene, or a portion thereof, and a fusion partner gene as listed in Table 3, or a portion thereof, wherein the cancer is the corresponding cancer as listed in Table 3; and
(c) detect, based on the analyzing, the CD274 nucleic acid molecule in the sample.

Exemplary Clause 222: A non-transitory computer readable storage medium comprising one or more programs executable by one or more computer processors for performing a method, comprising:
(a) obtaining, using the one or more processors, a plurality of sequence reads of one or more nucleic acid molecules, wherein the one or more nucleic acid molecules are derived from a sample obtained from an individual having a cancer;
(b) analyzing, using the one or more processors, the plurality of sequence reads for the presence of a CD274 nucleic acid molecule, wherein the CD274 nucleic acid molecule is a CD274 fusion nucleic acid molecule comprising a fusion between a CD274 gene, or a portion thereof, and a fusion partner gene as listed in Table 3, or a portion thereof, wherein the cancer is the corresponding cancer as listed in Table 3; and
(c) detecting, using the one or more processors and based on the analyzing, the CD274 nucleic acid molecule in the sample.

Exemplary Clause 223: The system of clause 221, or the non-transitory computer readable storage medium of clause 222, wherein the CD274 fusion nucleic acid molecule comprises a fusion between a CD274 gene, or a portion thereof, and a fusion partner gene as listed in Table 3, or a portion thereof, and the cancer is the corresponding cancer as listed in Table 3; and wherein the CD274 fusion nucleic acid molecule comprises or results from a corresponding CD274 Breakpoint and/or Fusion Partner Gene Breakpoint within the chromosomal coordinates as listed in Table 5.

Exemplary Clause 224: The system of any one of clauses 210, 212-221, and 223, or the non-transitory computer readable storage medium of any one of clauses 211-220 and 222-223, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Exemplary Clause 225: The system of any one of clauses 210, 212-221, and 223-224, wherein the one or more program instructions when executed by the one or more processors are further configured to generate, based at least in part on the detecting, a genomic profile for the sample.

Exemplary Clause 226: The non-transitory computer readable storage medium of any one of clauses 211-220 and 222-224, wherein the method further comprises generating, based at least in part on the detecting, a genomic profile for the sample.

Exemplary Clause 227: The system of clause 225, or the non-transitory computer readable storage medium of clause 226, wherein the individual is administered a treatment based at least in part on the genomic profile.

Exemplary Clause 228: The system of clause 225 or clause 227, or the non-transitory computer readable storage medium of clause 226 or clause 227, wherein the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Clause 229: The system of any one of clauses 225 and 227-228, or the non-transitory computer readable storage medium of any one of clauses 226-228, wherein the genomic profile further comprises results from a nucleic acid sequencing-based test.

Exemplary Clause 230: The system of any one of clauses 225 and 227-229, or the non-transitory computer readable storage medium of any one of clauses 226-229, wherein the genomic profile further comprises the level of tumor mutational burden (TMB), clonal TMB, indel TMB, or nonsense-mediated decay (NMD)-escape TMB; the presence or absence of a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof.

Exemplary Clause 231: The system of any one of clauses 225 and 227-230, wherein the one or more program instructions when executed by the one or more processors are further configured to generate a report indicating the presence or absence of the CD274 nucleic acid molecule in the sample.

Exemplary Clause 232: The non-transitory computer readable storage medium of any one of clauses 226-230, wherein the method further comprises generating a report indicating the presence or absence of the CD274 nucleic acid molecule in the sample.

Exemplary Clause 233: The system of clause 231 or the non-transitory computer readable storage medium of clause 232, wherein the report is generated by the one or more processors.

Exemplary Clause 234: The system of clause 231 or clause 233, or the non-transitory computer readable storage medium of clause 232 or clause 233, further comprising transmitting the report to a healthcare provider.

Exemplary Clause 235: The system or the non-transitory computer readable storage medium of clause 234, wherein the report is transmitted via a computer network or a peer-to-peer connection.

Exemplary Clause 236: An immune checkpoint inhibitor for use in a method of treating or delaying progression of cancer, wherein the method comprises administering the immune checkpoint inhibitor to an individual, wherein:
(a) a CD274 nucleic acid molecule that comprises or results from a rearrangement comprising a Breakpoint 1 and/or Breakpoint 2 within the chromosomal coordinates as listed in Table 1 is detected in a sample obtained from the individual;
(b) a CD274 fusion nucleic acid molecule comprising a fusion between a CD274 gene, or a portion thereof, and a fusion partner gene as listed in Table 2, or a portion thereof, is detected in a sample obtained from the individual; or
(c) a CD274 fusion nucleic acid molecule comprising a fusion between a CD274 gene, or a portion thereof, and a fusion partner gene as listed in Table 3, or a portion thereof, is detected in a sample from an individual having a corresponding cancer as listed in Table 3.

Exemplary Clause 237: An immune checkpoint inhibitor for use in the manufacture of a medicament for treating or delaying progression of cancer, wherein the medicament is to be administered to an individual, wherein:
(a) a CD274 nucleic acid molecule that comprises or results from a rearrangement comprising a Breakpoint 1 and/or Breakpoint 2 within the chromosomal coordinates as listed in Table 1 is detected in a sample obtained from the individual;
(b) a CD274 fusion nucleic acid molecule comprising a fusion between a CD274 gene, or a portion thereof, and a fusion partner gene as listed in Table 2, or a portion thereof, is detected in a sample obtained from the individual; or
(c) a CD274 fusion nucleic acid molecule comprising a fusion between a CD274 gene, or a portion thereof, and a fusion partner gene as listed in Table 3, or a portion thereof, is detected in a sample from an individual having a corresponding cancer as listed in Table 3.

Exemplary Clause 238: A method of selecting a treatment for an individual having a cancer, the method comprising detecting or acquiring knowledge of a cluster of differentiation 274 (CD274) nucleic acid molecule, or a programmed death-ligand 1 (PD-L1) polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
      wherein detecting or acquiring knowledge of the CD274 nucleic acid molecule, or the PD-L1 polypeptide encoded by the CD274 nucleic acid molecule, in the sample identifies the individual as one who may benefit from a treatment comprising an immune checkpoint inhibitor.

Exemplary Clause 239: A method of treating or delaying progression of cancer, comprising:
detecting or acquiring knowledge of a CD274 nucleic acid molecule, or a PD-L1 polypeptide encoded by the CD274 nucleic acid molecule, in a sample from an individual having a cancer, wherein:
   (a) the CD274 nucleic acid molecule comprises or results from a rearrangement comprising a Breakpoint 1 and/or Breakpoint 2 within the chromosomal coordinates as listed in Table 1; or
   (b) the CD274 nucleic acid molecule is a CD274 fusion nucleic acid molecule, wherein:
      (i) the CD274 fusion nucleic acid molecule comprises a fusion between a CD274 gene, or a portion thereof, and a fusion partner gene as listed in Table 2, or a portion thereof, or
      (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and
administering to the individual an effective amount of a treatment that comprises an immune checkpoint inhibitor responsive to detecting or acquiring knowledge of the CD274 nucleic acid molecule, or the PD-L1 polypeptide encoded by the CD274 nucleic acid molecule, in the sample.

Exemplary Clause 240: A method of detecting a CD274 nucleic acid molecule, the method comprising:
providing a plurality of nucleic acid molecules obtained from a sample from an individual having a cancer, wherein the plurality of nucleic acid molecules comprises nucleic acid molecules corresponding to a CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
   (i) the CD274 fusion nucleic acid molecule comprises a fusion between a CD274 gene, or a portion thereof, and a fusion partner gene as listed in Table 2, or a portion thereof, or
   (ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
ligating one or more adapters onto one or more nucleic acid molecules from the plurality of nucleic acid molecules;
amplifying one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequence reads correspond to the CD274 nucleic acid molecule;
analyzing the plurality of sequence reads; and based on the analysis, detecting the presence or absence of the CD274 nucleic acid molecule in the sample.

Exemplary Clause 241: The method of clause 240, wherein:
(a) the method further comprises receiving, at one or more processors, sequence read data for the plurality of sequence reads;
(b) the analyzing the plurality of sequence reads comprises identifying, using one or more processors, the presence or absence of sequence reads corresponding to the CD274 nucleic acid molecule;
(c) the amplified nucleic acid molecules are captured by hybridization with one or more bait molecules;
(d) the plurality of nucleic acid molecules comprises a mixture of cancer nucleic acid molecules and non-cancer nucleic acid molecules;
(e) the one or more adapters comprise amplification primers, flow cell adapter sequences, substrate adapter sequences, or sample index sequences;
(f) the amplifying comprises performing a polymerase chain reaction (PCR) amplification technique, a non-PCR amplification technique, or an isothermal amplification technique;
(g) the sequencing comprises use of a massively parallel sequencing (MPS) technique, whole genome sequencing (WGS), whole exome sequencing, targeted sequencing, direct sequencing, a Sanger sequencing technique, or next generation sequencing (NGS);
(h) the sequencer comprises a next generation sequencer;
(i) the method further comprises generating a genomic profile for the individual, based, at least in part, on detecting the presence or absence of the CD274 nucleic acid molecule in the sample; and/or
(j) the method further comprises generating a report indicating the presence or absence of the CD274 nucleic acid molecule in the sample.

Exemplary Clause 242: The method of clause 241, wherein:
(a) the genomic profile for the individual further comprises:
   (i) results from a comprehensive genomic profiling (CGP) test, a gene expression profiling test, a cancer hotspot panel test, a DNA methylation test, a DNA fragmentation test, an RNA fragmentation test, a nucleic acid sequencing-based test, or any combination thereof; and/or
   (ii) the level of tumor mutational burden (TMB), clonal TMB, indel TMB, or nonsense-mediated decay (NMD)-escape TMB; the presence or absence of a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof; and/or
(b) the method further comprises selecting a treatment, administering a treatment, or applying a treatment to the individual based on the generated genomic profile, wherein the treatment comprises an immune checkpoint inhibitor.

Exemplary Clause 243: The method of clause 238, wherein:
the CD274 nucleic acid molecule comprises or results from a rearrangement comprising a Breakpoint 1 and/or Breakpoint 2 within the chromosomal coordinates as listed in Table 1, or
the CD274 fusion nucleic acid molecule comprises a fusion between a CD274 gene, or a portion thereof, and a fusion partner gene as listed in Table 2, or a portion thereof, and
wherein the cancer is:
(a) a carcinoma, a sarcoma, a lymphoma, a leukemia, a myeloma, a germ cell cancer, or a blastoma;
(b) a solid tumor or a hematologic malignancy;
(c) a B cell cancer, melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor; or
(d) an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, unknown primary adenocarcinoma, gastric cancer or carcinoma, unknown primary carcinoma (CUP), unknown primary carcinoma (CUP) (NOS), unknown primary melanoma, stomach adenocarcinoma, or vagina squamous cell carcinoma (SCC).

Exemplary Clause 244: The method of clause 238, wherein:
(a) the CD274 nucleic acid molecule comprises or results from a rearrangement comprising a Breakpoint 1 and/or Breakpoint 2 within the chromosomal coordinates as listed in Table 1, and wherein the cancer is the corresponding cancer as listed in Table 6; or
(b) the CD274 fusion nucleic acid molecule comprises a fusion between a CD274 gene, or a portion thereof, and a fusion partner gene as listed in Table 2, or a portion thereof, and wherein the cancer is the corresponding cancer as listed in Table 7.

Exemplary Clause 245: The method of clause 238, wherein:
(a) the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4;
(b) the CD274 fusion nucleic acid molecule comprises a fusion between a CD274 gene, or a portion thereof, and a fusion partner gene as listed in Table 2, or a portion thereof, wherein the CD274 fusion nucleic acid molecule comprises or results from a corresponding CD274 Breakpoint and/or Fusion Partner Gene Breakpoint within the chromosomal coordinates as listed in Table 8, and wherein the cancer is the corresponding cancer as listed in Table 8; or
(c) the CD274 fusion nucleic acid molecule comprises a fusion between a CD274 gene, or a portion thereof, and a fusion partner gene as listed in Table 3, or a portion thereof, and the cancer is the corresponding cancer as listed in Table 3, and wherein the CD274 fusion nucleic acid molecule comprises or results from a corresponding CD274 Breakpoint and/or Fusion Partner Gene Breakpoint within the chromosomal coordinates as listed in Table 5.

Exemplary Clause 246: The method of clause 238, wherein the cancer is metastatic.

Exemplary Clause 247: The method of clause 238, wherein the immune checkpoint inhibitor:
(a) comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, or any combination thereof;
(b) is a PD-1-, or a PD-L1-targeted agent;
(c) is a PD-1 inhibitor, a PD-L1 inhibitor, or a CTLA-4 inhibitor; and/or
(d) is a monotherapy.

Exemplary Clause 248: The of clause 247, wherein:
(a) the PD-1 inhibitor comprises one or more of nivolumab, pembrolizumab, cemiplimab, or dostarlimab;
(b) the PD-L1-inhibitor comprises one or more of atezolizumab, avelumab, or durvalumab;
(c) the CTLA-4 inhibitor comprises ipilimumab; or
(d) the nucleic acid comprises a double-stranded RNA (dsRNA), a small interfering RNA (siRNA), or a small hairpin RNA (shRNA).

Exemplary Clause 249: The method of clause 238, wherein the treatment comprises:
(a) an additional anti-cancer therapy; and/or
(b) an immune checkpoint inhibitor in combination with one or more chemotherapeutic agents.

Exemplary Clause 250: The method of clause 249, wherein:
(a) the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof;
(b) the additional anti-cancer therapy comprises a cellular therapy, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy;
(c) the additional anti-cancer therapy comprises a nucleic acid, wherein the nucleic acid comprises a double-stranded RNA (dsRNA), a small interfering RNA (siRNA), or a small hairpin RNA (shRNA); and/or
(d) the one or more chemotherapeutic agents comprise a platinum-based chemotherapeutic agent, bevacizumab-Awwb, bevacizumab, carboplatin, paclitaxel, paclitaxel protein-bound, or pemetrexed.

Exemplary Clause 251: The method of clause 238, wherein the encoded PD-L1 polypeptide is oncogenic, and/or promotes cancer cell survival, angiogenesis, cancer cell proliferation, and any combination thereof.

Exemplary Clause 252: The method of clause 238, further comprising:
(a) acquiring knowledge of or detecting in a sample from the individual the presence or absence of a CD274 gene amplification;
(b) acquiring knowledge of or detecting in a sample from the individual the presence or absence of a base substitution, a short insertion/deletion (indel), a copy number alteration, or a genomic rearrangement in one or more genes;
(c) acquiring knowledge of or detecting in a sample from the individual the presence or absence of a genomic Epstein-Barr virus (EBV);
(d) acquiring knowledge of or detecting a microsatellite instability status of the cancer in a sample from the individual;
(e) acquiring knowledge of or determining tumor mutational burden (TMB) in a sample from the individual;
(f) acquiring knowledge of or determining the level of PD-L1 expression in a sample from the individual; and/or
(g) acquiring knowledge of or determining the clonality of the CD274 nucleic acid molecule in the cancer.

Exemplary Clause 253: The method of clause 252, wherein:
(a) the one or more genes comprise one or more of: TP53, PIK3CA, CDKN2A, KRAS, CDKN2B, CD274, MYC, JAK2, RB1, PDCD1LG2, APC, ARID1A, PTEN, BRAF, CREBBP, PBRM1, KMT2D, CCND1, KDM6A, BCL2L1, ERBB2, FBXW7, NF1, BCORL1, BRCA2, FGF19, FGFR1, MAP2K1, PRKC1, ATM, CDK12, CTNNB1, DNMT3A, FGF3, FGF4, GNAS, LYN, MET, NOTCH1, RNF43, STK11, TET2, VHL, ZNF217, ASXL1, BRCA1, EGFR, KDM5C, KIT, NFE2L2, NOTCH2, NOTCH3, PIK3R1, SOX9, TERC, ZNF703, MTAP, BRIP1, CDC73, ACVR1B, ATRX, MLH1, BRD4, SMAD4, PALB2, RAD21, GATA6, CTCF, MLH1, a mismatch repair gene, or any combination thereof;
(b) the cancer comprises a base substitution, a small insertion/deletion (indel), a copy number alteration, or a genomic rearrangement in the one or more genes;
(c) the EBV is HHV-4;
(d) the cancer or the individual comprises a genomic EBV or is positive for EBV;
(e) the cancer is microsatellite stable;
(f) the cancer has a TMB of less than 6 mutations per megabase (mut/Mb), between 6 and 20 mut/Mb, greater than 20 mut/Mb, a high TMB, a TMB of about 7.0 mut/Mb, or a TMB of at least about 10 mut/Mb;
(g) the cancer is PD-L1 positive, PD-L1-high positive, or PD-L1 negative;
(h) the cancer comprises a CD274 gene amplification, or the cancer does not comprise a CD274 gene amplification; and/or
(i) the CD274 nucleic acid molecule results from a clonal or a sub-clonal rearrangement of a CD274 gene in the cancer, or the CD274 nucleic acid molecule is clonal or sub-clonal in the cancer.

Exemplary Clause 254: The method of clause 252, wherein the cancer or the individual comprises a genomic EBV or is positive for EBV, and wherein:
the cancer is a gastric cancer, a gastric adenocarcinoma, or a stomach adenocarcinoma; and/or
the treatment comprises pembrolizumab.

Exemplary Clause 255: The method of clause 252, wherein clonality of the CD274 nucleic acid molecule is assessed by performing DNA sequencing on a sample obtained from the individual.

Exemplary Clause 256: The method of clause 255, wherein:
(a) clonality of the CD274 nucleic acid molecule is assessed based on sequencing coverage of a CD274 gene and the number of sequence read pairs spanning a breakpoint of the CD274 nucleic acid molecule; or
(b) clonality of the CD274 nucleic acid molecule is assessed based on the number of sequence read pairs spanning a breakpoint of the CD274 nucleic acid molecule.

Exemplary Clause 257: The method of clause 256, wherein:
(a) clonality of the CD274 nucleic acid molecule is assessed based on sequencing coverage of a CD274 gene and a threshold for the number of sequence read pairs spanning a breakpoint of the CD274 nucleic acid molecule, wherein the threshold is selected using a receiver operator characteristic (ROC) curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis;
(b) clonality of the CD274 nucleic acid molecule is assessed based on the number of sequence read pairs spanning a breakpoint of the CD274 nucleic acid molecule, wherein the clonality is assessed based on a threshold for the number of sequence read pairs spanning a breakpoint of the CD274 nucleic acid molecule, wherein the threshold is selected using a ROC curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis; and/or
(c) the sample is a bulk tumor sample derived from a single anatomic location.

Exemplary Clause 258: The method of clause 257, wherein:
(a) a sample from a tumor is PD-L1 high positive if it comprises a tumor proportion score (TPS) of at least about 50%; and/or
(b) the threshold for the number of sequence read pairs spanning a breakpoint of the CD274 nucleic acid molecule is at least about 20 read pairs, at least about 21 read pairs, at least about 22 read pairs, at least about 23 read pairs, at least about 24 read pairs, at least about 25 read pairs, at least about 26 read pairs, at least about 27 read pairs, at least about 28 read pairs, at least about 29 read pairs, at least about 30 read pairs, at least about 31 read pairs, at least about 32 read pairs, at least about 33 read pairs, at least about 34 read pairs, at least about 35 read pairs, at least about 36 read pairs, at least about 37 read pairs, at least about 38 read pairs, at least about 39 read pairs, at least about 40 read pairs, at least about 41 read pairs, at least about 42 read pairs, at least about 43 read pairs, at least about 44 read pairs, at least about 45 read pairs, at least about 46 read pairs, at least about 47 read pairs, at least about 48 read pairs, at least about 49 read pairs, or at least about 50 read pairs spanning a breakpoint of the CD274 nucleic acid molecule.

Exemplary Clause 259: The method of clause 253, wherein:
(a) responsive to acquiring knowledge of or determining that the CD274 nucleic acid molecule is clonal in the cancer or results from a clonal rearrangement of a CD274 gene in the cancer:
   (i) the individual is identified as likely to respond to a treatment comprising an immune checkpoint inhibitor;
   (ii) the individual is predicted to have longer survival when treated with a treatment comprising an immune checkpoint inhibitor, as compared to survival of: an individual whose cancer does not comprise a clonal CD274 nucleic acid molecule, or a CD274 nucleic acid molecule resulting from a clonal rearrangement of a CD274 gene; or an individual whose cancer comprises a sub-clonal CD274 nucleic acid molecule, or a CD274 nucleic acid molecule resulting from a sub-clonal rearrangement of a CD274 gene; and/or
   (iii) the individual is predicted to have an improved response to treatment with an immune checkpoint inhibitor, as compared to: an individual whose cancer does not comprise a clonal CD274 nucleic acid molecule, or a CD274 nucleic acid molecule resulting from a clonal rearrangement of a CD274 gene; or an individual whose cancer comprises a sub-clonal CD274 nucleic acid molecule, or a CD274 nucleic acid molecule resulting from a sub-clonal rearrangement of a CD274 gene; and/or
(b) acquiring knowledge of or determining that the CD274 nucleic acid molecule is clonal in the cancer or results from a clonal rearrangement of a CD274 gene in the cancer identifies the cancer as:
   (i) likely to be PD-L1 positive or PD-L1 high positive; and/or
   (ii) likely to have a TPS of at least about 50%, assessed based on an immunohistochemistry assay.

Exemplary Clause 260: The method of clause 238, wherein:
(a) the method further comprises obtaining the sample from the individual, and/or the sample is obtained from the cancer; and/or
(b) the sample:
   (i) comprises a tissue biopsy sample, a liquid biopsy sample, or a normal control,
   (ii) is from a tumor biopsy, tumor specimen, or circulating tumor cell,
   (iii) is a liquid biopsy sample and comprises blood, plasma, cerebrospinal fluid, sputum, stool, urine, or saliva,
   (iv) comprises cells and/or nucleic acids from the cancer,
   (v) comprises mRNA, DNA, circulating tumor DNA (ctDNA), cell-free DNA, or cell-free RNA from the cancer,
   (vi) is a liquid biopsy sample and comprises circulating tumor cells (CTCs), or
   (vii) is a liquid biopsy sample and comprises cell-free DNA (cfDNA), circulating tumor DNA (ctDNA), or any combination thereof.

Exemplary Clause 261: The method of clause 238, wherein:
(a) the method comprises acquiring knowledge of or detecting the CD274 nucleic acid molecule, or the PD-L1 polypeptide encoded by the CD274 nucleic acid molecule, in a tissue biopsy sample, in a liquid biopsy sample, or in both a tissue biopsy sample and a liquid biopsy sample, from the individual;
(b) acquiring knowledge of the CD274 nucleic acid molecule, or the PD-L1 polypeptide encoded by the CD274 nucleic acid molecule, comprises detecting the CD274 nucleic acid molecule, or the PD-L1 polypeptide encoded by the CD274 nucleic acid molecule, in the sample;
(c) detecting the CD274 nucleic acid molecule comprises detecting a fragment of the CD274 nucleic acid molecule comprising a breakpoint; and/or
(d) detecting the PD-L1 polypeptide encoded by the CD274 nucleic acid molecule comprises detecting a portion of the polypeptide that is encoded by a fragment of the CD274 nucleic acid molecule that comprises a breakpoint.

Exemplary Clause 262: The method of clause 238, wherein:
(a) the CD274 nucleic acid molecule is detected in the sample by:
   (i) one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing;
   (ii) sequencing using a massively parallel sequencing (MPS) technique, whole genome sequencing (WGS), whole exome sequencing, targeted sequencing, direct sequencing, a Sanger sequencing technique, next-generation sequencing (NGS), or RNA-sequencing (RNA-seq); or
   (iii) a polymerase chain reaction (PCR) amplification technique, a non-PCR amplification technique, an isothermal amplification technique, a reverse transcription PCR (RT-PCR), a quantitative real-time PCR (qPCR), or a reverse transcription quantitative real-time PCR (RT-qPCR) assay;
(b) the PD-L1 polypeptide encoded by the CD274 nucleic acid molecule is detected in the sample by one or more of: immunoblotting, enzyme linked immunosorbent assay (ELISA), immunohistochemistry, or mass spectrometry; and/or
(c) the CD274 nucleic acid molecule, or the PD-L1 polypeptide encoded by the CD274 nucleic acid molecule, is detected using a digital pathology method.

Exemplary Clause 263: The method of clause 238, wherein the method further comprises selectively enriching for one or more nucleic acid molecules in the sample comprising nucleotide sequences corresponding to the CD274 nucleic acid molecule, wherein the selectively enriching produces an enriched sample.

Exemplary Clause 264: The method of clause 263, wherein:
(a) the selectively enriching comprises:
   (i) combining one or more bait molecules with the sample, thereby hybridizing the one or more bait molecules to one or more nucleic acid molecules in the sample comprising nucleotide sequences corresponding to the CD274 nucleic acid molecule and producing nucleic acid hybrids, and isolating the nucleic acid hybrids to produce the enriched sample, or
   (ii) amplifying the one or more nucleic acid molecules comprising nucleotide sequences corresponding to the CD274 nucleic acid molecule using a polymerase chain reaction (PCR) to produce an enriched sample; and/or
(b) the method further comprises sequencing the enriched sample.

Exemplary Clause 265: The method of clause 264, wherein the one or more bait molecules comprise a capture nucleic acid molecule configured to hybridize to a nucleotide sequence corresponding to the CD274 nucleic acid molecule; and/or the one or more bait molecules are conjugated to an affinity reagent or to a detection reagent.

Exemplary Clause 266: The method of clause 265, wherein:
(a) the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides;
(b) the affinity reagent is an antibody, an antibody fragment, or biotin, or wherein the detection reagent is a fluorescent marker; and/or
(c) the capture nucleic acid molecule comprises a DNA, RNA, or mixed DNA/RNA molecule.

Exemplary Clause 267: The method of clause 238, wherein the individual is a human.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: CD274 Rearrangements as Biomarkers of Response to Immune Checkpoint Blockade Therapy

This Example describes the detection of CD274 rearrangements through comprehensive genomic profiling (CGP) and their use as predictive biomarkers of response to immune checkpoint inhibitors (ICIs).

### Materials and Methods

### Comprehensive Genomic Profiling

A total of 283,050 clinically-advanced cases of malignancy underwent comprehensive genomic profiling (CGP) (Frampton, G. M., et al. (2013) Nat. Biotechnol. 31(11): 1023-31). CGP on 0.8-1.1 megabases (Mb) of the coding genome was performed on hybridization-captured, adapter-ligation based libraries, to identify genomic alterations (base substitutions, small insertions/deletions, copy number alterations and rearrangements) in exons and select introns in up to 404 genes, tumor mutational burden (TMB), and microsatellite instability (MSI) status. The median sequencing depth of coverage was 830×.

TMB was calculated as the number of non-driver somatic coding mutations per megabase of genome sequenced (Chalmers, Z. R., et al. (2017) Genome Med. 9(1): 34). MSI status was determined by analyzing 114 intronic homopolymer repeat loci for length variability, and MSI high (MSI-H) was defined as previously described (Trabucco, S. E., et al. (2019) J. Mol. Diagnostics 21(6): 1053-1066).

PD-L1 immunohistochemistry (IHC) testing was performed according to the manufacturer instructions for a subset of specimens in the cohort (DAKO, PD-L1 IHC 22C3 pharmDx package insert, accessible at www.accessdata.fda.gov/cdrh_docs/pdf15/P150013c.pdf; VENTANA PD-L1 (SP142) Assay package insert, accessible at www.accessdata.fda.gov/cdrh_docs/pdf16/p160002s009c.pdf). DAKO's tumor proportion scoring (TPS) method was used to score these cases where TPS was equal to the proportion of PD-L1-positive tumor cells, defined as the number of PD-L1-positive tumor cells over the total number of tumor cells (PD-L1-positive and PD-L1-negative tumor cells).

### CD274 Rearrangement Calling

Functional rearrangements were defined as chromosomal translocation, deletion, duplication or inversion events with one breakpoint in CD274 (Transcript ID NM_014143) intron 5 to the 3′-UTR, and a second breakpoint outside of CD274. Such rearrangements were projected to incur loss of inhibitory microRNA binding site while leaving the Ig-like domains intact, leading to PD-L1 overexpression (Gong, A.-Y., et al. (2009) J. Immunol. 182(3): 1325-33). Breakpoint locations were determined by chimeric alignment of DNA sequences, which typically requires the number of supportive reads to be larger than 20.

### Genomic Viral Calling

The presence of oncogenic viruses in tumor specimens was determined by the identification of DNA sequences consistent with genomic viral DNA. Sequencing reads left unmapped to the human reference genome (hg19) were de novo assembled using Velvet (Zerbino, D. R. & Birney, E. (2008) Genome Res. 18(5): 821-9; Zerbino, D. R. (2010) Curr Protoc Bioinformatics, Chapter 11: Unit 11.5). The assembled contigs were competitively aligned using BLASTn (Altschul, S. F., et al. (1990), J. Mol. Biol. 215(3): 403-10) to the NCBI database of over 3 million known viral nucleotide sequences. A positive viral status was determined by contigs of at least 80 nucleotides in length and with at least 97% identity to the BLAST sequence (Knepper, T. C., et al. (2019) Clin. Cancer Res. 25(19): 5961-5971).

### Statistical Analysis

All statistical analyses were performed using the R software (R Foundation for Statistical Computing, v3.6.0) (Ihaka, R. & Gentleman, R. (1996) J. Comput. Graph. Stat. 5(3): 299-314). Proportions of categorical variables were compared using the Fisher exact test, while continuous variables were compared using the Wilcoxon rank sum test. All determined p-values were two-sided, and multiple hypothesis testing correction was performed using the Benjamini-Hochberg procedure to calculate the false discovery rate (FDR). Genomic coordinates were identified using hg19 on the UCSC Genome Browser (Kent, W. J., et al. (2002) Genome Res. 12(6): 996-1006).

### Clinico-Genomic Cohort

Retrospective longitudinal clinical data were derived from electronic health record (EHR) data, including patient-level structured and unstructured data, curated via technology-enabled abstraction, and linked to genomic data derived from CGP tests in a clinico-genomic database (CGDB) by de-identified, deterministic matching (Singal, G., et al. (2019), JAMA 321(14): 1391-1399; Birnbaum, B., et al. (2020) arXiv, arXiv: 2001.09765).

For CD274-rearranged patients treated with immune checkpoint inhibitor therapy (monotherapy or combination therapy; n=8), the duration of line of therapy (real-world time at risk of treatment discontinuation) was calculated as the time between the earliest and the last recorded instance of drug regimen administration or scheduled drug regimen administration. The treatment was considered as discontinued if there was a change in line of therapy or death of the patient, with all others considered as censored.

### Results

### Characteristics of CD274-Rearranged Tumors in a Large Pan-Cancer Cohort

Gene rearrangements in the CD274 gene, which encodes for programmed cell death ligand-1 (PD-L1), are positively associated with PD-L1 expression and may confer benefit to treatment with ICIs, but a pan-cancer characterization of these alterations is lacking.

To this end, the genomic landscape of CD274 rearrangements was assessed across 419 different tumor types that comprised 283,050 patient samples. In total, 145 samples (0.05%) with a functional rearrangement in CD274 were identified. This cohort was 52.4% female, with a median age of 64.5 years (range: 16.0-89+ years), and 44.2% of the specimens corresponded to metastatic tumor biopsies (Table 9).

TABLE 9
Demographic characteristics of the
CD274-rearranged and the CD274 wild-type cohorts.

| | | CD274- | CD274 | |
|---|---|---|---|---|
| | | rearranged | wild-type | |
| | | (N = 145) | (N = 282905) | p value |
| Sex: Males (%) | | 47.6 | 44.7 | 0.5 |
| Median age (Interquartile | | 64.5 | 63.0 | 0.2 |
| range; years) | | (56.0-73.0) | (54.0-71.0) | |
| Genomic | African | 7.5 | 9.6 | 0.5 |
| ancestry (%) | American | 14.5 | 8.6 | 0.02 |
| | East Asian | 2.1 | 3.7 | 0.4 |
| | European | 75.9 | 77.0 | 0.8 |
| | South Asian | 0.0 | 1.0 | 0.4 |
| Metastatic (%) | | 44.2 | 45.1 | 0.9 |
| | | (50/113) | (103451/229399) | |

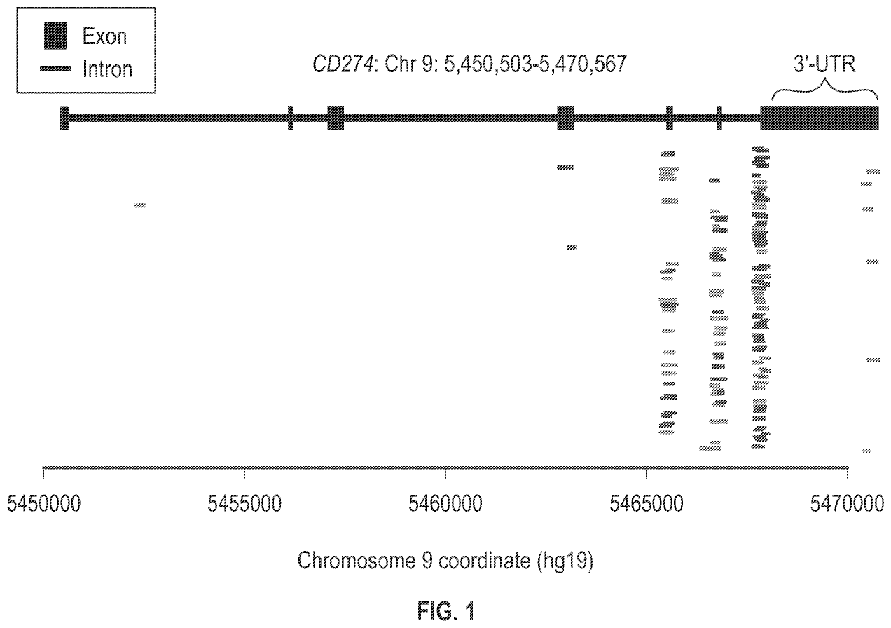

When compared to the CD274 wild type cohort, the CD274-rearranged cohort was significantly enriched for the admixed American genomic ancestry (p value=0.02; Table 9), with 8.6% of subjects in the wild-type cohort and 14.5% of subjects in the CD274-rearranged cohort belonging to this genomic ancestry. Nearly all of the CD274-rearranged samples harbored rearrangements that affected the 3′-UTR within 135 base pairs of the wild-type stop codon of CD274, with 138 of the 145 CD274-rearranged cases having a breakpoint within 5% of exon 7 (FIG. 1).

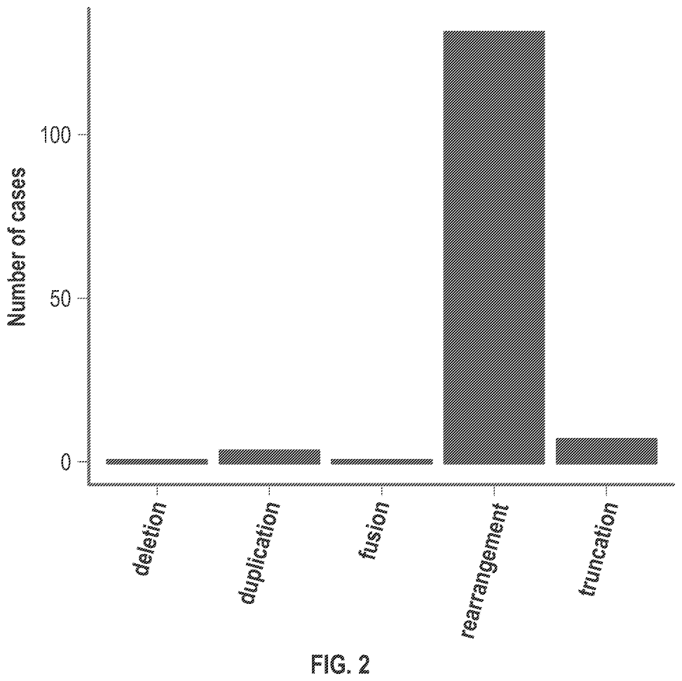

Of the types of genomic events identified, most were rearrangements, with a small number of focal duplications, deletions, and truncation events (FIG. 2).

CD274 amplifications occurred in 25% of the CD274-rearranged cohort (odds ratio (OR)=4.6, p value=5.3e-44).

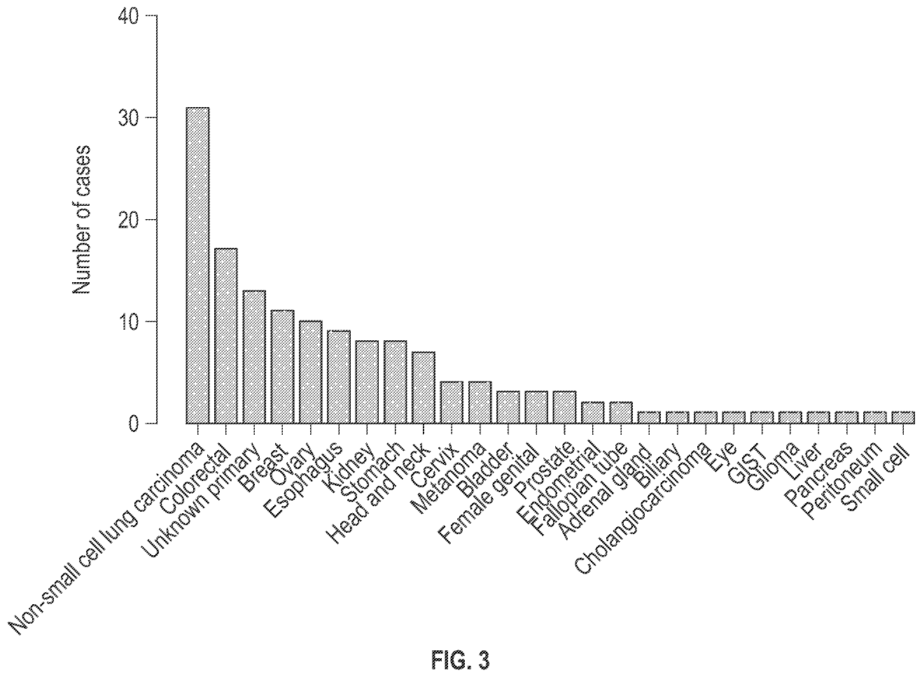

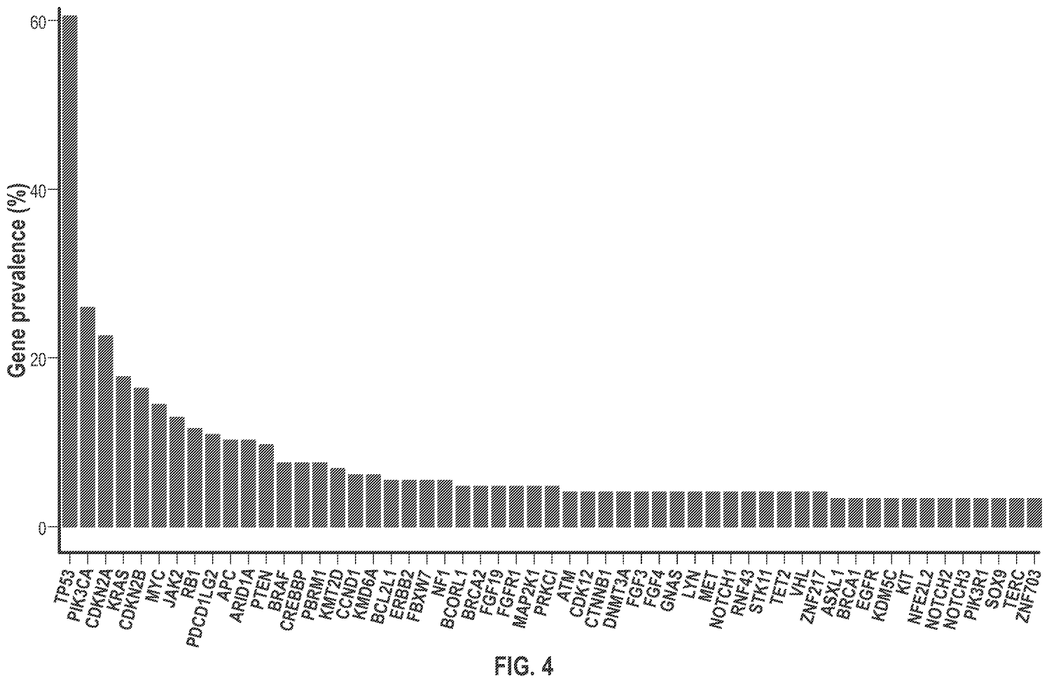

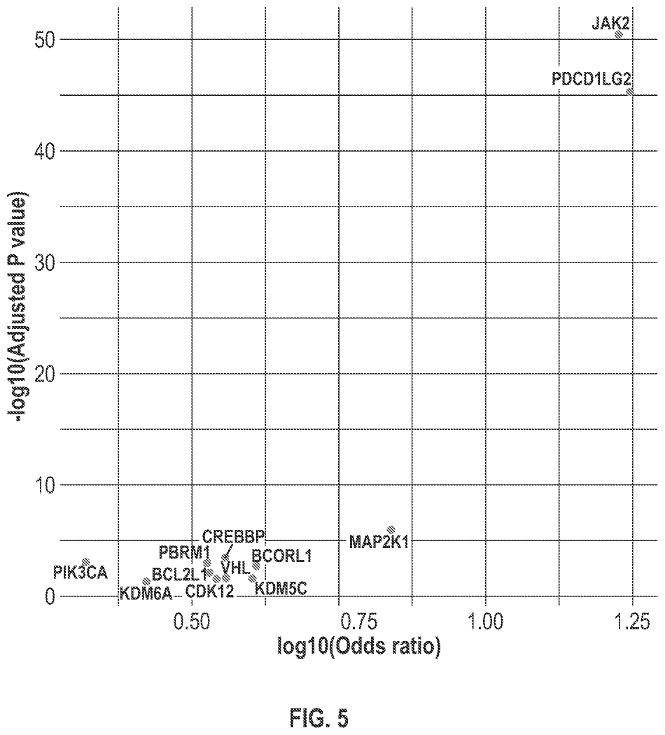

The most common tumor type harboring CD274 rearrangements was non-small cell lung cancer, followed by colorectal cancer and unknown primary cancers (FIG. 3). Within the CD274-rearranged cohort, GAs in TP53 were the most common (60.7%), followed by GAs in PIK3CA (26.2%), CDKN2A (22.8%), KRAS (17.9%), CDKN2B (16.6%), and MYC (14.5%) (FIG. 4). GAs in multiple genes co-occurred with CD274 rearrangements, including PIK3CA (OR=2.1; adjusted p value=0.001), JAK2 (OR=16.7; adjusted p value=3.6e-52), PDCD1LG2 (OR=17.8; adjusted p value=4.8e-46), CREBBP (OR=3.6; adjusted p value=4.2e-4) and PBRM1 (OR=3.4; adjusted p value=0.001) (FIG. 5). Only 13% and 11% of the CD274-rearranged cohort harbored alterations in JAK2 and PDCD1LG2, respectively.

Genomic human papillomavirus strain 16 (HPV-16) (OR=6.2; p value=1.1e-10), genomic Epstein-Barr virus (EBV) (OR=8.4; p value=0.003), and GAs in genes involved in mismatch repair such as MLH1 (OR=4.3; p value=0.05) also co-occurred with CD274 rearrangements.

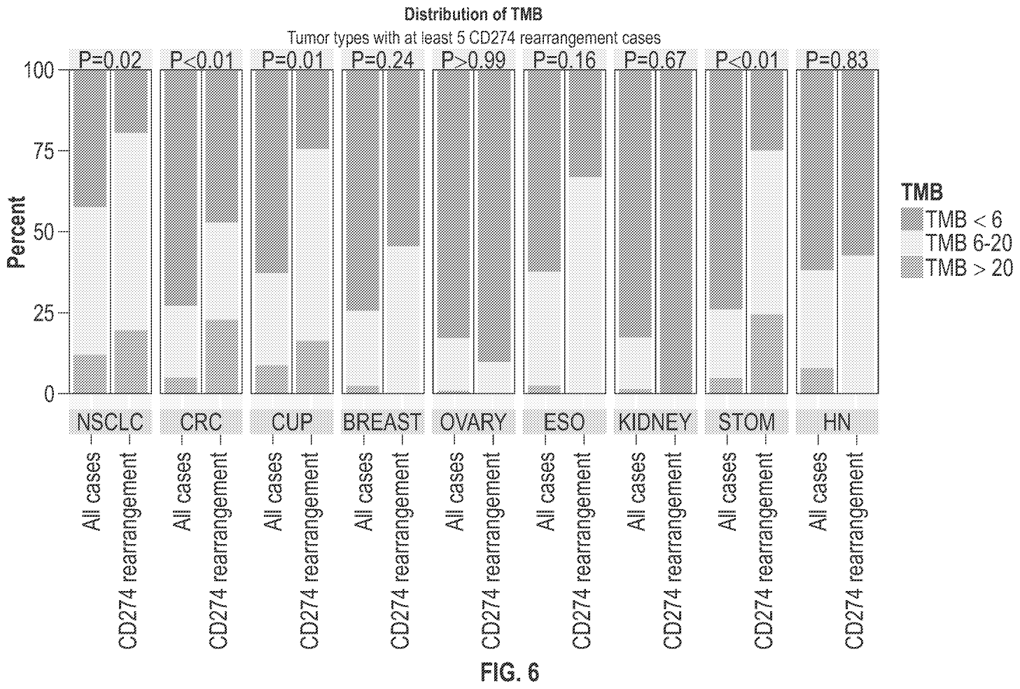

Within the CD274-rearranged cohort, there was also an enrichment for tumor types harboring relatively high TMB (FIG. 6), but 92.3% (n=131/142) of the cohort was microsatellite stable. The median TMB across the cases harboring CD274 rearrangements (7.0 mutations per megabase [muts/Mb]; n=145) was found to be significantly greater than that observed in the CD274 wild-type cohort (3.5 muts/Mb; n=282,905; p value=1.7e-11). When examined within disease groups, this relationship held true only in some disease types, including non-small cell lung, colorectal, unknown primary, and gastric carcinomas (FIG. 6).

### Association of CD274 Rearrangements and PD-L1 IHC

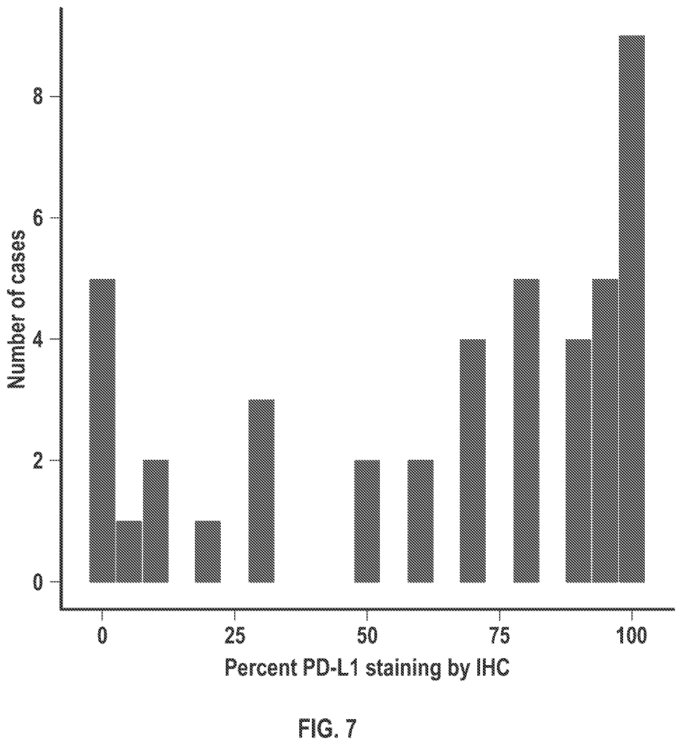

A cohort of 43 out of the 145 patients for whom both CGP and PD-L1 IHC data were available was further evaluated. Within this subgroup, PD-L1 staining scores were highly skewed towards positivity, with 90.7%, or 39 out of the 43 samples, exhibiting a score ≥1%; and 72.1%, or 31 out of the 43 samples, exhibiting a score of ≥50% (FIG. 7 and Table 10). In contrast, among the CD274 wild-type population (n=282.905), of which 55,380 of the samples were scored for PD-L1 staining. 38.7% (21,410 of the 55,380 samples) had a TPS ≥1% while 15.0% (or 8280 of the 55,380 samples) had a TPS ≥50%.

Time on ICI Therapy in CD274-Rearranged Patients from a Real World Clinico-Genomic Database

The association between the presence of CD274 rearrangements and clinical benefit from immune checkpoint inhibitors (ICIs) was investigated.

Eight CD274-rearranged cases across multiple tumor types treated with ICI were observed in a CGDB. These included 2 gastric adenocarcinomas, 2 triple negative breast cancers (TNBC), 1 colon adenocarcinoma, 1 lung squamous cell carcinoma (lung SCC), 1 lung non-squamous cell adenocarcinoma, and 1 serous ovarian carcinoma (Table 11).

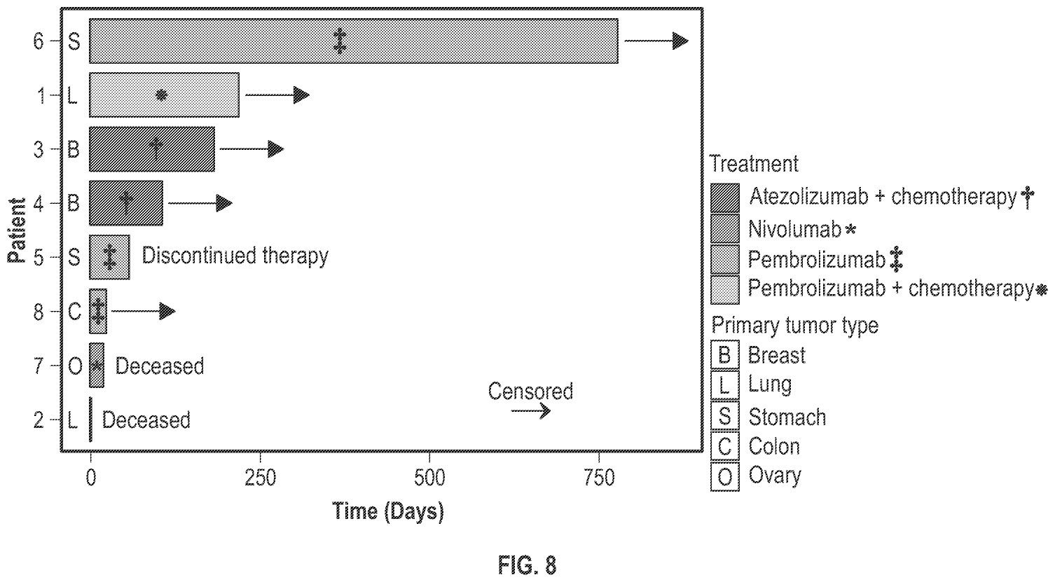

Fifty percent, or 4 out of the 8 cases, were on ICI treatment for at least 3 months, with 3 of these 4 cases having been on ICI treatment for over 6 months (FIG. 8). Sustained clinical benefit of over two years (778 days as of the last follow-up) was observed in one gastric adenocarcinoma case treated with second line pembrolizumab monotherapy. The gastric adenocarcinoma case was microsatellite stable (MSS), had a TMB of 1.25 muts/Mb, and was positive for genomic EBV. Two triple-negative breast cancer (TNBC) cases, one with a TMB of 0 muts/Mb, MSS, and PD-L1 positivity, and another case with no data on TMB, MSI or PD-L1 status, were on a combination of atezolizumab and paclitaxel for 183 days and 106 days, respectively, as of last follow-up. The lung adenocarcinoma case was observed to be PD-L1 high, and was on first line combination therapy of carboplatin, pemetrexed and pembrolizumab for 219 days, with a real-world partial response to treatment 3 months after start of therapy, as well as an observed real-world stable disease as of last follow up.

TABLE 10
Characteristics of CD274 rearrangements with PD-L1 IHC scores.

| | | | | Rearrange- | CD274 | Partner gene | | | PD-L1 | PD-L1 |
|---|---|---|---|---|---|---|---|---|---|---|
| | | | | ment | breakpoint | breakpoint | Rearrange- | PD-L1 | test | test |
| Age | Sex | Tumor type | Gene | partner gene | coordinates | coordinates | ment type | test | score | platform |
| 71 | Male | adrenal gland cortical | CD274 | N/A | chr9: 5466758- | chr9: [symbol] 819480- | rearrangement | Tumor | 0 | DAKO |
| | | carcinoma | | | 5466912 | 7819610 | | Stain | | |
| 70 | Female | lung small cell | CD274 | CFAP52 | chr9: 5466613- | chr17: 9496835- | rearrangement | Tumor | 0 | DAKO |
| | | undifferentiated | | | 5466970 | 9497088 | | Stain | | |
| | | carcinoma | | | | | | | | |
| 48 | Female | breast invasive ductal | CD274 | N/A | chr9: 5467780- | chr9: 6511502- | rearrangement | Tumor | 0 | DAKO |
| | | carcinoma (idc) | | | 5467988 | 6511679 | | Stain | | |
| 86 | Female | lung adenocarcinoma | CD274 | N/A | chr9: 5467646- | chr9: 5447459- | rearrangement | Tumor | 0 | DAKO |
| | | | | | 5467904 | 5447604 | | Stain | | |
| 62 | Male | colon adenocarcinoma | CD274 | MYLK | chr9: 5486373- | chr3: 123592212- | rearrangement | Tumor | 1 | DAKO |
| | | (crc) | | | 5465731 | 123592310 | | Stain | | |
| 75 | Male | colon adenocarcinoma | CD274 | N/A | chr9: 5467655- | chr9: 5489388- | rearrangement | Tumor | 5 | DAKO |
| | | (crc) | | | 5467913 | 5489533 | | Stain | | |
| 40 | Male | colon adenocarcinoma | CD274 | N/A | chr9: 5466698- | chr9: 5582855- | rearrangement | Tumor | 10 | DAKO |
| | | (crc) | | | 5466964 | 5583098 | | Stain | | |
| 65 | Female | lung non-small cell | CD274 | N/A | chr9: 5467714- | chr18: 28214456- | rearrangement | Tumor | 10 | DAKO |
| | | Lung carcinoma | | | 5467907 | 28214689 | | Stain | | |
| | | (nsclc) (nos) | | | | | | | | |
| 76 | Female | unknown primary | CD274 | PTPPD | chr9: 5467743- | chr: 9745527- | rearrangement | Tumor | 20 | DAKO |
| | | melanoma | | | 5467994 | 9745899 | | Stain | | |
| 81 | Male | lung squamous cell | CD274 | RIC1 | chr9: 5466596- | chr9: 5731619- | rearrangement | Tumor | 30 | DAKO |
| | | carcinoma (scc) | | | 5466853 | 5731896 | | Stain | | |
| 59 | Male | lung adenocarcinoma | CD274 | N/A | chr9: 5466689- | chr9: 6676528- | rearrangement | Tumor | 30 | DAKO |
| | | | | | 5466956 | 6676754 | | Stain | | |
| 76 | Male | eye lacrimal duct | CD274 | HPK2 | chr9: 5466598- | chr [symbol] : 139280432- | rearrangement | Tumor | 30 | DAKO |
| | | carcinoma | | | 5467002 | 139281022 | | Stain | | |
| 40 | Male | prostate acinar | CD274 | N/A | chr9: 5467714- | chr9: 4316216- | rearrangement | Tumor | 50 | DAKO |
| | | adenocarcinoma | | | 5467957 | 4316474 | | Stain | | |
| 80 | Male | lung adenocarcinoma | CD274 | N/A | chr9: 5467678- | chr9: 3590897- | rearrangement | Tumor | 50 | DAKO |
| | | | | | 5467931 | 3591102 | | Stain | | |
| [symbol] | Male | unknown primary | CD274 | PLGRKT | chr9: 5465378- | chr9: 5428506- | rearrangement | Tumor | 60 | DAKO |
| | | adenocarcinoma | | | 5465661 | 5428752 | | Stain | | |
| 74 | Male | lung non-small cell | CO274 | N/A | chr9: 5465440- | chr9: 5479019- | rearrangement | Tumor | 60 | DAKO |
| | | lung carcinoma | | | 5465671 | 5479384 | | Stain | | |
| | | (nsclc) (nos) | | | | | | | | |
| 61 | Male | lung squamous cell | CD274 | N/A | chr9: 5465342- | chr9: 5270435- | rearrangement | Tumor | 70 | DAKO |
| | | carcinoma (soc) | | | 5465658 | 5270947 | | Stain | | |
| 49 | Male | head and neck squamous | CD274 | N/A | chr9: 5467963- | chr9: 5500043- | rearrangement | Tumor | 70 | DAKO |
| | | cell carcinoma (hnscc) | | | 5468050 | 5500260 | | Stain | | |
| 32 | Male | bladder carcinoma (nos) | CD274 | CD274 | chr9: 5467755- | chr9: 5470492- | rearrangement | Tumor | 70 | DAKO |
| | | | | | 5467993 | 5470638 | | Stain | | |
| 69 | Male | lung non-small cell | CD274 | PTPRD | chr9: 5487716- | chr9: 8809122- | rearrangement | Tumor | 70 | DAKO |
| | | Lung carcinoma | | | 5467879 | 8809281 | | Stain | | |
| | | (nsclc) (nos) | | | | | | | | |
| 82 | Female | Skin melanoma | CD274 | PLGRKT | chr9: 5466638- | chr9: 5399714- | rearrangement | Tumor | 80 | DAKO |
| | | | | | 5466892 | 5400057 | | Stain | | |
| 58 | Female | cervix adenocarcinoma | CD274 | PLGRKT | chr9: 5465459- | chr9: 5421268- | rearrangement | Tumor | 80 | DAKO |
| | | | | | 5465671 | 5421464 | | Stain | | |
| 69 | Female | ovary epithelial | CD274 | N/A | chr9: 5467659- | chr2: 157519407- | rearrangement | Tumor | 80 | DAKO |
| | | carcinoma | | | 5467922 | 157519682 | | Stain | | |
| 55 | Female | lung non-small cell | CD274 | N/A | chr9: 5467787- | chr9: 5471671- | rearrangement | Tumor | 80 | DAKO |
| | | lung carcinoma | | | 5468003 | 5471863 | | Stain | | |
| | | (nsclc) (nos) | | | | | | | | |
| 47 | Female | ovary clear cell | CD274 | PLGRKT | chr9: 5467883- | chr9: 5426435- | rearrangement | Tumor | 80 | DAKO |
| | | carcinoma | | | 5468029 | 5426627 | | Stain | | |
| 42 | Female | cervix squamous cell | CD274 | PDCD1LG2 | chr9: 5466586- | chr9: 5543484- | rearrangement | Tumor | 90 | DAKO |
| | | carcinoma (soc) | | | 5466770 | 5543696 | | Stain | | |
| [symbol] | Female | ovary serous carcinoma | CD274 | N/A | chr9: 5467642- | chr9: 94722907- | rearrangement | Tumor | 90 | DAKO |
| | | | | | 5467900 | 94722813 | | Stain | | |
| 86 | Female | colon adenocarcinoma | CD274 | EPMP1 | chr9: 5467653- | chr9: 5789739- | rearrangement | Tumor | 90 | DAKO |
| | | (crc) | | | 5467891 | 5790096 | | Stain | | |
| 72 | Female | lung adenocarcinoma | CD274 | CHMPS | chr9: 5465378- | chr9: 33281569- | rearrangement | Tumor | 90 | DAKO |
| | | | | | 5465756 | 33281786 | | Stain | | |
| 61 | Female | lung adenocarcinoma | CD274 | N/A | chr9: 5467648- | chr18: 27844666- | rearrangement | Tumor | 95 | DAKO |
| | | | | | 5467991 | 27844942 | | Stain | | |
| 66 | Male | lung adenocarcinoma | CD274 | PTPRB | chr9: 5467734- | chr12: 70937973- | rearrangement | Tumor | 95 | DAKO |
| | | | | | 5467932 | 70938263 | | Stain | | |
| 76 | Female | lung adenocarcinoma | CD274 | PLGRKT | chr9: 5467639- | chr9: 5420662- | rearrangement | Tumor | 95 | DAKO |
| | | | | | 5467873 | 5421059 | | Stain | | |
| 67 | Female | liver hepatocellular | CD274 | N/A | chr9: 5466585- | chr9: 5201780- | rearrangement | Tumor | 95 | DAKO |
| | | carcinoma (hcc) | | | 5466876 | 5202054 | | Stain | | |
| 78 | Female | lung non-small cell | CD274 | N/A | chr9: 5467638- | chr9: 5441513- | duplication | Tumor | 95 | DAKO |
| | | lung carcinoma | | | 5467928 | 5441883 | | Stain | | |
| | | (nsclc) (nos) | | | | | | | | |
| 53 | Female | ovary serous carcinoma | CD274 | N/A | chr9: 5467681- | chr9: 5198653- | rearrangement | Tumor | 99 | DAKO |
| | | | | | 5467966 | 5198919 | | Stain | | |
| 46 | Female | vagina squamous cell | CD274 | JAK2 | chr9: 5467855- | chr9: 5005621- | rearrangement | Tumor | 99 | DAKO |
| | | carcinoma (scc) | | | 5467998 | 5005795 | | Stain | | |
| 89 | Female | unknown primary | CD274 | PLGRKT | chr9: 5467709- | chr9: 5429881- | rearrangement | Tumor | 100 | DAKO |
| | | carcinoma (cup) (nos) | | | 5467965 | 5430057 | | Stain | | |
| 53 | Male | esophagus | CD274 | N/A | chr9: 5466590- | chr9: 5149992- | duplication | Tumor | 100 | DAKO |
| | | adenocarcinoma | | | 5466996 | 5150287 | | Stain | | |
| 71 | Male | unknown primary | CD274 | PLGRKT | chr9: 5487661- | chr9: 5424930- | rearrangement | Tumor | 100 | DAKO |
| | | adenocarcinoma | | | 5467911 | 5425336 | | Stain | | |
| 57 | Male | kidney renal cell | CD274 | PLGRKT | chr9: 5467674- | chr9: 5425490- | rearrangement | Tumor | 100 | DAKO |
| | | carcinoma | | | 5467950 | 5425913 | | Stian | | |
| 66 | Male | esophagus squamous cell | CD274 | N/A | chr9: 5467660- | chr9: 5479655- | rearrangement | Tumor | 100 | DAKO |
| | | carcinoma (scc) | | | 5467942 | 5479937 | | Stain | | |
| 62 | Male | bladder urothelial | CD274 | N/A | chr9: 5465343- | chr9: 8143594- | rearrangement | Tumor | 100 | DAKO |
| | | ( [symbol] nal cell) | | | 5465699 | 8144014 | | Stain | | |
| | | carcinoma | | | | | | | | |
| 66 | Female | cervix squamous cell | CD274 | N/A | chr9: 5467637- | chr9: 5484662- | rearrangement | Tumor | 100 | DAKO |
| | | carcinoma (scc) | | | 5467886 | 5485082 | | Stain | | |

[symbol]

indicates data missing or illegible when filed

TABLE 11
Real-world patient treatment course in CD274-rearranged tumors.

| | | | | | Partner | CD274 |
|---|---|---|---|---|---|---|
| | | | Gene/ | CD274 | gene | Rearrangement |
| Age/ | | Sample | Partner | breakpoint | breakpoint | type/ |
| Sex¹ | Diagnosis | type | Gene | coordinates | coordinates | description |
| 75/M | Lung non- | Liquid | CD274/ | chr9; 5467965- | chr9: 5407366- | Rearrangement/ |
| | squmous | biopsy | PLGRKT | NA | NA | chr9 duplication |
| | cell lung | (blood) | | | | fragment |
| | adenocarcinoma, | | | | | CD274 |
| | stage IV | | | | | (NM_014143): [symbol] |
| | B | | | | | rearrangement |
| | | | | | | breakpoint exon |
| | | | | | | 7 |
| 61/M | Lung | Tissue | CD274/ | chr9: 5465342- | chr9: 5270435- | Rearrangement: |
| | squamous | biopsy | CD274 | 5466668 | 5270947 | chr9 duplication |
| | cell | | | | | fragment |
| | carcinoma, | | | | | (NM_014143): [symbol] |
| | stage IV | | | | | rearrangement |
| | | | | | | breakpoint |
| | | | | | | intron 5 |
| 73/F | Breast | Tissue | CD274/ | chr9: 5466636- | chr9: 5848064- | Trunication; chr9 |
| | carcinoma | biopsy | None | 5466908 | 5848343 | inversion |
| | (NOS), stage | | | | | fragment |
| | II B⁶ | | | | | CD274 |
| | | | | | | (NM_014143): [symbol] |
| | | | | | | rearrangement |
| | | | | | | breakpoint |
| | | | | | | intron6 |
| 55/F | Breast invasive | Liquid | CD274/ | chr9: 5467939- | chr9: 5486358- | Rearrangement: |
| | ductal carcinoma | biopsy | None | NA | NA | chr9 deletion |
| | (IDC), | (blood) | | | | fragment |
| | stage III C⁶ | | | | | CD274 |
| | | | | | | (NM_014143): [symbol] |
| | | | | | | rearrangement |
| | | | | | | breakpoint exon |
| | | | | | | 7. |
| | | | | | | Also seen |
| | | | | | | Translocation: |
| | | | | | | CD274 |
| | | | | | | (NM_014143): [symbol] |
| | | | | | | rearrangement |
| | | | | | | breakpoint exon |
| | | | | | | 7. |
| | | | | | | Also seen chr9 |
| | | | | | | deletion |
| | | | | | | fragment: [symbol] |
| | | | | | | JAK2(ex1-4 |
| | | | | | | NM_004972)- |
| | | | | | | CD274(ex7-7 |
| | | | | | | NM_014143) |
| 59/M | Stomach | Tissue | CD274/ | chr9: 5467786- | chr9: 5442048- | Rearrangement: |
| | adenocarcinoma, | biopsy | None | 5467955 | 5442165 | chr9 duplication |
| | stage III | | | | | fragment |
| | C | | | | | CD274 |
| | | | | | | (NM_014143): [symbol] |
| | | | | | | rearrangement |
| | | | | | | breakpoint exon |
| | | | | | | 7 |
| 44/F | Stomach | Tissue | CD274/ | chr9: 5467697- | chr4: 131103841- | Tran [symbol] |
| | adenocarcinoma, | biopsy | None | 5467980 | 131104134 | CD274 |
| | stage IV⁷ | | | | | (NM_014143): [symbol] |
| | | | | | | rearrangement |
| | | | | | | breakpoint exon |
| | | | | | | 7 |
| 77/F | Ovary serous | Liquid | CD274/ | chr9: 5467704- | chr9: 5470872- | Rearrangement: |
| | carcinoma, | biopsy | None | 5467951 | 5471004 | chr9 deletion |
| | stage IIIC | (blood) | | | | fragment: |
| | | | | | | CD274 |
| | | | | | | (NM_014143): [symbol] |
| | | | | | | deletion, |
| | | | | | | breakpoint in |
| | | | | | | exon 7 UTR |
| 84/F | Colon | Tissue | CD274/ | chr9: 5467653- | chr9: 5789739- | Rearrangement: |
| | adenocarcinoma, | biopsy | ERMP1 | 5467891 | 5790086 | chr9 deletion |
| | stage IV A | | | | | fragment: |
| | | | | | | CD274 |
| | | | | | | (NM_014143): [symbol] |
| | | | | | | rearrangement |
| | | | | | | breakpoint exon |
| | | | | | | 7 |

| | | | | | | Duration |
|---|---|---|---|---|---|---|
| | | | PD-L1 | | | on |
| Age/ | TMB/ | PD-L1 | test | Therapy/ | Clinical | Treatment |
| Sex¹ | MSI² | test | score³ | line # | response ⁴ | (days)⁵ |
| 75/M | NA/U | Tumor | High | Car [symbol] | Par [symbol] | 219 |
| | | Stain | Positive | pembro [symbol] | response | |
| | | | (DAKO | line | (May | |
| | | | 22C3), | | 2020), | |
| | | | 60% | | Progressive | |
| | | | 59% | | disease | |
| | | | | | (August | |
| | | | | | 2020), | |
| | | | | | Stable | |
| | | | | | disease | |
| | | | | | (September | |
| | | | | | 2020) | |
| 61/M | 17.5/ | Tumor | High | Atezolizumab, | N/A | 0 |
| | MSS | Stain | Positive | bevacizumab- | | |
| | | | (DAKO | Awwb, | | |
| | | | 22C3), | [symbol] | | |
| | | | 70% | [symbol] | | |
| | | | | [symbol] | | |
| 73/F | 0/ | Combined | Positive | Atezolizumab, | N/A | 183 |
| | MSS | Stain | (Ven [symbol] | pac [symbol] | | |
| | | | SP142), | I protein- | | |
| | | | ICS 1% | bound | | |
| 55/F | NA/ | NA | NA | Atezolizumab, | N/A | 106 |
| | NA | | | pac [symbol] | | |
| | | | | I protein- | | |
| | | | | pound/ | | |
| | | | | [symbol] line | | |
| 59/M | 13.75/ | NA | NA | Pembrolizumab; | N/A | 57 |
| | MSI-H | | | 2^{nd} line | | |
| 44/F | 1.25/ | NA | NA | Pembrolizumab; | N/A | 778 |
| | MSS | | | 2^{nd} line | | |
| 77/F | NA/ | NA | NA | [symbol] | N/A | 17 |
| | NA | | | | | |
| 84/F | 7.5/ | Tumor | High | Pembrolizumab; | N/A | 22 |
| | MSS | Stain | positive | 2^{nd} line | | |
| | | | (DAKO | | | |
| | | | 22C3) | | | |
| | | | 90% | | | |

¹Age at diagnosis.
²TMB. tumor mutational burden; MSS, microsatellite stable; MSI-H, mi [symbol] llite instability (MSI) high; NA, notavailable; U, unknown.
³Assay used provided in parentheses.
⁴ Clinical responses based on RECIST (Response Evaluation Criteria in Solid Tumors) refers to a set of standardized rules for defining tumor progression during a course of treatment. Additional information on the RECIST rules can be found at https://recist[dot]eo [symbol] [dot]org/.
⁵Corresponds to time at risk of treatment discontinuation (days): see. e.g. Campbell et al., Cancers (Basel). 2020; 12(8): 2311 for additional information.
⁶This tumor was also Estrogen Receptor-negative (ER−), Progesterone Receptor-negative (PR−), and human epidermal growth factor receptor 2-negative (HER2−).
⁷This tumor was also found to be positive for genomic Epstein Barr Virus (HHV-4). [symbol]
indicates data missing or illegible when filed

### Determination of CD274 Rearrangement Clonality

Quantifying the clonal nature of CD274 rearrangements can help identify if the genomic event is the major event driving tumor growth, as well as further fine-tune the ability to predict responses to ICI. Patients with clonal CD274 rearrangements may respond better to ICI than patients with sub-clonal CD274 rearrangements. The clonality of CD274 rearrangements was determined through single-site bulk-tumor CGP of solid tumor specimens during routine clinical care, using the sequencing coverage at the CD274 gene region and the number of sequencing read pairs that spanned the rearrangement break point for the analysis.

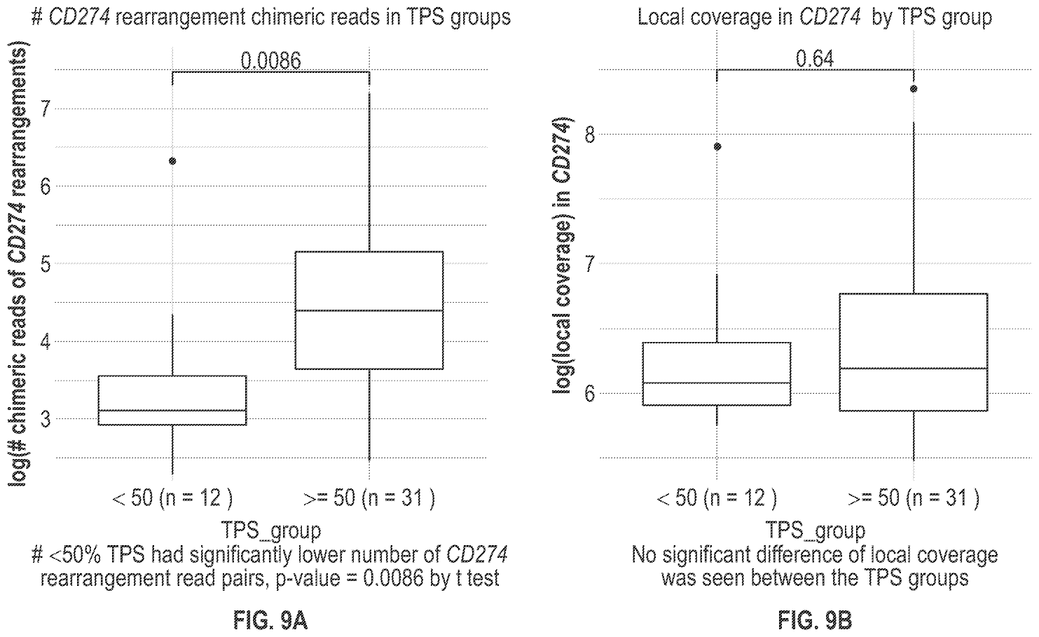

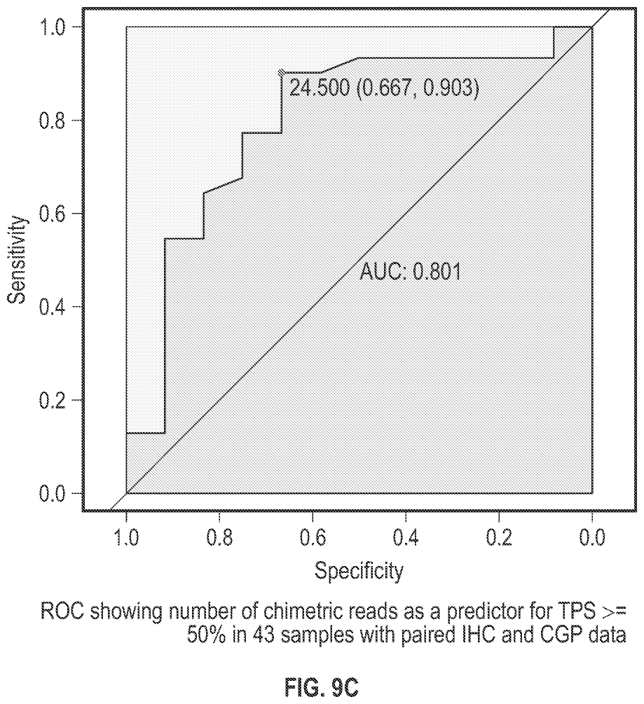

Based on these analyses, CD274-rearranged samples with TPS <50% were found to have significantly lower numbers of chimeric CD274 rearrangement reads (p value=0.009), while total number of reads aligned to CD274 in these samples was comparable to that of samples with TPS ≥50% (FIGS. 9A-9B). From this observation, it was inferred that sub-clonal CD274 rearrangements and genotype heterogeneity could contribute to low TPS. Consistent with this, a receiver operating curve (ROC) analysis indicated that the number of chimeric CD274 reads was a non-trivial predictor of TPS ≥ 50% in the 43 samples analyzed (area under the curve (AUC)=0.80, FIG. 9C).

### CONCLUSIONS

The results described in this Example demonstrated that CD274 gene rearrangements were associated with increased PD-L1 IHC scores, higher TMB, and clinical benefit of ICI treatment in various cancer types.

What is claimed is:

1. A method of selecting a treatment for an individual having a cancer, the method comprising detecting or acquiring knowledge of a cluster of differentiation 274 (CD274) nucleic acid molecule, or a programmed death-ligand 1 (PD-L1) polypeptide encoded by the CD274 nucleic acid molecule, in a sample from the individual, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
(i) the CD274 fusion nucleic acid molecule comprises a fusion between a CD274 gene, or a portion thereof, and a fusion partner gene as listed in Table 2, or a portion thereof, or
(ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
wherein detecting or acquiring knowledge of the CD274 nucleic acid molecule, or the PD-L1 polypeptide encoded by the CD274 nucleic acid molecule, in the sample identifies the individual as one who may benefit from a treatment comprising an immune checkpoint inhibitor.

2. A method of treating or delaying progression of cancer, comprising:
detecting or acquiring knowledge of a CD274 nucleic acid molecule, or a PD-L1 polypeptide encoded by the CD274 nucleic acid molecule, in a sample from an individual having a cancer, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
(i) the CD274 fusion nucleic acid molecule comprises a fusion between a CD274 gene, or a portion thereof, and a fusion partner gene as listed in Table 2, or a portion thereof, or
(ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3; and
administering to the individual an effective amount of a treatment that comprises an immune checkpoint inhibitor responsive to detecting or acquiring knowledge of the CD274 nucleic acid molecule, or the PD-L1 polypeptide encoded by the CD274 nucleic acid molecule, in the sample.

3. A method of detecting a CD274 nucleic acid molecule, the method comprising:
providing a plurality of nucleic acid molecules obtained from a sample from an individual having a cancer, wherein the plurality of nucleic acid molecules comprises nucleic acid molecules corresponding to a CD274 nucleic acid molecule, wherein:
(a) the CD274 nucleic acid molecule comprises or results from a rearrangement comprising a Breakpoint 1 and/or Breakpoint 2 within the chromosomal coordinates as listed in Table 1; or
(b) the CD274 nucleic acid molecule is a CD274 fusion nucleic acid molecule, wherein:
(i) the CD274 fusion nucleic acid molecule comprises a fusion between a CD274 gene, or a portion thereof, and a fusion partner gene as listed in Table 2, or a portion thereof, or
(ii) the CD274 fusion nucleic acid molecule comprises a fusion between a CD274 gene, or a portion thereof, and a fusion partner gene as listed in Table 3, or a portion thereof, wherein the cancer is the corresponding cancer as listed in Table 3;
ligating one or more adapters onto one or more nucleic acid molecules from the plurality of nucleic acid molecules;
amplifying one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequence reads correspond to the CD274 nucleic acid molecule;
analyzing the plurality of sequence reads; and
based on the analysis, detecting the presence or absence of the CD274 nucleic acid molecule in the sample.

4. The method of claim 3, wherein:
(a) the method further comprises receiving, at one or more processors, sequence read data for the plurality of sequence reads;
(b) the analyzing the plurality of sequence reads comprises identifying, using one or more processors, the presence or absence of sequence reads corresponding to the CD274 nucleic acid molecule;
(c) the amplified nucleic acid molecules are captured by hybridization with one or more bait molecules;
(d) the plurality of nucleic acid molecules comprises a mixture of cancer nucleic acid molecules and non-cancer nucleic acid molecules;
(e) the one or more adapters comprise amplification primers, flow cell adapter sequences, substrate adapter sequences, or sample index sequences;
(f) the amplifying comprises performing a polymerase chain reaction (PCR) amplification technique, a non-PCR amplification technique, or an isothermal amplification technique;
(g) the sequencing comprises use of a massively parallel sequencing (MPS) technique, whole genome sequencing (WGS), whole exome sequencing, targeted sequencing, direct sequencing, a Sanger sequencing technique, or next generation sequencing (NGS);
(h) the sequencer comprises a next generation sequencer;
(i) the method further comprises generating a genomic profile for the individual, based, at least in part, on detecting the presence or absence of the CD274 nucleic acid molecule in the sample; and/or
(j) the method further comprises generating a report indicating the presence or absence of the CD274 nucleic acid molecule in the sample.

5. The method of claim 4, wherein:
(a) the genomic profile for the individual further comprises:
(i) results from a comprehensive genomic profiling (CGP) test, a gene expression profiling test, a cancer hotspot panel test, a DNA methylation test, a DNA fragmentation test, an RNA fragmentation test, a nucleic acid sequencing-based test, or any combination thereof; and/or
(ii) the level of tumor mutational burden (TMB), clonal TMB, indel TMB, or nonsense-mediated decay (NMD)-escape TMB; the presence or absence of a tobacco signature, an ultraviolet (UV) signature, an apolipoprotein B mRNA editing enzyme, catalytic polypeptide-line (APOBEC) signature, or a T cell inflamed gene expression profiling (GEP) signature; information on the sex of the individual; gene expression levels of CD274 or PD-L1, CD8A, and/or CXCL9; or any combination thereof; and/or
(b) the method further comprises selecting a treatment, administering a treatment, or applying a treatment to the individual based on the generated genomic profile, wherein the treatment comprises an immune checkpoint inhibitor.

6. The method of claim 1, wherein:
the CD274 nucleic acid molecule comprises or results from a rearrangement comprising a Breakpoint 1 and/or Breakpoint 2 within the chromosomal coordinates as listed in Table 1, or
the CD274 fusion nucleic acid molecule comprises a fusion between a CD274 gene, or a portion thereof, and a fusion partner gene as listed in Table 2, or a portion thereof, and
wherein the cancer is:
(a) a carcinoma, a sarcoma, a lymphoma, a leukemia, a myeloma, a germ cell cancer, or a blastoma;
(b) a solid tumor or a hematologic malignancy;
(c) a B cell cancer, melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor; or
(d) an adrenal gland cortical carcinoma, bladder carcinoma not otherwise specified (NOS), bladder urothelial (transitional cell) carcinoma, breast invasive ductal carcinoma (IDC), breast carcinoma not otherwise specified (NOS), cervix adenocarcinoma, cervix squamous cell carcinoma (SCC), colon adenocarcinoma, esophagus adenocarcinoma, esophagus squamous cell carcinoma (SCC), eye lacrimal duct carcinoma, head and neck squamous cell carcinoma (HNSCC), kidney renal cell carcinoma, liver hepatocellular carcinoma (HCC), lung adenocarcinoma, lung non-small cell lung carcinoma (NSCLC), lung non-small cell lung carcinoma (NSCLC) (NOS), lung small cell undifferentiated carcinoma, lung squamous cell carcinoma (SCC), lung non-squamous cell lung adenocarcinoma, ovary clear cell carcinoma, ovary epithelial carcinoma, ovary serous carcinoma, prostate acinar adenocarcinoma, skin melanoma, unknown primary adenocarcinoma, gastric cancer or carcinoma, unknown primary carcinoma (CUP), unknown primary carcinoma (CUP) (NOS), unknown primary melanoma, stomach adenocarcinoma, or vagina squamous cell carcinoma (SCC).

7. The method of claim 1, wherein:
(a) the CD274 nucleic acid molecule comprises or results from a rearrangement comprising a Breakpoint 1 and/or Breakpoint 2 within the chromosomal coordinates as listed in Table 1, and wherein the cancer is the corresponding cancer as listed in Table 6; or
(b) the CD274 fusion nucleic acid molecule comprises a fusion between a CD274 gene, or a portion thereof, and a fusion partner gene as listed in Table 2, or a portion thereof, and wherein the cancer is the corresponding cancer as listed in Table 7.

8. The method of claim 1, wherein:
(a) the CD274 fusion nucleic acid molecule comprises a fusion between a CD274 gene, or a portion thereof, and a fusion partner gene as listed in Table 2, or a portion thereof, and wherein the CD274 fusion nucleic acid molecule comprises or results from a corresponding CD274 Breakpoint and/or Fusion Partner Gene Breakpoint within the chromosomal coordinates as listed in Table 4;
(b) the CD274 fusion nucleic acid molecule comprises a fusion between a CD274 gene, or a portion thereof, and a fusion partner gene as listed in Table 2, or a portion thereof, wherein the CD274 fusion nucleic acid molecule comprises or results from a corresponding CD274 Breakpoint and/or Fusion Partner Gene Breakpoint within the chromosomal coordinates as listed in Table 8, and wherein the cancer is the corresponding cancer as listed in Table 8; or
(c) the CD274 fusion nucleic acid molecule comprises a fusion between a CD274 gene, or a portion thereof, and a fusion partner gene as listed in Table 3, or a portion thereof, and the cancer is the corresponding cancer as listed in Table 3, and wherein the CD274 fusion nucleic acid molecule comprises or results from a corresponding CD274 Breakpoint and/or Fusion Partner Gene Breakpoint within the chromosomal coordinates as listed in Table 5.

9. The method of claim 1, wherein the cancer is metastatic.

10. The method of claim 1, wherein the immune checkpoint inhibitor:
(a) comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, or any combination thereof;
(b) is a PD-1-, or a PD-L1-targeted agent;
(c) is a PD-1 inhibitor, a PD-L1 inhibitor, or a CTLA-4 inhibitor; and/or
(d) is a monotherapy.

11. The method of claim 10, wherein:
(a) the PD-1 inhibitor comprises one or more of nivolumab, pembrolizumab, cemiplimab, or dostarlimab;
(b) the PD-L1-inhibitor comprises one or more of atezolizumab, avelumab, or durvalumab;
(c) the CTLA-4 inhibitor comprises ipilimumab; or
(d) the nucleic acid comprises a double-stranded RNA (dsRNA), a small interfering RNA (siRNA), or a small hairpin RNA (shRNA).

12. The method of claim 1, wherein the treatment comprises:
(a) an additional anti-cancer therapy; and/or
(b) an immune checkpoint inhibitor in combination with one or more chemotherapeutic agents.

13. The method of claim 12, wherein:
(a) the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof;
(b) the additional anti-cancer therapy comprises a cellular therapy, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy;
(c) the additional anti-cancer therapy comprises a nucleic acid, wherein the nucleic acid comprises a double-stranded RNA (dsRNA), a small interfering RNA (siRNA), or a small hairpin RNA (shRNA); and/or
(d) the one or more chemotherapeutic agents comprise a platinum-based chemotherapeutic agent, bevacizumab-Awwb, bevacizumab, carboplatin, paclitaxel, paclitaxel protein-bound, or pemetrexed.

14. The method of claim 1, wherein the encoded PD-L1 polypeptide is oncogenic, and/or promotes cancer cell survival, angiogenesis, cancer cell proliferation, and any combination thereof.

15. The method of claim 1, further comprising:
(a) acquiring knowledge of or detecting in a sample from the individual the presence or absence of a CD274 gene amplification;
(b) acquiring knowledge of or detecting in a sample from the individual the presence or absence of a base substitution, a short insertion/deletion (indel), a copy number alteration, or a genomic rearrangement in one or more genes;
(c) acquiring knowledge of or detecting in a sample from the individual the presence or absence of a genomic Epstein-Barr virus (EBV);
(d) acquiring knowledge of or detecting a microsatellite instability status of the cancer in a sample from the individual;
(e) acquiring knowledge of or determining tumor mutational burden (TMB) in a sample from the individual;
(f) acquiring knowledge of or determining the level of PD-L1 expression in a sample from the individual; and/or
(g) acquiring knowledge of or determining the clonality of the CD274 nucleic acid molecule in the cancer.

16. The method of claim 15, wherein:
(a) the one or more genes comprise one or more of: TP53, PIK3CA, CDKN2A, KRAS, CDKN2B, CD274, MYC, JAK2, RB1, PDCD1LG2, APC, ARID1A, PTEN, BRAF, CREBBP, PBRM1, KMT2D, CCND1, KDM6A, BCL2L1, ERBB2, FBXW7, NF1, BCORL1, BRCA2, FGF19, FGFR1, MAP2K1, PRKC1, ATM, CDK12, CTNNB1, DNMT3A, FGF3, FGF4, GNAS, LYN, MET, NOTCH1, RNF43, STK11, TET2, VHL, ZNF217, ASXL1, BRCA1, EGFR, KDM5C, KIT, NFE2L2, NOTCH2, NOTCH3, PIK3R1, SOX9, TERC, ZNF703, MTAP, BRIP1, CDC73, ACVR1B, ATRX, MLH1, BRD4, SMAD4, PALB2, RAD21, GATA6, CTCF, MLH1, a mismatch repair gene, or any combination thereof;
(b) the cancer comprises a base substitution, a small insertion/deletion (indel), a copy number alteration, or a genomic rearrangement in the one or more genes;
(c) the EBV is HHV-4;
(d) the cancer or the individual comprises a genomic EBV or is positive for EBV;
(e) the cancer is microsatellite stable;
(f) the cancer has a TMB of less than 6 mutations per megabase (mut/Mb), between 6 and 20 mut/Mb, greater than 20 mut/Mb, a high TMB, a TMB of about 7.0 mut/Mb, or a TMB of at least about 10 mut/Mb;
(g) the cancer is PD-L1 positive, PD-L1-high positive, or PD-L1 negative;
(h) the cancer comprises a CD274 gene amplification, or the cancer does not comprise a CD274 gene amplification; and/or
(i) the CD274 nucleic acid molecule results from a clonal or a sub-clonal rearrangement of a CD274 gene in the cancer, or the CD274 nucleic acid molecule is clonal or sub-clonal in the cancer.

17. The method of claim 15, wherein the cancer or the individual comprises a genomic EBV or is positive for EBV, and wherein:
the cancer is a gastric cancer, a gastric adenocarcinoma, or a stomach adenocarcinoma; and/or
the treatment comprises pembrolizumab.

18. The method of claim 15, wherein clonality of the CD274 nucleic acid molecule is assessed by performing DNA sequencing on a sample obtained from the individual.

19. The method of claim 18, wherein:
(a) clonality of the CD274 nucleic acid molecule is assessed based on sequencing coverage of a CD274 gene and the number of sequence read pairs spanning a breakpoint of the CD274 nucleic acid molecule; or
(b) clonality of the CD274 nucleic acid molecule is assessed based on the number of sequence read pairs spanning a breakpoint of the CD274 nucleic acid molecule.

20. The method of claim 19, wherein:
(a) clonality of the CD274 nucleic acid molecule is assessed based on sequencing coverage of a CD274 gene and a threshold for the number of sequence read pairs spanning a breakpoint of the CD274 nucleic acid molecule, wherein the threshold is selected using a receiver operator characteristic (ROC) curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis;
(b) clonality of the CD274 nucleic acid molecule is assessed based on the number of sequence read pairs spanning a breakpoint of the CD274 nucleic acid molecule, wherein the clonality is assessed based on a threshold for the number of sequence read pairs spanning a breakpoint of the CD274 nucleic acid molecule, wherein the threshold is selected using a ROC curve analysis for predicting that a sample from a tumor is PD-L1 high positive based on the number of sequence read pairs spanning a breakpoint of a CD274 nucleic acid molecule present in the sample, and wherein the threshold is selected such that it maximizes the sum of sensitivity and specificity in the ROC curve analysis; and/or
(c) the sample is a bulk tumor sample derived from a single anatomic location.

21. The method of claim 20, wherein:
(a) a sample from a tumor is PD-L1 high positive if it comprises a tumor proportion score (TPS) of at least about 50%; and/or
(b) the threshold for the number of sequence read pairs spanning a breakpoint of the CD274 nucleic acid molecule is at least about 20 read pairs, at least about 21 read pairs, at least about 22 read pairs, at least about 23 read pairs, at least about 24 read pairs, at least about 25 read pairs, at least about 26 read pairs, at least about 27 read pairs, at least about 28 read pairs, at least about 29 read pairs, at least about 30 read pairs, at least about 31 read pairs, at least about 32 read pairs, at least about 33 read pairs, at least about 34 read pairs, at least about 35 read pairs, at least about 36 read pairs, at least about 37 read pairs, at least about 38 read pairs, at least about 39 read pairs, at least about 40 read pairs, at least about 41 read pairs, at least about 42 read pairs, at least about 43 read pairs, at least about 44 read pairs, at least about 45 read pairs, at least about 46 read pairs, at least about 47 read pairs, at least about 48 read pairs, at least about 49 read pairs, or at least about 50 read pairs spanning a breakpoint of the CD274 nucleic acid molecule.

22. The method of claim 16, wherein:
(a) responsive to acquiring knowledge of or determining that the CD274 nucleic acid molecule is clonal in the cancer or results from a clonal rearrangement of a CD274 gene in the cancer:
(i) the individual is identified as likely to respond to a treatment comprising an immune checkpoint inhibitor;
(ii) the individual is predicted to have longer survival when treated with a treatment comprising an immune checkpoint inhibitor, as compared to survival of: an individual whose cancer does not comprise a clonal CD274 nucleic acid molecule, or a CD274 nucleic acid molecule resulting from a clonal rearrangement of a CD274 gene; or an individual whose cancer comprises a sub-clonal CD274 nucleic acid molecule, or a CD274 nucleic acid molecule resulting from a sub-clonal rearrangement of a CD274 gene; and/or
(iii) the individual is predicted to have an improved response to treatment with an immune checkpoint inhibitor, as compared to: an individual whose cancer does not comprise a clonal CD274 nucleic acid molecule, or a CD274 nucleic acid molecule resulting from a clonal rearrangement of a CD274 gene; or an individual whose cancer comprises a sub-clonal CD274 nucleic acid molecule, or a CD274 nucleic acid molecule resulting from a sub-clonal rearrangement of a CD274 gene; and/or
(b) acquiring knowledge of or determining that the CD274 nucleic acid molecule is clonal in the cancer or results from a clonal rearrangement of a CD274 gene in the cancer identifies the cancer as:
(i) likely to be PD-L1 positive or PD-L1 high positive; and/or
(ii) likely to have a TPS of at least about 50%, assessed based on an immunohistochemistry assay.

23. The method of claim 1, wherein:
(a) the method further comprises obtaining the sample from the individual, and/or the sample is obtained from the cancer; and/or
(b) the sample:
(i) comprises a tissue biopsy sample, a liquid biopsy sample, or a normal control,
(ii) is from a tumor biopsy, tumor specimen, or circulating tumor cell,
(iii) is a liquid biopsy sample and comprises blood, plasma, cerebrospinal fluid, sputum, stool, urine, or saliva,
(iv) comprises cells and/or nucleic acids from the cancer,
(v) comprises mRNA, DNA, circulating tumor DNA (ctDNA), cell-free DNA, or cell-free RNA from the cancer,
(vi) is a liquid biopsy sample and comprises circulating tumor cells (CTCs), or
(vii) is a liquid biopsy sample and comprises cell-free DNA (cfDNA), circulating tumor DNA (ctDNA), or any combination thereof.

24. The method of claim 1, wherein:
(a) the method comprises acquiring knowledge of or detecting the CD274 nucleic acid molecule, or the PD-L1 polypeptide encoded by the CD274 nucleic acid molecule, in a tissue biopsy sample, in a liquid biopsy sample, or in both a tissue biopsy sample and a liquid biopsy sample, from the individual;
(b) acquiring knowledge of the CD274 nucleic acid molecule, or the PD-L1 polypeptide encoded by the CD274 nucleic acid molecule, comprises detecting the CD274 nucleic acid molecule, or the PD-L1 polypeptide encoded by the CD274 nucleic acid molecule, in the sample;
(c) detecting the CD274 nucleic acid molecule comprises detecting a fragment of the CD274 nucleic acid molecule comprising a breakpoint; and/or
(d) detecting the PD-L1 polypeptide encoded by the CD274 nucleic acid molecule comprises detecting a portion of the polypeptide that is encoded by a fragment of the CD274 nucleic acid molecule that comprises a breakpoint.

25. The method of claim 1, wherein:
(a) the CD274 nucleic acid molecule is detected in the sample by:
(i) one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing;
(ii) sequencing using a massively parallel sequencing (MPS) technique, whole genome sequencing (WGS), whole exome sequencing, targeted sequencing, direct sequencing, a Sanger sequencing technique, next-generation sequencing (NGS), or RNA-sequencing (RNA-seq); or
(iii) a polymerase chain reaction (PCR) amplification technique, a non-PCR amplification technique, an isothermal amplification technique, a reverse transcription PCR (RT-PCR), a quantitative real-time PCR (qPCR), or a reverse transcription quantitative real-time PCR (RT-qPCR) assay;
(b) the PD-L1 polypeptide encoded by the CD274 nucleic acid molecule is detected in the sample by one or more of: immunoblotting, enzyme linked immunosorbent assay (ELISA), immunohistochemistry, or mass spectrometry; and/or
(c) the CD274 nucleic acid molecule, or the PD-L1 polypeptide encoded by the CD274 nucleic acid molecule, is detected using a digital pathology method.

26. The method of claim 1, wherein the method further comprises selectively enriching for one or more nucleic acid molecules in the sample comprising nucleotide sequences corresponding to the CD274 nucleic acid molecule, wherein the selectively enriching produces an enriched sample.

27. The method of claim 26, wherein:
(a) the selectively enriching comprises:
(i) combining one or more bait molecules with the sample, thereby hybridizing the one or more bait molecules to one or more nucleic acid molecules in the sample comprising nucleotide sequences corresponding to the CD274 nucleic acid molecule and producing nucleic acid hybrids, and isolating the nucleic acid hybrids to produce the enriched sample, or
(ii) amplifying the one or more nucleic acid molecules comprising nucleotide sequences corresponding to the CD274 nucleic acid molecule using a polymerase chain reaction (PCR) to produce an enriched sample; and/or
(b) the method further comprises sequencing the enriched sample.

28. The method of claim 27, wherein the one or more bait molecules comprise a capture nucleic acid molecule configured to hybridize to a nucleotide sequence corresponding to the CD274 nucleic acid molecule; and/or the one or more bait molecules are conjugated to an affinity reagent or to a detection reagent.

29. The method of claim 28, wherein:
(a) the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides;
(b) the affinity reagent is an antibody, an antibody fragment, or biotin, or wherein the detection reagent is a fluorescent marker; and/or
(c) the capture nucleic acid molecule comprises a DNA, RNA, or mixed DNA/RNA molecule.

30. The method of claim 1, wherein the individual is a human.
